(19) United States
(12) Patent Application Publication
(10) Pub. No.: US 2011/0195929 A1
(43) Pub. Date: Aug. 11, 2011

(54) COMPOUNDS FOR THE TREATMENT OF FLAVIVIRAL INFECTIONS

(75) Inventors: Olivier De Moor, Oxford (GB); Graeme Horne, Wantage (GB); Penny Jane Middleton, Didcot (GB); Frank Schroer, Wantage (GB); Stephen Paul Wren, Aylesbury (GB); Maria Ines Passos Eleuterio, Didcot (GB); Renate Van Well, Didcot (GB); Colin Richard Dorgan, Abingdon (GB); Francis Xavier Wilson, Welwyn Garden City (GB); Robert Nash, Ystrad Meurig (GB); Richard Storer, Folkestone (GB); Graham Michael Wynne, Bicester (GB); Alan Geoffrey Roach, Ashwell (GB); Akane Kawamura, Oxford (GB); Jonathon Mark Tinsley, Didcot (GB)

(73) Assignee: SUMMIT CORPORATION PLC, Oxfordshire (GB)

(21) Appl. No.: 13/057,557

(22) PCT Filed: Aug. 4, 2009
(86) PCT No.: PCT/GB2009/001917

§ 371 Date: Apr. 25, 2011

(30) Foreign Application Priority Data
Aug. 5, 2008 (GB) 0814216.8
Sep. 24, 2008 (GB) 0817437.7
Oct. 24, 2008 (GB) 0819518.2
Apr. 9, 2009 (GB) 0906210.0
May 20, 2009 (GB) 0908672.9

(51) Int. Cl. A61K 31/695; C07D 487/04; A61K 31/407; C07D 207/12; A61K 31/40; C07D 211/46; A61K 31/445; C07D 207/16; C07D 451/06; A61K 31/46; C07D 471/04; A61K 31/437; C07D 405/04; A61K 31/453; C07D 265/32; A61K 31/5375; C07D 491/113; A61K 31/438; C07D 223/08; A61K 31/55; C07F 7/18; C07D 491/056; A61K 31/397; A61P 31/14

(52) U.S. Cl. 514/63; 548/453; 514/413; 548/556; 514/425; 546/242; 514/315; 548/531; 514/423; 546/131; 514/304; 546/183; 514/299; 546/207; 514/326; 544/173; 514/238.8; 546/18; 514/278; 540/604; 514/212.01; 546/14; 540/593; 514/215; 514/210.01

(57) ABSTRACT

Described are various compounds and methods for the treatment of flaviviral infections. In particular, alkaloids and imino sugars in arabinose and/or lyxose stereochemical configuration with antiflaviviral activity are described.

### FIELD OF THE INVENTION

This invention relates to certain compounds, in particular iminosugars, for the treatment of infections with, or diseases caused by, a flavivirus. In particular, the invention relates to certain compounds for use in the treatment of hepatitis C virus (HCV) infection and/or diseases caused thereby.

### BACKGROUND OF THE INVENTION

### Flaviviruses

The flavivirus group (family Flaviviridae) comprises the genera Flavivirus, Pestivirus and Hepacivirus and includes the causative agents of numerous human diseases and a variety of animal diseases which cause significant losses to the livestock industry.

The family Flaviviridae (members of which are referred to herein as flaviviruses) include the genera Flavivirus (e.g. yellow fever virus, dengue viruses, Japanese encephalitis virus, Murray Valley encephalitis virus, West Nile fever virus, Rocio virus, St. Louis encephalitis virus, Louping ill virus, Powassan virus, Omsk hemorrhagic fever virus, Kyasanur forest disease virus and tick-borne encephalitis virus), Pestivirus (e.g. bovine viral diarrhoea virus, rubella virus, classical swine fever virus, hog cholera virus and border disease virus), Hepacivirus (hepatitis C virus) and currently unclassified members of the Flaviviridae (e.g. GB virus types A, B and C).

The full list of members of the Flaviviridae are defined in detail by the International Committee on Taxonomy of Viruses (the currently accepted taxanomic definition is described in: Virus Taxonomy: The Classification and Nomenclature of Viruses. The Seventh Report of the International Committee on Taxonomy of Viruses (M. H. V. van Regenmortel, C. M. Fauquet, D. H. L. Bishop, E. B. Carstens, M. K. Estes, S. M. Lemon, J. Maniloff, M. A. Mayo, D. J. McGeoch, C. R. Pringle, R. B. Wickner (2000). Virus Taxonomy, VIIth report of the ICTV. Academic Press, SanDiego), the content of which relating to the constitution of the family Flaviviridae is hereby incorporated by reference.

One particularly important flavivirus is the hepatitis C virus (HCV). HCV is an enveloped plus-strand RNA virus belonging to the Flaviviridae family, but classified as a distinct genus Hepacivirus. It was first identified in 1989 and it has since become clear that this virus is responsible for most cases of post-transfusion non-A, non-B hepatitis. Indeed, HCV is now recognised as one of the commonest infections causing chronic liver disease and the World Health Organisation estimates that 170 million people are chronically infected. HCV infection results in a chronic infection in 85% of infected patients and approximately 20-30% of these will progress to cirrhosis and end stage liver disease, frequently complicated by hepatocellular carcinoma.

The hepatitis C virus species is classified into six genotypes (1 to 6). Each genotype is further subclassified into distinct subtypes (represented by letters). These subtypes are then further broken down into quasispecies based on genetic characteristics. The preponderance and distribution of HCV genotypes varies globally. For example, in North America, genotype 1a predominates followed by 1b, 2a, 2b, and 3a. In Europe, genotype 1b is predominant followed by 2a, 2b, 2c, and 3a. Genotypes 4 and 5 are found almost exclusively in Africa.

The HCV genome consists of a single long open reading frame which encodes a 3000 amino acid residue polyprotein. This polyprotein is processed co- and post translationally into at least 10 different products including two N-linked glycosylated proteins E1 and E2. The genome carries at the 5′ and 3′ ends non-translated regions (NTRs) that form stable secondary and tertiary structures. The 5′ NTR carries an internal ribosome entry site (IRES) permitting the direct binding of ribosomes in close proximity to the start codon of the ORF. Thus translation of HCV RNA is mediated by the IRES, rather than the CAP-dependent mechanism typically used by cellular mRNA.

Within the polyprotein, cleavage products are ordered as follows: core (C), envelope protein 1 (E1), E2, p7, non-structural protein 2 (NS2), NS3, NS4A, NS4B, NS5A and NS5B. The core protein is a highly basic RNA binding protein forming the major constituent of the nucleocapsid. The envelope proteins E1 and E2 are highly glycosylated type 1 membrane proteins anchored through the carboxy-terminal region. They are embedded into the lipid envelope of the virus particle and associate to form stable heterodimers. The cleavage product p7 is a small hydrophobic peptide of unknown function. The non-structural proteins are involved in viral replication and possess protease (NS2/NS3), helicase (NS3) and RNA polymerase activities (NS5B). Binding to the host cell probably requires the interaction of E2 or the E1/E2 complex with a receptor that is present on the cell surface.

The study of HCV has been hampered by the inability to propagate the virus efficiently in cell culture. However, in the absence of a suitable cell culture system able to support replication of human HCV, BVDV is an accepted cell culture model. HCV and BVDV share a significant degree of local protein homology, a common replication strategy and probably the same subcellular location for viral envelopment. Such studies have suggested a model wherein initial virion morphogenesis occurs by budding into intracellular vesicles from the ER. It is thought that mature E1-E2 heterodimers do not leave the ER, and ER retention signals have been identified in the C-terminal regions of both E1 and E2. In this case the virus would be exported via the constitutive secretory pathway. In agreement with this assumption, complex N-linked glycans were found on the surface of partially purified virus particles suggesting that the virus transits through the Golgi.

Until recently, interferon-α (IFN-α) was the only therapy with proven benefit for the treatment of HCV infection. Using IFN-α up to 50% of patients show a response to treatment, but this is not sustainable in the majority of patients and there are considerable associated side effects. More recently, a combination of pegylated IFN-α (Pegasys™ and PEG-Intron™) and the antiviral drug ribavirin have been used. However, this treatment is associated with severe side effects, including anaemia, cardiovascular events and psychiatric problems.

There is therefore a need for improved anti-flaviviral drugs in general, and anti-HCV drugs in particular.

### Glycoproteins and Viral Development

Glycoproteins are classified into two major classes according to the linkage between sugar and amino acid of the protein. The most common and extensively studied is N-glycosidic linkage between an asparagine of the protein and an N-acetyl-D-glucosamine residue of the oligosaccharide. N-linked oligosaccharides, following attachment to a polypeptide backbone, are processed by a series of specific enzymes in the endoplasmic reticulum (ER) and this processing pathway has been well characterised.

In the ER, α-glucosidase I is responsible for the removal of the terminal α-1,2 glucose residue from the precursor oligosaccharide and α-glucosidase II removes the two remaining α-1,3 linked glucose residues, prior to removal of mannose residues by mannosidases and further processing reactions involving various transferases. These oligosaccharide “trimming” reactions enable glycoproteins to fold correctly and to interact with chaperone proteins such as calnexin and calreticulin for transport through the Golgi apparatus.

Inhibitors of key enzymes in this biosynthetic pathway, particularly those blocking α-glucosidases and α-mannosidase, have been shown to prevent replication of several enveloped viruses. Such inhibitors may act by interfering with the folding of the viral envelope glycoprotein, so preventing the initial virus-host cell interaction or subsequent fusion. They may also prevent viral duplication by preventing the construction of the proper glycoprotein required for the completion of the viral membrane.

For example, it has been reported that the nonspecific glycosylation inhibitors 2-deoxy-D-glucose and β-hydroxy-norvaline inhibit expression of HIV glycoproteins and block the formation of syncytia (Blough et al., Biochem. Biophys. Res. Comm., 141(1), 33-38 (1986)). Viral multiplication of HIV-infected cells treated with these agents is stopped, presumably because of the unavailability of glycoprotein required for viral membrane formation.

In another report, the glycosylation inhibitor 2-deoxy-2-fluoro-D-mannose was found to exhibit antiviral activity against influenza infected cells by preventing the glycosylation of viral membrane protein (McDowell et al., Biochemistry, 24(27), 8145-52 (1985)). This report also studied the antiviral activity of 2-deoxyglucose and 2-deoxy-2-fluoroglucose and found that each inhibits viral protein glycosylation by a different mechanism.

Lu et al. (1995) present evidence that N-linked glycosylation is necessary for hepatitis B virus secretion (Virology 213: 660-665) while Block et al. (1994) show that secretion of human hepatitis B virus is inhibited by the iminosugar N-butyldeoxynojirimycin (Proc. Nat. Acad. Sci. 91: 2235-2239). See also WO9929321.

Taylor et al. (1988) demonstrate the loss of cytomegalovirus infectivity after treatment with castanospermine or other plant alkaloids and relate this to abberant glycoprotein synthesis (Antiviral Res. 10: 11-26). See also U.S. Pat. No. 5,004,746.

Taylor et al. (1994) show that inhibition of α-glucosidase I of the glycoprotein processing enzymes by 6-O-butanoyl castanospermine has consequences in human immunodeficiency virus-infected T-cells (Antimicrob. Agents Chemother. 38: 1780-1787) while Sunkara et al. (1989) describe anti-HIV activity of castanospermine analogues (Lancet I I 1206). See also U.S. Pat. No. 5,004,746.

U.S. Pat. No. 5,385,911 discloses anti-herpes activity in certain castanospermine esters.

However, many other known glycosylation inhibitors have been found to have no antiviral activity. Thus the antiviral activity against enveloped viruses, in general, and the anti-flaviviral activity, specifically, of glycosylation inhibitors is quite unpredictable.

### Iminosugar Glycosidase Inhibitors

It has long been recognized that many iminosugars are pharmacologically active, and humans have been using iminosugars (typically in the form of plant extracts) as poisons, narcotics, stimulants and medicines for thousands of years. The therapeutic applications of polyhydroxylated alkaloids have been comprehensively reviewed in Watson et al. (2001) Phytochemistry 56: 265-295: applications include cancer therapy, immune stimulation, the treatment of diabetes, the treatment of infections (especially viral infections), therapy of glycosphingolipid lysosomal storage diseases and the treatment of autoimmune disorders (such as arthritis and sclerosis).

It is also known that certain iminosugars, such as deoxynojirimycin (DNJ), are ER α-glucosidase inhibitors and both potently inhibit the early stages of glycoprotein processing. However, their effects differ substantially depending on the system to which they are applied and they may exhibit quite different specificities, castanospermine being relatively specific for α-glucosidase I.

Branza-Nichita et al., (2001) J. Virol 75(8): 3527-3536 showed that the iminosugar N-butyldeoxynojirimycin has an antiviral effect against the pestivirus BVDV. However, the authors make clear that while treatment with α-glucosidase inhibitors may affect the life cycles of this and other enveloped viruses, it is not possible to generalize to other viruses since the effects may depend crucially on the particular folding pathway used by the viral proteins.

Courageot et al. (2000) J. Virol. 74(1): 564-572 report that the α-glucosidase inhibitors castanospermine and DNJ reduce dengue virus production in an in vitro mouse neuroblastoma model.

WO 99/29321 discloses the use of various iminosugar α-glucosidase inhibitors in the treatment of inter alia HCV infections.

The use of iminosugars containing the glucose analogue DNJ as antiviral agents against different viruses has been suggested since the late 1980s. While the action of two of them, DNJ and NB-DNJ, has been extensively described in the literature, the discovery of the antiviral action of a longer-alkyl-chain derivative of DNJ, N,N-DNJ, was reported only relatively recently (see Zitzmann et al. (1999) Proc. Nat. Acad. Sci. 96: 11878-11882).

DNJ and its N-alkylated derivatives have been shown to inhibit α-glucosidase I and/or α-glucosidase II, so preventing the interaction of calnexin (CNX) and/or calreticulin (CRT) with folding glycoproteins. N-alkylation of DNJ has been shown to increase its inhibitory potency: N-nonyl-DNJ (N,N-DNJ), a 9-carbon alkyl derivative of DNJ, has been found to be at least 20 times more potent than the non-alkylated DNJ in inhibiting hepatitis B virus (HBV) and bovine viral diarrhoea virus (BVDV) in cell based assays. Other N-substituted DNJ derivatives (including N-methoxy-nonyl-DNJ and N-butyl-cyclohexyl DNJ) have also been shown to have improved potency (the N-methoxy analogue being the most potent, exhibiting micromolar antiviral activity).

However, ER α-glucosidase inhibition does not correlate precisely with antiviral activity: the less active NB-DNJ is a more effective ER α-glucosidase inhibitor than N,N-DNJ. Moreover, the short-chain N-butyl-DGJ (NB-DGJ) exhibits no antiviral activity, whereas its long-chain derivative N,N-DGJ is a potent antiviral. Thus, an additional mechanism of action appears to be associated with the length of the N-alkyl side chain, and it has recently been suggested that this may be based on the inhibition of an ion channel formed by the HCV p7 protein (Pavlovic et al., (2003) Proc. Nat. Acad. Sci. 100(10): 6104-6108; see also WO2004/047719). However, further studies (Mehta et al., (2004) Antimicrobial Agents and Chemotherapy 48(6): 2085-2090) have shown that at least one alkovir (N-9-oxadecyl-6-methyl-DGJ) inhibits HCV under conditions where p7 is not present, suggesting that p7 inhibition may not be the sole mechanism of alkoviral activity.

Iminosugars mediating an antiviral effect via α-glucosidase inhibition (for example, DNJ and NB-DNJ) have been dubbed glucovirs, whereas those (such as N,N-DGJ and N-7-oxanonyl-6-deoxy-DGJ) mediating an antiviral effect independently of α-glucosidase inhibition (for example by interfering with viral p7 protein as described infra) have been dubbed alkovirs (see Block and Jordan (2001) Antivir. Chem. Chemother. 12(6): 317-325).

The use of current iminosugar α-glucosidase inhibitors in general (and DNJ and other piperidine derivatives in particular) as antiviral drugs is limited by toxicity arising from coinhibition of gastrointestinal α-glycosidases at the concentrations required for therapeutic effects. There is therefore much interest in alkovirs, since toxicity arising from co-inhibition of gastrointestinal α-glycosidases may be avoided by members of this class. Indeed, the N-substituted iminosugar N-7-oxanonyl-6-deoxy-DGJ (N-7-oxanonyl-6-methyldeoxygalactonojirimycin; N-7-oxanonyl-6-MeDGJ) was entered into phase I clinical studies (as UT 231-B) in 2002.

The present inventors have now surprisingly discovered that certain iminosugars exhibit antiviral activity against members of the Flaviviridae (including HCV). Moreover, they have found that the therapeutic index is unexpectedly superior to that exhibited by other α-glucosidase inhibitors of the iminosugar class.

### SUMMARY OF THE INVENTION

According to a first aspect of the present invention there is provided a compound of Formula (1)

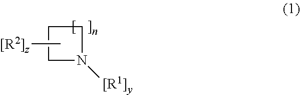

in which
n represents an integer from 1 to 7, provided that where n>1 the ring may also contain at least one unsaturated C—C bond
z represents an integer from 1 to (n+2)
y represents 1 or 2
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂; N3; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
   or a pharmaceutically acceptable salt or derivative thereof, for the treatment of infection with, or a disease caused by, a flavivirus.

In a second aspect, the invention provides a compound of Formula (2)

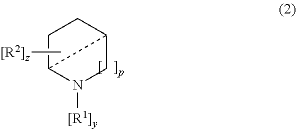

in which
p represents an integer from 1 to 2
z represents an integer from 1 to (p+7)
y represents 1 or 2
the broken line represents a bridge containing 2 or 3 carbon atoms between any two different ring carbon atoms, any or all of which bridge or bridgehead carbon atoms being optionally substituted with R²
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
   or pharmaceutically acceptable salt or derivative thereof, for the treatment of infection with, or a disease caused by, a flavivirus.

In a third aspect, the invention provides a compound of Formula (3)

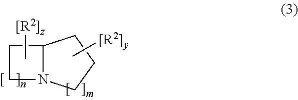

in which
n represents an integer from 1 to 7, for example 1 to 5, provided that where n>1 the ring may also contain at least one unsaturated C—C bond
m represents an integer from 1 to 3 and the ring may also contain at least one unsaturated C—C bond
z represents an integer from 0 to (n+2), provided that where z=0 then y≧1
y represents an integer from 0 to (m+2), provided that where y=0 then z≧1
the endocyclic nitrogen atom may be bonded to an oxygen or an oxygen containing group such that the compound is an N-oxide,
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N3, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
optionally wherein the compound has three, four or more rings
   or pharmaceutically acceptable salt or derivative thereof, for the treatment of infection with, or a disease caused by, a flavivirus.

In a further aspect, the invention provides an iminosugar as herein defined for the treatment of infection with, or a disease caused by, a flavivirus.

In a yet further aspect, the invention provides a compound selected from compounds 1 to 892 of Table 1 (or a pharmaceutically acceptable salt or derivative thereof) for the treatment of infection with, or a disease caused by, a flavivirus.

Other aspects and preferred embodiments of the invention are defined and described in the claims set out below.

The invention also contemplates adjunctive use of the compounds of the invention with various adjunctive agents. The adjunctive agent may comprise an antiviral compound, for example an anti-HCV drug. Particularly preferred are adjunctive therapeutics comprising interferon-α and/or ribavirin.

Thus, in another aspect, the invention provides a composition comprising a compound of the invention in combination with: (a) compounds which inhibit the binding to and/or infection of cells by HCV. These include antibodies (e.g. monoclonal antibodies) against, for example, HCV E1 and/or E2 proteins) and glucosaminoglycans (such as heparan sulphate and suramin); (b) compounds which inhibit the release of viral RNA from the viral capsid or the function of HCV gene products, including inhibitors of the IRES, protease (e.g. serine protease) inhibitors, helicase inhibitors and inhibitors of the viral polymerase/replicase; (c) compounds which perturb cellular functions involved in or influencing viral replication, including inhibitors of inosine monophosphate dehydrogenase (e.g. Ribavirin, mycophenolic acid and VX497) and inhibitors of glycoprotein processing such as DNJ and its derivatives; (d) compounds which act to alter immune function (e.g. thymosin alpha and interferons such as α interferons and β interferons) and (e) compounds which act to modulate the symptoms and effects of HCV infection (e.g. antioxidants such as the flavinoids).

In addition the invention provides a composition comprising a compound of the invention in combination with compounds used in the treatment of frequently found co-infections (such as hepatitis B virus and the human retroviruses such as human immunodeficiency viruses types 1 and 2 and human T-cell lymphotrophic viruses types 1 and 2). Examples of such compounds include nucleotide/nucleoside RT inhibitors (e.g. Lamivudine (3TC), zidovudine, stavudine, didanosine, adefovir dipivoxil and abacavir), non-nucleoside RT inhibitors (e.g. nevirapine) and protease inhibitors (e.g. saquinavir, indinavir and ritonavir).

Preferably, the interferon is interferon-α (IFN-α), though other interferons may also be used (for example an interferon produced by expression of a cloned human interferon gene).

In another aspect, the invention provides a pharmaceutical kit of parts comprising a compound of the invention in combination with: (a) compounds which inhibit the binding to and/or infection of cells by HCV; (b) compounds which inhibit the release of viral RNA from the viral capsid or the function of HCV gene products; (c) compounds which perturb cellular functions involved in or influencing viral replication; (d) compounds which act to alter immune function, and (e) compounds which act to modulate the symptoms and effects of HCV infection, as described above.

The kit may also further comprise instructions for use in the treatment of a flaviviral disease (for example in the flaviviral diseases described herein).

In the compositions of the invention the compound of the invention and the adjunctive therapeutic(s) may act in a complementary or synergistic fashion. Particularly preferred are compositions and methods comprising both the compound of the invention and interferon which act in a synergistic fashion in the treatment of HCV infection.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### DEFINITIONS AND GENERAL PREFERENCES

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The phrase “consisting essentially of” is used herein to require the specified integer(s) or steps as well as those which do not materially affect the character or function of the claimed invention.

As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “flavivirus” refers to any virus of the family Flaviviridae, including in particular any virus of the genera Flavivirus, Pestivirus and Hepacivirus and so including in particular the hepatitis C virus (HCV).

As used herein, the term “flaviviral disease” refers to any state or condition that involves (e.g. is caused, exacerbated, associated with or characterized by the presence of) a virus of the family Flaviviridae residing and/or replicating in the cells (or within the body) of said patient.

As used herein, the term “flaviviral infection” is used to define a condition in which a subject is infected with a virus of the family Flaviviridae (i.e. is infected with a flavivirus as hereinbefore defined). The infection may be symptomatic or asymptomatic. In the latter case, the subject may be identified as infected on the basis of various tests, including for example serological analyses (e.g. using HCV antibodies and/or antigens).

As used herein, the term “treatment” or “treating” refers to an intervention (e.g. the administration of an agent to a subject) which cures, ameliorates or lessens the symptoms of a disease or removes (or lessens the impact of) its cause(s) (for example, the causitive pathogen in the case of infectious diseases). In this case, the term is used synonymously with the term “therapy”. Thus, the treatment of flaviviral infection according to the invention may be characterized by the (direct or indirect) virostatic and/or virocidal action of the compounds of the invention.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

The term “intervention” is a term of art used herein to define any agency which effects a physiological change at any level. Thus, the intervention may comprise the induction or repression of any physiological process, event, biochemical pathway or cellular/biochemical event. The interventions of the invention typically effect (or contribute to) the treatment (i.e. therapy or prophylaxis as herein defined) of a disease and typically involve the administration of an agent to a subject.

In this context “subject” (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In preferred embodiments, the subject is a human.

As used herein, an effective amount or a therapeutically effective amount of a compound defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “adjunctive” as applied to the use of the compounds of the invention in therapy or prophylaxis defines uses in which the compound is administered together with one or more other drugs, interventions, regimens or treatments (such as surgery and/or irradiation). Such adjunctive therapies may comprise the concurrent, separate or sequential administration/application of the materials of the invention and the other treatment(s). Thus, in some embodiments, adjunctive use of the materials of the invention is reflected in the formulation of the pharmaceutical compositions of the invention. For example, adjunctive use may be reflected in a specific unit dosage, or in formulations in which the compound of the invention is present in admixture with the other drug(s) with which it is to be used adjunctively (or else physically associated with the other drug(s) within a single unit dose). In other embodiments, adjunctive use of the compounds or compositions of the invention may be reflected in the composition of the pharmaceutical kits of the invention, wherein the compound of the invention is co-packaged (e.g. as part of an array of unit doses) with the other drug(s) with which it is to be used adjunctively. In yet other embodiments, adjunctive use of the compounds of the invention may be reflected in the content of the information and/or instructions co-packaged with the compound relating to formulation and/or posology.

As used herein, the term “combination”, as applied to two or more compounds and/or agents (also referred to herein as the components), is intended to define material in which the two or more compounds/agents are associated. The terms “combined” and “combining” in this context are to be interpreted accordingly.

The association of the two or more compounds/agents in a combination may be physical or non-physical. Examples of physically associated combined compounds/agents include:
compositions (e.g. unitary formulations) comprising the two or more compounds/agents in admixture (for example within the same unit dose);
compositions comprising material in which the two or more compounds/agents are chemically/physicochemically linked (for example by crosslinking, molecular agglomeration or binding to a common vehicle moiety);
compositions comprising material in which the two or more compounds/agents are chemically/physicochemically co-packaged (for example, disposed on or within lipid vesicles, particles (e.g. micro- or nanoparticles) or emulsion droplets);
pharmaceutical kits, pharmaceutical packs or patient packs in which the two or more compounds/agents are co-packaged or co-presented (e.g. as part of an array of unit doses);

Examples of non-physically associated combined compounds/agents include:
material (e.g. a non-unitary formulation) comprising at least one of the two or more compounds/agents together with instructions for the extemporaneous association of the at least one compound/agent to form a physical association of the two or more compounds/agents;
material (e.g. a non-unitary formulation) comprising at least one of the two or more compounds/agents together with instructions for combination therapy with the two or more compounds/agents;
material comprising at least one of the two or more compounds/agents together with instructions for administration to a patient population in which the other(s) of the two or more compounds/agents have been (or are being) administered;
material comprising at least one of the two or more compounds/agents in an amount or in a form which is specifically adapted for use in combination with the other(s) of the two or more compounds/agents.

As used herein, the term “combination therapy” is intended to define therapies which comprise the use of a combination of two or more compounds/agents (as defined above). Thus, references to “combination therapy”, “combinations” and the use of compounds/agents “in combination” in this application may refer to compounds/agents that are administered as part of the same overall treatment regimen. As such, the posology of each of the two or more compounds/agents may differ: each may be administered at the same time or at different times. It will therefore be appreciated that the compounds/agents of the combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. The posologies of each of the two or more compounds/agents in a combination therapy may also differ with respect to the route of administration.

As used herein, the term “pharmaceutical kit” defines an array of one or more unit doses of a pharmaceutical composition together with dosing means (e.g. measuring device) and/or delivery means (e.g. inhaler or syringe), optionally all contained within common outer packaging. In pharmaceutical kits comprising a combination of two or more compounds/agents, the individual compounds/agents may unitary or non-unitary formulations. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. In pharmaceutical packs comprising a combination of two or more compounds/agents, the individual compounds/agents may unitary or non-unitary formulations. The unit dose(s) may be contained within a blister pack. The pharmaceutical pack may optionally further comprise instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

The combinations of the invention may produce a therapeutically efficacious effect relative to the therapeutic effect of the individual compounds/agents when administered separately.

The term iminosugar defines a saccharide analogue in which the ring oxygen is replaced by a nitrogen. The term is used herein sensu lato to include isoiminosugars, these being aza-carba analogues of sugars in which the C-1 carbon is replaced by nitrogen and the ring oxygen is replaced by a carbon atom, as well as azasugars in which an endocyclic carbon is replaced with a nitrogen atom. 1-Azasugars (with the N in the anomeric position) in which the ring oxygen is substituted with a carbon atom are isoiminosugars (as herein defined), but 1-azasugars in which the ring oxygen remains unsubstituted (oxazines) or is substituted with a nitrogen atom (hydrazines) are also of particular importance. In all cases, one or more endocyclic carbon atoms may be substituted with a sulphur, oxygen or nitrogen atom.

As used herein, the term glycosylation modulator encompasses any agent which alters N-linked or O-linked oligosaccharide structures on viral envelope glycoproteins. Preferably the glycosylation modulator is a glucosidase I or glycosidase I inhibitor. Particularly preferred glycosylation inhibitors are glycovirs. Most preferred glycosylation inhibitors are glucovirs.

The term alkovir is a term of art (see Block and Jordan (2001) Antivir. Chem. Chemother. 12(6): 317-325) and is used herein to define a family of iminosugars which exert antiviral activity independently of ER α-glucosidase inhibition. Alkovirs therefore include iminosugars which act to inhibit antiviral activity by mechanisms which are wholly independent of ER α-glucosidase inhibition (such alkovirs not being ER α-glucosidase inhibitors), as well as iminosugars which exert antiviral activity by a combination of ER α-glucosidase inhibition and one or more other modes of action (for example, interference with viral p7 protein or by immunomodulatory activity).

The term glucovir is a term of art (see Block and Jordan (2001) Antivir. Chem. Chemother. 12(6): 317-325) and is used herein to define a family of iminosugars which exert antiviral activity, at least in part, by ER α-glucosidase inhibition. Glucovirs therefore include iminosugars which act to inhibit antiviral activity by ER α-glucosidase inhibition, as well as iminosugars which exert antiviral activity by a combination of ER α-glucosidase inhibition and one or more other modes of action (for example, interference with viral p7 protein or by immunomodulatory activity). Thus, the alkovir and glucovir iminosugar families as herein defined partially overlap.

The analogous term glycovir is used herein as a more generic term than glucovir (as defined above) to define a class of iminosugars which exert antiviral activity, at least in part, by glycosidase inhibition. Thus, glucovirs form a subclass of the broader glycovir class of antiviral iminosugars. Thus, glycovirs and glucovirs suitable for use according to the invention may be glycosylation modulators as herein defined.

As used herein, the term polyhydroxylated iminosugar defines a class of oxygenated iminosugars. Typically these have at least 2, 3, 4, 5, 6 or 7 (preferably 3, 4 or 5) hydroxyl groups (or alkyl groups with one or more hydroxy substituent(s)) on the ring system nucleus.

The term iminosugar acid defines mono- or bicyclic sugar acid analogues in which the ring oxygen is replaced by a nitrogen. The term N-acid ISA defines an iminosugar acid in which the carboxylic acid group is located on the ring nitrogen.

Preferred ISAs are selected from the following structural classes: piperidine (including (poly)hydroxypipecolic acids); pyrroline; pyrrolidine (including (poly)hydroxyprolines); pyrrolizidine; indolizidine and nortropane.

As used herein, the term polyhydroxylated as applied to iminosugar acids defines an ISA having at least 2 (preferably at least 3) hydroxyl groups (or alkyl groups with one or more hydroxy substituent(s)) on the ring system nucleus.

As used herein, the term bicyclic polyhydroxylated iminosugar defines a class of highly oxygenated iminosugars having a double or fused ring nucleus (i.e. having two or more cyclic rings in which two or more atoms are common to two adjoining rings). Typically, such iminosugars have at least 3, 4, 5, 6 or 7 (preferably 3, 4 or 5) free hydroxyl groups on the ring system nucleus.

The term pharmacoperone is a term of art (from “pharmacological chaperone”) used to define a class of biologically active small molecules (sometimes also referred to in the art as “chemical chaperones”) that serve as molecular scaffolds, causing otherwise misfolded mutant proteins to fold and route correctly within the cell.

The term ligand as used herein in relation to the compounds of the invention is intended to define those compounds which can act as binding partners for a biological target molecule in vivo (for example, an enzyme or receptor, such as a PRR). Such ligands therefore include those which bind (or directly physically interact) with the target in vivo irrespective of the physiological consequences of that binding. Thus, the ligands of the invention may bind the target as part of a cellular signalling cascade in which the target forms a part. Alternatively, they may bind the target in the context of some other aspect of cellular physiology. In the latter case, the ligands may for example bind the target at the cell surface without triggering a signalling cascade, in which case the binding may affect other aspects of cell function. Thus, the ligands of the invention may bind the target and thereby effect an increase in the concentration of functional target at the cell surface (for example mediated via an increase in target stability, absolute receptor numbers and/or target activity). Alternatively, the iminosugar ligands may bind target (or target precursors) intracellularly, in which case they may act as molecular chaperones to increase the expression of active target.

The term PRR ligand as used herein in relation to the compounds for use according to the invention defines compounds which can act as binding partners for a PRR. Such compounds therefore include those which bind (or directly physically interact) with a PRR in vivo irrespective of the physiological consequences of that binding. Thus, the ligands of the invention may bind a PRR as part of a cellular signalling cascade in which the PRR forms a part. Alternatively, they may bind PRR in the context of some other aspect of cellular physiology. In the latter case, the ligands may for example bind PRR at the cell surface without triggering a signalling cascade, in which case the binding may affect other aspects of cell function. Thus, the ligands of the invention may bind PRRs and thereby effect an increase in the concentration of functional PRR at the cell surface (for example mediated via an increase in PRR stability, absolute receptor numbers and/or PRR activity). Alternatively, the ligands may bind PRR (or PRR precursors) intracellularly, in which case they may act as molecular chaperones to increase the expression of active PRR.

In preferred embodiments, the PRR ligands of the invention are PRR agonists. The term agonist is used herein in relation to the PRR ligands of the invention to define a subclass of ligands which productively bind PRR to trigger the cellular signalling cascade of which the PRR forms a part.

The term bioisostere (or simply isostere) is a term of art used to define drug analogues in which one or more atoms (or groups of atoms) have been substituted with replacement atoms (or groups of atoms) having similar steric and/or electronic features to those atoms which they replace. The substitution of a hydrogen atom or a hydroxyl group with a fluorine atom is a commonly employed bioisosteric replacement. Sila-substitution (C/Si-exchange) is a relatively recent technique for producing isosteres. This approach involves the replacement of one or more specific carbon atoms in a compound with silicon (for a review, see Tacke and Zilch (1986) Endeavour, New Series 10: 191-197). The sila-substituted isosteres (silicon isosteres) may exhibit improved pharmacological properties, and may for example be better tolerated, have a longer half-life or exhibit increased potency (see for example Englebienne (2005) Med. Chem., 1(3): 215-226). Similarly, replacement of an atom by one of its isotopes, for example hydrogen by deuterium, may also lead to improved pharmacological properties, for example leading to longer half-life (see for example Kushner et al (1999) Can J Physiol Pharmacol. 77(2):79-88). In its broadest aspect, the present invention contemplates all bioisosteres (and specifically, all silicon bioisosteres) of the compounds of the invention.

In its broadest aspect, the present invention contemplates all optical isomers, racemic forms and diastereoisomers of the compounds described herein. Those skilled in the art will appreciate that, owing to the asymmetrically substituted carbon atoms present in the compounds of the invention, the compounds may be produced in optically active and racemic forms. If a chiral centre or another form of isomeric centre is present in a compound of the present invention, all forms of such isomer or isomers, including enantiomers and diastereoisomers, are intended to be covered herein. Compounds of the invention containing a chiral centre (or multiple chiral centres) may be used as a racemic mixture, an enantiomerically enriched mixture, or the racemic mixture may be separated using well-known techniques and an individual enantiomer may be used alone. Thus, references to the compounds (e.g. iminosugars) of the present invention encompass the products as a mixture of diastereoisomers, as individual diastereoisomers, as a mixture of enantiomers as well as in the form of individual enantiomers.

Therefore, the present invention contemplates all optical isomers and racemic forms thereof of the compounds of the invention, and unless indicated otherwise (e.g. by use of dash-wedge structural formulae) the compounds shown herein are intended to encompass all possible optical isomers of the compounds so depicted. In cases where the stereochemical form of the compound is important for pharmaceutical utility, the invention contemplates use of an isolated eutomer.

The terms derivative and pharmaceutically acceptable derivative as applied to the compounds of the invention define compounds which are obtained (or obtainable) by chemical derivatization of the parent compound of the invention. The pharmaceutically acceptable derivatives are therefore suitable for administration to or use in contact with the tissues of humans without undue toxicity, irritation or allergic response (i.e. commensurate with a reasonable benefit/risk ratio). Preferred derivatives are those obtained (or obtainable) by alkylation, esterification or acylation of the parent compounds.

The pharmaceutically acceptable derivatives of the invention may retain some or all of the biological activities described herein. In some cases, the biological activity (e.g. chaperone activity) is increased by derivatization. The derivatives may act as pro-drugs, and one or more of the biological activities described herein (e.g. pharmacoperones activity) may arise only after in vivo processing. Particularly preferred pro-drugs are ester derivatives which are esterified at one or more of the free hydroxyls and which are activated by hydrolysis in vivo. Derivatization may also augment other biological activities of the compound, for example bioavailability and/or glycosidase inhibitory activity and/or glycosidase inhibitory profile. For example, derivatization may increase glycosidase inhibitory potency and/or specificity and/or CNS penetration (e.g. penetration of the blood-brain barrier).

The term pharmaceutically acceptable salt as applied to the iminosugars of the invention defines any non-toxic organic or inorganic acid addition salt of the free base which are suitable for use in contact with the tissues of humans and lower animals without undue toxicity, irritation, allergic response and which are commensurate with a reasonable benefit/risk ratio. Suitable pharmaceutically acceptable salts are well known in the art. Examples are the salts with inorganic acids (for example hydrochloric, hydrobromic, sulphuric and phosphoric acids), organic carboxylic acids (for example acetic, propionic, glycolic, lactic, pyruvic, malonic, succinic, fumaric, malic, tartaric, citric, ascorbic, maleic, hydroxymaleic, dihydroxymaleic, benzoic, phenylacetic, 4-aminobenzoic, 4-hydroxybenzoic, anthranilic, cinnamic, salicylic, 2-phenoxybenzoic, 2-acetoxybenzoic and mandelic acid) and organic sulfonic acids (for example methanesulfonic acid and p-toluenesulfonic acid).

These salts and the free base compounds can exist in either a hydrated or a substantially anhydrous form. Crystalline forms, including all polymorphic forms, of the iminosugars of the invention are also contemplated and in general the acid addition salts of the compounds are crystalline materials which are soluble in water and various hydrophilic organic solvents and which in comparison to their free base forms, demonstrate higher melting points and an increased solubility.

In the present specification the term “alkyl” defines a straight or branched saturated hydrocarbon chain. The term “C₁-C₆ alkyl” refers to a straight or branched saturated hydrocarbon chain having one to six carbon atoms. The term “C₁-C₉ alkyl” refers to a straight or branched saturated hydrocarbon chain having one to nine carbon atoms. The term “C₁-C₁₅ alkyl” refers to a straight or branched saturated hydrocarbon chain having one to fifteen carbon atoms. Preferred is C₁-C₆ alkyl. Examples include methyl, ethyl, n-propyl, isopropyl, t-butyl, n-hexyl. The alkyl groups of the invention may be optionally substituted by one or more halogen atoms.

In the present specification the term “alkenyl” defines a straight or branched hydrocarbon chain having containing at least one carbon-carbon double bond. The term “C₁-C₆ alkenyl” refers to a straight or branched unsaturated hydrocarbon chain having one to six carbon atoms. The term “C₁-C₉ alkenyl” refers to a straight or branched unsaturated hydrocarbon chain having one to nine carbon atoms. The term “C₁-C₁₅ alkenyl” refers to a straight or branched unsaturated hydrocarbon chain having one to fifteen carbon atoms. Preferred is C₁-C₆ alkenyl. Examples include ethenyl, 2-propenyl, and 3-hexenyl. The alkenyl groups of the invention may be optionally substituted by one or more halogen atoms.

In the present specification the term “alkynyl” defines a straight or branched hydrocarbon chain having containing at least one carbon-carbon triple bond. The term “C₁-C₆ alkynyl” refers to a straight or branched unsaturated hydrocarbon chain having one to six carbon atoms. The term “C₁-C₉ alkynyl” refers to a straight or branched unsaturated hydrocarbon chain having one to nine carbon atoms. The term “C₁-C₁₅ alkynyl” refers to a straight or branched unsaturated hydrocarbon chain having one to fifteen carbon atoms. Preferred is C₁-C₆ alkynyl. Examples include ethynyl, 2-propynyl, and 3-hexynyl. The alkynyl groups of the invention may be optionally substituted by one or more halogen atoms.

As used herein, the term “carbocyclyl” means a mono- or polycyclic residue containing 3 or more (e.g. 3-10 or 3-8) carbon atoms. The carbocyclyl residues of the invention may be optionally substituted by one or more halogen atoms. Mono- and bicyclic carbocyclyl residues are preferred. The carbocyclyl residues can be saturated or partially unsaturated.

Saturated carbocyclyl residues are preferred and are referred to herein as “cycloalkyls” and the term “cycloalkyl” is used herein to define a saturated 3 to 14 membered carbocyclic ring including fused bicyclic or tricyclic systems. Examples of such groups include cyclopropyl, cyclobutyl, cyclopentyl, cyclohexyl and also bridged systems such as norbornyl and adamantyl. The cycloalkyl residues of the invention may be optionally substituted by one or more halogen atoms.

In the present specification the term “aryl” defines a 5-14 (e.g. 5-10) membered aromatic mono-, bi- or tricyclic group at least one ring of which is aromatic. Thus, bicyclic aryl groups may contain only one aromatic ring. As used herein, the term “aryl” includes heteroaryls containing heteroatoms (e.g. nitrogen, sulphur and/or oxygen) being otherwise as defined above. The aryl groups of the invention may optionally be substituted by one or more halogen atoms. Examples of aromatic moieties are benzene, naphthalene, imidazole and pyridine.

In the present specification, “halo” refers to fluoro, chloro, bromo or iodo.

### Flaviviral Targets of the Compounds of the Invention

The compounds of the invention find general application in the treatment of infections with any virus of the family Flaviviridae (i.e. a flavivirus, as herein defined). The invention therefore contemplates the use of the compounds of the invention for the treatment of any disease arising from infection with any virus of the family Flaviviridae.

Thus, the invention finds application in the treatment of infection with (and disease caused by) any virus of the family Flaviviridae including any virus from the genera Flavivirus, Pestivirus and Hepacivirus. Thus, the invention finds application in the treatment of numerous human diseases and a variety of animal diseases which cause significant losses to the livestock industry.

Thus, the invention finds application in the treatment of infection with (and disease caused by) a virus selected from the genera Flavivirus (e.g. yellow fever virus, dengue viruses, Japanese encephalitis virus, Murray Valley encephalitis virus, West Nile fever virus, Rocio virus, St. Louis encephalitis virus, Louping ill virus, Powassan virus, Omsk hemorrhagic fever virus, Kyasanur forest disease virus and tick-borne encephalitis virus), Pestivirus (e.g. bovine viral diarrhoea virus, rubella virus, classical swine fever virus, hog cholera virus and border disease virus), Hepacivirus (hepatitis C virus) and currently unclassified members of the Flaviviridae (e.g. GB virus types A, B and C).

In preferred embodiments, the compound of the invention is for the treatment of infection with (and disease caused by) a member of the genus Hepacivirus. In a particularly preferred embodiment the hepacivirus is the hepatitis C virus (HCV). In such embodiments, the HCV virus may be selected from genotype 1, 2, 3, 4, 5 or 6). Any and all subtypes and quasispecies may be treated according to the invention, but particularly preferred is the treatment of infection with HCV genotypes 1a, 1b, 2a, 2b, 2c, 3a, 4 and/or 5.

Thus, the compounds of the invention may find application in the treatment of a disease selected from hepatitis C, yellow fever, dengue fever, Japanese encephalitis, Murray Valley encephalitis, Rocio virus infection, West Nile fever, St. Louis encephalitis, tick-borne encephalitis, Louping ill virus infection, Powassan virus infection, Omsk hemorrhagic fever, Kyasanur forest disease, bovine diarrhoea, classical swine fever, border disease and hog cholera.

### Compounds for Use According to the Invention

Certain compounds as described below (e.g. those compounds of Formula (1), (2) or (3) described in Section A(I) and/or the iminosugars described in Section A(II)) are novel.

According to the invention, those compounds which are novel are claimed as compounds per se, together with processes for their preparation, compositions containing them, as well as their use as pharmaceuticals (for example in any of the particular medical uses described herein).

Moreover, to the extent that certain of the compounds as described below (e.g. those compounds of Formula (1), (2) or (3) described in Section A(I) and/or the iminosugars described in Section A(II)) are known as such but not as pharmaceuticals, those compounds are claimed for use as pharmaceuticals (for example in any of the particular medical uses described herein).

### A. Structural Considerations

### (I) Compounds of Formula (1), (2) or (3)

The compounds for use according to the invention may comprise a nucleus selected from those shown below and numbered (1), (2) and (3):

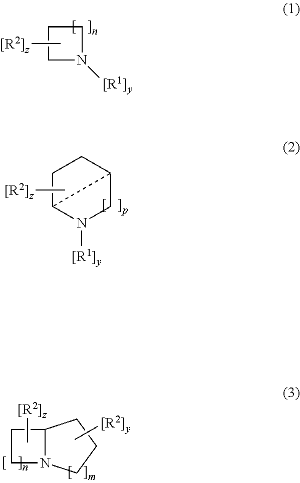

### (i) Compounds of Formula (1)

The compounds for use according to the invention may be of Formula (1)

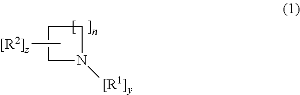

in which
n represents an integer from 1 to 7, provided that where n>1 the ring may also contain at least one unsaturated C—C bond
z represents an integer from 1 to (n+2)
y represents 1 or 2
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N3, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
   or a pharmaceutically acceptable salt or derivative thereof.

In preferred embodiments, the compound of Formula (1) is selected from any one of the Formulae shown below:

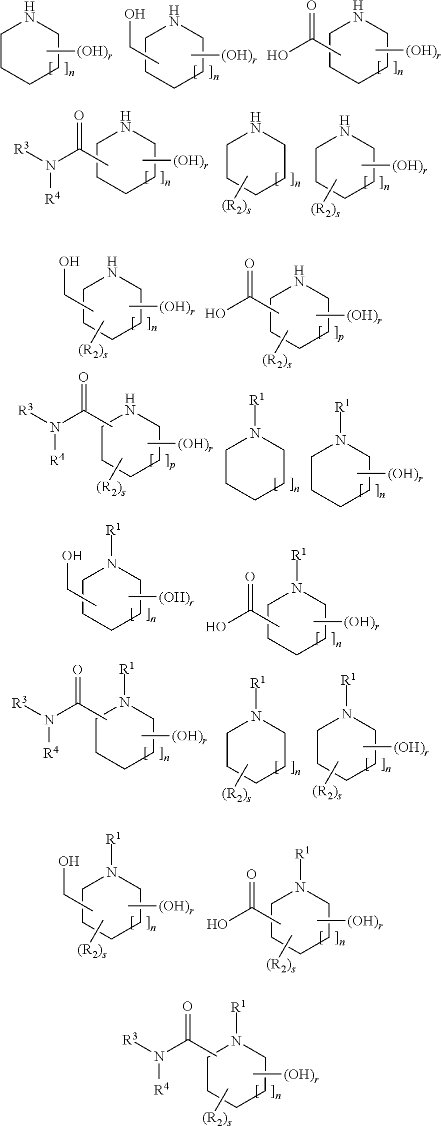

wherein:
r represents an integer from 1 to (n+4)
s represents an integer from 1 to (n+4)
n represents an integer from 0 to 2
R¹ represents C1-9 alkyl, optionally substituted with up to 6 OH, NR³R⁴, aryl, O—C1-3 alkyl, O—C1-3 alkenyl, CO₂H, NH(NH)NH₂, CONR³R⁴; C(O)OR³; C(O)NR³R⁴; SO₂NR³
R² represents ═O; C1-9 alkyl, C1-9 alkenyl, aryl, optionally substituted with up to 6 OH, NR³R⁴, aryl, O—C1-3 alkyl, CONR³R⁴, C(O)OR³; C(O)NR³R⁴; SO₂NR³; NH(NH)NH₂; NR⁴C(O)R³; NR⁴SO₂R³, N₃; F; Cl
R³ represents H; C1-6 alkyl, optionally substituted with up to 4 OH; aryl or C1-3 alkyl optionally substituted with aryl
R⁴ represents H; C1-6 alkyl, optionally substituted with up to 4 OH
R³ and R⁴ may optionally form a 4 to 8 membered ring, containing 0 to 1 O, S or NR³ groups.

### (ii) Compounds of Formula (2)

The compounds for use according to the invention may be of Formula (2)

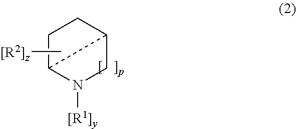

in which
p represents an integer from 1 to 2
z represents an integer from 1 to (p+7)
y represents 1 or 2
the broken line represents a bridge containing 2 or 3 carbon atoms between any two different ring carbon atoms, any or all of which bridge or bridgehead carbon atoms being optionally substituted with R²
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N3, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
   or pharmaceutically acceptable salt or derivative thereof.

In preferred embodiments, the compound of Formula (2) is selected from any one of the Formulae shown below:

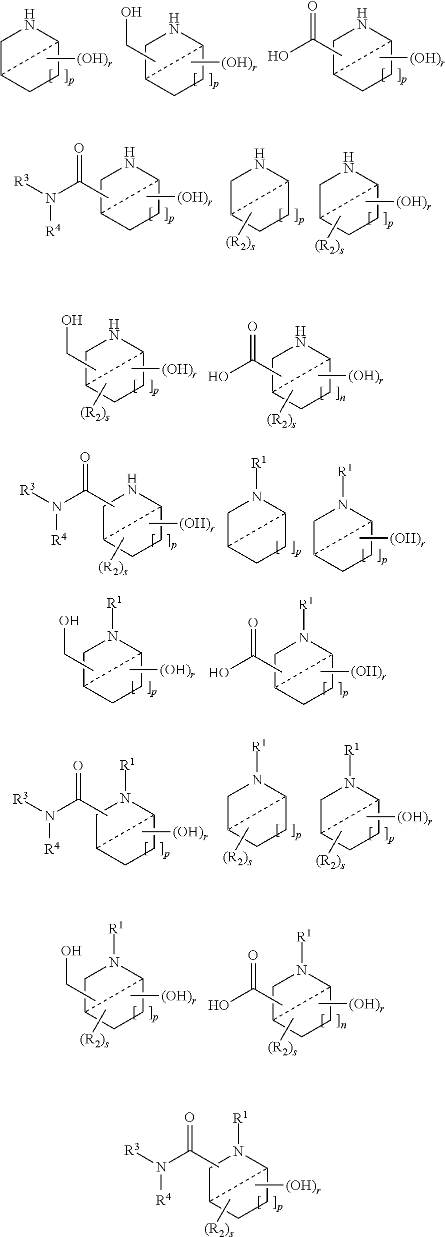

wherein:
r represents an integer from 1 to (n+4)
s represents an integer from 1 to (n+4)
p represents an integer from 1 to 2
R¹ represents C1-9 alkyl, optionally substituted with up to 6 OH, NR³R⁴, aryl, O—C1-3 alkyl, O—C1-3 alkenyl, CO₂H, NH(NH)NH₂, CONR³R⁴; C(O)OR³; C(O)NR³R⁴; SO₂NR³
R² represents ═O; C1-9 alkyl, C1-9 alkenyl, aryl, optionally substituted with up to 6 OH, NR³R⁴, aryl, O—C1-3 alkyl, CONR³R⁴, C(O)OR³; C(O)NR³R⁴; SO₂NR³; NH(NH)NH₂; NR⁴C(O)R³; NR⁴SO₂R³, N₃; F; Cl
R³ represents H; C1-6 alkyl, optionally substituted with up to 4 OH; aryl or C1-3 alkyl optionally substituted with aryl
R⁴ represents H; C1-6 alkyl, optionally substituted with up to 4 OH R³ and R⁴ may optionally form a 4 to 8 membered ring, containing 0 to 1 O, S or NR³ groups.
   (iii) Compounds of Formula (3)

The compounds for use according to the invention may be of Formula (3)

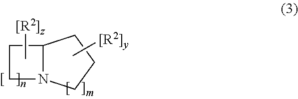

in which
n represents an integer from 1 to 7, for example 1 to 5, provided that where n>1 the ring may also contain at least one unsaturated C—C bond
m represents an integer from 1 to 3 and the ring may also contain at least one unsaturated C—C bond
z represents an integer from 0 to (n+2), provided that where z=0 then y≧1
y represents an integer from 0 to (m+2), provided that where y=0 then z≧1
the endocyclic nitrogen atom may be bonded to an oxygen or an oxygen containing group such that the compound is an N-oxide,
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N3, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
optionally wherein the compound has three, four or more rings
   or pharmaceutically acceptable salt or derivative thereof.

In preferred embodiments, the compound of Formula (3) is selected from any one of the Formulae shown below:

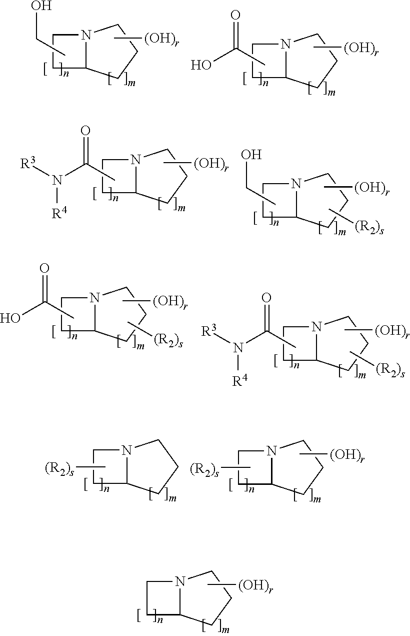

wherein:
r represents an integer from 1 to (n+m+4)
s represents an integer from 1 to (n+m+4)
n represents an integer from 1 to 3
m represents an integer from 1 to 3
R² represents ═O; C1-9 alkyl, C1-9 alkenyl, aryl, optionally substituted with up to 6 OH, NR³R⁴, aryl, O—C1-3 alkyl, CONR³R⁴, C(O)OR³; C(O)NR³R⁴; SO₂NR³; NH(NH)NH₂; NR⁴C(O)R³; NR⁴SO₂R³, N3; F; Cl
R³ represents H; C1-6 alkyl, optionally substituted with up to 4 OH; aryl or C1-3 alkyl optionally substituted with aryl
R⁴ represents H; C1-6 alkyl, optionally substituted with up to 4 OH
R³ and R⁴ may optionally form a 4 to 8 membered ring, containing 0 to 1 O, S or NR³ groups
the endocyclic nitrogen atom may be bonded to an oxygen or an oxygen containing group such that the compound is an N-oxide.

In all of the above compounds, one or more endocyclic carbon atoms may be substituted with a sulphur, oxygen or nitrogen atom.

It will be appreciated that the compounds of Formula (1), (2) and (3) may comprise compounds having three, four or more rings.

Preferred are compounds of Formula (1), (2) or (3) which are polyhydroxylated, having 2, 3 or more hydroxyl residues on the ring system nucleus.

Also preferred are oligomers (e.g. dimers, trimers etc.) of the above-defined compounds. Such compounds may be di- and/or oligosaccharide mimetics (as described below), and they may be linked, for example, at C6 and C2, 3 or 4. Oligomers of the above-defined compounds are preferably imino-C-disaccharides and analogues as described in Section II(b)(vi), below.

Certain compounds of Formula (1), (2) or (3) are novel. According to the invention, those compounds of Formula (1), (2) or (3) which are novel are claimed as compounds per se, together with processes for their preparation, compositions containing them, as well as their use as pharmaceuticals (for example in any of the particular medical uses described herein).

Moreover, to the extent that certain of the compounds falling within the scope of Formula (1), (2) or (3) are known, as such, but not as pharmaceuticals, those compounds are claimed for use as pharmaceuticals (for example in any of the particular medical uses described herein).

The compounds of Formula (1), (2) or (3) may be, but not necessarily are, iminosugars as defined in Section A(II) (below).

### (II) Iminosugars

The compounds for use according to the invention may be iminosugars, as hereinbefore defined.

Thus, the compounds for use according to the invention may be selected from:
iminosugars sensu stricto, being saccharide analogues in which the ring oxygen is replaced by a nitrogen; or
isoiminosugars, being aza-carba analogues of sugars in which the C-1 carbon is replaced by nitrogen and the ring oxygen is replaced by a carbon atom; and
azasugars in which an endocyclic carbon is replaced with a nitrogen atom.

In embodiments where the iminosugar for use according to the invention is an azasugar as defined above, then the iminosugar may be selected from:
1-Azasugars in which the N is in the anomeric position;
oxazines in which the ring oxygen remains unsubstituted; and
hydrazines in which the ring oxygen is substituted with a nitrogen atom.

In all of the above iminosugars, one or more endocyclic carbon atoms may be substituted with a sulphur, oxygen or nitrogen atom.

The iminosugars for use according to the invention may be of Formula (1), (2) or (3) as defined in Section A(I) (above).

The iminosugars as defined above for use according to the invention may be of any structural class or subclass, including the classes described below:

### (a) Principal Structural Iminosugar Classes

The compounds for use according to the invention may be an iminosugar (as herein defined). The iminosugars for use according to the invention may be of a structural class selected from:
(a) a piperidine;
(b) a pyrroline;
(c) a pyrrolidine;
(d) a pyrrolizidine;
(e) an indolizidine;
(f) a quinolizidine;
(g) a nortropane;
(h) ring-open iminosugars;
(i) 5,7 fused;
(j) an azepane;
(k) an azetidine;
(l) mixtures of any two or more of (a) to (k).

The iminosugars of any of the foregoing structural classes may be polyhydroxylated, as hereinbefore defined. As used herein, the term polyhydroxylated piperidine iminosugar defines an oxygenated iminosugar (e.g. having at least 2 (preferably at least 3) free hydroxyl groups (or alkyl groups with one or more OH substituents) on the ring system nucleus) that comprises the nucleus:

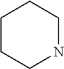

As used herein, the term polyhydroxylated pyrrolidine iminosugar defines an oxygenated iminosugar (e.g. having at least 2 (preferably at least 3) free hydroxyl groups (or alkyl groups with one or more OH substituents) on the ring system nucleus) that comprises the nucleus:

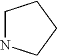

As used herein, the term polyhydroxylated pyrrolizidine iminosugar defines an oxygenated iminosugar (e.g. having at least 3, 4, 5, 6 or 7 (preferably 3, 4 or 5) free hydroxyl groups (or alkyl groups with one or more OH substituents) on the ring system nucleus) that comprises the nucleus:

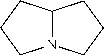

As used herein, the term polyhydroxylated indolizidine iminosugar defines an oxygenated iminosugar (e.g. having at least 3, 4, 5, 6 or 7 (preferably 3, 4 or 5) free hydroxyl groups (or alkyl groups with one or more OH substituents) on the ring system nucleus) that comprises the nucleus:

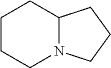

As used herein, the term polyhydroxylated quinolizidine iminosugar defines an oxygenated iminosugar (e.g. having at least 3, 4, 5, 6 or 7 (preferably 3, 4, 5 or 6) free hydroxyl groups (or alkyl groups with one or more OH substituents) on the ring system nucleus) that comprises the nucleus:

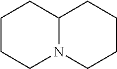

In each of the above iminosugar nuclei, it is to be understood that one or more endocyclic carbon atoms may be substituted with a sulphur, oxygen or nitrogen atom.

### (i) Piperidine Iminosugars

Piperidine iminosugars comprise the nucleus:

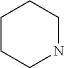

Preferred are polyhydroxylated piperidine iminosugars as hereinbefore defined comprising the above nucleus and having at least 2 (preferably at least 3) hydroxyl groups (or alkyl groups with one or more hydroxy substituent(s)) on the ring system nucleus.

### (ii) Pyrroline Iminosugars

Pyrroline iminosugars comprise one of the following three nuclei:

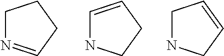

Preferred are polyhydroxylated pyrroline iminosugars as hereinbefore defined having at least 2 hydroxyl groups (or alkyl groups with one or more hydroxy substituent(s)) on the ring system nucleus.

(iii) Pyrrolidine Iminosugars

Pyrrolidine iminosugars comprise the nucleus:

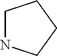

Preferred are polyhydroxylated pyrrolidine iminosugars as hereinbefore defined comprising the above nucleus and having at least 2 (for example at least 3) hydroxyl groups (or alkyl groups with one or more hydroxy substituent(s)) on the ring system nucleus.

### (iv) Pyrrolizidine Iminosugars

Pyrrolizidine iminosugars comprise the nucleus:

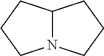

Preferred are polyhydroxylated pyrrolizidine iminosugars as hereinbefore defined comprising the above nucleus and having at least 2, 3, 4, 5, 6 or 7 (preferably 3, 4 or 5) hydroxyl groups (or alkyl groups with one or more hydroxy substituent(s)) on the ring system nucleus.

(v) Indolizidine Iminosugars Indolizidine iminosugars comprise the nucleus:

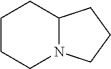

Preferred are polyhydroxylated indolizidine iminosugars as hereinbefore defined comprising the above nucleus and having at least 2, 3, 4, 5, 6 or 7 (preferably 3, 4 or 5) hydroxyl groups (or alkyl groups with one or more hydroxy substituent(s)) on the ring system nucleus.

### (vi) Quinolizidine Iminosugars

Quinolizidine iminosugars comprise the nucleus:

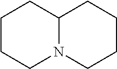

Preferred are polyhydroxylated quinolizidine iminosugars as hereinbefore defined comprising the above nucleus and having at least 2, 3, 4, 5, 6 or 7 (preferably 3, 4, 5 or 6) hydroxyl groups (or alkyl groups with one or more hydroxy substituent(s)) on the ring system nucleus.

(vii) Nortropanes

Nortropane iminosugars comprise the nucleus:

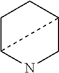

wherein the dotted line represents a bridge containing 2 or 3 carbon atoms between any two different ring carbon atoms.

Preferred are polyhydroxylated nortropane iminosugars as hereinbefore defined comprising the above nucleus and having at least 3 (preferably at least 4) hydroxyl groups (or alkyl groups with one or more hydroxy substituent(s)) on the ring system nucleus.

A preferred class of nortropane iminosugar for use according to the invention are calystegines. These are polyhdroxylated nor-tropanes which have been reported to inhibit β-glucosidases, β-xylosidases and α-galactosidases (Asano et al., 1997, Glycobiology 7: 1085-1088). The calystegines are common in foods belonging to the Solanaceae family of plants that includes potatoes and aubergines (egg plant). The calystegines have been shown to inhibit mammalian glycosidases including human, rat and bovine liver enzymes. Attaching sugars to the calystegines such as in 3-O-β-D-glucopyranoside of 1α,2β,3α,6α-tetrahydroxy-nor-tropane (Calystegine B₁) (Griffiths, et al., 1996, Tetrahedron Letters 37: 3207-3208) can alter the glycosidase inhibition to include α-glucosidases and β-galactosidases.

(viii) 5-7 Fused

These iminosugars comprise the nucleus:

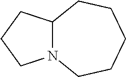

Preferred are polyhydroxylated 5-7 fused iminosugars as hereinbefore defined comprising the above nucleus and having at least 2, 3, 4, 5, 6 or 7 (preferably 3, 4 or 5) hydroxyl groups (or alkyl groups with one or more hydroxy substituent(s)) on the ring system nucleus.

### (ix) Azepanes

Azepane iminosugars comprise the nucleus:

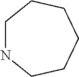

Preferred are polyhydroxylated azepane iminosugars as hereinbefore defined comprising the above nucleus and having at least 2 (preferably at least 3 or 4) hydroxyl groups (or alkyl groups with one or more hydroxy substituent(s)) on the ring system nucleus.

In each of the above iminosugar nuclei described in subsections (i) to (ix), it is to be understood that one or more endocyclic carbon atoms may be substituted with a sulphur, oxygen or nitrogen atom.

It will also be appreciated that iminosugars comprising the various nuclei described in subsections (i) to (ix) comprise compounds having three, four or more rings.

### (x) Ring-Open Iminosugars

Also considered are amino sugars acids formed by the opening of the imino ring such as compound P1 and P2 (found in Cucurbita spp.) and P3. Such compounds may also be the biological precursors of the iminosugar acids.

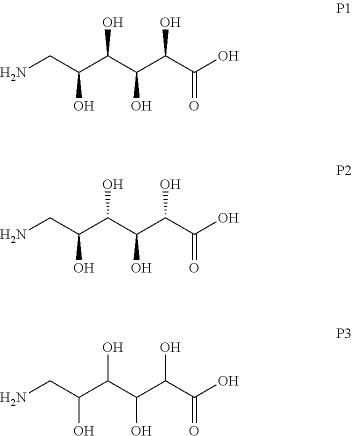

### (b) Iminosugar Structural Subclasses

The principal structural classes described above can be further categorized into various subclasses, for example on the basis of the presence of various functional groups, as described below.

The iminosugars for use according to the invention may therefore be further characterized on the basis of their structural subclass, for example being selected from:

### (i) Iminosugar Acids

The iminosugar acids (ISAs) are mono- or bicyclic analogues of sugar acids in which the ring oxygen is replaced by a nitrogen. Although iminosugars are widely distributed in plants (Watson et al. (2001) Phytochemistry 56: 265-295), the iminosugar acids are much less widely distributed.

Iminosugar acids can be classified structurally on the basis of the configuration of the N-heterocycle. Examples include piperidine, pyrroline, pyrrolidine, pyrrolizidine, indolizidine and nortropanes iminosugar acids (see FIGS. 1-7 of Watson et al. (2001), the disclosure of which is incorporated herein by reference).

Particularly preferred are iminosugar acids selected from the following structural classes:
(a) piperidine ISAs (including (poly)hydroxypipecolic acids);
(b) pyrroline ISAs;
(c) pyrrolidine ISAs (including (poly)hydroxyprolines);
(d) pyrrolizidine ISAs;
(e) indolizidine ISAs; and
(f) nortropane ISAs.

The ISAs for use according to the invention may be N-acid ISAs (as hereinbefore defined).

ISA mixtures or combinations containing two or more different ISAs representative of one or more of the classes listed above may also be used.

Preferred are polyhydroxylated ISAs. Particularly preferred are ISAs having a small molecular weight, since these may exhibit desirable pharmacokinetics. Thus, the ISA may have a molecular weight of 100 to 400 Daltons, preferably 150 to 300 Daltons and most preferably 200 to 250 Daltons.

Also preferred are ISAs, which are analogues of hydroxymethyl-substituted iminosugars in which one or more hydroxymethyl groups are replaced with carboxyl groups.

### Exemplary Piperidine Iminosugar Acids

The ISA of the invention may be a piperidine ISA having at least 3 free hydroxyl (or hydroxyalkyl) groups on the ring system nucleus. Exemplary piperidine ISAs are hydroxypipecolic acids. Particularly preferred hydroxypipecolic acids are polyhydroxypipecolic acids having at least two (e.g. 3) free hydroxyl (or hydroxyalkyl) groups on the ring system nucleus.

### Exemplary Pyrrolidine Iminosugar Acids

The ISA of the invention may be a pyrrolidine ISAs having at least 2 (preferably at least 3) free hydroxyl (or hydroxyalkyl) groups on the ring system nucleus. Preferred pyrrolidine ISAs are hydroxyprolines. Particularly preferred hydroxyprolines are polyhydroxyprolines having at least two (e.g. at least 3) free hydroxyl (or hydroxyalkyl) groups on the ring system nucleus.

### Exemplary Pyrrolizidine Iminosugar Acids

The ISA of the invention may be a pyrrolizidine ISA having at least 2 (preferably at least 3, 4 or 5) free hydroxyl (or hydroxyalkyl) groups on the ring system nucleus.

### Exemplary Indolizidine Iminosugar Acids

The ISA of the invention may be an indolizidine ISA having at least 2 (preferably at least 3, 4 or 5) free hydroxyl (or hydroxyalkyl) groups on the ring system nucleus.

### Exemplary Nortropane Iminosugar Acids

The ISA of the invention may be a nortropane ISA having at least 2 (preferably at least 3) free hydroxyl (or hydroxyalkyl) groups on the ring system nucleus.

### (ii) 1-N-Iminosugars (Isoiminosugars)

Isoiminosugars are carbohydrate mimics in which the anomeric carbon is replaced by a nitrogen atom and the ring oxygen is replaced by a carbon atom (for example, a methylene group in the case of monocyclic piperidine and pyrrolidine compounds).

(iii) Iminosugar Conjugates

Carbohydrates are often conjugated to other biomolecules in vivo, including lipids, proteins, nucleosides and phosphate groups. Thus, of particular interest as a subclass of the various principal classes of iminosugar described above are iminosugar conjugates. These include:
Iminosugar-based glycopeptide analogues
Iminosugar phosphonate analogues
Iminosugar nucleotide analogues and oligomers thereof
Iminosugar glycolipid analogues (e.g. C- or N-alkyl iminosugar derivatives)

### (iv) Iminosugar C-Glycosides

Imino-analogues of glycosides in which an aglycone moiety is attached to the anomeric (C-1) carbon via an O-glycosidic bond are of limited utility as drugs due to the lability of the N,O-acetal function. Replacement of the oxygen atom of the N,O-acetal by a methylene group yields iminosugar C-glycosides, which are stable analogues of glycoconjugates. The endocyclic nitrogen is preferably unsubstituted in such C-glycosides, so that the compounds may comprise a nucleus selected from those listed below:

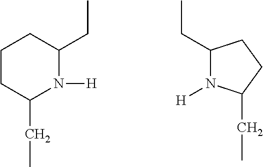

Iminosugars of this structural subclass are described by Compain (2007) In “Iminosugars: From synthesis to therapeutic applications”, Wiley ISBN 978-0-470-03391-3; Compain and Martin (Eds.) 63-86 (the disclosure of which is hereby incorporated by reference).

### (v) N-Substituted Iminosugars

N-substituted iminosugars may be considered as analogues of the iminosugar C-glycosides described above in which the aglycone moiety is positioned on the endocyclic nitrogen rather than the “anomeric” C-1 carbon atom.

### (vi) Imino-C-Disaccharides and Analogues

Imino-C-disaccharides and analogues for use according to the invention may fall into any one of the three structural subclasses described by Vogel et al. (2007) In “Iminosugars: From synthesis to therapeutic applications”, Wiley ISBN 978-0-470-03391-3; Compain and Martin (Eds.) 63-130 the disclosure of which is hereby incorporated herein by reference. For example, they may be: (a) linear (1→1)-C-linked; (b) linear (1→ω)-C-linked; or (c) branched (1→n)-C-linked (see FIG. 5.1 of Vogel et al. (2007), op. cit.).

(vii) Iminosugar Lactams

Iminosugar lactams for use according to the invention may for example comprise a nucleus selected from:

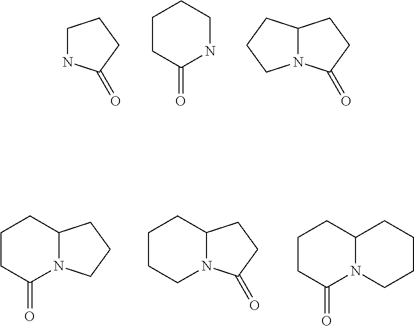

in which the ═O group may be on both rings of the bicyclic nuclei.

In each of the above iminosugar lactam nuclei, it is to be understood that one or more endocyclic carbon atoms may be substituted with a sulphur, oxygen or nitrogen atom.

(viii) Branched Iminosugars

The iminosugars for use according to the invention may be a branched iminosugar. Branched iminosugars are as defined in sections (i) to (x) (above) but are distinguished by the presence of two non-H substituents (e.g. two alkyl groups, two hydroxyalkyl groups, a hydroxy and hydroxyalkyl group or a hydroxy and alkyl group) on any one or more endocyclic carbon atom.

It will be appreciated that iminosugars with features characteristic of two or more of the foregoing subclasses (i) to (x) may also find application according to the invention.

### (c) Iminosugar Carbohydrate Mimetics

As described above, the iminosugars for use according to the invention may be of any structural class and/or subclass, including the classes and subclasses described above in Sections II(a) and II(b). In addition to this structural classification, the iminosugars for use according to the invention may also be further structurally and/or functionally defined by reference to the carbohydrate(s) they mimic, as described below:

### (i) General Considerations

An iminosugar carbohydrate mimetic is an iminosugar that mimics one or more carbohydrates (for example, a mono- or disaccharide) through replication of one or more structural motifs of the carbohydrate scaffold. Thus, iminosugar carbohydrate mimetics share absolute/relative stereochemical motifs with the carbohydrate(s) they mimic.

This structural mimicry may be associated with functional mimicry: the shared absolute/relative stereochemical motifs may give rise to shared functional attributes. In such cases the compound may be defined as a functional sugar mimetic (as discussed in more detail in Section B, below). However, since the sugar mimics of the carbohydrate may also contain new functional groups, a new scaffold, or both, they may also exhibit functional attributes which are distinct from those of the carbohydrate(s) mimicked.

Thus, iminosugar carbohydrate mimetics correspond structurally to one or more carbohydrates and this structural mimicry may be accompanied by functional mimicry (e.g. at the level of interaction with a biological target in vivo) or other functional attributes related to, but distinct from, those of the carbohydrate they mimic (for example, the ability to competitively inhibit an enzyme for which the carbohydrate mimicked is a substrate in vivo).

For example, and considering the following pentose (3 contiguous chiral centres) and hexose (4 contiguous chiral centres) stereochemistries (Scheme 1, below):

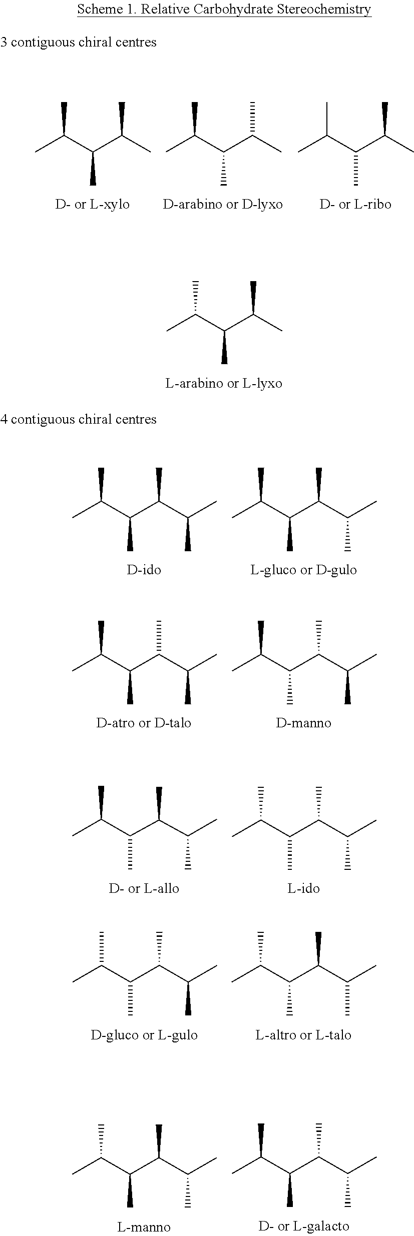

The above analysis is non-limiting, and intended to be illustrative only of a wider principle. A similar analysis can readily be extended to lower sugars (e.g. tetroses) and higher sugars (e.g. heptoses), as well as to ketoses and the like.

An iminosugar can be considered as being a structural mimetic of a particular reference monosaccharide, disaccharide or oligosaccharide unit when stereochemical comparisons between the iminosugar and the relative carbohydrate stereochemistry exhibited by the carbohydrate scaffold reveal shared stereochemical motifs. For the purposes of the analysis, the stereochemical comparison relates to consideration of contiguous C-het stereocentres (these being C—O, C—N etc.)

For example in the case of two simple monocyclic iminosugars IS1 and IS2 (shown below) the relative stereochemical relationship to the reference monosaccharide units (D-arabinose and D-glucose respectively) can be seen:

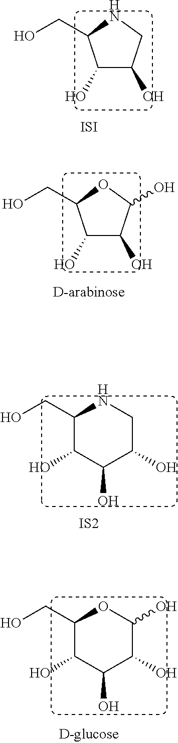

Thus, IS1 is a D-arabinose mimetic while IS2 is a D-glucose mimetic.

However, as monosaccharides can exist in both acyclic and several cyclic forms, the relative stereochemical relationship between the iminosugar and the parent monosaccharide is not necessarily fixed to one structural class or type or to the contiguous sequence depicted.

For example, D-arabinose can exist in the following cyclic forms:

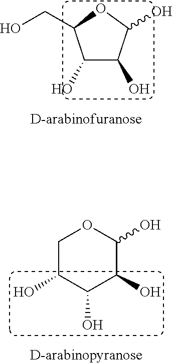

Exemplary iminosugar mimetics include the iminosugars IS1 and IS3, respectively, as shown below:

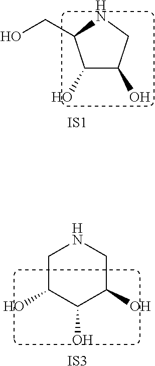

Note that unlike their monosaccharide counterparts these compounds generally cannot interconvert and are chemically distinct from each other. Thus, IS1 is a D-arabinofuranose mimetic while IS3 is a D-arabinopyranose mimetic.

However, in the case of IS3 the stereochemistry represents that not just of D-arabinopyranose but also that of D-lyxose:

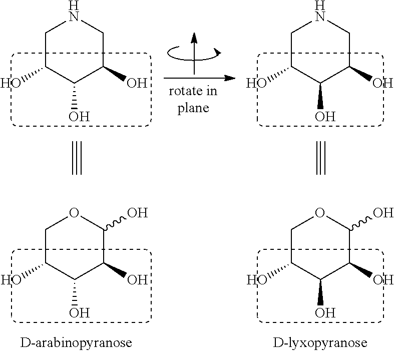

This is a consequence of the stereochemical sequence overlap that exists amongst carbohydrate sequences. For these purposes the carbon backbone with the most contiguous chiral centres is selected primarily. When considering cyclic iminosugars the ring nitrogen is included amongst the primary contiguous chiral centres.

For example, the iminosugar IS4 exhibits the following stereochemical sequences:

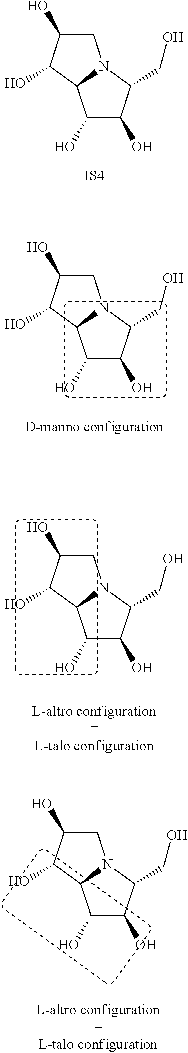

The iminosugar IS5 exhibits the following stereochemical sequences:

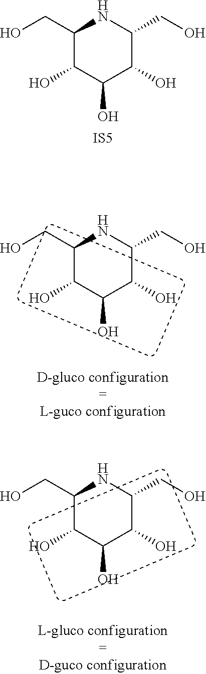

The iminosugar IS6 exhibits the following stereochemical sequences:

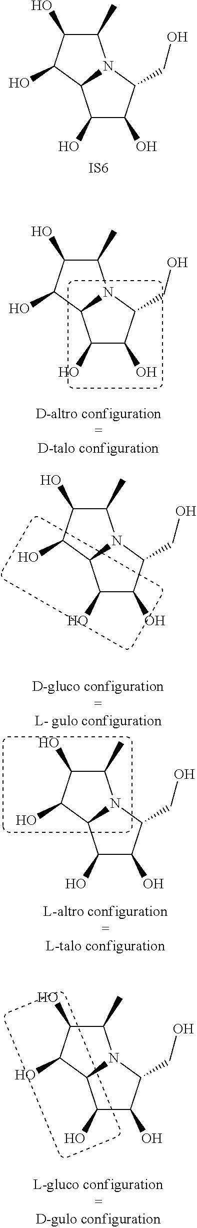

However, although an iminosugar may present more than one stereochemical sequence it is not necessarily a carbohydrate mimetic for each and every stereochemical sequence exhibited.

For example, the 2,5-imino pyrrolidine IS7 exhibits both D-gluco and L-gulo stereochemistry and can be considered as both a glucose and gulose mimetic:

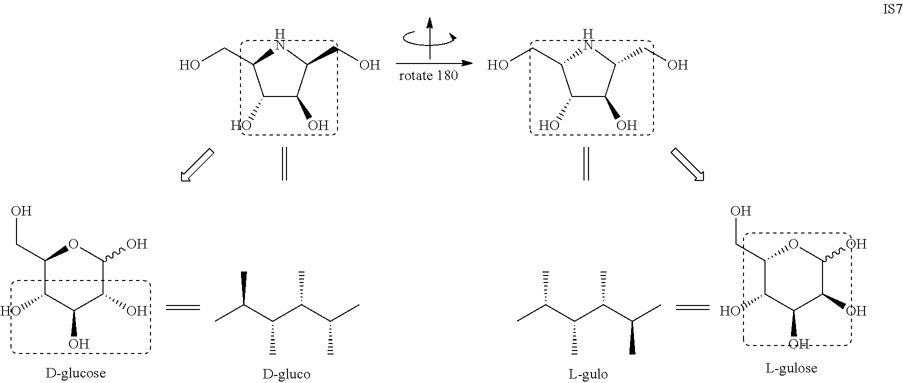

Note that an alternative, but chemically distinct isomer of IS7, not the 2,5-pyrrolidine but the 1,4-pyrrolidine IS8, also exhibits both D-gluco and L-gulo stereochemistries but is considered a D-glucose mimetic only. This is by virtue of the structural constraints enforced by the cyclic nature of IS8 leading to presentation of the structural motifs of D-glucose only. Note that in chemical terms IS7 and IS8 are distinct and cannot interconvert.

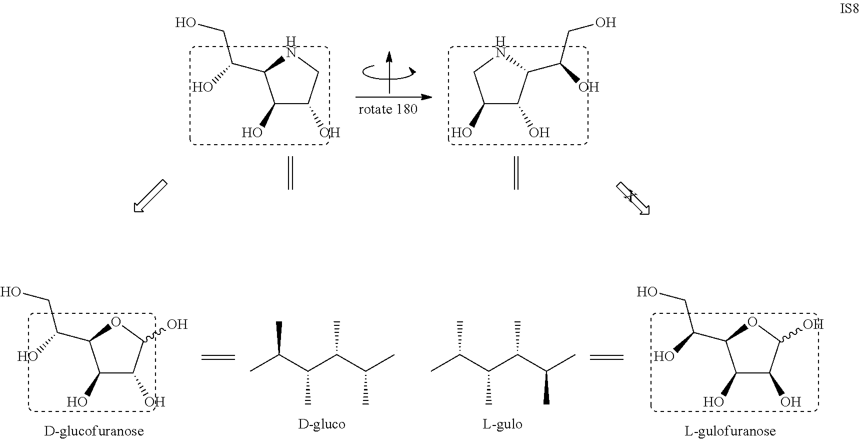

### (ii) Deoxysugar Mimetics and Further Substitution

Where an iminosugar mimics a deoxy sugar, this may also be considered as mimicry (albeit partial) of the cognate (fully oxygenated) monosaccharide. For example, the mimetic properties of iminosugar IS9 can be analysed as follows:

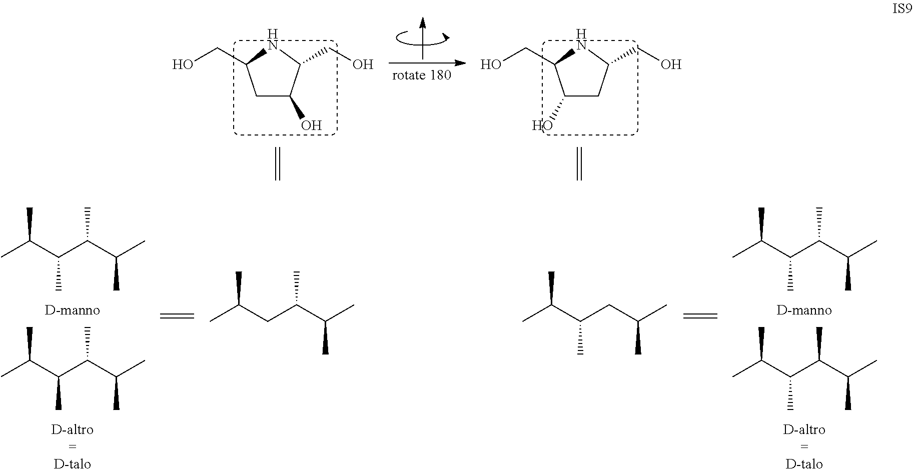

Moreover, replacement of hydroxyl groups with hydroxyl isosteres (e.g. similarly sized atoms or groups such as Me, Cl and F) may also generates iminosugars which are mimetics of a monosaccaride. For example, IS10 is a D-arabinofuranose mimetic, as shown below:

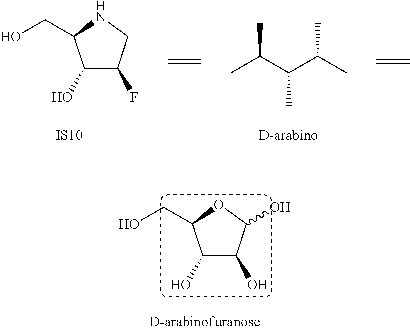

However, it should be noted that where the stereochemical configuration of the iminosugar matches one or more monosaccharides, but the group is not OH or an isostere (e.g. OBn, CO₂H or N₃) this would also be considered a mimetic for the purposes of the present invention. For example, the iminosugar IS11 is considered to be a mimetic of D-arabinofuranose, as shown below:

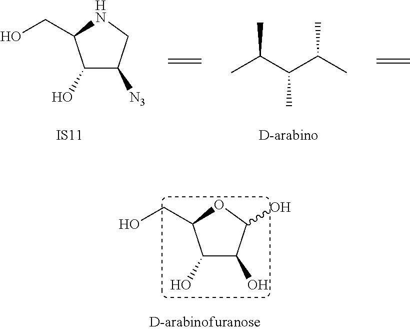

(iii) Quaternary Centres

Where these are present only the stereochemically defined groups on adjacent carbon atoms are considered when assigning matches, as shown below in the case of iminosugar IS12:

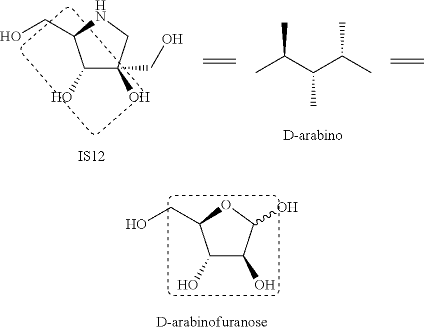

### (iv) Disaccharides and Oligosaccharides

Appropriately substituted iminosugars may also be considered as mimics of di- or oligosaccharides. In the case the same general principles described above are applied, with the caveat being that the iminosugar must contain two or more non-overlapping carbohydrate mimics.

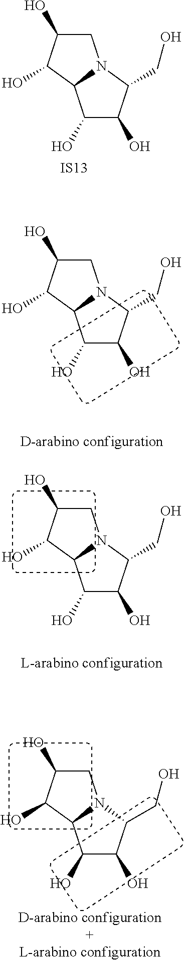

### (v) D- and L-Sugar Mimicry

Iminosugars may mimic either D- or L-forms of sugars. In the example below it can be seen that IS14 is a mimic of D-glucose, whereas its enantiomer IS15 is a mimic of L-glucose. This principle is generally applicable.

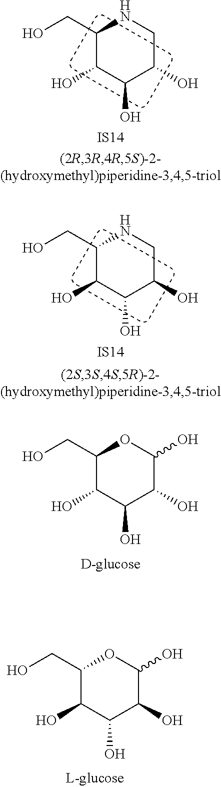

Thus, the iminosugars for use according to the invention may be of any structural class and/or subclass, including the classes and subclasses described above in Sections II(a) and II(b), and may be further characterized on the basis of the stereochemical configuration as follows:
Iminosugars of D- or L-gluco configuration;
Iminosugars of D- or L-galacto configuration;
Iminosugars of D- or L-manno configuration;
Iminosugars of D- or L-allo configuration;
Iminosugars of D- or L-altro configuration;
Iminosugars of D- or L-ido configuration;
Iminosugars of D- or L-gulo configuration;
Iminosugars of D- or L-talo configuration;
Iminosugars of D- or L-arabino configuration;
Iminosugars of D- or L-ribo configuration;
Iminosugars of D- or L-xylo configuration; and/or
Iminosugars of D- or L-lyxo configuration.

Alternatively, or in addition, the iminosugars for use according to the invention may be classified according to their stereochemical configuration in combination with other structural characteristics by reference to the sugars mimicked, as follows:
D- or L-glucose;
D- or L-galactose;
D- or L-mannose;
D- or L-allose;
D- or L-altrose;
D- or L-idose;
D- or L-gulose;
D- or L-talose;
D- or L-arabinose;
D- or L-ribose;
D- or L-deoxyribose;
D- or L-xylose;
D- or L-lyxose;
D- or L-psicose;
D- or L-fructose;
D- or L-sorbose;
D- or L-tagatose;
D- or L-ribulose;
D- or L-xylulose;
D- or L-fucose;
D- or L-fuculose;
D- or L-rhamnose;
D- or L-seduheptulose;
Sucrose;
Lactose;
Trehalose;
Maltose;
Acarbose;
Raffinose;
Melezitose;
Maltotriose;
Stachyose;
Glycogen;
Cellulose;
Chitin;
Starch;
Dextrin;
Glucan;
Glycosaminoglycans; and/or
Other oligosaccharides.

### B. Functional Considerations

The compounds for use according to the invention (including the compounds having the general formulae defined in section A(I) and the iminosugars described in section A(II), above) may have various functional properties. Any such functional properties may or may not contribute to the claimed in vivo activity, therapeutic activity or mode of action.

Thus, in some cases the compound for use according to the present invention may have one or more of the functional characteristics described below, wherein the functional characteristic(s) do not contribute to the claimed therapeutic activity and are purely incidental. In other cases, the compound for use according to the present invention may have one or more of the functional characteristics described below, wherein the functional characteristic(s) are responsible, wholly or partly, for the claimed therapeutic activity.

### (I) Glycosidase Ligands

The compounds for use according to the invention may act as a ligand for one or more enzyme(s) of the following glycosidase classes in vitro and/or in vivo:
α-glucosidases;
β-glucosidases;
α-galactosidases;
β-galactosidases;
α-mannosidases;
α-fucosidases; or
α-iduronidases; or
β-glucuronidases; or
β-mannosidases; or
hexosaminidases; or
α-N-acetylglucosaminidases; or
α-N-acetylgalactosaminidases; or
β-N-acetylglucosaminidases; or
β-N-acetylgalactosaminidases; or
sialidases; or
heparinases; or
neuraminidases; or
hyaluronidase; or
amylases; or
two or more of the foregoing enzyme classes.

The glycosidase ligands for use according to the invention may function as:
Inhibitors (competitive or non-competitive) of the target enzyme (e.g. by binding to the catalytic site of the enzyme);
Activators (e.g. by binding to an allosteric site of the enzyme);
Allosteric site ligands (e.g. acting as inhibitors or activators of enzyme activity);
Catalytic site ligands (e.g. acting as competitive inhibitor);
Pharmacoperones for the target enzyme, for example by binding to: (i) the catalytic site; (ii) an allosteric site; (iii), a site outside the catalytic site; and/or (d) a site outside an allosteric site (see also Section III, below); or
Two or more of the foregoing.

The compounds for use according to the invention preferably do not inhibit enzymes involved in metabolism of xenobiotics as this could lead to drug-drug interactions. Thus, the compounds of the invention preferably do not inhibit one or more of the following enzymes: CYP3A3/4 (most abundant isoenzyme in humans and responsible for metabolism of widest range of drugs), CYP1A, CYP2D6, CYP2C9/10 and CYP2C19.

The compounds for use according to the invention preferably do not inhibit digestive disaccharidases (unless such inhibition is desirable in order to, for example, modify sugar metabolism in the treatment of metabolic disorders).

Preferred compounds are glycosylation modulators. Glycosylation modulators may be identified by standard enzymological assays. Preferred are compounds which specifically inhibit ER α-glucosidases (for example, which specifically inhibit ER α-glucosidase I and/or ER α-glucosidase II, relative to other mammalian glycosidase enzymes). Most preferably, the compounds of the invention inhibit ER α-glucosidase I and/or ER α-glucosidase II with a degree of specificity such that gastrointestinal toxicity via disaccharidase inhibition on administration at antiviral concentrations in humans is absent (or present at clinically acceptable or subtoxic levels).

### (II) Glycosyltransferase Ligands

The compounds for use according to the invention may act as a ligand for a glycosyltransferase. Such compounds may act as a ligand for any glycosyltransferase, but preferred are compounds which are ligands for one or more enzyme(s) of the following glycosyltransferase enzyme classes in vitro and/or in vivo:
Fucosyltransferase;
Chitin synthetase;
Ceramide glucosyltransferase;
β-1,4-galactosyltransferase;
α-1,3-galactosyltransferase;
arabinofuranosyl transferase;
galactofuranosyltransferase; or
two or more of the foregoing enzyme classes.

The glycosyltransferase ligands for use according to the invention may function as:
Inhibitors (competitive or non-competitive) of the target enzyme (e.g. by binding to the catalytic site of the enzyme);
Activators (e.g. by binding to an allosteric site of the enzyme);
Allosteric site ligands (e.g. acting as inhibitors or activators of enzyme activity);
Catalytic site ligands (e.g. acting as competitive inhibitor);
Pharmacoperones for the target enzyme, for example by binding to: (i) the catalytic site; (ii) an allosteric site; (iii), a site outside the catalytic site; and/or (d) a site outside an allosteric site (see also Section III, below); or
Two or more of the foregoing.

### (III) Other Enzyme Ligands

The compounds for use according to the invention may act as a ligand for one or more enzyme(s) of the following classes in vitro and/or in vivo:
Matrix metalloproteinases;
Nucleoside processing enzymes;
UDP Gal mutases;
Glycogen phosphorylases;
ATPases;
GTPases;
Kinases (e.g. protein kinases, for example selected from serine/threonine specific, tyrosine specific, receptor tyrosine, histidine specific, aspartic acid/glutamic acid specific and mixed protein kinase classes);
Phosphatases;
Enzymes involved in nucleic acid synthesis; and
Two or more of the foregoing.

The above enzyme ligands for use according to the invention may function as:
Inhibitors (competitive or non-competitive) of the target enzyme (e.g. by binding to the catalytic site of the enzyme);
Activators (e.g. by binding to an allosteric site of the enzyme);
Allosteric site ligands (e.g. acting as inhibitors or activators of enzyme activity);
Catalytic site ligands (e.g. acting as competitive inhibitor);
Pharmacoperones for the target enzyme, for example by binding to: (i) the catalytic site; (ii) an allosteric site; (iii), a site outside the catalytic site; and/or (d) a site outside an allosteric site (see also Section III, below); or
Two or more of the foregoing.

The compounds for use according to the invention may act as a ligand for one or more G-protein coupled receptor(s) in vitro and/or in vivo.

### (IV) PRR Ligands

The innate immune response has evolved to recognize a few, highly conserved structures present in diverse groups of microorganisms. These highly conserve structures are known as pathogen-associated molecular patterns (PAMPs). They are recognized by a class of receptors known as pathogen-(or pattern-)recognition receptors (PRRs), which are expressed on various effector cells of the innate immune system, including the professional antigen-presenting cells, macrophages and dendritic cells.

The best-studied class of PRR is the Toll-like receptor class (TLRs). Mammalian TLRs comprise at least 10 members, designated TLR1-10, and may be expressed as homodimers or heterodimers (TLR1 plus TLR2 or TLR6 plus TLR2). It seems that different classes of pathogen are recognized by different TLRs. For example, TLR4 appears to be responsible for the detection of Gram-negative bacteria, its cognate PAMP being lipopolysaccharide (LPS). TLR2 appears to have several ligands, including peptidoglycan of Gram-positive bacteria, lipoproteins from Mycobacterium tuberculosis, and certain components of Saccharomyces cerevisiae zymosan, as well as highly purified Porphyromonas gingivalis LPS. TLR3 recognizes dsRNA, while TLR5 binds flagellin and TLR6 cooperates with TLR2 in detecting a subset of bacterial peptidoglycan. TLR7 can be triggered by imidazoquinolines, as well as ssRNA, and may thus be involved in the detection of viral infection. TLR9 detects bacterial and viral DNA sequences containing unmethylated cytosine-guanosine dinucleotides (CpGs). Other members of the mammalian TLR family may be specific for PAMPs characteristic of other classes of pathogens such as fungi (mannan, glucan and mycobacteria (via lipoarabinomannan and/or muramyldipeptide as cognate PAMPs)).

Another major class of PRR are the C-type lectins (reviewed by Figdor et al. (2002) Nat. Rev. Immunol. 2: 77-84). These PRRs share a conserved domain (the carbohydrate recognition domain or CRD) which was first characterized in animal lectins and which appears to function as a calcium-dependent carbohydrate-recognition domain. This consists of about 110 to 130 residues and contains four cysteines which are involved in two disulfide bonds. This domain may be present in multiple copies in some C-type lectin PRRs (for example, the mannose receptor contains eight CRDs).

Examples of C-type lectins include DC-SIGN (Dendritic Cell Specific ICAM-3 Grabbing Nonintegrin, or CD209), which can signal in response to Mycobacterium tuberculosis, synergising with LPS to induce IL-10 production by monocyte-derived DCs. The mannose receptor (MR) is involved in recognition of mycobacteria, fungi and protozoa. Dectin-1 acts as a PRR for β-glucan. Other C-type lectins are expressed in DCs (e.g. blood dendritic cell antigen-2 (BDCA-2), dendritic cell immunoactivating receptor (DCAR) and can also act as signalling receptors, though their role in PAMP recognition has yet to be established.

Preferred compounds for use according to the invention are PRR ligands (as defined herein). Such PRR ligands may be readily identified by screening assays which detect: (a) binding to a PRR (for example, TLR, C-type lectin or NOD-protein); and/or (b) the stimulation of PRR (for example, TLR, C-type lectin or NOD-protein) signalling. In the former case, the assays may involve competitive binding assays using an isolated PRR and a known cognate PAMP ligand as test reagents. Such competitive binding assays are routine in the art, and those skilled in the art will readily be able to identify appropriate conditions and formats for such assays. In the latter case, assays for PRR (for example C-type lectin) signalling activity may involve the use of PRR (for example C-type lectin)-bearing immune cells (typically DCs) as test reagent. Those skilled in the art will readily be able to identify appropriate conditions and formats for such assays, including inter alia the nature and number of the dendritic cells, the relative concentrations of compound and cells, the duration of stimulation with the compound and the methods used to detect signalling (for example by immunoassay for cytokine release).

The PRR ligands of the invention may bind any PRR, including any TLR, C-type lectin or NOD-protein. Preferably, the compounds for use according to the invention bind to PRRs displayed on/expressed by neutrophils, though they may bind to PRRs in, on or secreted by other cells including other cells of the innate immune system as well as to PRRs in, on or secreted by, for example, DCs, macrophages and/or T-cells.

### (a) NOD-Protein Ligands

The NOD-proteins (also known as the caterpillar family and NOD-LRR family) are cytosolic proteins that have a role in various innate and adaptive immune responses to cytosolic pathogens. Particularly preferred NOD-protein ligands for use according to the invention are NOD1 and/or NOD2 ligands. These latter proteins bind structures derived from peptidoglycan that are not TLR ligands.

NOD-protein PRRs comprise C-terminal leucine-rich repeats (LRRs), a central nucleotide-binding oligomerization domain (NOD), and N-terminal protein-protein interaction motifs, such as caspase recruitment domains (CARDs), pyrin domains or a TIR domain.

### (b) Toll-Like Receptor (TLR) Ligands

The PRR ligands of the invention may bind to any TLR receptor. Thus, the PRRs of the invention may bind to one or more of TLR1, TLR2, TLR3, TLR4, TLR5, TLR6, TLR7, TLR8, TLR9, TLR10 and TLR11.

Preferably, the TLR ligands for use according to the invention bind to:
(a) a TLR coupled with the MyD88 adaptor signalling pathway; and/or
(b) a TLR coupled with the TRIF adaptor signalling pathway; and/or
(c) a cell-surface TLR; and/or
(d) an endosomal TLR (e.g. TLR7, TLR8 and/or TLR9);
(e) an intracellular TLR (e.g. TLR3).

Particularly preferred are TLR9 or TLR4 ligands.

### (c) Lectin Ligands

As used herein, the term “lectin” defines a proteins which specifically binds (or crosslinks) a carbohydrate. Many lectins are multivalent carbohydrate-binding proteins or glycoproteins (excluding enzymes and antibodies). Preferred compounds for use according to the invention are ligands for C-type lectins. However, the compounds for use according to the invention may bind to any lectin, for example to any of the lectins described in Figdor et al. (2002) Nat. Rev. Immunol. 2: 77-84 (the disclosure of which relating to the identification of various lectins is incorporated herein by reference). Thus, the compounds of the invention may be ligands for type I and/or type II C-type lectins.

The compounds of the invention may be ligands for lectins selected from:
(a) MMR (CD206, macrophage mannose receptor); and/or
(b) DEC-205; and/or
(c) Dectin 1; and/or
(d) Dectin 2; and/or
(e) Langerin; and/or
(f) DC-SIGN; and/or
(g) BDCA-2; and/or
(h) DCIR; and/or
(i) DLEC; and/or
(j) CLEC; and/or
(k) a rhamnose-binding C-type lectin; and/or
(l) asialoglycoprotein receptor; and/or
(m) collectins; and/or
(n) selectins; and/or
(O) galectins; and/or
(p) annexins; and/or
(q) lecticans; and/or
(r) I-type lectins (for example, siglecs (sialic acid-binding immunoglobulin superfamily lectins); and/or
(s) P-type lectins.

The PRR or lectin (for example C-type lectin) ligands (as defined herein) may be identified by assays for PRR/lectin (for example C-type lectin) binding. These may involve competitive binding assays using an isolated PRR/lectin (for example C-type lectin) and a known cognate PAMP ligand as test reagents. Such competitive binding assays are routine in the art, and those skilled in the art will readily be able to identify appropriate conditions and formats for such assays.

### (V) Pharmacoperones

It has recently been discovered that certain small molecules can serve as molecular scaffolds and cause otherwise-misfolded mutant proteins to fold and route correctly within the cell. Such molecules have been dubbed “chemical chaperones”, “pharmaceutical chaperones”, “pharmacological chaperones” or “pharmacoperones”.

The term pharmacoperone is a term of art (from “pharmacological chaperone”) used to define a class of biologically active small molecules (sometimes also referred to in the art as “chemical chaperones”) that serve as molecular scaffolds, causing otherwise misfolded mutant proteins to fold and route correctly within the cell.

The compounds of the invention may be pharmacoperones as defined above.

In particular, it has been recognised that certain iminosugars can act as competitive inhibitors of the mutant enzymes implicated in various lysosomal storage disorders can, at subinhibitory concentrations, act as “Active-Site-Specific Chaperones” or ASSCs by either inducing or stabilizing the proper conformation of the mutant enzyme by specific binding to the catalytic site (see Fan (2007) Iminosugars as active-site-specific chaperones for the treatment of lysosomal storage disorders, in Iminosugars From Synthesis to Therapeutic Applications: Compain, Philippe/Martin, Olivier R. (eds.) ISBN-13: 978-0-470-03391-3-John Wiley & Sons, pages 225-247). Thus, the compounds for use according to the invention may be ASSCs as defined above.

### (VI) Immunomodulators

### (a) General Considerations

The compounds of the invention may be immunomodulatory. The term immunomodulatory is used in this context in relation to the compounds for use according to the invention to define a compound (e.g. a compound as described in section A(I) above or an iminosugar as described in Section A(II), above) which can stimulate and/or suppress one or more components or activities of the immune system (e.g. the mammalian immune system) in vivo or in vitro. Preferred immunomodulatory compounds for use according to the invention are capable of stimulating the activity of one or more cytokine(s) in a PRR-bearing cell. Such alkaloids are said to exhibit a cytokine stimulation profile in that PRR-bearing cell. Typically, the immunomodulatory alkaloids of the invention are capable of stimulating the activity of one or more cytokines in macrophages and/or dendritic cells. This stimulatory activity may be observable in vitro and/or in vivo. The stimulation may occur directly or indirectly via any mechanism and at any level (e.g. at the level of transcription, translation, post-translational modification, secretion, activation, shedding, stabilization or sequestration). Typically, the stimulation comprises an increase in the production of the cytokine(s) by the PRR-bearing cell. Typically, the one or more cytokine(s) stimulated by the immunomodulatory alkaloids for use according to the invention comprise one or more Th1 cytokines (as herein defined and described). Particularly preferred are immunomodulatory alkaloids that stimulate IL-2 and/or IL-12 in dendritic cells and/or macrophages (in vivo and/or in vitro).

Immunomodulatory compounds for use according to the invention may be readily identified by screening assays designed to detect the induction of one or more cytokine(s) (for example, IL-12 production in dendritic cells) in vitro. Such assays conveniently involve immune assays or microarray analysis (the latter being especially useful in embodiments where immunomodulatory compounds which stimulate a large number of different cytokines or which differentially stimulate a specific subclass of cytokines (e.g. Th1 cytokines) are to be selected). Those skilled in the art will readily be able to identify appropriate conditions for such assays, including inter alia the nature, source and number of the PRR-bearing cell (e.g. macrophages or dendritic cells), the relative concentrations of compound and cells, the duration of stimulation with the compound and the methods used to detect the induction of the cytokine(s).

Immunomodulatory activity may be determined by in vitro cytokine release assays (for example using one or more immune cells, e.g. macrophage, dendritic or spleen cells). Preferred immunomodulatory compounds of the invention stimulate the release of one or more cytokines (e.g. IL-12) in vitro (for example, in spleen cells, macrophages and/or dendritic cells). They may act as PRR ligands, a term used herein in relation to certain preferred compounds for use according to the invention to define compounds which can act as binding partners for a PRR. Such immunomodulatory compounds therefore include those which bind (or directly physically interact) with a PRR in vivo irrespective of the physiological consequences of that binding. Thus, the PRR ligands of the invention may bind a PRR as part of a cellular signalling cascade in which the PRR forms a part. Alternatively, they may bind PRR in the context of some other aspect of cellular physiology. In the latter case, the ligands may for example bind PRR at the cell surface without triggering a signalling cascade, in which case the binding may affect other aspects of cell function. Thus, the ligands of the invention may bind PRRs and thereby effect an increase in the concentration of functional PRR at the cell surface (for example mediated via an increase in PRR stability, absolute receptor numbers and/or PRR activity). Alternatively, the ligands may bind PRR (or PRR precursors) intracellularly, in which case they may act as molecular chaperones to increase the expression of active PRR.

### (b) PRR Agonists

In preferred embodiments, the PRR ligands of the invention are PRR agonists. The term agonist is used herein in relation to the PRR ligands of the invention to define a subclass of ligands which productively bind PRR to trigger the cellular signalling cascade of which the PRR forms a part.

As used herein, the term PRR-bearing cell defines any cell which expresses one or more pathogen-(or pattern-) recognition receptors (PRRs). The term PRR is a term of art used to define a class of receptors which are expressed on various cells (e.g. epithelial cells and effector cells of the innate immune system, including the professional antigen-presenting cells, macrophages and dendritic cells) and which recognize a few, highly conserved structures present in diverse groups of microorganisms known as pathogen-associated molecular patterns (PAMPs). Thus, PRR-bearing cells as described herein may comprise epithelial cells, macrophages, neutrophils, dendritic cells or other effector cells of the innate immune system. In preferred embodiments, the PRR-bearing cell for use in relation to the invention are dendritic cells and/or macrophages. Thus, those functional attributes of the immunomodulatory compounds of the invention that are defined by reference to inter alia a PRR-bearing cell are to be understood to relate to any of a wide variety of different PRR-bearing cells of diverse cytological properties and biological functions, including inter alia epithelial cells, dendritic cells, macrophages, various APCs, natural killer (NK) cells and other cells of the innate immune system (including e.g. neutrophils, granulocytes and monocytes). Preferably, however, the PRR-bearing cells described herein (and used for example to define a parameter of the reference conditions under which the functional properties of the immunomodulatory compound are manifest) are macrophages or dendritic cells.

The term cytokine stimulatory is used herein to define a subclass of immunomodulatory compounds for use according to the invention which are capable of stimulating the activity of one or more cytokine(s) in a PRR-bearing cell. Such compounds are said to exhibit a cytokine stimulation profile in that PRR-bearing cell. Typically, the immunomodulatory compounds of the invention are capable of stimulating the activity of one or more cytokines in macrophages and/or dendritic cells. This stimulatory activity may be observable in vitro and/or in vivo. The stimulation may occur directly or indirectly via any mechanism and at any level (e.g. at the level of transcription, translation, post-translational modification, secretion, activation, shedding, stabilization or sequestration). Preferred cytokine stimulatory compounds for use according to the invention are PRR ligands (as herein defined). Typically, the stimulation comprises an increase in the production of the cytokine(s) by the PRR-bearing cell. Typically, the one or more cytokine(s) stimulated by the immunomodulatory compounds for use according to the invention comprise one or more Th1 cytokines (as herein defined and described). Particularly preferred are immunomodulatory compounds that stimulate IL-2 and/or IL-12 in dendritic cells and/or macrophages (in vivo and/or in vitro).

Some iminosugars have immunomodulatory activity that is independent of any glycosidase inhibitory activity. Examples of such compounds are described, for example, in WO2004/064715, WO2005/070415 and WO2005/070418. It is thought that this immunomodulatory activity may arise from the stimulation of secretion of various cytokines (e.g. IL-12 and/or IL-2) by immune cells (e.g. dendritic cells and/or macrophages). As described in WO2004/064715, WO2005/070415 and WO2005/070418 (the content of which relating to the structure of the various compounds described and their biological activity is hereby incorporated herein by reference), the immunomodulatory activity of such compounds can itself confer antiviral activity.

(c) Cytokine stimulation

The compounds for use according to the invention may be cytokine stimulatory compounds capable of stimulating the activity of one or more cytokine(s) in a PRR-bearing cell. In preferred embodiments, the compound may stimulate one or more Th1 cytokine(s) in a PRR-bearing cell, for example IL-12 and/or IL-2.

IL-2 is a Th1 cytokine involved in mediating type-1 responses. It appears to be involved not only in T cell activation but also in the activation of inter alia NK cells, so functioning to regulate and link innate and adaptive immunity. Thus, the induced expression of IL-2 by the compounds for use according to the invention may directly potentiate a Th1 response and so increase the Th1:Th2 response ratio. The induced expression of IL-2 may also indirectly potentiate a Th1 response (and so increase the Th1:Th2 response ratio) by stimulating the activity of endogenous dendritic cells, which cells then trigger responses by other classes of lymphocytes (CTL, B, NK, and NKT cells) and also elicit T cell memory (a critical goal of vaccination).

The induced expression of IL-2 may also indirectly potentiate a Th1 response (and so increase the Th1:Th2 response ratio) by stimulating the activity of endogenous dendritic cells, which cells then trigger responses by other classes of lymphocytes (CTL, B, NK, and NKT cells) and also elicit T cell memory (a critical goal of vaccination).

The compounds for use according to the invention may stimulate the expression of IL-12 in PRR-bearing cells (for example in dendritic cells and/or macrophages). IL-12 is the primary mediator of type-1 immunity (the Th1 response). It induces natural killer (NK) cells to produce IFN-γ as part of the innate immune response and promotes the expansion of CD4⁺ Th1 cells and cytotoxic CD8⁺ cells which produce IFN-γ. It therefore increases T-cell invasion of tumours as well as the susceptibility of tumour cells to T-cell invasion.

Thus, without wishing to be bound by any theory, the immunomodulatory activity of certain preferred compounds for use according to the invention may arise from the stimulation of one or more cytokines (for example one or more Th1 cytokines, e.g. IL-12 and/or IL-2) in PRR-bearing cells (e.g. neutrophils, macrophages or dendritic cells). This leads to the stimulation of NK cells to produce IFN-γ and induces the development of CD4⁺ Th1 cells. The induced Th1 cells then produce IFN-γ and IL-2. The stimulated cytokine(s) (e.g. IL-12 and/or IL-2) then enhances further proliferation of Th1 cells and the differentiation of pathogen (e.g. tumour and virus)-specific CD8⁺ T cells. The cytokine(s) also stimulate the cytolytic activity of NK cells of the innate immune system.

The term cytokine stimulation profile is used herein to define a functional attribute of certain immunomodulatory compounds for use according to the invention which is characterized by reference to the identity of one or more cytokines stimulated (and optionally the identity of one or more cytokines unstimulated) in a PRR-bearing cell when contacted with the relevant immunomodulatory compound. Preferably, the cytokine stimulation profile is characterized by reference to the presence or absence of stimulation of two or more cytokines, more preferably four or more. Even more preferably, the cytokine stimulation profile is characterized by reference to the presence or absence of stimulation of one or more Th1 cytokines and/or one or more Th2 cytokines. Alternatively, or in addition, the stimulation profiles which functionally define the immunomodulatory compounds may be characterized by the degree of stimulation of one or more reference cytokine(s) (or classes thereof). The degree of stimulation may be expressed as an induction ratio with respect to: (a) the levels of the reference cytokine(s) (or markers thereof, such as encoding nucleic acids) in the PRR-bearing cell in the absence of the relevant test immunomodulatory compound; and/or (b) the level of one or more other cytokine(s) (or classes thereof) also present in the PRR-bearing cell (whether stimulated or not by the immunomodulatory compound). The cytokine stimulation profile of the immunomodulatory compounds for use according to the invention is preferably characterized by the stimulation of one or more Th1 cytokines (and optionally the absence of stimulation of one or more Th2 cytokines).

The term Th1 cytokine (or Type-1 cytokine) is a term of art used to define those cytokines produced by Th1 T-helper cells. Th1 cytokines include, for example, IL2, IFN-γ, IFN-α/β, IL12, IL-18, IL-27 and TNF-β. The term Th2 cytokine (or Type-2 cytokine) is a term of art used to define those cytokines produced by Th2 T-helper cells. Th2 cytokines include, for example, IL-4, IL-5, IL-9, IL-13, IL-25 and TSLP. The term Treg cytokine is a term of art used to define those cytokines produced by regulatory T-cells. Treg cytokines include, for example, IL-10, TGF-β and TSP1.

Immunomodulatory compounds for use according to the invention are preferably cytokine stimulatory compounds capable of stimulating the activity of one or more cytokine(s) in a PRR-bearing cell. In preferred embodiments, the compound may stimulate one or more Th1 cytokine(s) in a PRR-bearing cell, for example IL-12 and/or IL-2.

Immunomodulatory compounds for use according to the invention may also be able to reduce the overproduction of Th 1 cytokines such as IFN-γ via regulating production of IL-2 or IL-12 directly or by stimulating production of Th 2 cytokines such as IL-4. The compounds of the invention may also affect the production of glucosylated cytokines such as IFN-γ such that any overproduction is reduced or IFN-γ produced becomes less active or inactive as proposed for deoxynojirimycin and N-methyl-deoxynojirimycin in isolated splenocyte studies by Kosuge et al. (2000) Biol. Pharm. Bull. 23 (1): 1-5. Therapeutic improvements to iminosugars for therapeutic applications involving reduction of overproduction of IFN-γ would be increased glycosidase specificity to avoid inhibition of off-target glucosidases caused by DNJ and N-methyl-DNJ.

### (VII) Functional Sugar Mimicry

### (a) General Considerations

As described in Section A(II)(c) (above), the iminosugars for use according to the invention may be structural sugar mimetics and in many cases this structural mimicry is reflected in shared functional properties. Such functional sugar mimetics, as defined above, are compounds which share some or all of the functional properties of the sugar mimicked. For example, functional sugar mimetics may share some of the binding properties of the sugar mimicked in vivo (without necessarily sharing all of the attendant functional properties thereof).

Certain sugar mimetics may be identified by assays for saccharase inhibitory activity. Such enzyme assays are routine in the art, and those skilled in the art will readily be able to identify appropriate conditions and formats for such assays. For example, many polyhydroxylated iminosugars are potent and highly selective glycosidase inhibitors. These compounds can mimic the number, position and configuration of hydroxyl groups present in pyranosyl or furanosyl moieties and so bind to the active site of a cognate glycosidase, thereby inhibiting it. This area is reviewed in Legler (1990) Adv. Carbohydr. Chem. Biochem. 48: 319-384 and in Asano et al. (1995) J. Med. Chem. 38: 2349-2356.

In yet other embodiments, the functional sugar mimetic binds to a sugar receptor PRR. Such binding per se need not necessarily trigger a sugar receptor-mediated signalling pathway (i.e. initiate the cellular signalling cascade in which the sugar receptor forms a part): other co-stimulatory events may be required. Moreover, the binding may occur in the context of some other aspect of cellular physiology. In the latter case, the compounds of the invention may act as ligands as hereinbefore defined and may for example bind a sugar receptor at the cell surface without triggering a signalling cascade, in which case the binding may affect other aspects of cell function. Thus, the functional sugar mimetics of the invention may bind to a sugar receptor and thereby effect an increase in the concentration of functional sugar receptor at the cell surface (for example mediated via an increase in receptor stability, absolute receptor numbers and/or receptor activity). Alternatively, the function sugar mimetics may bind a sugar receptors (or a sugar receptor precursor) intracellularly, in which case they may act as molecular chaperones to increase the expression of active PRR.

### (b) Glucose Mimetics

The compounds for use according to the invention may be glucose mimetics. Such compounds may share some or all of the binding properties of glucose in vivo (without necessarily sharing all of the attendant functional properties thereof).

Such glucose mimetics may be identified by assays for glucosidase inhibitory activity. Such enzyme assays are routine in the art, and those skilled in the art will readily be able to identify appropriate conditions and formats for such assays.

Examples of such compounds are described in e.g. WO9929321 (the disclosure of which relating to specific piperidine iminosugars and their structure is hereby incorporated by reference). An example of such a glucose mimetic the iminosugar designated 1,5-dideoxy-1,5-imino-D-glucitol (alternately designated deoxynojirimycin), hereinafter “DNJ.” Numerous DNJ derivatives have been described. DNJ and its alkyl derivatives are potent inhibitors of the N-linked oligosaccharide processing enzymes, alpha-glucosidase I and alpha-glucosidase II (Saunier et al. (1982) J Biol Chem 257:14155-14161; Elbein (1987) Ann Rev Biochem 56:497534). These glucosidases are associated with the endoplasmic reticulum of mammalian cells. The N-butyl and N-nonyl derivatives of DNJ may also inhibit glucosyltransferases associated with the Golgi.

(c) Mannose and/or Rhamnose Mimetics

For example, the compounds of the invention may be mannose and/or rhamnose mimetics. Such compounds may share some or all of the binding properties of mannose and/or rhamnose in vivo (without necessarily sharing all of the attendant functional properties thereof).

Such sugar mimetics may be identified by assays for mannosidase and/or rhamnosidase inhibitory activity. Such enzyme assays are routine in the art, and those skilled in the art will readily be able to identify appropriate conditions and formats for such assays.

Thus, preferred rhamnose mimetics for use according to the invention are iminosugars which exhibit inhibitory activity against one or more rhamnosidase enzyme(s). Similarly, preferred mannose mimetics for use according to the invention are iminosugars which exhibit inhibitory activity against one or more mannosidase enzyme(s).

In yet other embodiments, preferred iminosugars may be rhamnose mimetics which bind to the rhamnose receptor PRR (see Grillon, Monsigny and Kieda (1990) Glycobiology 1(1): 33-8). Such binding per se need not necessarily trigger the rhamnose receptor-mediated signalling pathway (i.e. initiate the cellular signalling cascade in which the rhamnose receptor forms a part): other co-stimulatory events may be required. Moreover, the binding may occur in the context of some other aspect of cellular physiology. In the latter case, the iminosugars may act as ligands as hereinbefore defined and may for example bind rhamnose receptor at the cell surface without triggering a signalling cascade, in which case the binding may effect other aspects of cell function. Thus, the rhamnose mimetics of the invention may bind to the rhamnose receptor and thereby effect an increase in the concentration of functional rhamnose receptor at the cell surface (for example mediated via an increase in receptor stability, absolute receptor numbers and/or receptor activity). Alternatively, the rhamnose mimetics may bind rhamnose receptors (or rhamnose receptor precursors) intracellularly, in which case they may act as molecular chaperones to increase the expression of active PRR.

Similarly, other preferred iminosugars may be mannose mimetics which bind to the mannose receptor PRR. Again, such binding per se need not necessarily trigger the mannose receptor-mediated signalling pathway (i.e. initiate the cellular signalling cascade in which the mannose receptor forms a part): other co-stimulatory events may be required. Moreover, the binding may occur in the context of some other aspect of cellular physiology. In the latter case, the iminosugars may act as ligands as hereinbefore defined and may for example bind mannose receptor at the cell surface without triggering a signalling cascade, in which case the binding may effect other aspects of cell function. Thus, the mannose mimetics of the invention may bind to the mannose receptor and thereby effect an increase in the concentration of functional mannose receptor at the cell surface (for example mediated via an increase in receptor stability, absolute receptor numbers and/or receptor activity). Alternatively, the mannose mimetics may bind mannose receptors (or mannose receptor precursors) intracellularly, in which case they may act as molecular chaperones to increase the expression of active PRR.

### (VIII) Glycosylation Modulators, Alkovirs and Glycovirs

The compounds for use according to the invention may be glycosylation modulators, alkovirs and/or glycovirs, as hereinbefore defined.

Preferred glycosylation modulators can alter (e.g. eliminate, truncate, uncouple or debranch) N-linked or O-linked oligosaccharide structures on viral envelope glycoproteins. Preferred glycosylation modulators are glycosylation inhibitors. The glycosylation inhibitors of the invention may eliminate, truncate or debranch/uncouple oligosaccharide structures on viral envelope proteins.

The glycosylation modulators may modulate the activity of one or more glycosidase(s). Preferred are glycosylation inhibitors which inhibit the activity of one or more glycosidase(s). Particularly preferred are glycosylation modulators or inhibitors which modulate or inhibit the activity of glycosidase I (particularly glucosidase I).

Particularly preferred compounds are glycosylation inhibitors which are glycovirs, and more particularly glucovirs (as described and defined herein).

Glycosylation modulators may be identified by standard enzymological assay. Preferred are agents which specifically inhibit ER α-glucosidases (for example, which specifically inhibit ER α-glucosidase I and/or ER α-glucosidase II, relative to other mammalian glycosidase enzymes). Most preferably, the glycosylation modulators of the invention inhibit ER α-glucosidase I and/or ER α-glucosidase II with a degree of specificity such that gastrointestinal toxicity via disaccharidase inhibition on administration at antiviral concentrations in humans is absent (or present at clinically acceptable or subtoxic levels).

Preferred compounds for use according to the invention: (a) are glycosylation modulators as defined herein and described in the previous section; (b) are alkovirs, glycovirs or glucovirs as herein defined; and/or (c) have immunomodulatory activity (e.g. being an immunomodulatory or cytokine activating alkaloid as herein defined).

Glycosylation modulators, glucovirs and glycovirs may be identified by standard enzymological assay. Preferred are alkaloids which specifically inhibit ER α-glucosidases (for example, which specifically inhibit ER α-glucosidase I and/or ER α-glucosidase II, relative to other mammalian glycosidase enzymes). Most preferably, the compounds of the invention inhibit ER α-glucosidase I and/or ER α-glucosidase II with a degree of specificity such that gastrointestinal toxicity via disaccharidase inhibition on administration at antiviral concentrations in humans is absent (or present at clinically acceptable or subtoxic levels).

### (IX) Viral p7 Protein Inhibition and Ion Channel Interference

Alternatively, or in addition, the compounds may inhibit the activity of a viral p7 protein (for example, acting as viral ion channel blockers). Such compounds may be identified by the methods described for example in Pavlovic et al., (2003) Proc. Nat. Acad. Sci. 100(10): 6104-6108 (the relevant methodological disclosure of which is incorporated herein by reference).

In such embodiments, the compounds of the invention may not inhibit ER α-glucosidases at physiologically significant levels in vivo (and may not exhibit significant ER α-glucosidase I or II inhibitory activity in vitro). Indeed, in such embodiments the compounds of the invention may exhibit poor glucosidase inhibitory activity (relative to castanospermine and DNJ as reference glucosidase inhibitors) and may therefore exhibit levels of glucosidase inhibition which are so low as to permit viral glycoprotein processing on administration at antiviral concentrations in humans (the antiviral activity in such embodiments being mediated independently of glucosidase inhibition).

Without wishing to be bound by any theory, it is thought that antiviral activity in such embodiments of the invention may arise from: (a) direct interaction of the compounds of the invention with viral p7molecules, either blocking the p7-derived ion channels or preventing them from forming and/or opening; and/or (b) effecting a change to the membrane bilayer (for example by accumulating therein), so preventing p7 molecules from assembling into channel-forming pores.

In this embodiment, the invention finds particular application in the treatment or prevention of any infection mediated by p7-viroporin viruses, which include pestiviruses and hepaciviruses (so including the treatment or prevention of infections involving members of the genera Pestivirus and Hepacivirus, including the HCV and BVDV viruses, as discussed infra).

### (X) Other Activities

Alternatively, or in addition, the compounds may exert antiviral activity independently of α-glucosidase inhibition or p7 interference. For example, the compounds of the invention may exert an antiviral effect mediated by an immunomodulatory activity (as proposed in Mehta et al. (2004) Antimicrobial Agents and Chemotherapy 48(6): 2085-2090), for example by activating components of the innate immune system by a TLR-distinct or NF-κB-independent mechanism, by inducing interferon expression or by acting as interferon surrogates in vivo.

The compounds of the invention may exert an antiviral effect mediated by inhibition of other enzymes, for example viral enzymes involved or required for viral pathogenicity (for example neuraminidase).

### C. General Physicochemical Considerations

The compounds for use according to the invention (including the compounds having the general formulae defined in section A(I) and the iminosugars described in section A(II), above) may have various physicochemical properties.

The compounds for use according to the invention are preferably crystalline materials. Also preferred are compounds which are water soluble, or which are soluble in pharmaceutically acceptable excipients and formulations used in oral or i.v. administration (e.g. those described below). Also preferred are compounds which are subject to efficient passive or active transport to the desired site of action in vivo.

Preferred are iminosugars having a small molecular weight, since these may exhibit desirable pharmacokinetics. Thus, the iminosugar may have a molecular weight of 100 to 400 Daltons, preferably 150 to 300 Daltons and most preferably 200 to 250 Daltons.

Also preferred are non-metabolizable iminosugars. Such sugars may exhibit extended tissue residence durations, and so exhibit favourable pharmacokinetics.

### D. Specific Examples

Particular examples of compounds suitable for use according to the invention are listed in Table 1 (below). References to particular compound numbers herein refer to the numbers in this list.

| | | | Stereochemistry | | | | | | | | | | | |
|---|---|---|---|---|---|---|---|---|---|---|---|---|---|---|
| Compound # | Chemical Name | Compound Class | Allose | Altrose | Arabinose | Galactose | Glucose | Gulose | Idose | Lyxose | Mannose | Ribose | Talose | Xylose |
| 1 | (1R,2R,3S,6S,7R,7aS)- | pyrrolizidine | | y | | | y | y | y | | | | y | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 2 | (2R,3R,4R)-2- | pyrrolidine | | | y | | | | | y | | | | |
| | (hydroxymethyl)pyrrolidine- | | | | | | | | | | | | | |
| | 3,4-diol | | | | | | | | | | | | | |
| 3 | (2R,3R,4R,5S)-2- | piperidine | | | | | y | y | | | | | | |
| | (hydroxymethyl)-1- | | | | | | | | | | | | | |
| | methylpiperidine-3,4,5- | | | | | | | | | | | | | |
| | triol | | | | | | | | | | | | | |
| 4 | (3R,4R)-4-hydroxy-1,1- | pyrrolidine | | | | | | | | | | | | |
| | dimethylpyrrolidinium-3- | | | | | | | | | | | | | |
| | carboxylate | | | | | | | | | | | | | |
| 5 | (2R,3S,4S)-4-hydroxy- | pyrrolidine | | | | | | | | | | | | y |
| | 2-(4- | | | | | | | | | | | | | |
| | methoxybenzyl)pyrrolidin- | | | | | | | | | | | | | |
| | 3-yl acetate | | | | | | | | | | | | | |
| 6 | (2S,4R)-4-hydroxy-1,1- | pyrrolidine | | | | | | | | | | y | | y |
| | dimethylpyrrolidinium-2- | | | | | | | | | | | | | |
| | carboxylate | | | | | | | | | | | | | |
| 7 | (2S,3R,4R,5S)-3,4,5- | piperidine | | | | | y | y | | | | | | |
| | trihydroxypiperidine-2- | | | | | | | | | | | | | |
| | carboxylic acid | | | | | | | | | | | | | |
| 8 | (1R,5S,8R)-1,8- | other | | | y | | | | | y | | | | |
| | dihydroxy-6-oxa-3- | | | | | | | | | | | | | |
| | azabicyclo[3.2.1]octan- | | | | | | | | | | | | | |
| | 2-one | | | | | | | | | | | | | |
| 9 | (3R,4R,5S)-3- | piperidine | | | y | | | | | y | | | | |
| | (hydroxymethyl)piperidine- | | | | | | | | | | | | | |
| | 3,4,5-triol | | | | | | | | | | | | | |
| 10 | (1S,2R,3S,4R,5S)-8- | nortropane | | | | | | | | | | | | y |
| | methyl-8- | | | | | | | | | | | | | |
| | azabicyclo[3.2.1]octane- | | | | | | | | | | | | | |
| | 1,2,3,4-tetraol | | | | | | | | | | | | | |
| 11 | (2R,3R,4R,5R)-2-((R)- | pyrrolidine | | | | | | | | | y | | | |
| | 1,2-dihydroxyethyl)-5- | | | | | | | | | | | | | |
| | (hydroxymethyl)pyrrolidine- | | | | | | | | | | | | | |
| | 3,4-diol | | | | | | | | | | | | | |
| 12 | (1S,7S,8S,8aR)- | indolizidine | | | | y | | | | | y | | | |
| | octahydroindolizine- | | | | | | | | | | | | | |
| | 1,2,7,8-tetraol | | | | | | | | | | | | | |
| 13 | (2R,3R,4R,5R)-2,5- | pyrrolidine | | | | | | | | | y | | | |
| | bis(hydroxymethyl)pyrrolidine- | | | | | | | | | | | | | |
| | 3,4-diol | | | | | | | | | | | | | |
| 14 | (1R,2R,3R,5R,7aR)-3- | pyrrolizidine | | | | | | | | | y | | | |
| | (hydroxymethyl)-5- | | | | | | | | | | | | | |
| | methylhexahydro-1H- | | | | | | | | | | | | | |
| | pyrrolizine-1,2-diol | | | | | | | | | | | | | |
| 15 | (2R,3S,4R,5R,6R)-2,6- | piperidine | | | | | y | y | y | | | | | |
| | bis(hydroxymethyl)piperidine- | | | | | | | | | | | | | |
| | 3,4,5-triol | | | | | | | | | | | | | |
| 16 | (2R,3R,4S,5S,6R)-2- | piperidine | | | | | y | y | y | | | | | |
| | (hydroxymethyl)-6- | | | | | | | | | | | | | |
| | (((2R,3R,4S,5S,6R)- | | | | | | | | | | | | | |
| | 3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)methyl)piperidine- | | | | | | | | | | | | | |
| | 3,4,5-triol | | | | | | | | | | | | | |
| 17 | (1R,2R,3R,7S,7aS)-3- | pyrrolizidine | | | | | y | y | | | | | | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 18 | (3aR,3a1R,4R,5S,8aS)- | pyrrolizidine | | | | y | y | y | | | | | | |
| | 5-(hydroxymethyl)-2,2- | | | | | | | | | | | | | |
| | dimethylhexahydro- | | | | | | | | | | | | | |
| | 3aH-[1,3]dioxino[4,5,6- | | | | | | | | | | | | | |
| | gh]pyrrolizin-4-ol | | | | | | | | | | | | | |
| 19 | loline | pyrrolizidine | | | y | | | | | y | | | | |
| 20 | (1R,2S,3R,5R)-8- | nortropane | | | y | | | | | y | | | | |
| | azabicyclo[3.2.1]octane- | | | | | | | | | | | | | |
| | 1,2,3-triol | | | | | | | | | | | | | |
| 21 | (1R,2S,3R,4S,5R)-8- | nortropane | | | | | | | | | | | | y |
| | azabicyclo[3.2.1]octane- | | | | | | | | | | | | | |
| | 1,2,3,4-tetraol | | | | | | | | | | | | | |
| 22 | (1R,2R,3R,5S,7S,7aR)- | pyrrolizidine | | | | y | | | | | y | | | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 23 | (2S,3R,4S,5S,6S)-2- | piperidine | | | | | y | y | y | | | | | |
| | ethyl-6- | | | | | | | | | | | | | |
| | (hydroxymethyl)piperidine- | | | | | | | | | | | | | |
| | 3,4,5-triol | | | | | | | | | | | | | |
| 24 | (1S,2R,3R,5R,6S,7R,7aR)- | pyrrolizidine | | y | | y | y | y | | | | | y | |
| | 3-(hydroxymethyl)-5- | | | | | | | | | | | | | |
| | methylhexahydro-1H- | | | | | | | | | | | | | |
| | pyrrolizine-1,2,6,7- | | | | | | | | | | | | | |
| | tetraol | | | | | | | | | | | | | |
| 25 | (2R,3R,4R,5R)-1-(2- | pyrrolidine | | | | | | | | | y | | | |
| | hydroxyethyl)-2,5- | | | | | | | | | | | | | |
| | bis(hydroxymethyl)pyrrolidine- | | | | | | | | | | | | | |
| | 3,4-diol | | | | | | | | | | | | | |
| 26 | (2R,3R,4R,5R)-2-(3- | pyrrolidine | | | | | | | | | y | | | |
| | hydroxy-4- | | | | | | | | | | | | | |
| | methoxyphenyl)-5- | | | | | | | | | | | | | |
| | (hydroxymethyl)pyrrolidine- | | | | | | | | | | | | | |
| | 3,4-diol | | | | | | | | | | | | | |
| 27 | (2R,3R,4R,5R)-2- | pyrrolidine | | | | | | | | | y | | | |
| | (hydroxymethyl)-5-(4- | | | | | | | | | | | | | |
| | hydroxyphenyl)pyrrolidine- | | | | | | | | | | | | | |
| | 3,4-diol | | | | | | | | | | | | | |
| 28 | (1R,2R,3R,6S,7S,7aS)- | pyrrolizidine | | y | | | | | | | y | | y | |
| | 3-(hydroxymethyl)-6- | | | | | | | | | | | | | |
| | (3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)hexahydro-1H- | | | | | | | | | | | | | |
| | pyrrolizine-1,2,7-triol | | | | | | | | | | | | | |
| 29 | (2S,3S,4R)-1-(2- | pyrrolidine | | | | | | | | | | y | | |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 30 | (1R,2S,6R,7R,8R,8aR)- | indolizidine | | | | | | | | | y | | | |
| | octahydroindolizine- | | | | | | | | | | | | | |
| | 1,2,6,7,8-pentaol | | | | | | | | | | | | | |
| 31 | (1R,2R,3R,7aR)-3- | pyrrolizidine | | | | | | | | | y | | | |
| | (hydroxymethyl)-5- | | | | | | | | | | | | | |
| | (3,10,11- | | | | | | | | | | | | | |
| | trihydroxyundecyl)hexahydro- | | | | | | | | | | | | | |
| | 1H-pyrrolizine- | | | | | | | | | | | | | |
| | 1,2,6-triol | | | | | | | | | | | | | |
| 32 | (1S,6S,7R,8R,8aR)-8- | indolizidine | | | | | y | y | | | | | | |
| | (3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)octahydroindolizine- | | | | | | | | | | | | | |
| | 1,6,7-triol | | | | | | | | | | | | | |
| 33 | (1R,2S,3R,4S,5R,6R)-8- | nortropane | | | | | | | | | | | | y |
| | azabicyclo[3.2.1]octane- | | | | | | | | | | | | | |
| | 1,2,3,4,6-pentaol | | | | | | | | | | | | | |
| 34 | (2R,3R,4R,6R)-6-butyl- | piperidine | | | y | | | | | y | | | | |
| | 2- | | | | | | | | | | | | | |
| | (hydroxymethyl)piperidine- | | | | | | | | | | | | | |
| | 3,4-diol | | | | | | | | | | | | | |
| 35 | (1R,2R,3S,6S,7R,7aR)- | pyrrolizidine | | | | | y | y | | | y | | | |
| | 3- | | | | | | | | | | | | | |
| | (butyryloxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetrabutyrate | | | | | | | | | | | | | |
| 36 | (1S,2R,8R,8aR)- | indolizidine | | | | | | | | | y | | | |
| | octahydroindolizine- | | | | | | | | | | | | | |
| | 1,2,8-triol | | | | | | | | | | | | | |
| 37 | (1S,2R,6R,7S)- | pyrrolizidine | | y | | | | | | | y | | y | |
| | hexahydro-1H- | | | | | | | | | | | | | |
| | pyrrolizine-1,2,6,7- | | | | | | | | | | | | | |
| | tetraol | | | | | | | | | | | | | |
| 38 | (1R,2R,3S,6S,7R,7aR)- | pyrrolizidine | | | | | y | y | | | y | | | |
| | 7-amino-3- | | | | | | | | | | | | | |
| | (hydroxymethyl)hexahydro- | | | | | | | | | | | | | |
| | 1H-pyrrolizine-1,2,6- | | | | | | | | | | | | | |
| | triol | | | | | | | | | | | | | |
| 39 | (2R,3R,4R,5R)-2-((1R)- | pyrrolidine | | | | | | | | | y | | | |
| | 2-(3,4-dihydroxy-4- | | | | | | | | | | | | | |
| | (hydroxymethyl)tetrahydrofuran- | | | | | | | | | | | | | |
| | 2-yloxy)-1- | | | | | | | | | | | | | |
| | hydroxyethyl)-5- | | | | | | | | | | | | | |
| | (hydroxymethyl)pyrrolidine- | | | | | | | | | | | | | |
| | 3,4-diol | | | | | | | | | | | | | |
| 40 | (2R,3R,4R)-2- | piperidine | | | y | | | | | y | | | | |
| | (hydroxymethyl)piperidine- | | | | | | | | | | | | | |
| | 3,4-diol | | | | | | | | | | | | | |
| 41 | (1R,2S,6S,7R,8R,8aS)- | indolizidine | | | | | y | y | | | y | | | |
| | 2-(3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)octahydroindolizine- | | | | | | | | | | | | | |
| | 1,6,7,8-tetraol | | | | | | | | | | | | | |
| 42 | (2R,3R,4R,5R)-2-((Z)-5- | pyrrolidine | | | | | | | | | y | | | |
| | hydrazono-4- | | | | | | | | | | | | | |
| | iminopentyl)-5- | | | | | | | | | | | | | |
| | (hydroxymethyl)pyrrolidine- | | | | | | | | | | | | | |
| | 3,4-diol | | | | | | | | | | | | | |
| 43 | (1S,2R,3S,5R)-8- | nortropane | | | y | | | | | y | | | | |
| | azabicyclo[3.2.1]octane- | | | | | | | | | | | | | |
| | 1,2,3,6-tetraol | | | | | | | | | | | | | |
| 44 | (1S,3R,4R,5S)-8- | nortropane | | | y | | | | | y | | | | |
| | azabicyclo[3.2.1]octane- | | | | | | | | | | | | | |
| | 1,3,4-triol | | | | | | | | | | | | | |
| 45 | (4R,5R,6S)-4,5- | oxazilidine | | | y | | | | | y | | | | |
| | dihydroxy-6- | | | | | | | | | | | | | |
| | (hydroxymethyl)morpholin- | | | | | | | | | | | | | |
| | 2-ium | | | | | | | | | | | | | |
| 46 | (1S,6S,7S,8R)-1,7,8- | indolizidine | | | | | y | y | | | | | | |
| | trihydroxyoctahydroindolizin- | | | | | | | | | | | | | |
| | 6-yl butyrate | | | | | | | | | | | | | |
| 47 | (1R,2R,3R,6S,7S,7aR)- | pyrrolizidine | | y | | | | | | | y | | y | |
| | 3- | | | | | | | | | | | | | |
| | (acetoxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetraacetate | | | | | | | | | | | | | |
| 48 | (2R,3R,4S)-2-((R)-1,2- | pyrrolidine | | y | | | | | | | | | y | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 49 | (2R,3R,4R)-1-butyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 50 | 2-((2R,3R,4R)-3- | piperidine | | | y | | | | | y | | | | |
| | hydroxy-2- | | | | | | | | | | | | | |
| | (hydroxymethyl)piperidin- | | | | | | | | | | | | | |
| | 4-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 51 | (2R,3R,4R,5S,6R)-2- | piperidine | | | | | y | y | y | | | | | |
| | (hydroxymethyl)-6- | | | | | | | | | | | | | |
| | methylpiperidine-3,4,5- | | | | | | | | | | | | | |
| | triol | | | | | | | | | | | | | |
| 52 | (2R,3R,4S,5S)-2,5- | piperidine | | | | | | | | | y | | | |
| | bis(hydroxymethyl)piperidine- | | | | | | | | | | | | | |
| | 3,4,5-triol | | | | | | | | | | | | | |
| 53 | 2-((S)-2-((2S,3S,4S,5S)- | pyrrolidine | | | | | | | | | y | | | |
| | 3,4-dihydroxy-5- | | | | | | | | | | | | | |
| | (hydroxymethyl)pyrrolidin- | | | | | | | | | | | | | |
| | 2-yl)-2- | | | | | | | | | | | | | |
| | hydroxyethoxy)tetrahydro- | | | | | | | | | | | | | |
| | 2H-pyran-3,4,5-triol | | | | | | | | | | | | | |
| 54 | (1S,2R,3R,7aR)-3- | pyrrolizidine | | y | | | | | | | | | y | |
| | (hydroxymethyl)hexahydro- | | | | | | | | | | | | | |
| | 1H-pyrrolizine-1,2- | | | | | | | | | | | | | |
| | diol | | | | | | | | | | | | | |
| 55 | (1R,2R,3R,6S,7S,7aR)- | pyrrolizidine | | y | | | | | | | y | | y | |
| | 3-((3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)methyl)hexahydro- | | | | | | | | | | | | | |
| | 1H-pyrrolizine-1,2,6,7- | | | | | | | | | | | | | |
| | tetraol | | | | | | | | | | | | | |
| 56 | (1R,2R,3S,7S,7aR)- | pyrrolizidine | | | | y | | | | | y | | | |
| | 1,2,7- | | | | | | | | | | | | | |
| | trihydroxyhexahydro- | | | | | | | | | | | | | |
| | 1H-pyrrolizine-3- | | | | | | | | | | | | | |
| | carboxylic acid | | | | | | | | | | | | | |
| 57 | (2R,3S)-2- | pyrrolidine | | | | | | | | | | | | |
| | (hydroxymethyl)pyrrolidin- | | | | | | | | | | | | | |
| | 3-ol | | | | | | | | | | | | | |
| 58 | (3S,4S,5R,6S)-3,4,5- | piperidine | | | | | y | y | | | | | | |
| | trihydroxy-3,6- | | | | | | | | | | | | | |
| | bis(hydroxymethyl)piperidin- | | | | | | | | | | | | | |
| | 2-one | | | | | | | | | | | | | |
| 59 | (1S,2R,3R,5S,7aR)-5- | pyrrolizidine | | y | | | | | | | | | y | |
| | ((1R)-1,3- | | | | | | | | | | | | | |
| | dihydroxybutyl)-3- | | | | | | | | | | | | | |
| | (hydroxymethyl)hexahydro- | | | | | | | | | | | | | |
| | 1H-pyrrolizine-1,2- | | | | | | | | | | | | | |
| | diol | | | | | | | | | | | | | |
| 60 | (2S,3S,4S,5S)-2-(4- | pyrrolidine | | | | | | | | | y | | | |
| | aminopentyl)-5- | | | | | | | | | | | | | |
| | (hydroxymethyl)pyrrolidine- | | | | | | | | | | | | | |
| | 3,4-diol | | | | | | | | | | | | | |
| 61 | 4-((2S,3S,4R,5R)-3,4- | piperidine | | | | | y | y | | | | | | |
| | dihydroxy-2- | | | | | | | | | | | | | |
| | (hydroxymethyl)-5- | | | | | | | | | | | | | |
| | (3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)piperidin-1- | | | | | | | | | | | | | |
| | yl)butanoic acid | | | | | | | | | | | | | |
| 62 | (2R,3R,4R,5R)-2- | pyrrolidine | | | | | | | | | y | | | |
| | (hydroxymethyl)-5-((R)- | | | | | | | | | | | | | |
| | 1- | | | | | | | | | | | | | |
| | hydroxypropyl)pyrrolidine- | | | | | | | | | | | | | |
| | 3,4-diol | | | | | | | | | | | | | |
| 63 | (2R,3R,4R,5R)-2-((1R)- | pyrrolidine | | | | | | | | | y | | | |
| | 1,2-dihydroxypropyl)-5- | | | | | | | | | | | | | |
| | (hydroxymethyl)pyrrolidine- | | | | | | | | | | | | | |
| | 3,4-diol | | | | | | | | | | | | | |
| 64 | (2R,3R,4R,5R)-1-(2- | pyrrolidine | | | | | | | | | y | | | |
| | acetoxyethyl)-2,5- | | | | | | | | | | | | | |
| | bis(acetoxymethyl)pyrrolidine- | | | | | | | | | | | | | |
| | 3,4-diyl diacetate | | | | | | | | | | | | | |
| 65 | (2S,4R)-4-hydroxy-1- | pyrrolidine | | | | | | | | | | y | | y |
| | methylpyrrolidine-2- | | | | | | | | | | | | | |
| | carboxylic acid | | | | | | | | | | | | | |
| 66 | (1S,2R,3R,5R,6S,7aR)- | pyrrolizidine | | | | | | | | | y | | | |
| | 5-(3-hydroxybutyl)-3- | | | | | | | | | | | | | |
| | (hydroxymethyl)hexahydro- | | | | | | | | | | | | | |
| | 1H-pyrrolizine-1,2,6- | | | | | | | | | | | | | |
| | triol | | | | | | | | | | | | | |
| 67 | (1S,2R,3R,5S,7aR)-5- | pyrrolizidine | | | | | | | | | y | | | |
| | (3-hydroxybutyl)-3- | | | | | | | | | | | | | |
| | (hydroxymethyl)hexahydro- | | | | | | | | | | | | | |
| | 1H-pyrrolizine-1,2- | | | | | | | | | | | | | |
| | diol | | | | | | | | | | | | | |
| 68 | (1S,2R,3R,5S,7R,7aR)- | pyrrolizidine | | | | | y | y | | | y | | | |
| | 3,5- | | | | | | | | | | | | | |
| | bis(hydroxymethyl)hexahydro- | | | | | | | | | | | | | |
| | 1H-pyrrolizine- | | | | | | | | | | | | | |
| | 1,2,7-triol | | | | | | | | | | | | | |
| 69 | (2S,3S,4S,5R,6S)-2- | piperidine | | | | | y | y | y | | | | | |
| | (acetoxymethyl)-6- | | | | | | | | | | | | | |
| | ethylpiperidine-3,4,5- | | | | | | | | | | | | | |
| | triyl triacetate | | | | | | | | | | | | | |
| 70 | (2S,3R,4S)-2-((R)-1,2- | pyrrolidine | | | | | | | | | y | | | |
| | dihydroxyethyl)pyrrolidine- | | | | | | | | | | | | | |
| | 3,4-diol | | | | | | | | | | | | | |
| 71 | (2R,3S)-3-hydroxy-1,1- | pyrrolidine | | | | | | | | | | | | |
| | dimethylpyrrolidinium-2- | | | | | | | | | | | | | |
| | carboxylate | | | | | | | | | | | | | |
| 72 | (2S,3S,4S,5R)-2- | piperidine | | | | | y | y | | | | | | |
| | ethylpiperidine-3,4,5- | | | | | | | | | | | | | |
| | triol | | | | | | | | | | | | | |
| 73 | (2S,3S,4R)-1-benzyl-2- | pyrrolidine | y | | | | | | | | | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 74 | (2S,3S,4R)-1-butyl-2- | pyrrolidine | | | | | | | | | | | | |
| | (hydroxymethyl)pyrrolidine- | | | | | | | | | | | | | |
| | 3,4-diol | | | | | | | | | | | | | |
| 75 | (2S,3R,4S)-2-(1,2- | pyrrolidine | | | | | | | | | y | | | |
| | dihydroxypropyl)pyrrolidine- | | | | | | | | | | | | | |
| | 3,4-diol | | | | | | | | | | | | | |
| 76 | (2S,3S,4S,5S)-2-(3,6- | pyrrolidine | | | | | | | | | y | | | |
| | dihydroxyheptyl)-5- | | | | | | | | | | | | | |
| | (hydroxymethyl)pyrrolidine- | | | | | | | | | | | | | |
| | 3,4-diol | | | | | | | | | | | | | |
| 77 | (2S,3R,4R,5R,6R)-5- | piperidine | | | | y | | | | | y | | | |
| | (3,4-dihydroxy-2,5- | | | | | | | | | | | | | |
| | bis(hydroxymethyl)tetra | | | | | | | | | | | | | |
| | hydrofuran-2-yloxy)-2,6- | | | | | | | | | | | | | |
| | bis(hydroxymethyl)piperidine- | | | | | | | | | | | | | |
| | 3,4-diol | | | | | | | | | | | | | |
| 78 | (2R,3R,4R,5S)-2- | piperidine | | | | | y | y | | | | | | |
| | ((3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)methyl)piperidine- | | | | | | | | | | | | | |
| | 3,4,5-triol | | | | | | | | | | | | | |
| 79 | 2-((2R,3R,4R,5R)-4- | pyrrolidine | | | | | | | | | y | | | |
| | hydroxy-2,5- | | | | | | | | | | | | | |
| | bis(hydroxymethyl)pyrrolidin- | | | | | | | | | | | | | |
| | 3-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 80 | (2S,3R,4R)-3,4- | pyrrolidine | | | y | | | | | y | | | | |
| | dihydroxy-1,1- | | | | | | | | | | | | | |
| | dimethylpyrrolidinium-2- | | | | | | | | | | | | | |
| | carboxylate | | | | | | | | | | | | | |
| 81 | (1R,2R,3S,4R,6S,7R,7aR)- | pyrrolizidine | | | | | y | y | | | y | | | |
| | 1,2,6,7-tetrahydroxy- | | | | | | | | | | | | | |
| | 3- | | | | | | | | | | | | | |
| | (hydroxymethyl)octahydropyrrolizine | | | | | | | | | | | | | |
| | 4-oxide | | | | | | | | | | | | | |
| 82 | (2S,3R,4S,5R)-2,3- | piperidine | | | | y | | | | | | | | |
| | dimethylpiperidine- | | | | | | | | | | | | | |
| | 3,4,5-triol | | | | | | | | | | | | | |
| 83 | (3R,4R,5S)-3,4,5- | piperidine | | | y | | | | | y | | | | |
| | trihydroxy-3- | | | | | | | | | | | | | |
| | (hydroxymethyl)piperidin- | | | | | | | | | | | | | |
| | 2-one | | | | | | | | | | | | | |
| 84 | (3R,4S)-2,2- | pyrrolidine | | | y | | | | | y | | y | | |
| | bis(hydroxymethyl)pyrrolidine- | | | | | | | | | | | | | |
| | 3,4-diol | | | | | | | | | | | | | |
| 85 | (2S,4S)-4- | pyrrolidine | | | y | | | | | y | | | | |
| | (hydroxymethyl)-1- | | | | | | | | | | | | | |
| | methylpyrrolidine-2- | | | | | | | | | | | | | |
| | carboxylic acid | | | | | | | | | | | | | |
| 86 | (2R,3S,4R)-2-((S)-1,2- | pyrrolidine | | | | | | | | | y | | | |
| | dihydroxyethyl)-4- | | | | | | | | | | | | | |
| | methylpyrrolidine-3,4- | | | | | | | | | | | | | |
| | diol | | | | | | | | | | | | | |
| 87 | (3R,4R,5R)-3,4,5- | piperidine | | | y | | | | | y | | | | |
| | trihydroxypiperidine-3- | | | | | | | | | | | | | |
| | carboxylic acid | | | | | | | | | | | | | |
| 88 | (2R,3S,4S)-2-((S)-1- | pyrrolidine | | | | | y | y | y | | | | | |
| | hydroxyethyl)pyrrolidine- | | | | | | | | | | | | | |
| | 3,4-diol | | | | | | | | | | | | | |
| 89 | (3S,4R)-1-(allyloxy)-2,2- | pyrrolidine | | | y | | | | | y | | y | | |
| | bis(hydroxymethyl)pyrrolidine- | | | | | | | | | | | | | |
| | 3,4-diol | | | | | | | | | | | | | |
| 90 | N-((1S,7aR)-hexahydro- | pyrrolizidine | | | | | | | | | | | | |
| | 1H-pyrrolizin-1-yl)-2- | | | | | | | | | | | | | |
| | methylbutanamide | | | | | | | | | | | | | |
| 91 | (3S,4R)-2,2- | pyrrolidine | | | y | | | | | y | | y | | |
| | bis(hydroxymethyl)-1- | | | | | | | | | | | | | |
| | propoxypyrrolidine-3,4- | | | | | | | | | | | | | |
| | diol | | | | | | | | | | | | | |
| 92 | (2S,3S,4R)-2- | pyrrolidine | | | | | | | | | | y | | |
| | (hydroxymethyl)-2- | | | | | | | | | | | | | |
| | methylpyrrolidine-3,4- | | | | | | | | | | | | | |
| | diol | | | | | | | | | | | | | |
| 93 | (1R,2S,6S,8S,8aS)-6- | indolizidine | | | | | | | | | y | | | |
| | methyloctahydroindolizine- | | | | | | | | | | | | | |
| | 1,2,8-triol | | | | | | | | | | | | | |
| 94 | (2R,3R,4R)-3,4- | pyrrolidine | | | y | | | | | y | | | | |
| | dihydroxy-1-(2- | | | | | | | | | | | | | |
| | hydroxyethyl)-2- | | | | | | | | | | | | | |
| | (hydroxymethyl)pyrrolidine | | | | | | | | | | | | | |
| | 1-oxide | | | | | | | | | | | | | |
| 95 | (2R,3R,4R)-1-butyl-3,4- | pyrrolidine | | | y | | | | | y | | | | |
| | dihydroxy-2- | | | | | | | | | | | | | |
| | (hydroxymethyl)pyrrolidine | | | | | | | | | | | | | |
| | 1-oxide | | | | | | | | | | | | | |
| 96 | (2S,3R,4S)-1-butyl-2- | pyrrolidine | | | | | y | y | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 97 | (S)-1-((3aS,4R,6aS)-6a- | pyrrolidine | | | | | | | | | y | | | |
| | (hydroxymethyl)-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)ethane-1,2-diol | | | | | | | | | | | | | |
| 98 | (2R,3S,4S)-2-((S)-1,2- | pyrrolidine | | | | | | | | | y | | | |
| | dihydroxyethyl)-4- | | | | | | | | | | | | | |
| | (hydroxymethyl)pyrrolidine- | | | | | | | | | | | | | |
| | 3,4-diol | | | | | | | | | | | | | |
| 99 | (2S,3S,4R)-1-(2- | pyrrolidine | | | | | | | | | | y | | |
| | hydroxyethyl)-2- | | | | | | | | | | | | | |
| | (hydroxymethyl)-2- | | | | | | | | | | | | | |
| | methylpyrrolidine-3,4- | | | | | | | | | | | | | |
| | diol | | | | | | | | | | | | | |
| 100 | (2S,3R,4S,5R)-1-butyl- | piperidine | | | | y | | | | | | | | |
| | 2,3-dimethylpiperidine- | | | | | | | | | | | | | |
| | 3,4,5-triol | | | | | | | | | | | | | |
| 101 | N-((3R,5R)-1-benzyl-5- | pyrrolidine | y | y | | | | | | | | | y | |
| | ((S)-1,2- | | | | | | | | | | | | | |
| | dihydroxyethyl)pyrrolidin- | | | | | | | | | | | | | |
| | 3-yl)acetamide | | | | | | | | | | | | | |
| 102 | (3R,4S,5R)-5,6- | piperidine | | | y | | | | | y | | | | |
| | dimethyl-2,3,4,5- | | | | | | | | | | | | | |
| | tetrahydropyridine- | | | | | | | | | | | | | |
| | 3,4,5-triol | | | | | | | | | | | | | |
| 103 | (3R,4r,5S)-piperidine- | piperidine | | | | | | | | | | | | y |
| | 3,4,5-triol | | | | | | | | | | | | | |
| 104 | (1S,6S,7R,8R,8aR)- | indolizidine | | | | | y | y | | | | | | |
| | octahydroindolizine- | | | | | | | | | | | | | |
| | 1,6,7,8-tetraol | | | | | | | | | | | | | |
| 105 | (1R,2S,3S,4S,5R)-4- | nortropane | | | | | | | | | | | | y |
| | (3,4,5-trihydroxy-6- | | | | | | | | | | | | | |
| | (hydroxymethyl)tetrahydro- | | | | | | | | | | | | | |
| | 2H-pyran-2-yloxy)-8- | | | | | | | | | | | | | |
| | azabicyclo[3.2.1]octane- | | | | | | | | | | | | | |
| | 1,2,3-triol | | | | | | | | | | | | | |
| 106 | (1S,2S,3R,4S,5S)-5- | nortropane | | y | | | | | | | y | | y | |
| | methyl-8-oxa-6- | | | | | | | | | | | | | |
| | azabicyclo[3.2.1]octane- | | | | | | | | | | | | | |
| | 2,3,4-triol | | | | | | | | | | | | | |
| 107 | (7aS,7a1R,10aR,18aR, | pyrrolizidine | | y | | | y | y | | | | | y | |
| | 18bS)- | | | | | | | | | | | | | |
| | 2,2,6,6,13,13,17,17- | | | | | | | | | | | | | |
| | octamethyltetradecahydrobis[1, | | | | | | | | | | | | | |
| | 5]dioxecino[2,3- | | | | | | | | | | | | | |
| | b:2′,3′,4′-gh]pyrrolizine- | | | | | | | | | | | | | |
| | 4,15(7aH,7a1H)-dione | | | | | | | | | | | | | |
| 108 | (2S,3S,4R,5R,6R)-2- | piperidine | | | | | y | y | | | | | | |
| | butyl-6- | | | | | | | | | | | | | |
| | (hydroxymethyl)piperidine- | | | | | | | | | | | | | |
| | 3,4,5-triol | | | | | | | | | | | | | |
| 109 | (2R,3R,4S,5R)-2- | piperidine | | y | | | | | | | | | | y |
| | methylpiperidine-3,4,5- | | | | | | | | | | | | | |
| | triol | | | | | | | | | | | | | |
| 110 | 2-((2R,3R,4R,5S,6R)- | piperidine | | | | | y | y | y | | | | | |
| | 4,5-dihydroxy-2,6- | | | | | | | | | | | | | |
| | bis(hydroxymethyl)piperidin- | | | | | | | | | | | | | |
| | 3-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 111 | 1-((2S,3R,4R,5R)-3,4- | pyrrolidine | | | | | y | y | | | | | | |
| | dihydroxy-5- | | | | | | | | | | | | | |
| | (hydroxymethyl)pyrrolidin- | | | | | | | | | | | | | |
| | 2-yl)-2-methoxy-1H- | | | | | | | | | | | | | |
| | imidazole-4,5-diol | | | | | | | | | | | | | |
| 112 | (3S,4S,5R,6R)-3,4,5- | piperidine | | | | | | | | | y | | | |
| | trihydroxy-6- | | | | | | | | | | | | | |
| | (hydroxymethyl)piperidin- | | | | | | | | | | | | | |
| | 2-one | | | | | | | | | | | | | |
| 113 | 2-((1S,5R,6R,7R,7aS)- | pyrrolizidine | | | | y | | | | | y | | | |
| | 6,7-dihydroxy-5- | | | | | | | | | | | | | |
| | (hydroxymethyl)hexahydro- | | | | | | | | | | | | | |
| | 1H-pyrrolizin-1- | | | | | | | | | | | | | |
| | yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 114 | (2S,3S,4R,5R,6R)-2- | piperidine | | | | | y | y | | | | | | |
| | pentyl-6-((3,4,5- | | | | | | | | | | | | | |
| | trihydroxytetrahydro-2H- | | | | | | | | | | | | | |
| | pyran-2- | | | | | | | | | | | | | |
| | yloxy)methyl)piperidine- | | | | | | | | | | | | | |
| | 3,4,5-triol | | | | | | | | | | | | | |
| 115 | (2R,4R)-2- | piperidine | | | | | | | | | | y | | y |
| | carboxypiperidinium-4-yl | | | | | | | | | | | | | |
| | sulfate | | | | | | | | | | | | | |
| 116 | (1S,2R,3R,5R,7aR)-3,5- | pyrrolizidine | | y | | | | | | | | | y | |
| | bis(hydroxymethyl)hexahydro- | | | | | | | | | | | | | |
| | 1H-pyrrolizine- | | | | | | | | | | | | | |
| | 1,2-diol | | | | | | | | | | | | | |
| 117 | (2R,3R,4R,5R)-3,4- | pyrrolidine | | | | | | | | | y | | | |
| | dihydroxy-2-methyl-1- | | | | | | | | | | | | | |
| | oxo-5- | | | | | | | | | | | | | |
| | phenylpyrrolidinium | | | | | | | | | | | | | |
| 118 | (2R,3R,4R,5S)-1-butyl- | piperidine | | | | | y | y | | | | | | |
| | 2- | | | | | | | | | | | | | |
| | (hydroxymethyl)piperidine- | | | | | | | | | | | | | |
| | 3,4,5-triol | | | | | | | | | | | | | |
| 119 | (2S,3S,4S,5R)-2- | pyrrolidine | | | | | y | y | | | | | | |
| | (hydroxymethyl)-5- | | | | | | | | | | | | | |
| | methylpyrrolidine-3,4- | | | | | | | | | | | | | |
| | diol | | | | | | | | | | | | | |
| 120 | 2-((2R,3R,4R,5S)-3,5- | piperidine | | | | | y | y | | | | | | |
| | dihydroxy-2- | | | | | | | | | | | | | |
| | (hydroxymethyl)piperidin- | | | | | | | | | | | | | |
| | 4-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 121 | 2-((3S,4S,5R,6R)-4,5- | piperidine | | | | | y | y | | | | | | |
| | dihydroxy-6- | | | | | | | | | | | | | |
| | (hydroxymethyl)piperidin- | | | | | | | | | | | | | |
| | 3-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 122 | 2-((3S,4S,5R,6R)-4,5- | piperidine | | | | | y | y | | | | | | |
| | dihydroxy-6- | | | | | | | | | | | | | |
| | (hydroxymethyl)-1- | | | | | | | | | | | | | |
| | methylpiperidin-3- | | | | | | | | | | | | | |
| | yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 123 | 2-((3R,4R,5R)-4- | pyrrolidine | | | y | | | | | y | | | | |
| | hydroxy-5- | | | | | | | | | | | | | |
| | (hydroxymethyl)pyrrolidin- | | | | | | | | | | | | | |
| | 3-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 124 | (2R,3R,4R,5S,6R)-2,6- | piperidine | | | | | y | y | y | | | | | |
| | bis(hydroxymethyl)-1- | | | | | | | | | | | | | |
| | methylpiperidine-3,4,5- | | | | | | | | | | | | | |
| | triol | | | | | | | | | | | | | |
| 125 | (3aR,6S,7R,7aS)- | piperidine | | | | | | | y | | | | | |
| | hexahydrospiro[[1,3]dioxolo[4, | | | | | | | | | | | | | |
| | 5-b]pyridine-2,1′- | | | | | | | | | | | | | |
| | cyclohexane]-6,7-diol | | | | | | | | | | | | | |
| 126 | (3S)-2,3-dihydroxy-3- | pyrrolidine | | | | | | | | | y | | | |
| | ((2R,3R,4R)-2,3,4- | | | | | | | | | | | | | |
| | trihydroxypyrrolidin-2- | | | | | | | | | | | | | |
| | yl)propanoic acid | | | | | | | | | | | | | |
| 127 | (1R,2R,3S,7S,7aR)-3- | pyrrolizidine | | | | y | y | y | | | | | | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 128 | (1R,2R,3R,7S,7aR)-3- | pyrrolizidine | | | | y | | | | | y | | | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 129 | (1R,2R,3S,6S,7S,7aR)- | pyrrolizidine | | y | | | y | y | | | | | y | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 130 | (1S,2S,6S,7S,8S,8aS)- | indolizidine | | | | | y | y | | | y | | | |
| | octahydroindolizine- | | | | | | | | | | | | | |
| | 1,2,6,7,8-pentaol | | | | | | | | | | | | | |
| 131 | (1R,2R,3S,6R,7R,7aR)- | pyrrolizidine | | | | | y | y | | | | | | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 132 | (1R,2R,3R,6S,7S,7aR)- | pyrrolizidine | | y | | | | | | | y | | y | |
| | 3- | | | | | | | | | | | | | |
| | (butyryloxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetrabutyrate | | | | | | | | | | | | | |
| 133 | (2R,3S,4S,5S,6R)-2- | piperidine | | | | y | y | y | | | | | | |
| | (hydroxymethyl)-6- | | | | | | | | | | | | | |
| | methylpiperidine-3,4,5- | | | | | | | | | | | | | |
| | triol | | | | | | | | | | | | | |
| 134 | (2R,3R,4R,5R,6S)-2- | piperidine | | | | y | | | | | y | | | |
| | (hydroxymethyl)-6- | | | | | | | | | | | | | |
| | methylpiperidine-3,4,5- | | | | | | | | | | | | | |
| | triol | | | | | | | | | | | | | |
| 135 | (2S,3R,4S,5S)-2,5- | piperidine | | | | | y | y | | | | | | |
| | bis(hydroxymethyl)piperidine- | | | | | | | | | | | | | |
| | 3,4,5-triol | | | | | | | | | | | | | |
| 136 | (1R,2R,3S,6S,7S,7aR)- | pyrrolizidine | | y | | | y | y | | | | | y | |
| | 3- | | | | | | | | | | | | | |
| | (acetoxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetraacetate | | | | | | | | | | | | | |
| 137 | (1R,2R,3R,6S,7S,7aR)- | pyrrolizidine | | y | | | | | | | y | | y | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 138 | (1R,2R,3R,4S,5R)-8- | nortropane | | | y | | | | | y | | | | |
| | azabicyclo[3.2.1]octane- | | | | | | | | | | | | | |
| | 1,2,3,4-tetraol | | | | | | | | | | | | | |
| 139 | (1S,2R,3R,7S,7aR)-3- | pyrrolizidine | | y | | | y | y | | | | | y | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 140 | (2R,3R,4S)-2- | piperidine | | | | | | | | | | y | | |
| | (hydroxymethyl)piperidine- | | | | | | | | | | | | | |
| | 3,4-diol | | | | | | | | | | | | | |
| 141 | (1R,2S,3R,4R,5R)-8- | nortropane | | | y | | | | | y | | | | |
| | azabicyclo[3.2.1]octane- | | | | | | | | | | | | | |
| | 1,2,3,4-tetraol | | | | | | | | | | | | | |
| 142 | (2R,3R,4R)-1-(2- | pyrrolidine | | | y | | | | | y | | | | |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 143 | (1S,2R,3R,5R,7R,7aR)- | pyrrolizidine | | y | | | | | | | y | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 144 | (1R,2R,3S,6S,7R,7aR)- | pyrrolizidine | | | | | y | y | | | y | | | |
| | 3- | | | | | | | | | | | | | |
| | (acetoxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetraacetate | | | | | | | | | | | | | |
| 145 | (2R,3S,4R)-2-((S)-1,2- | pyrrolidine | | | | | | | | | y | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 146 | (1R,2R,3S,6S,7R,7aS)- | pyrrolizidine | | y | | | | | y | | | | y | |
| | 3- | | | | | | | | | | | | | |
| | (acetoxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetraacetate | | | | | | | | | | | | | |
| 147 | (1S,2S,3S,6R,7R,7aS)- | pyrrolizidine | | y | | | | | | | y | | y | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 148 | (1S,2S,3S,6S,7S,7aS)- | pyrrolizidine | | | | | y | y | | | y | | | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 149 | (1S,2R,3R,5S,7R,7aR)- | pyrrolizidine | | y | | | | | | | y | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 150 | (1S,2R,3R,5R,7aR)-3- | pyrrolizidine | | y | | | | | | | | | y | |
| | (hydroxymethyl)-5- | | | | | | | | | | | | | |
| | methylhexahydro-1H- | | | | | | | | | | | | | |
| | pyrrolizine-1,2-diol | | | | | | | | | | | | | |
| 151 | (1R,2S,3R,5R,7aR)-3- | pyrrolizidine | | y | | | | | | | | | y | |
| | (hydroxymethyl)-5- | | | | | | | | | | | | | |
| | methylhexahydro-1H- | | | | | | | | | | | | | |
| | pyrrolizine-1,2-diol | | | | | | | | | | | | | |
| 152 | (1S,2R,3R,5S,6R,7S,7aR)- | pyrrolizidine | y | y | | | | | | | y | | y | |
| | 3-(hydroxymethyl)-5- | | | | | | | | | | | | | |
| | methylhexahydro-1H- | | | | | | | | | | | | | |
| | pyrrolizine-1,2,6,7- | | | | | | | | | | | | | |
| | tetraol | | | | | | | | | | | | | |
| 153 | (1R,2S,8S,8aS)- | indolizidine | | | | | | | | | y | | | |
| | octahydroindolizine- | | | | | | | | | | | | | |
| | 1,2,8-triol | | | | | | | | | | | | | |
| 154 | (2R,3R,4S)-1-(2- | pyrrolidine | | | | | | | | | | y | | |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 155 | (2S,3R,4S)-2-((S)-1,2- | pyrrolidine | | | | | y | y | | | | | | |
| | dihydroxyethyl)pyrrolidine- | | | | | | | | | | | | | |
| | 3,4-diol | | | | | | | | | | | | | |
| 156 | (2S,3S,4R)-2-((R)-1,2- | pyrrolidine | y | | | | | | | | | | | |
| | dihydroxyethyl)pyrrolidine- | | | | | | | | | | | | | |
| | 3,4-diol | | | | | | | | | | | | | |
| 157 | (2S,3R,4R)-1-butyl-2- | pyrrolidine | | | | | | | | | | | | y |
| | (hydroxymethyl)pyrrolidine- | | | | | | | | | | | | | |
| | 3,4-diol | | | | | | | | | | | | | |
| 158 | (1R,2S,3R,5S,7S,7aR)- | pyrrolizidine | | y | | | | | | | | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 159 | (2S,3R,4S)-1-benzyl-2- | pyrrolidine | | | | | y | y | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 160 | (2S,3S,4S)-2- | pyrrolidine | | | y | | | | | y | | | | |
| | (hydroxymethyl)pyrrolidine- | | | | | | | | | | | | | |
| | 3,4-diol | | | | | | | | | | | | | |
| 161 | (2S,3S,4S)-1-(2- | pyrrolidine | | | y | | | | | y | | | | |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 162 | (2S,3R,4S)-1-butyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 163 | (2S,3S,4S)-1-butyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 164 | (1S,2R,3S,5S,7S,7aR)- | pyrrolizidine | | | | y | y | y | | | | | | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 165 | (1S,2R,3S,4S)-1-butyl- | pyrrolidine | | | y | | | | | y | | | | |
| | 3,4-dihydroxy-2- | | | | | | | | | | | | | |
| | (hydroxymethyl)pyrrolidine | | | | | | | | | | | | | |
| | 1-oxide | | | | | | | | | | | | | |
| 166 | (2S,3S,4R)-2- | pyrrolidine | | | | | | | | | | | y | |
| | (hydroxymethyl)pyrrolidine- | | | | | | | | | | | | | |
| | 3,4-diol | | | | | | | | | | | | | |
| 167 | (2S,3R,4S)-2- | pyrrolidine | | | y | | | | | y | | | | |
| | (hydroxymethyl)pyrrolidine- | | | | | | | | | | | | | |
| | 3,4-diol | | | | | | | | | | | | | |
| 168 | (2R,3R,4S,5R)-1-(2- | pyrrolidine | | y | | | | | | | | | | y |
| | hydroxyethyl)-2,5- | | | | | | | | | | | | | |
| | bis(hydroxymethyl)pyrrolidine- | | | | | | | | | | | | | |
| | 3,4-diol | | | | | | | | | | | | | |
| 169 | (1R,2R,3R,6S,7S,7aR)- | pyrrolizidine | | y | | | | | | | y | | | y |
| | 1,2,6,7-tetrahydroxy-3- | | | | | | | | | | | | | |
| | (hydroxymethyl)octahydropyrrolizine | | | | | | | | | | | | | |
| | 4-oxide | | | | | | | | | | | | | |
| 170 | (3R,4R,5R)-3,4,5- | piperidine | | | | | | | | | | | y | |
| | trihydroxy-3- | | | | | | | | | | | | | |
| | (hydroxymethyl)piperidin- | | | | | | | | | | | | | |
| | 2-one | | | | | | | | | | | | | |
| 171 | (2S,3R,4R)-2- | pyrrolidine | | | | | | | | | | | | y |
| | (hydroxymethyl)pyrrolidine- | | | | | | | | | | | | | |
| | 3,4-diol | | | | | | | | | | | | | |
| 172 | (1R,2S,3S,7S,7aR)-3- | pyrrolizidine | y | y | | | | | | | | | y | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 173 | (1R,2S,3R,5R,7S,7aR)- | pyrrolizidine | | y | | | | | | | | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 174 | (2S,4R)-4- | pyrrolidine | | | | | | | | | | y | | y |
| | (hydroxymethyl)-1- | | | | | | | | | | | | | |
| | methylpyrrolidine-2- | | | | | | | | | | | | | |
| | carboxylic acid | | | | | | | | | | | | | |
| 175 | (1R,2S,6R,8S,8aS)-6- | indolizidine | | | | | | | | | y | | | |
| | methyloctahydroindolizine- | | | | | | | | | | | | | |
| | 1,2,8-triol | | | | | | | | | | | | | |
| 176 | (2S,3R,4S)-2-((S)-1,2- | pyrrolidine | | | | | y | y | | | | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 177 | (1R,2R,3S,6S,7R,7aS)- | pyrrolizidine | | y | | | | | y | | | | y | |
| | 1,2,6,7-tetrahydroxy-3- | | | | | | | | | | | | | |
| | (hydroxymethyl)octahydropyrrolizine | | | | | | | | | | | | | |
| | 4-oxide | | | | | | | | | | | | | |
| 178 | (1R,2S,3R,4R)-1-butyl- | pyrrolidine | | | | | | | | | | | | y |
| | 3,4-dihydroxy-2- | | | | | | | | | | | | | |
| | (hydroxymethyl)pyrrolidine | | | | | | | | | | | | | |
| | 1-oxide | | | | | | | | | | | | | |
| 179 | (2S,3S,4S)-2-((S)-1,2- | pyrrolidine | | y | | | | | | | | | y | |
| | dihydroxyethyl)-4- | | | | | | | | | | | | | |
| | (hydroxymethyl)pyrrolidine- | | | | | | | | | | | | | |
| | 3,4-diol | | | | | | | | | | | | | |
| 180 | (2S,3S,4R,5S)-2,3- | piperidine | | y | | | | | | | | | y | |
| | dimethylpiperidine- | | | | | | | | | | | | | |
| | 3,4,5-triol | | | | | | | | | | | | | |
| 181 | (2R,3S,4R,5S)-2,3- | piperidine | | | | y | | | | | | | | |
| | dimethylpiperidine- | | | | | | | | | | | | | |
| | 3,4,5-triol | | | | | | | | | | | | | |
| 182 | (2R,3R,4S,5R)-2,3- | piperidine | | y | | | | | | | | | y | |
| | dimethylpiperidine- | | | | | | | | | | | | | |
| | 3,4,5-triol | | | | | | | | | | | | | |
| 183 | (2R,3R,4S,5R)-2,5- | pyrrolidine | | y | | | | | | | | | y | |
| | bis(hydroxymethyl)pyrrolidine- | | | | | | | | | | | | | |
| | 3,4-diol | | | | | | | | | | | | | |
| 184 | (2R,3R,4S,5R,6R)-2- | piperidine | | y | | | | | | | y | | y | |
| | (hydroxymethyl)-6- | | | | | | | | | | | | | |
| | methylpiperidine-3,4,5- | | | | | | | | | | | | | |
| | triol | | | | | | | | | | | | | |
| 185 | (1R,2R,3R,7R,7aR)-3- | pyrrolizidine | | y | | | | | | | y | | y | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 186 | (1S,6R,7R,8R,8aR)- | indolizidine | | | | y | | | | | y | | | |
| | octahydroindolizine- | | | | | | | | | | | | | |
| | 1,6,7,8-tetraol | | | | | | | | | | | | | |
| 187 | (2R,3R,4S,5S)-2- | piperidine | y | | | | | | | | | | | |
| | (hydroxymethyl)piperidine- | | | | | | | | | | | | | |
| | 3,4,5-triol | | | | | | | | | | | | | |
| 188 | (2R,3R,4R,5S,6S)-2- | piperidine | | | | | y | y | | | | | | |
| | (hydroxymethyl)-6- | | | | | | | | | | | | | |
| | methylpiperidine-3,4,5- | | | | | | | | | | | | | |
| | triol | | | | | | | | | | | | | |
| 189 | (2R,3S,5S,6R)-2,6- | piperidine | | | | y | y | y | | | | | | |
| | bis(hydroxymethyl)piperidine- | | | | | | | | | | | | | |
| | 3,4,5-triol | | | | | | | | | | | | | |
| 190 | (2R,3R,4R,5R)-2- | pyrrolidine | | | | | | | | | y | | | |
| | (hydroxymethyl)-5- | | | | | | | | | | | | | |
| | methylpyrrolidine-3,4- | | | | | | | | | | | | | |
| | diol | | | | | | | | | | | | | |
| 191 | (1R,2R,3R,5R,7R,7aR)- | pyrrolizidine | | y | | | | | | | y | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 192 | (1R,2R,3S,6S,7R,7aR)- | pyrrolizidine | | | | | y | y | | | y | | | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 193 | (2R,3R,4R,5S)-2- | piperidine | | | | | y | y | | | | | | |
| | (hydroxymethyl)piperidine- | | | | | | | | | | | | | |
| | 3,4,5-triol | | | | | | | | | | | | | |
| 194 | (2R,3R,4R,5R)-2-(2- | pyrrolidine | | | | | | | | | y | | | |
| | hydroxyethyl)-5- | | | | | | | | | | | | | |
| | (hydroxymethyl)pyrrolidine- | | | | | | | | | | | | | |
| | 3,4-diol | | | | | | | | | | | | | |
| 195 | (2S,3R,4R,5R)-2-(3- | pyrrolidine | | | | | y | y | | | | | | |
| | hydroxy-4- | | | | | | | | | | | | | |
| | methoxyphenyl)-5- | | | | | | | | | | | | | |
| | (hydroxymethyl)pyrrolidine- | | | | | | | | | | | | | |
| | 3,4-diol | | | | | | | | | | | | | |
| 196 | (2R,3R,4R,5S)-2- | pyrrolidine | | | | | y | y | | | | | | |
| | (hydroxymethyl)-5-(4- | | | | | | | | | | | | | |
| | hydroxyphenyl)pyrrolidine- | | | | | | | | | | | | | |
| | 3,4-diol | | | | | | | | | | | | | |
| 197 | (1R,2S,6S,7S,8R,8aR)- | indolizidine | | | | | y | y | | | y | | | |
| | 6- | | | | | | | | | | | | | |
| | methyloctahydroindolizine- | | | | | | | | | | | | | |
| | 1,2,6,7,8-pentaol | | | | | | | | | | | | | |
| 198 | (1R,2S,6R,7R,8R,8aR)- | indolizidine | | y | | | | | | | y | | y | |
| | 6- | | | | | | | | | | | | | |
| | methyloctahydroindolizine- | | | | | | | | | | | | | |
| | 1,2,6,7,8-pentaol | | | | | | | | | | | | | |
| 199 | (2S,3S,4R)-1-benzyl-2- | pyrrolidine | | | | | | | | | y | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 200 | (2S,3S,4R)-2-((R)-1,2- | pyrrolidine | | y | | | | | | | | | y | |
| | dihydroxyethyl)-4- | | | | | | | | | | | | | |
| | (hydroxymethyl)pyrrolidine- | | | | | | | | | | | | | |
| | 3,4-diol | | | | | | | | | | | | | |
| 201 | (2R,3R,4S,5R)-2- | piperidine | | | | | | | | | y | | | |
| | (hydroxymethyl)-5- | | | | | | | | | | | | | |
| | methylpiperidine-3,4,5- | | | | | | | | | | | | | |
| | triol | | | | | | | | | | | | | |
| 202 | (2S,3S,4S)-2,4- | pyrrolidine | | | | | | | | | | y | | |
| | bis(hydroxymethyl)pyrrolidine- | | | | | | | | | | | | | |
| | 3,4-diol | | | | | | | | | | | | | |
| 203 | (2S,3S,4S)-2-((S)-1,2- | pyrrolidine | | y | | | | | | | | | y | |
| | dihydroxyethyl)-4- | | | | | | | | | | | | | |
| | (hydroxymethyl)pyrrolidine- | | | | | | | | | | | | | |
| | 3,4-diol | | | | | | | | | | | | | |
| 204 | 3-((2R,3R,4R,5S)-3,4,5- | piperidine | | | | | y | y | | | | | | |
| | trihydroxy-2- | | | | | | | | | | | | | |
| | (hydroxymethyl)piperidin- | | | | | | | | | | | | | |
| | 1-yl)propanoic acid | | | | | | | | | | | | | |
| 205 | (2S,3R,4R,5S)-butyl 1- | piperidine | | | | | y | y | | | | | | |
| | butyl-3,4,5- | | | | | | | | | | | | | |
| | trihydroxypiperidine-2- | | | | | | | | | | | | | |
| | carboxylate | | | | | | | | | | | | | |
| 206 | (1S,6R,7R,7aS)-7- | pyrrolizidine | | | | y | | | | | | | | |
| | (methylamino)hexahydro- | | | | | | | | | | | | | |
| | 1H-pyrrolizine-1,6-diol | | | | | | | | | | | | | |
| 207 | 2-((2S,3S,4S,5S)-3,4- | pyrrolidine | | | | | | | | | y | | | |
| | dihydroxy-2,5- | | | | | | | | | | | | | |
| | bis(hydroxymethyl)pyrrolidin- | | | | | | | | | | | | | |
| | 1-yl)acetic acid | | | | | | | | | | | | | |
| 208 | (1R,2R)-1-((2R,3R,4S)- | pyrrolidine | | | | y | | | | | y | | | |
| | 3,4-dihydroxypyrrolidin- | | | | | | | | | | | | | |
| | 2-yl)propane-1,2,3-triol | | | | | | | | | | | | | |
| 209 | (2S,3R,4R,5S)-3,4,5- | piperidine | | | | | y | y | | | | | | |
| | trihydroxy-1-(2- | | | | | | | | | | | | | |
| | hydroxyethyl)piperidine- | | | | | | | | | | | | | |
| | 2-carboxylic acid | | | | | | | | | | | | | |
| 210 | 2-((2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl)acetic acid | | | | | | | | | | | | | |
| 211 | (2S,3S,4R)-2-((R)-1,2- | pyrrolidine | y | | | | | | | | | | | |
| | dihydroxyethyl)-4- | | | | | | | | | | | | | |
| | methylpyrrolidine-3,4- | | | | | | | | | | | | | |
| | diol | | | | | | | | | | | | | |
| 212 | (2S,3S,4R)-2- | pyrrolidine | | | | | | | | | | y | | |
| | (hydroxymethyl)-4- | | | | | | | | | | | | | |
| | methylpyrrolidine-3,4- | | | | | | | | | | | | | |
| | diol | | | | | | | | | | | | | |
| 213 | (1S,5R,8S)-6-oxa-3- | piperidine | | | y | | | | | y | | | | |
| | azabicyclo[3.2.1]octane- | | | | | | | | | | | | | |
| | 1,8-diol | | | | | | | | | | | | | |
| 214 | 2-((2R,3R,4R,5S)-3,4,5- | piperidine | | | | | y | y | | | | | | |
| | trihydroxy-2- | | | | | | | | | | | | | |
| | (hydroxymethyl)piperidin- | | | | | | | | | | | | | |
| | 1-yl)acetic acid | | | | | | | | | | | | | |
| 215 | (2R,3S,4R,5S)-2- | piperidine | | | | y | | | | | | | | |
| | (hydroxymethyl)piperidine- | | | | | | | | | | | | | |
| | 3,4,5-triol | | | | | | | | | | | | | |
| 216 | (2S,3S,4S,5R)-2- | piperidine | | | | | y | y | | | | | | |
| | (hydroxymethyl)piperidine- | | | | | | | | | | | | | |
| | 3,4,5-triol | | | | | | | | | | | | | |
| 217 | (3aS,4R,6aR)—N-benzyl- | pyrrolidine | | | | | | | | | | y | | |
| | 2,2,4- | | | | | | | | | | | | | |
| | trimethyltetrahydro-3aH- | | | | | | | | | | | | | |
| | [1,3]dioxolo[4,5- | | | | | | | | | | | | | |
| | c]pyrrole-4-carboxamide | | | | | | | | | | | | | |
| 218 | (2R,3S,4R)—N-benzyl- | pyrrolidine | | | | | | | | | | y | | |
| | 3,4-dihydroxy-2- | | | | | | | | | | | | | |
| | methylpyrrolidine-2- | | | | | | | | | | | | | |
| | carboxamide | | | | | | | | | | | | | |
| 219 | (3R,4S,5S)-5- | piperidine | | | y | | | | | y | | | | |
| | (hydroxymethyl)piperidine- | | | | | | | | | | | | | |
| | 3,4-diol | | | | | | | | | | | | | |
| 220 | (2S,3S,4R)-1-butyl-2- | pyrrolidine | | | | | | | | | | y | | |
| | (hydroxymethyl)-2- | | | | | | | | | | | | | |
| | methylpyrrolidine-3,4- | | | | | | | | | | | | | |
| | diol | | | | | | | | | | | | | |
| 221 | (2S,3S,4S,5R)-2- | piperidine | | | | | y | y | | | | | | |
| | (hydroxymethyl)piperidine- | | | | | | | | | | | | | |
| | 3,4,5-triol | | | | | | | | | | | | | |
| 222 | (2R,3R,4R,5R)-2-(3,4- | pyrrolidine | | | | | | | | | y | | | |
| | dimethoxyphenyl)-5- | | | | | | | | | | | | | |
| | (hydroxymethyl)pyrrolidine- | | | | | | | | | | | | | |
| | 3,4-diol | | | | | | | | | | | | | |
| 223 | (2S,3R,4S,5R)-2- | piperidine | | | | y | | | | | | | | |
| | (hydroxymethyl)piperidine- | | | | | | | | | | | | | |
| | 3,4,5-triol | | | | | | | | | | | | | |
| 224 | (3R,4R,5R,6S)-2,2- | azepane | | | | | y | y | | | | | | |
| | bis(hydroxymethyl)azepane- | | | | | | | | | | | | | |
| | 3,4,5,6-tetraol | | | | | | | | | | | | | |
| 225 | 1-hydroxy-13- | pyrrolidine | | y | | | | | | | | | y | |
| | ((2R,3R,4S,5R)-4- | | | | | | | | | | | | | |
| | hydroxy-5- | | | | | | | | | | | | | |
| | (hydroxymethyl)-3- | | | | | | | | | | | | | |
| | (3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)pyrrolidin-2- | | | | | | | | | | | | | |
| | yl)tridecan-5-one | | | | | | | | | | | | | |
| 226 | (R)-13-((2R,3R,4R,5R)- | pyrrolidine | | | | | | | | | y | | | |
| | 3,4-dihydroxy-5- | | | | | | | | | | | | | |
| | (hydroxymethyl)pyrrolidin- | | | | | | | | | | | | | |
| | 2-yl)-1,13- | | | | | | | | | | | | | |
| | dihydroxytridecan-5-one | | | | | | | | | | | | | |
| 227 | (2R,3S,4R,5S)-2- | piperidine | | | | | | | | | y | | | |
| | (aminomethyl)piperidine- | | | | | | | | | | | | | |
| | 3,4,5-triol | | | | | | | | | | | | | |
| 228 | (4S,5S)-phenyl 3- | piperidine | | | y | | | | | y | | | | |
| | bromo-4,5- | | | | | | | | | | | | | |
| | dihydroxypiperidine-1- | | | | | | | | | | | | | |
| | carboxylate | | | | | | | | | | | | | |
| 229 | (2R,3R,4R,5R)-2- | piperidine | | | | | | | | | y | | | |
| | (hydroxymethyl)piperidine- | | | | | | | | | | | | | |
| | 3,4,5-triol | | | | | | | | | | | | | |
| 230 | (4S,5S)-phenyl 3- | piperidine | | | | | | | | | | y | | |
| | bromo-4,5- | | | | | | | | | | | | | |
| | dihydroxypiperidine-1- | | | | | | | | | | | | | |
| | carboxylate | | | | | | | | | | | | | |
| 231 | (3R,4s,5S)-1- | piperidine | | | | | | | | | | y | | |
| | nonylpiperidine-3,4,5- | | | | | | | | | | | | | |
| | triol | | | | | | | | | | | | | |
| 232 | (3R,4r,5S)-1- | piperidine | | | | | | | | | | | | y |
| | butylpiperidine-3,4,5- | | | | | | | | | | | | | |
| | triol | | | | | | | | | | | | | |
| 233 | (3aS,4R,8R,8aS)-4,8- | azepane | | | | y | | | | | | | | |
| | dihydroxy-2,2- | | | | | | | | | | | | | |
| | dimethyltetrahydro-3aH- | | | | | | | | | | | | | |
| | [1,3]dioxolo[4,5- | | | | | | | | | | | | | |
| | d]azepin-5(4H)-one | | | | | | | | | | | | | |
| 234 | (2S,3S,4S,5S)-4,5- | piperidine | | | | y | | | | | | | | |
| | bis(tert- | | | | | | | | | | | | | |
| | butyldimethylsilyloxy)-2- | | | | | | | | | | | | | |
| | ((tert- | | | | | | | | | | | | | |
| | butyldimethylsilyloxy)methyl)piperidin-3-ol | | | | | | | | | | | | | |
| 235 | 1-((2S,3S,4S)-2-((S)- | pyrrolidine | | | | y | | | | | | | | |
| | 1,2-dihydroxyethyl)-3,4- | | | | | | | | | | | | | |
| | dihydroxypyrrolidin-1- | | | | | | | | | | | | | |
| | yl)ethanone | | | | | | | | | | | | | |
| 236 | (2S,3R)-3,4- | pyrrolidine | | | y | | | | | y | | | | |
| | dihydroxypyrrolidine-2- | | | | | | | | | | | | | |
| | carboxylic acid | | | | | | | | | | | | | |
| 237 | (3aR,6S,7S,7aR)-7- | piperidine | | | | | | | | | y | | | |
| | hydroxy-2,2,6- | | | | | | | | | | | | | |
| | trimethyltetrahydro- | | | | | | | | | | | | | |
| | [1,3]dioxolo[4,5- | | | | | | | | | | | | | |
| | c]pyridin-4(3aH)-one | | | | | | | | | | | | | |
| 238 | (3aS,6R,7R,7aS)-6- | piperidine | | | | | | | | | y | | | |
| | ((tert- | | | | | | | | | | | | | |
| | butyldimethylsilyloxy)methyl)- | | | | | | | | | | | | | |
| | 7-hydroxy-2,2- | | | | | | | | | | | | | |
| | dimethyltetrahydro- | | | | | | | | | | | | | |
| | [1,3]dioxolo[4,5- | | | | | | | | | | | | | |
| | c]pyridin-4(3aH)-one | | | | | | | | | | | | | |
| 239 | (S)-1-((2S,3S,4S)-3,4- | pyrrolidine | | | | y | | | | | | | | |
| | bis(benzyloxy)pyrrolidin- | | | | | | | | | | | | | |
| | 2-yl)ethane-1,2-diol | | | | | | | | | | | | | |
| 240 | 1-((3aS,4S,8R,8aS)-8- | azepane | | y | | | | | | | | | y | |
| | hydroxy-4,7-anhydro- | | | | | | | | | | | | | |
| | 2,2,4-trimethyl-3aH- | | | | | | | | | | | | | |
| | [1,3]dioxolo[4,5- | | | | | | | | | | | | | |
| | c]azepin- | | | | | | | | | | | | | |
| | 5(4H,6H,7H,8H,8aH)- | | | | | | | | | | | | | |
| | yl)ethanone | | | | | | | | | | | | | |
| 241 | (3aS,7R,8R,8aS)-7,8- | azepane | | | | | | | | | y | | | |
| | dihydroxy-2,2- | | | | | | | | | | | | | |
| | dimethyltetrahydro-3aH- | | | | | | | | | | | | | |
| | [1,3]dioxolo[4,5- | | | | | | | | | | | | | |
| | c]azepin-4(5H)-one | | | | | | | | | | | | | |
| 242 | (3S,4S,5R)-3,4- | pyrrolidine | | | | | y | y | | | | | | |
| | bis(benzyloxy)-5-((S)- | | | | | | | | | | | | | |
| | 1,2- | | | | | | | | | | | | | |
| | dihydroxyethyl)pyrrolidin- | | | | | | | | | | | | | |
| | 2-one | | | | | | | | | | | | | |
| 243 | (3S,4S,5R,6R)-3,4,5- | piperidine | | | | | | | | | y | | | |
| | trihydroxy-6- | | | | | | | | | | | | | |
| | methylpiperidin-2-one | | | | | | | | | | | | | |
| 244 | (3aR,4S,7R,7aS)-6- | piperidine | | | | y | y | y | | | | | | |
| | (hydroxymethyl)-2,2,4- | | | | | | | | | | | | | |
| | trimethylhexahydro- | | | | | | | | | | | | | |
| | [1,3]dioxolo[4,5- | | | | | | | | | | | | | |
| | c]pyridin-7-ol | | | | | | | | | | | | | |
| 245 | (2R,3S,4R,5S,6R)—N- | piperidine | | y | | | y | y | | | | | y | |
| | butyl-3,4,5-trihydroxy-6- | | | | | | | | | | | | | |
| | methylpiperidine-2- | | | | | | | | | | | | | |
| | carboxamide | | | | | | | | | | | | | |
| 246 | (2S,3R,4S,5R,6R)- | piperidine | | y | | | | | | | y | | y | |
| | 3,4,5-trihydroxy-6- | | | | | | | | | | | | | |
| | (hydroxymethyl)-N- | | | | | | | | | | | | | |
| | methylpiperidine-2- | | | | | | | | | | | | | |
| | carboxamide | | | | | | | | | | | | | |
| 247 | (2R,3S,4R,5S,6R)—N- | piperidine | | y | | | y | y | | | | | y | |
| | benzyl-3,4,5-trihydroxy- | | | | | | | | | | | | | |
| | 6-methylpiperidine-2- | | | | | | | | | | | | | |
| | carboxamide | | | | | | | | | | | | | |
| 248 | (2S,3R,4S,5R,6R)- | piperidine | | y | | | | | | | y | | y | |
| | 3,4,5-trihydroxy-6- | | | | | | | | | | | | | |
| | (hydroxymethyl)piperidine- | | | | | | | | | | | | | |
| | 2-carboxylic acid | | | | | | | | | | | | | |
| 249 | (2R,3S,4R,5S,6R)- | piperidine | | y | | | y | y | | | | | y | |
| | 3,4,5-trihydroxy-N,6- | | | | | | | | | | | | | |
| | dimethylpiperidine-2- | | | | | | | | | | | | | |
| | carboxamide | | | | | | | | | | | | | |
| 250 | methyl 2-((7R)-7- | piperidine | | | | | | | | | y | | | |
| | hydroxy-2,2-dimethyl-4- | | | | | | | | | | | | | |
| | oxohexahydro- | | | | | | | | | | | | | |
| | [1,3]dioxolo[4,5- | | | | | | | | | | | | | |
| | c]pyridin-6-yl)acetate | | | | | | | | | | | | | |
| 251 | (3aS,4R,8R,8aR,8bS)- | pyrrolizidine | | y | | | | | | | y | | y | |
| | 4-(benzyloxymethyl)-8- | | | | | | | | | | | | | |
| | hydroxy-2,2- | | | | | | | | | | | | | |
| | dimethyltetrahydro-3aH- | | | | | | | | | | | | | |
| | [1,3]dioxolo[4,5- | | | | | | | | | | | | | |
| | a]pyrrolizin-6(4H)-one | | | | | | | | | | | | | |
| 252 | (2S,3R,4R)-1-butyl-2- | piperidine | | | y | | | | | y | | | | |
| | (hydroxymethyl)piperidine- | | | | | | | | | | | | | |
| | 3,4-diol | | | | | | | | | | | | | |
| 253 | (3R,4r,5S)-1- | piperidine | | | | | | | | | | | | y |
| | methylpiperidine-3,4,5- | | | | | | | | | | | | | |
| | triol | | | | | | | | | | | | | |
| 254 | (3R,4r,5S)-1- | piperidine | | | | | | | | | | | | y |
| | nonylpiperidine-3,4,5- | | | | | | | | | | | | | |
| | triol | | | | | | | | | | | | | |
| 255 | (2S,3S,4S)-1-benzyl-2- | pyrrolidine | | | | y | | | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 256 | (2S,5S)-2- | piperidine | | y | | | | | | | y | | y | |
| | (hydroxymethyl)-6- | | | | | | | | | | | | | |
| | methylpiperidine-3,4,5- | | | | | | | | | | | | | |
| | triol | | | | | | | | | | | | | |
| 257 | (2S,3R,4S,5R)-2- | piperidine | | | | y | | | | | | | | |
| | methylpiperidine-3,4,5- | | | | | | | | | | | | | |
| | triol | | | | | | | | | | | | | |
| 258 | (2R,3S,4R,5S)-2- | piperidine | | | | y | | | | | | | | |
| | methylpiperidine-3,4,5- | | | | | | | | | | | | | |
| | triol | | | | | | | | | | | | | |
| 259 | (2R,3R,4R,5R)-2- | piperidine | | | | | | | | | y | | | |
| | methylpiperidine-3,4,5- | | | | | | | | | | | | | |
| | triol | | | | | | | | | | | | | |
| 260 | (3S,4S,5R,6S)-3,4,5- | piperidine | | | | | y | y | | | | | | |
| | trihydroxy-6- | | | | | | | | | | | | | |
| | (hydroxymethyl)piperidin- | | | | | | | | | | | | | |
| | 2-one | | | | | | | | | | | | | |
| 261 | (2R,4S,5R)-2-(2- | pyrrolidine | | | | | y | y | | | | | | |
| | hydroxyethyl)-5- | | | | | | | | | | | | | |
| | (hydroxymethyl)pyrrolidine- | | | | | | | | | | | | | |
| | 3,4-diol | | | | | | | | | | | | | |
| 262 | (2S,3R,4R)-3,4- | pyrrolidine | | | y | | | | | y | | | | |
| | dihydroxypyrrolidine-2- | | | | | | | | | | | | | |
| | carboxylic acid | | | | | | | | | | | | | |
| 263 | (2R,3S,4R,5S,6R)-2- | piperidine | | | | y | y | y | | | | | | |
| | (hydroxymethyl)-6- | | | | | | | | | | | | | |
| | methylpiperidine-3,4,5- | | | | | | | | | | | | | |
| | triol | | | | | | | | | | | | | |
| 264 | (2S,3S,4R,5S,6R)-2- | piperidine | | y | | | y | y | | | | | y | |
| | (hydroxymethyl)-6- | | | | | | | | | | | | | |
| | methylpiperidine-3,4,5- | | | | | | | | | | | | | |
| | triol | | | | | | | | | | | | | |
| 265 | (3S,4R,5S,6S)—N-butyl- | piperidine | | y | | | | | | | y | | y | |
| | 3,4,5-trihydroxy-6- | | | | | | | | | | | | | |
| | methylpiperidine-2- | | | | | | | | | | | | | |
| | carboxamide | | | | | | | | | | | | | |
| 266 | (1R,2S,6R,7S,7ar)- | pyrrolizidine | y | | | | | | | | | | | |
| | hexahydro-1H- | | | | | | | | | | | | | |
| | pyrrolizine-1,2,6,7- | | | | | | | | | | | | | |
| | tetraol | | | | | | | | | | | | | |
| 267 | (3S,4R,5S,6S)—N- | piperidine | | y | | | | | | | y | | y | |
| | benzyl-3,4,5-trihydroxy- | | | | | | | | | | | | | |
| | 6-methylpiperidine-2- | | | | | | | | | | | | | |
| | carboxamide | | | | | | | | | | | | | |
| 268 | (2S,3S,4S,5S)-2- | piperidine | | | | | | | | | y | | | |
| | methylpiperidine-3,4,5- | | | | | | | | | | | | | |
| | triol | | | | | | | | | | | | | |
| 269 | (2S,3S,4R,5S,6S)-2- | piperidine | | | | y | | | | | y | | | |
| | (hydroxymethyl)-6- | | | | | | | | | | | | | |
| | methylpiperidine-3,4,5- | | | | | | | | | | | | | |
| | triol | | | | | | | | | | | | | |
| 270 | 1- | azepane | | | y | | | | | y | | | | |
| | ((1R,2S,3S,4S,5S,7R)- | | | | | | | | | | | | | |
| | 2,3,4-trihydroxy-5- | | | | | | | | | | | | | |
| | methyl-7- | | | | | | | | | | | | | |
| | ((2R,3S,4R,5S,6S)- | | | | | | | | | | | | | |
| | 3,4,5-trihydroxy-6- | | | | | | | | | | | | | |
| | (hydroxymethyl)tetrahydro- | | | | | | | | | | | | | |
| | 2H-pyran-2-yloxy)-8- | | | | | | | | | | | | | |
| | oxa-6- | | | | | | | | | | | | | |
| | azabicyclo[3.2.1]octan- | | | | | | | | | | | | | |
| | 6-yl)ethanone | | | | | | | | | | | | | |
| 271 | (1R,2R,3R,6S,7S,7aR)- | pyrrolizidine | | y | | | | | | | y | | y | |
| | 5-gem-dideuterio-3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 272 | (3S,4s,5R)-1- | piperidine | | | | | | | | | | y | | |
| | butylpiperidine-3,4,5- | | | | | | | | | | | | | |
| | triol | | | | | | | | | | | | | |
| 273 | (3R,5R)-piperidine- | piperidine | | | y | | | | | y | | | | |
| | 3,4,5-triol | | | | | | | | | | | | | |
| 274 | ((2S,4S)-4- | pyrrolidine | | | y | | | | | y | | | | |
| | azidopyrrolidin-2- | | | | | | | | | | | | | |
| | yl)methanol | | | | | | | | | | | | | |
| 275 | ((2S,4S)-4-azido-1- | pyrrolidine | | | y | | | | | y | | | | |
| | butylpyrrolidin-2- | | | | | | | | | | | | | |
| | yl)methanol | | | | | | | | | | | | | |
| 276 | (2R,3R,4R,5S)-1-(4- | piperidine | | | | | y | y | | | | | | |
| | hydroxybutyl)-2- | | | | | | | | | | | | | |
| | (hydroxymethyl)piperidine- | | | | | | | | | | | | | |
| | 3,4,5-triol | | | | | | | | | | | | | |
| 277 | 2-((2S,4S)-4-azido-2- | pyrrolidine | | | y | | | | | y | | | | |
| | (hydroxymethyl)pyrrolidin- | | | | | | | | | | | | | |
| | 1-yl)ethanol | | | | | | | | | | | | | |
| 278 | (2R,3R,3aR,5S,6R,7R,7aS)- | piperidine | | y | | | | | | | | | y | |
| | 3-((R)-1- | | | | | | | | | | | | | |
| | hydroxybutyl)-5- | | | | | | | | | | | | | |
| | (hydroxymethyl)octahydrofuro[3, | | | | | | | | | | | | | |
| | 2-b]pyridine- | | | | | | | | | | | | | |
| | 2,6,7-triol | | | | | | | | | | | | | |
| 279 | (3R,4R,5R)-5- | | | | | | | | | | | | | y |
| | (hydroxymethyl)piperidine- | | | | | | | | | | | | | |
| | 3,4-diol | | | | | | | | | | | | | |
| 280 | (3R,5S)-1-(2- | pyrrolidine | | | | | | | | | | y | | y |
| | hydroxyethyl)-5- | | | | | | | | | | | | | |
| | (hydroxymethyl)pyrrolidin- | | | | | | | | | | | | | |
| | 3-ol | | | | | | | | | | | | | |
| 281 | (3R,5R)-3,4,5- | piperidine | | | y | | | | | y | | | | |
| | trihydroxypiperidine-1- | | | | | | | | | | | | | |
| | carbaldehyde | | | | | | | | | | | | | |
| 282 | (3S,5S)-piperidine- | piperidine | | | y | | | | | y | | | | |
| | 3,4,5-triol | | | | | | | | | | | | | |
| 283 | (3R,5S)-5- | pyrrolidine | | | | | | | | | | y | | y |
| | (hydroxymethyl)pyrrolidin- | | | | | | | | | | | | | |
| | 3-ol | | | | | | | | | | | | | |
| 284 | ((2S,4S)-4-azido-1- | pyrrolidine | | | y | | | | | y | | | | |
| | nonylpyrrolidin-2- | | | | | | | | | | | | | |
| | yl)methanol | | | | | | | | | | | | | |
| 285 | (3R,5S)-5- | pyrrolidine | | | | | | | | | | y | | y |
| | (aminomethyl)-1-(2- | | | | | | | | | | | | | |
| | hydroxyethyl)pyrrolidin- | | | | | | | | | | | | | |
| | 3-ol | | | | | | | | | | | | | |
| 286 | (3R,5S)-5- | pyrrolidine | | | | | | | | | | y | | y |
| | (azidomethyl)-1- | | | | | | | | | | | | | |
| | butylpyrrolidin-3-ol | | | | | | | | | | | | | |
| 287 | (3R,5S)-5- | pyrrolidine | | | | | | | | | | y | | y |
| | (azidomethyl)-1-(2- | | | | | | | | | | | | | |
| | hydroxyethyl)pyrrolidin- | | | | | | | | | | | | | |
| | 3-ol | | | | | | | | | | | | | |
| 288 | (2R,3R,4R,5S)-2- | piperidine | | | | | y | y | | | | | | |
| | (hydroxymethyl)-1-(3- | | | | | | | | | | | | | |
| | phenoxypropyl)piperidine- | | | | | | | | | | | | | |
| | 3,4,5-triol | | | | | | | | | | | | | |
| 289 | (3R,5S)-5- | pyrrolidine | | | | | | | | | | y | | y |
| | (aminomethyl)-1- | | | | | | | | | | | | | |
| | butylpyrrolidin-3-ol | | | | | | | | | | | | | |
| 290 | 2-((2S,4S)-4-amino-2- | pyrrolidine | | | y | | | | | y | | | | |
| | (hydroxymethyl)pyrrolidin- | | | | | | | | | | | | | |
| | 1-yl)ethanol | | | | | | | | | | | | | |
| 291 | diethyl 3-((2S,4S)-4- | pyrrolidine | | | y | | | | | y | | | | |
| | azido-2- | | | | | | | | | | | | | |
| | (hydroxymethyl)pyrrolidin- | | | | | | | | | | | | | |
| | 1- | | | | | | | | | | | | | |
| | yl)propylphosphonate | | | | | | | | | | | | | |
| 292 | ((2S,4S)-4-amino-1- | pyrrolidine | | | y | | | | | y | | | | |
| | butylpyrrolidin-2- | | | | | | | | | | | | | |
| | yl)methanol | | | | | | | | | | | | | |
| 293 | (3R,5S)-1-(2- | pyrrolidine | | | | | | | | | | y | | y |
| | hydroxyethyl)-5- | | | | | | | | | | | | | |
| | (morpholinomethyl)pyrrolidin- | | | | | | | | | | | | | |
| | 3-ol | | | | | | | | | | | | | |
| 294 | (3R,5S)-5- | pyrrolidine | | | | | | | | | | y | | y |
| | (hydroxymethyl)-1- | | | | | | | | | | | | | |
| | nonylpyrrolidin-3-ol | | | | | | | | | | | | | |
| 295 | ((2S,4S)-4-amino-1- | pyrrolidine | | | y | | | | | y | | | | |
| | nonylpyrrolidin-2- | | | | | | | | | | | | | |
| | yl)methanol | | | | | | | | | | | | | |
| 296 | (3R,5R)-1- | piperidine | | | y | | | | | y | | | | |
| | butylpiperidine-3,4,5- | | | | | | | | | | | | | |
| | triol | | | | | | | | | | | | | |
| 297 | (3R,5R)-1- | piperidine | | | y | | | | | y | | | | |
| | methylpiperidine-3,4,5- | | | | | | | | | | | | | |
| | triol | | | | | | | | | | | | | |
| 298 | (3S,5S)-1- | piperidine | | | y | | | | | y | | | | |
| | butylpiperidine-3,4,5- | | | | | | | | | | | | | |
| | triol | | | | | | | | | | | | | |
| 299 | (3S,5S)-1- | piperidine | | | y | | | | | y | | | | |
| | methylpiperidine-3,4,5- | | | | | | | | | | | | | |
| | triol | | | | | | | | | | | | | |
| 300 | (2S,3S,4S,5S)-2- | pyrrolidine | | | | | | | | | y | | | |
| | (hydroxymethyl)-5- | | | | | | | | | | | | | |
| | methylpyrrolidine-3,4- | | | | | | | | | | | | | |
| | diol | | | | | | | | | | | | | |
| 301 | (3S,4s,5R)-piperidine- | piperidine | | | | | | | | | | y | | |
| | 3,4,5-triol | | | | | | | | | | | | | |
| 302 | (3S,5S)-1- | piperidine | | | y | | | | | y | | | | |
| | nonylpiperidine-3,4,5- | | | | | | | | | | | | | |
| | triol | | | | | | | | | | | | | |
| 303 | (3R,5R)-tert-butyl 3,4,5- | piperidine | | | y | | | | | y | | | | |
| | trihydroxypiperidine-1- | | | | | | | | | | | | | |
| | carboxylate | | | | | | | | | | | | | |
| 304 | (2R,3S,4S)-1-(2- | pyrrolidine | | | | | | | | | | | | y |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 305 | (2R,3S,4S)-2- | pyrrolidine | | | | | | | | | | | | y |
| | (hydroxymethyl)pyrrolidine- | | | | | | | | | | | | | |
| | 3,4-diol | | | | | | | | | | | | | |
| 306 | (2R,3S,4S)-1-butyl-2- | pyrrolidine | | | | | | | | | | | | y |
| | (hydroxymethyl)pyrrolidine- | | | | | | | | | | | | | |
| | 3,4-diol | | | | | | | | | | | | | |
| 307 | (2R,3R,4S)-1-benzyl-2- | pyrrolidine | y | | | | | | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 308 | (2S,3S,4S)-4-azido-1- | pyrrolidine | | | y | | | | | y | | | | |
| | benzyl-2- | | | | | | | | | | | | | |
| | (hydroxymethyl)pyrrolidin- | | | | | | | | | | | | | |
| | 3-ol | | | | | | | | | | | | | |
| 309 | N-((3S,4R,5S)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 310 | (2R,3R,4S)-2- | pyrrolidine | | | | | | | | | | y | | |
| | (hydroxymethyl)pyrrolidine- | | | | | | | | | | | | | |
| | 3,4-diol | | | | | | | | | | | | | |
| 311 | (2R,3R,4S)-1-benzyl-2- | pyrrolidine | | | | | | | | | | y | | |
| | (hydroxymethyl)pyrrolidine- | | | | | | | | | | | | | |
| | 3,4-diol | | | | | | | | | | | | | |
| 312 | (2S,3R,4S)-4-amino-1- | pyrrolidine | | | y | | | | | y | | | | |
| | benzyl-2- | | | | | | | | | | | | | |
| | (hydroxymethyl)pyrrolidin- | | | | | | | | | | | | | |
| | 3-ol | | | | | | | | | | | | | |
| 313 | (2S,3R,4S)-4- | pyrrolidine | | | y | | | | | y | | | | |
| | acetamido-2- | | | | | | | | | | | | | |
| | (acetoxymethyl)-1- | | | | | | | | | | | | | |
| | benzylpyrrolidin-3-yl | | | | | | | | | | | | | |
| | acetate | | | | | | | | | | | | | |
| 314 | (2S,3S,4R)-1-butyl-2- | pyrrolidine | y | | | | | | | | | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 315 | (2R,3R,4S)-2-((S)-1,2- | pyrrolidine | y | | | | | | | | | | | |
| | dihydroxyethyl)pyrrolidine- | | | | | | | | | | | | | |
| | 3,4-diol | | | | | | | | | | | | | |
| 316 | (2S,3S,4R)-2-((R)-1,2- | pyrrolidine | y | | | | | | | | | | | |
| | dihydroxyethyl)-1- | | | | | | | | | | | | | |
| | nonylpyrrolidine-3,4-diol | | | | | | | | | | | | | |
| 317 | (2R,3R,4S)-1-butyl-2- | pyrrolidine | | | | | | | | | | y | | |
| | (hydroxymethyl)pyrrolidine- | | | | | | | | | | | | | |
| | 3,4-diol | | | | | | | | | | | | | |
| 318 | N-((3S,4R,5S)-4- | pyrrolidine | | | y | | | | | y | | | | |
| | hydroxy-5- | | | | | | | | | | | | | |
| | (hydroxymethyl)pyrrolidin- | | | | | | | | | | | | | |
| | 3-yl)acetamide | | | | | | | | | | | | | |
| 319 | N-((3S,4R,5S)-1-butyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 320 | (2S,3R,4R)-1- | pyrrolidine | | | | | | | | | | | | y |
| | (cyclohexylmethyl)-2- | | | | | | | | | | | | | |
| | (hydroxymethyl)pyrrolidine- | | | | | | | | | | | | | |
| | 3,4-diol | | | | | | | | | | | | | |
| 321 | (2S,3R,4R)-1-(2- | pyrrolidine | | | | | | | | | | | | y |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 322 | (1R,2R)-1-((2R,3R,4S)- | pyrrolidine | | | | y | | | | | y | | | |
| | 1-butyl-3,4- | | | | | | | | | | | | | |
| | dihydroxypyrrolidin-2- | | | | | | | | | | | | | |
| | yl)propane-1,2,3-triol | | | | | | | | | | | | | |
| 323 | (1R,2R)-1-((2R,3R,4S)- | pyrrolidine | | | | y | | | | | y | | | |
| | 3,4-dihydroxypyrrolidin- | | | | | | | | | | | | | |
| | 2-yl)propane-1,2,3-triol | | | | | | | | | | | | | |
| 324 | (1S,2R)-1-((2R,3R,4S)- | pyrrolidine | y | | | | | | | | | | | |
| | 3,4-dihydroxy-1- | | | | | | | | | | | | | |
| | nonylpyrrolidin-2- | | | | | | | | | | | | | |
| | yl)propane-1,2,3-triol | | | | | | | | | | | | | |
| 325 | (1S,2R)-1-((2R,3R,4S)- | pyrrolidine | y | | | | | | | | | | | |
| | 1-butyl-3,4- | | | | | | | | | | | | | |
| | dihydroxypyrrolidin-2- | | | | | | | | | | | | | |
| | yl)propane-1,2,3-triol | | | | | | | | | | | | | |
| 326 | (1S,2R)-1-((2R,3R,4S)- | pyrrolidine | y | | | | | | | | | | | |
| | 3,4-dihydroxy-1-(2- | | | | | | | | | | | | | |
| | hydroxyethyl)pyrrolidin- | | | | | | | | | | | | | |
| | 2-yl)propane-1,2,3-triol | | | | | | | | | | | | | |
| 327 | (1R,2R)-1-((2R,3R,4S)- | pyrrolidine | | | | y | | | | | y | | | |
| | 3,4-dihydroxy-1-(2- | | | | | | | | | | | | | |
| | hydroxyethyl)pyrrolidin- | | | | | | | | | | | | | |
| | 2-yl)propane-1,2,3-triol | | | | | | | | | | | | | |
| 328 | (1R,2R)-1-((2R,3R,4S)- | pyrrolidine | | | | y | | | | | y | | | |
| | 1-benzyl-3,4- | | | | | | | | | | | | | |
| | dihydroxypyrrolidin-2- | | | | | | | | | | | | | |
| | yl)propane-1,2,3-triol | | | | | | | | | | | | | |
| 329 | (1S,2R)-1-((2R,3R,4S)- | pyrrolidine | y | | | | | | | | | | | |
| | 1-benzyl-3,4- | | | | | | | | | | | | | |
| | dihydroxypyrrolidin-2- | | | | | | | | | | | | | |
| | yl)propane-1,2,3-triol | | | | | | | | | | | | | |
| 330 | ((2S,4S)-4-acetamido-1- | pyrrolidine | | | y | | | | | y | | | | |
| | (2- | | | | | | | | | | | | | |
| | acetoxyethyl)pyrrolidin- | | | | | | | | | | | | | |
| | 2-yl)methyl acetate | | | | | | | | | | | | | |
| 331 | ((2S,4S)-4-acetamido-1- | pyrrolidine | | | y | | | | | y | | | | |
| | butylpyrrolidin-2- | | | | | | | | | | | | | |
| | yl)methyl acetate | | | | | | | | | | | | | |
| 332 | ((2S,4S)-4-acetamido-1- | pyrrolidine | | | y | | | | | y | | | | |
| | nonylpyrrolidin-2- | | | | | | | | | | | | | |
| | yl)methyl acetate | | | | | | | | | | | | | |
| 333 | (1R,2S,8R,8aR)- | indolizidine | y | | | | | | | | | | | |
| | octahydroindolizine- | | | | | | | | | | | | | |
| | 1,2,8-triol | | | | | | | | | | | | | |
| 334 | (2S,3S,4R)-2-((R)-1,2- | pyrrolidine | y | | | | | | | | | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 335 | N-((3S,4R,5S)-4- | pyrrolidine | | | y | | | | | y | | | | |
| | hydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 336 | (1S,2R)-1-((2S,3R,4S)- | pyrrolidine | y | | | | | | | | | | | |
| | 3,4-dihydroxypyrrolidin- | | | | | | | | | | | | | |
| | 2-yl)propane-1,2,3-triol | | | | | | | | | | | | | |
| 337 | (1R,2S,8R,8aS)-1,2,8- | indolizidine | y | | | | | | | | | | | |
| | trihydroxyhexahydroindolizin- | | | | | | | | | | | | | |
| | 5(1H)-one | | | | | | | | | | | | | |
| 338 | N-((3S,4R,5S)-4- | pyrrolidine | | | y | | | | | y | | | | |
| | hydroxy-5- | | | | | | | | | | | | | |
| | (hydroxymethyl)-1- | | | | | | | | | | | | | |
| | nonylpyrrolidin-3- | | | | | | | | | | | | | |
| | yl)acetamide | | | | | | | | | | | | | |
| 339 | (2R,3R,4S)-1-butyl-2- | pyrrolidine | y | | | | | | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 340 | (2R,3R,4S)-2-((S)-1,2- | pyrrolidine | y | | | | | | | | | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 341 | 2-((2S,3S,4R)-2-((R)- | pyrrolidine | y | | | | | | | | | | | |
| | 1,2-dihydroxyethyl)-3,4- | | | | | | | | | | | | | |
| | dihydroxypyrrolidin-1- | | | | | | | | | | | | | |
| | yl)acetic acid | | | | | | | | | | | | | |
| 342 | (2S,3S,4R)-1-benzyl-2- | pyrrolidine | | | | | | | | | | y | | |
| | (hydroxymethyl)pyrrolidine- | | | | | | | | | | | | | |
| | 3,4-diol | | | | | | | | | | | | | |
| 343 | (2R,3R,4R,5R)-2,5- | pyrrolidine | | | | | | | | | y | | | |
| | bis(hydroxymethyl)-1-(3- | | | | | | | | | | | | | |
| | phenoxypropyl)pyrrolidine- | | | | | | | | | | | | | |
| | 3,4-diol | | | | | | | | | | | | | |
| 344 | ((2S,4S)-4- | pyrrolidine | | | y | | | | | y | | | | |
| | aminopyrrolidin-2- | | | | | | | | | | | | | |
| | yl)methanol | | | | | | | | | | | | | |
| 345 | (2S,4S)-4- | pyrrolidine | | | y | | | | | y | | | | |
| | azidopyrrolidine-2- | | | | | | | | | | | | | |
| | carboxylic acid | | | | | | | | | | | | | |
| 346 | N-((3S,5S)-5- | pyrrolidine | | | y | | | | | y | | | | |
| | (hydroxymethyl)pyrrolidin- | | | | | | | | | | | | | |
| | 3-yl)acetamide | | | | | | | | | | | | | |
| 347 | N-((3S,5S)-1-(2- | pyrrolidine | | | y | | | | | y | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 348 | N-((3S,5S)-5- | pyrrolidine | | | y | | | | | y | | | | |
| | (hydroxymethyl)-1- | | | | | | | | | | | | | |
| | nonylpyrrolidin-3- | | | | | | | | | | | | | |
| | yl)acetamide | | | | | | | | | | | | | |
| 349 | (2R,3R,4R)-2- | pyrrolidine | | | y | | | | | y | | | | |
| | (hydroxymethyl)-1-(3- | | | | | | | | | | | | | |
| | phenoxypropyl)pyrrolidine- | | | | | | | | | | | | | |
| | 3,4-diol | | | | | | | | | | | | | |
| 350 | N-((3S,5S)-1-butyl-5- | pyrrolidine | | | y | | | | | y | | | | |
| | (hydroxymethyl)pyrrolidin- | | | | | | | | | | | | | |
| | 3-yl)acetamide | | | | | | | | | | | | | |
| 351 | (2S,3R,4S)-1-benzyl-2- | pyrrolidine | | | | | | | | | y | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 352 | (1S,2S,6S,7R,8R,8aR)- | indolizidine | | y | | | y | y | | | | | y | |
| | octahydroindolizine- | | | | | | | | | | | | | |
| | 1,2,6,7,8-pentaol | | | | | | | | | | | | | |
| 353 | N-((3S,4R,5S)-4,5- | piperidine | | | y | | | | | y | | | | |
| | dihydroxypiperidin-3- | | | | | | | | | | | | | |
| | yl)acetamide | | | | | | | | | | | | | |
| 354 | (3R,5R)-benzyl 3,4,5- | piperidine | | | y | | | | | y | | | | |
| | trihydroxypiperidine-1- | | | | | | | | | | | | | |
| | carboxylate | | | | | | | | | | | | | |
| 355 | 2-((2S,4S)-4-azido-2- | pyrrolidine | | | y | | | | | y | | | | |
| | (hydroxymethyl)pyrrolidin- | | | | | | | | | | | | | |
| | 1-yl)acetic acid | | | | | | | | | | | | | |
| 356 | (1R,2S,3S,7R,7aR)-3- | pyrrolizidine | y | | | | | | | | | | | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 357 | (2R,3S,4S)-2- | piperidine | | | | | | | | | | | | y |
| | (hydroxymethyl)piperidine- | | | | | | | | | | | | | |
| | 3,4-diol | | | | | | | | | | | | | |
| 358 | 2-((2S,3R,4S)-4- | pyrrolidine | | | y | | | | | y | | | | |
| | acetamido-3-hydroxy-2- | | | | | | | | | | | | | |
| | (hydroxymethyl)pyrrolidin- | | | | | | | | | | | | | |
| | 1-yl)acetic acid | | | | | | | | | | | | | |
| 359 | 2-((2R,3R,4S)-2-((S)- | pyrrolidine | y | | | | | | | | | | | |
| | 1,2-dihydroxyethyl)-3,4- | | | | | | | | | | | | | |
| | dihydroxypyrrolidin-1- | | | | | | | | | | | | | |
| | yl)acetic acid | | | | | | | | | | | | | |
| 360 | (2S,3S,4R)-1-butyl-2- | pyrrolidine | | | | | | | | | | y | | |
| | (hydroxymethyl)pyrrolidine- | | | | | | | | | | | | | |
| | 3,4-diol | | | | | | | | | | | | | |
| 361 | 2-((2R,3R,4S)-3,4- | pyrrolidine | | | | | | | | | | y | | |
| | dihydroxy-2- | | | | | | | | | | | | | |
| | (hydroxymethyl)pyrrolidin- | | | | | | | | | | | | | |
| | 1-yl)acetic acid | | | | | | | | | | | | | |
| 362 | (2R,3S,4R)-4- | pyrrolidine | | | y | | | | | y | | | | |
| | acetamido-2- | | | | | | | | | | | | | |
| | (acetoxymethyl)-1- | | | | | | | | | | | | | |
| | benzylpyrrolidin-3-yl | | | | | | | | | | | | | |
| | acetate | | | | | | | | | | | | | |
| 363 | (2R,3R,4R)-4-azido-1- | pyrrolidine | | | y | | | | | y | | | | |
| | benzyl-2- | | | | | | | | | | | | | |
| | (hydroxymethyl)pyrrolidin- | | | | | | | | | | | | | |
| | 3-ol | | | | | | | | | | | | | |
| 364 | N-((3R,4S,5R)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 365 | N-((3R,4S,5R)-4- | pyrrolidine | | | y | | | | | y | | | | |
| | hydroxy-5- | | | | | | | | | | | | | |
| | (hydroxymethyl)pyrrolidin- | | | | | | | | | | | | | |
| | 3-yl)acetamide | | | | | | | | | | | | | |
| 366 | 2-((2R,3S,4R)-4- | pyrrolidine | | | y | | | | | y | | | | |
| | acetamido-3-hydroxy-2- | | | | | | | | | | | | | |
| | (hydroxymethyl)pyrrolidin- | | | | | | | | | | | | | |
| | 1-yl)acetic acid | | | | | | | | | | | | | |
| 367 | N-((3R,4S,5R)-4- | pyrrolidine | | | y | | | | | y | | | | |
| | hydroxy-5- | | | | | | | | | | | | | |
| | (hydroxymethyl)-1- | | | | | | | | | | | | | |
| | isopropylpyrrolidin-3- | | | | | | | | | | | | | |
| | yl)acetamide | | | | | | | | | | | | | |
| 368 | 2-((2S,3S,4R)-3,4- | pyrrolidine | | | | | | | | | | y | | |
| | dihydroxy-2- | | | | | | | | | | | | | |
| | (hydroxymethyl)pyrrolidin- | | | | | | | | | | | | | |
| | 1-yl)acetic acid | | | | | | | | | | | | | |
| 369 | N-((3R,4S,5R)-4- | pyrrolidine | | | y | | | | | y | | | | |
| | hydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 370 | (2R,3S,4R)-4-amino-1- | pyrrolidine | | | y | | | | | y | | | | |
| | benzyl-2- | | | | | | | | | | | | | |
| | (hydroxymethyl)pyrrolidin- | | | | | | | | | | | | | |
| | 3-ol | | | | | | | | | | | | | |
| 371 | (2S,3S,4S,5S)-3,4- | pyrrolidine | | | | | | | | | y | | | |
| | dihydroxy-2,5- | | | | | | | | | | | | | |
| | bis(hydroxymethyl)pyrrolidine- | | | | | | | | | | | | | |
| | 1-carbaldehyde | | | | | | | | | | | | | |
| 372 | N-((3R,4R,5S)-1-benzyl- | pyrrolidine | | | | | | | | | | y | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 373 | (2S,3R,4R)-4-amino-1- | pyrrolidine | | | | | | | | | | y | | |
| | benzyl-2- | | | | | | | | | | | | | |
| | (hydroxymethyl)pyrrolidin- | | | | | | | | | | | | | |
| | 3-ol | | | | | | | | | | | | | |
| 374 | (2S,3S,4R)-4-azido-1- | pyrrolidine | | | | | | | | | | y | | |
| | benzyl-2- | | | | | | | | | | | | | |
| | (hydroxymethyl)pyrrolidin- | | | | | | | | | | | | | |
| | 3-ol | | | | | | | | | | | | | |
| 375 | N-((3R,4R,5S)-4- | pyrrolidine | | | | | | | | | | y | | |
| | hydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 376 | (2S,3R,4R)-4- | pyrrolidine | | | | | | | | | | y | | |
| | acetamido-2- | | | | | | | | | | | | | |
| | (acetoxymethyl)-1- | | | | | | | | | | | | | |
| | benzylpyrrolidin-3-yl | | | | | | | | | | | | | |
| | acetate | | | | | | | | | | | | | |
| 377 | 2-((2S,3R,4R)-4- | pyrrolidine | | | | | | | | | | y | | |
| | acetamido-3-hydroxy-2- | | | | | | | | | | | | | |
| | (hydroxymethyl)pyrrolidin- | | | | | | | | | | | | | |
| | 1-yl)acetic acid | | | | | | | | | | | | | |
| 378 | N-((3R,4R,5S)-4- | pyrrolidine | | | | | | | | | | y | | |
| | hydroxy-5- | | | | | | | | | | | | | |
| | (hydroxymethyl)pyrrolidin- | | | | | | | | | | | | | |
| | 3-yl)acetamide | | | | | | | | | | | | | |
| 379 | N-((3R,4R,5S)-1-butyl- | pyrrolidine | | | | | | | | | | y | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 380 | (2R,3R,4S,5S,6S)-2- | piperidine | | | | | y | y | | | | | | |
| | (but-3-enyl)-6- | | | | | | | | | | | | | |
| | (hydroxymethyl)piperidine- | | | | | | | | | | | | | |
| | 3,4,5-triol | | | | | | | | | | | | | |
| 381 | (3R,5S)-5- | pyrrolidine | | | | | | | | | | y | | y |
| | (azidomethyl)pyrrolidin- | | | | | | | | | | | | | |
| | 3-ol | | | | | | | | | | | | | |
| 382 | (2R,3R,4R,5S)-3,4,5- | piperidine | | | | | | | y | | | | | |
| | trihydroxy-N- | | | | | | | | | | | | | |
| | methylpiperidine-2- | | | | | | | | | | | | | |
| | carboxamide | | | | | | | | | | | | | |
| 383 | (5R)-3-hydroxy-5- | pyrrolidine | | | | | | | | | | | | |
| | (hydroxymethyl)pyrrolidine- | | | | | | | | | | | | | |
| | 3-carboxylic acid | | | | | | | | | | | | | |
| 384 | (2R,3S,4R)-2- | pyrrolidine | | | y | | | | | y | | | | |
| | (hydroxymethyl)pyrrolidine- | | | | | | | | | | | | | |
| | 3,4-diol | | | | | | | | | | | | | |
| 385 | (2R,3S,4R)-1-benzyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 386 | (2S,3R,4S)-1-benzyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 387 | (3R,5R)-1- | piperidine | | | y | | | | | y | | | | |
| | nonylpiperidine-3,4,5- | | | | | | | | | | | | | |
| | triol | | | | | | | | | | | | | |
| 388 | (2R,3R,4R,5S)-2- | piperidine | | | | | y | y | | | | | | |
| | methylpiperidine-3,4,5- | | | | | | | | | | | | | |
| | triol | | | | | | | | | | | | | |
| 389 | (2R,3S,4S)-4- | pyrrolidine | | | | | | | | | | y | | |
| | acetamido-2- | | | | | | | | | | | | | |
| | (acetoxymethyl)-1- | | | | | | | | | | | | | |
| | benzylpyrrolidin-3-yl | | | | | | | | | | | | | |
| | acetate | | | | | | | | | | | | | |
| 390 | (2R,3S,4S)-4-amino-1- | pyrrolidine | | | | | | | | | | y | | |
| | benzyl-2- | | | | | | | | | | | | | |
| | (hydroxymethyl)pyrrolidin- | | | | | | | | | | | | | |
| | 3-ol | | | | | | | | | | | | | |
| 391 | N-((3S,4S,5R)-1-butyl- | pyrrolidine | | | | | | | | | | y | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 392 | 2-((2R,3S,4S)-4- | pyrrolidine | | | | | | | | | | y | | |
| | acetamido-3-hydroxy-2- | | | | | | | | | | | | | |
| | (hydroxymethyl)pyrrolidin- | | | | | | | | | | | | | |
| | 1-yl)acetic acid | | | | | | | | | | | | | |
| 393 | (R)-5-((1R,2S,3S)- | pyrrolidine | | | | | y | y | | | | | | |
| | 1,2,3,4- | | | | | | | | | | | | | |
| | tetrahydroxybutyl)pyrrolidin- | | | | | | | | | | | | | |
| | 2-one | | | | | | | | | | | | | |
| 394 | (2R,3R,4S)-4-azido-1- | pyrrolidine | | | | | | | | | | y | | |
| | benzyl-2- | | | | | | | | | | | | | |
| | (hydroxymethyl)pyrrolidin- | | | | | | | | | | | | | |
| | 3-ol | | | | | | | | | | | | | |
| 395 | N-((3S,4S,5R)-4- | pyrrolidine | | | | | | | | | | y | | |
| | hydroxy-5- | | | | | | | | | | | | | |
| | (hydroxymethyl)pyrrolidin- | | | | | | | | | | | | | |
| | 3-yl)acetamide | | | | | | | | | | | | | |
| 396 | N-((3S,4S,5R)-1-benzyl- | pyrrolidine | | | | | | | | | | y | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 397 | N-((3S,4S,5R)-4- | pyrrolidine | | | | | | | | | | y | | |
| | hydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 398 | 2-((2S,3R,4S)-2-((R)- | pyrrolidine | | | | | | | | | y | | | |
| | 1,2-dihydroxyethyl)-3,4- | | | | | | | | | | | | | |
| | dihydroxypyrrolidin-1- | | | | | | | | | | | | | |
| | yl)acetic acid | | | | | | | | | | | | | |
| 399 | (1R,2S,5S,8R,8aS)-5- | indolizidine | y | | | | | | | | | | | |
| | methyloctahydroindolizine- | | | | | | | | | | | | | |
| | 1,2,8-triol | | | | | | | | | | | | | |
| 400 | (1R,2S,6S,7R,8R,8aR)- | indolizidine | | | | | y | y | | | y | | | |
| | octahydroindolizine- | | | | | | | | | | | | | |
| | 1,2,6,7,8-pentaol | | | | | | | | | | | | | |
| 401 | (1R,2S,6R,7S,8R,8aR)- | indolizidine | | | | | y | y | | | y | | | |
| | 1,2,6,7,8- | | | | | | | | | | | | | |
| | pentahydroxyhexahydroindolizin- | | | | | | | | | | | | | |
| | 5(1H)-one | | | | | | | | | | | | | |
| 402 | (1R,2S,8S,8aS)-1,2,8- | indolizidine | | | | | | | | | y | | | |
| | trihydroxyhexahydroindolizin- | | | | | | | | | | | | | |
| | 5(1H)-one | | | | | | | | | | | | | |
| 403 | (2S,3R,4S)-1-butyl-2- | pyrrolidine | | | | | | | | | y | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 404 | (1R,2R)-1-((2R,3R,4S)- | pyrrolidine | | | | y | | | | | y | | | |
| | 3,4-dihydroxy-1- | | | | | | | | | | | | | |
| | nonylpyrrolidin-2- | | | | | | | | | | | | | |
| | yl)propane-1,2,3-triol | | | | | | | | | | | | | |
| 405 | (2S,3R,4S)-2-((R)-1,2- | pyrrolidine | | | | | | | | | y | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 406 | (1R,2S,5R,8S,8aS)-5- | indolizidine | | | | | | | | | y | | | |
| | methyloctahydroindolizine- | | | | | | | | | | | | | |
| | 1,2,8-triol | | | | | | | | | | | | | |
| 407 | (S)-5-((1S,2R,3R)- | pyrrolidine | | | | | y | y | | | | | | |
| | 1,2,3,4- | | | | | | | | | | | | | |
| | tetrahydroxybutyl)pyrrolidin- | | | | | | | | | | | | | |
| | 2-one | | | | | | | | | | | | | |
| 408 | (S)-4-((2S,3R,4S)-1- | pyrrolidine | | | | | | | | | y | | | |
| | benzyl-3,4- | | | | | | | | | | | | | |
| | dihydroxypyrrolidin-2- | | | | | | | | | | | | | |
| | yl)-4- | | | | | | | | | | | | | |
| | hydroxybutanenitrile | | | | | | | | | | | | | |
| 409 | (3aS,6R,9S,9aS,9bR)- | indolizidine | | | | | | | | | y | | | |
| | 2,2-diethyl-6- | | | | | | | | | | | | | |
| | methyloctahydro- | | | | | | | | | | | | | |
| | [1,3]dioxolo[4,5- | | | | | | | | | | | | | |
| | a]indolizin-9-ol | | | | | | | | | | | | | |
| 410 | (1R,2S,5R,8S,8aR)-8- | indolizidine | | | | | | | | | y | | | |
| | methoxy-5- | | | | | | | | | | | | | |
| | methyloctahydroindolizine- | | | | | | | | | | | | | |
| | 1,2-diol | | | | | | | | | | | | | |
| 411 | (1R,2S,3S)-1-((R)-1- | pyrrolidine | | | | | y | y | | | | | | |
| | butylpyrrolidin-2- | | | | | | | | | | | | | |
| | yl)butane-1,2,3,4-tetraol | | | | | | | | | | | | | |
| 412 | (3aR,5R,6R,6aS)-4- | pyrrolidine | | y | | | | | | | | | y | |
| | benzyl-6-hydroxy-5- | | | | | | | | | | | | | |
| | (hydroxymethyl)hexahydro- | | | | | | | | | | | | | |
| | 2H-furo[3,2-b]pyrrol- | | | | | | | | | | | | | |
| | 2-one | | | | | | | | | | | | | |
| 413 | (1R,2S,3S)-1-((R)- | pyrrolidine | | | | | y | y | | | | | | |
| | pyrrolidin-2-yl)butane- | | | | | | | | | | | | | |
| | 1,2,3,4-tetraol | | | | | | | | | | | | | |
| 414 | (1R,2S,3S)-1-((R)-1-(2- | pyrrolidine | | | | | y | y | | | | | | |
| | hydroxyethyl)pyrrolidin- | | | | | | | | | | | | | |
| | 2-yl)butane-1,2,3,4- | | | | | | | | | | | | | |
| | tetraol | | | | | | | | | | | | | |
| 415 | (S)-5-((1R,2S,3S)- | pyrrolidine | | | | | | | y | | | | | |
| | 1,2,3,4- | | | | | | | | | | | | | |
| | tetrahydroxybutyl)pyrrolidin- | | | | | | | | | | | | | |
| | 2-one | | | | | | | | | | | | | |
| 416 | (1R,2S,3S)-1-((S)-1-(2- | pyrrolidine | | | | | | | y | | | | | |
| | hydroxyethyl)pyrrolidin- | | | | | | | | | | | | | |
| | 2-yl)butane-1,2,3,4- | | | | | | | | | | | | | |
| | tetraol | | | | | | | | | | | | | |
| 417 | N-((3S,4R,5S)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)-2,2,2- | | | | | | | | | | | | | |
| | trifluoroacetamide | | | | | | | | | | | | | |
| 418 | (3S,4S,5S)-1-butyl-5- | piperidine | | | | | | | | | | | | y |
| | (hydroxymethyl)piperidine- | | | | | | | | | | | | | |
| | 3,4-diol | | | | | | | | | | | | | |
| 419 | (3R,4R,5R)-1-butyl-5- | piperidine | | | | | | | | | | | | y |
| | (hydroxymethyl)piperidine- | | | | | | | | | | | | | |
| | 3,4-diol | | | | | | | | | | | | | |
| 420 | (3R,4R,5R)-1-(2- | piperidine | | | | | | | | | | | | y |
| | hydroxyethyl)-5- | | | | | | | | | | | | | |
| | (hydroxymethyl)piperidine- | | | | | | | | | | | | | |
| | 3,4-diol | | | | | | | | | | | | | |
| 421 | (1R,2S,3S)-1-((S)- | pyrrolidine | | | | | | | y | | | | | |
| | pyrrolidin-2-yl)butane- | | | | | | | | | | | | | |
| | 1,2,3,4-tetraol | | | | | | | | | | | | | |
| 422 | (1S,2R,3R)-1-((S)- | pyrrolidine | | | | | y | y | | | | | | |
| | pyrrolidin-2-yl)butane- | | | | | | | | | | | | | |
| | 1,2,3,4-tetraol | | | | | | | | | | | | | |
| 423 | (2S,3S,4R)-1-benzyl-2- | pyrrolidine | | y | | | | | | | | | y | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 424 | (2R,3R,4R,5S)-tert-butyl | piperidine | | | | | y | y | | | | | | |
| | 3,4,5-trihydroxy-2- | | | | | | | | | | | | | |
| | (hydroxymethyl)piperidine- | | | | | | | | | | | | | |
| | 1-carboxylate | | | | | | | | | | | | | |
| 425 | (2S,3S,4R)-2-((S)-1,2- | pyrrolidine | | y | | | | | | | | | y | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 426 | (2S,3S,4R)-1-butyl-2- | pyrrolidine | | y | | | | | | | | | y | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 427 | (2S,3S,4R)-2-((S)-1,2- | pyrrolidine | | y | | | | | | | | | y | |
| | dihydroxyethyl)-1- | | | | | | | | | | | | | |
| | nonylpyrrolidine-3,4-diol | | | | | | | | | | | | | |
| 428 | (2S,3S,4R)-2-((S)-1,2- | pyrrolidine | | y | | | | | | | | | y | |
| | dihydroxyethyl)-1- | | | | | | | | | | | | | |
| | methylpyrrolidine-3,4- | | | | | | | | | | | | | |
| | diol | | | | | | | | | | | | | |
| 429 | (2S,3S,4R)-2-((S)-1,2- | pyrrolidine | | y | | | | | | | | | y | |
| | dihydroxyethyl)pyrrolidine- | | | | | | | | | | | | | |
| | 3,4-diol | | | | | | | | | | | | | |
| 430 | (3aR,4R,6aS)-4- | pyrrolidine | | | | | | | | | | y | | |
| | (azidomethyl)-5-benzyl- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrole | | | | | | | | | | | | | |
| 431 | N-((3S,4R,5S)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)butyramide | | | | | | | | | | | | | |
| 432 | (2R,3R,4S)-2- | pyrrolidine | | | | | | | | | | y | | |
| | (azidomethyl)-1- | | | | | | | | | | | | | |
| | benzylpyrrolidine-3,4- | | | | | | | | | | | | | |
| | diol | | | | | | | | | | | | | |
| 433 | (2S,3S,4R)-2-((S)-1,2- | pyrrolidine | | y | | | | | | | | | y | |
| | dihydroxyethyl)-1-(9- | | | | | | | | | | | | | |
| | hydroxynonyl)pyrrolidine- | | | | | | | | | | | | | |
| | 3,4-diol | | | | | | | | | | | | | |
| 434 | 2-((2S,3S,4R)-2-((S)- | pyrrolidine | | y | | | | | | | | | y | |
| | 1,2-dihydroxyethyl)-3,4- | | | | | | | | | | | | | |
| | dihydroxypyrrolidin-1- | | | | | | | | | | | | | |
| | yl)acetonitrile | | | | | | | | | | | | | |
| 435 | (2S,3S,4R)-2-((S)-1,2- | pyrrolidine | | y | | | | | | | | | y | |
| | dihydroxyethyl)-1-(2-(2- | | | | | | | | | | | | | |
| | methoxyethoxy)ethyl)pyrrolidine- | | | | | | | | | | | | | |
| | 3,4-diol | | | | | | | | | | | | | |
| 436 | (3R,4R,4aR,7S,8R,8aR)- | piperidine | | | | | y | y | | | | | | |
| | octahydro-2H- | | | | | | | | | | | | | |
| | pyrano[3,2-b]pyridine- | | | | | | | | | | | | | |
| | 3,4,7,8-tetrol | | | | | | | | | | | | | |
| 437 | 6-[(2S,4R)-4-hydroxy-2- | pyrrolidine | | | | | | | | | | y | | y |
| | (hydroxymethyl)pyrrolidin- | | | | | | | | | | | | | |
| | 1-yl]hexanoic acid | | | | | | | | | | | | | |
| 438 | 2-{[(2S,3S,4R,5S)-4- | pyrrolidine | | y | | | y | y | | | | | y | |
| | hydroxy-5- | | | | | | | | | | | | | |
| | (hydroxymethyl)-2- | | | | | | | | | | | | | |
| | [(2S,4Z)-undec-4-ene- | | | | | | | | | | | | | |
| | 1,2,11-triol]pyrrolidin-3- | | | | | | | | | | | | | |
| | yl]oxy}-6- | | | | | | | | | | | | | |
| | (hydroxymethyl)tetrahydro- | | | | | | | | | | | | | |
| | 2H-pyran-3,4,5-triol | | | | | | | | | | | | | |
| 439 | 3-[(2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl]propanoic acid | | | | | | | | | | | | | |
| 440 | [(2S,3R,4R)-3,4- | pyrrolidine | | | | | | | | | | | | y |
| | dihydroxy-2- | | | | | | | | | | | | | |
| | (hydroxymethyl)pyrrolidin- | | | | | | | | | | | | | |
| | 1-yl]acetic acid | | | | | | | | | | | | | |
| 441 | (2S,4S,5S)-4,5- | piperidine | | | | y | y | y | | | | | | |
| | dihydroxypiperidine-2- | | | | | | | | | | | | | |
| | carboxylic acid | | | | | | | | | | | | | |
| 442 | (2R,3R,4R,5S)-3,4,5- | piperidine | | | | | | | y | | | | | |
| | trihydroxypiperidine-2- | | | | | | | | | | | | | |
| | carboxylic acid | | | | | | | | | | | | | |
| 443 | (3aR,6R,7R,8R,8aS,8bS)- | pyrrolizidine | | | | | y | y | y | | | | | |
| | 7,8-dihydroxy-6- | | | | | | | | | | | | | |
| | (hydroxymethyl)-2,2- | | | | | | | | | | | | | |
| | dimethylhexahydro-4H- | | | | | | | | | | | | | |
| | [1,3]dioxolo[4,5- | | | | | | | | | | | | | |
| | a]pyrrolizin-4-one | | | | | | | | | | | | | |
| 444 | (2S,5S,6S,7S,8R,8aS)- | indolizidine | | | | | y | y | | | | | | |
| | 6,7,8-trihydroxy-5- | | | | | | | | | | | | | |
| | (hydroxymethyl)-3- | | | | | | | | | | | | | |
| | oxooctahydroindolizine- | | | | | | | | | | | | | |
| | 2-carboxylic acid | | | | | | | | | | | | | |
| 445 | (2S,5R,6R,7R,8R,8aR)- | indolizidine | | | | | | | | | y | | | |
| | 6,7,8-trihydroxy-5- | | | | | | | | | | | | | |
| | (hydroxymethyl)-3- | | | | | | | | | | | | | |
| | oxooctahydroindolizine- | | | | | | | | | | | | | |
| 446 | 2-carboxylic acid | piperidine | | | | | | | y | | | | | |
| | (3S,4R,5S,6R)-3,4,5- | | | | | | | | | | | | | |
| | trihydroxy-6- | | | | | | | | | | | | | |
| | (hydroxymethyl)piperidin- | | | | | | | | | | | | | |
| | 2-one | | | | | | | | | | | | | |
| 447 | (2R,3S,4S,5R)-2- | piperidine | | | | | | | y | | | | | |
| | (hydroxymethyl)piperidine- | | | | | | | | | | | | | |
| | 3,4,5-triol | | | | | | | | | | | | | |
| 448 | (1R,2S,5R,6S,7S,7aS)- | pyrrolizidine | | | | | | | y | | | | | |
| | 1,2,6,7-tetrahydroxy-5- | | | | | | | | | | | | | |
| | (hydroxymethyl)hexahydro- | | | | | | | | | | | | | |
| | 3H-pyrrolizin-3-one | | | | | | | | | | | | | |
| 449 | (2R,3R,4R,5S)-benzyl | piperidine | | | | | y | y | | | | | | |
| | 3,4,5-trihydroxy-2- | | | | | | | | | | | | | |
| | (hydroxymethyl)piperidine- | | | | | | | | | | | | | |
| | 1-carboxylate | | | | | | | | | | | | | |
| 450 | 2-((3R,4R,5R)-3,4- | piperidine | | | | | | | | | | | | y |
| | dihydroxy-5- | | | | | | | | | | | | | |
| | (hydroxymethyl)piperidin- | | | | | | | | | | | | | |
| | 1-yl)acetic acid | | | | | | | | | | | | | |
| 451 | 2-((3S,4S,5S)-3,4- | piperidine | | | | | | | | | | | | y |
| | dihydroxy-5- | | | | | | | | | | | | | |
| | (hydroxymethyl)piperidin- | | | | | | | | | | | | | |
| | 1-yl)acetic acid | | | | | | | | | | | | | |
| 452 | (2R,3S,4R)-3,4- | pyrrolidine | | | | | | | | | | y | | |
| | dihydroxy-2- | | | | | | | | | | | | | |
| | methylpyrrolidine-2- | | | | | | | | | | | | | |
| | carboxylic acid | | | | | | | | | | | | | |
| 453 | 8-Aza- | nortropane | | | | | | | | | | | | |
| | bicyclo[3,2,1]octan-3-ol | | | | | | | | | | | | | |
| 454 | (R)-3-Hydroxypiperidine | piperidine | | | | | | | | | | | | |
| 455 | 4-Hydroxypiperidine | piperidine | | | | | | | | | | | | |
| 456 | cis-L-3-Hydroxyproline pyrrolidine | | | | | | | | | | | | | |
| 457 | (R)-3- | pyrrolidine | | | | | | | | | | | | |
| | Hydroxypyrrolidine | | | | | | | | | | | | | |
| 458 | cis-4-Hydroxy-D-proline | pyrrolidine | | | y | | | | | y | | | | |
| 459 | 4-hydroxy-2- | pyrrolidine | | | | | | | | | | | | |
| | Pyrrolidinecarboxamide | | | | | | | | | | | | | |
| 460 | 2-methyl-4-Piperidinol | piperidine | | | | | | | | | | | | |
| 461 | L-beta- | pyrrolidine | | | | | | | | | | y | | y |
| | Homohydroxyproline | | | | | | | | | | | | | |
| 462 | (R)-5-Hydroxy-piperidin- | piperidine | | | | | | | | | | | | |
| | 2-one | | | | | | | | | | | | | |
| 463 | (S)-(−)-4-Hydroxy-2- | pyrrolidine | | | | | | | | | | | | |
| | pyrrolidinone | | | | | | | | | | | | | |
| 464 | Nojirimycin-1-Sulfonic | piperidine | | | | | y | y | | | | | | |
| | Acid | | | | | | | | | | | | | |
| 465 | Siastatin B microbial | piperidine | y | | | | | | | | | | | |
| 466 | D-Glucaro-delta-lactam | piperidine | | | | | y | y | | | | | | |
| 467 | 4-hydroxy-4- | piperidine | | | | | | | | | | | | |
| | Piperidinecarboxylic | | | | | | | | | | | | | |
| | acid | | | | | | | | | | | | | |
| 468 | Labumine | pyrrolizidine | | | | | | | | | | | | |
| 469 | 1-Deoxy-L- | piperidine | | | | | | | y | | | | | |
| | idonojirimycin | | | | | | | | | | | | | |
| 470 | 2,5-Anhydro-2,5-imino- | pyrrolidine | | | | | y | y | | | | | | |
| | D-glucitol | | | | | | | | | | | | | |
| 471 | 1,4-Dideoxy-1,4-imino- | pyrrolidine | | | | | | | | | y | | | |
| | D-mannitol | | | | | | | | | | | | | |
| 472 | (2S,5S)- | pyrrolidine | | | | | | | y | | | | | |
| | Bishydroxymethyl- | | | | | | | | | | | | | |
| | (3R,4R)- | | | | | | | | | | | | | |
| | bishydroxypyrrolidine | | | | | | | | | | | | | |
| 473 | 4-hydroxy-2- | pyrrolidine | | | | | | | | | | | | |
| | Pyrrolidinemethanol | | | | | | | | | | | | | |
| 474 | (R)-3-Hydroxypiperidine | piperidine | | | | | | | | | | | | |
| 475 | cis-L-3-Hydroxyproline | pyrrolidine | | | | | | | | | | | | |
| 476 | (S)-3-Hydroxypyrrolidine | pyrrolidine | | | | | | | | | | | | |
| 477 | trans-4-Hydroxy-D- | pyrrolidine | | | | | | | | | | y | | y |
| | proline | | | | | | | | | | | | | |
| 478 | trans-4-Hydroxy-D- | pyrrolidine | | | | | | | | | | y | | y |
| | proline | | | | | | | | | | | | | |
| 479 | (R)-(+)-4-Hydroxy-2- | pyrrolidine | | | | | | | | | | | | |
| | pyrrolidinone | | | | | | | | | | | | | |
| 480 | S)-3-Hydroxy-pyrrolidin- | pyrrolidine | | | | | | | | | | | | |
| | 2-one | | | | | | | | | | | | | |
| 481 | N-(((3aR,4R,6aS)-5- | pyrrolidine | | | | | | | | | | y | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 482 | N-(((2R,3R,4S)-1- | pyrrolidine | | | | | | | | | | y | | |
| | benzyl-3,4- | | | | | | | | | | | | | |
| | dihydroxypyrrolidin-2- | | | | | | | | | | | | | |
| | yl)methyl)acetamide | | | | | | | | | | | | | |
| 483 | ((3aR,4R,6aS)-5- | pyrrolidine | | | | | | | | | | y | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methanamine | | | | | | | | | | | | | |
| 484 | N-(((2R,3R,4S)-3,4- | pyrrolidine | | | | | | | | | | y | | |
| | dihydroxypyrrolidin-2- | | | | | | | | | | | | | |
| | yl)methyl)acetamide | | | | | | | | | | | | | |
| 485 | N-((3S,4R,5S)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)benzamide | | | | | | | | | | | | | |
| 486 | (2R,3R,4S)-2- | pyrrolidine | | | | | | | | | | y | | |
| | (aminomethyl)-1- | | | | | | | | | | | | | |
| | benzylpyrrolidine-3,4- | | | | | | | | | | | | | |
| | diol | | | | | | | | | | | | | |
| 487 | 2-((2S,3S,4R)-2-((S)- | pyrrolidine | | y | | | | | | | | | y | |
| | 1,2-dihydroxyethyl)-3,4- | | | | | | | | | | | | | |
| | dihydroxypyrrolidin-1- | | | | | | | | | | | | | |
| | yl)acetic acid | | | | | | | | | | | | | |
| 488 | (2R,3R,4S,5R,6R)-2- | piperidine | | y | | | | | | | y | | y | |
| | butyl-6- | | | | | | | | | | | | | |
| | (hydroxymethyl)piperidine- | | | | | | | | | | | | | |
| | 3,4,5-triol | | | | | | | | | | | | | |
| 489 | (1R,2S,8R,8aR)-1,2,8- | indolizidine | y | | | | | | | | | | | |
| | trihydroxy-6-(2- | | | | | | | | | | | | | |
| | hydroxyethyl)hexahydro | | | | | | | | | | | | | |
| | indolizin-5(1H)-one | | | | | | | | | | | | | |
| 490 | (1R,2S,8R,8aR)-1,2,8- | indolizidine | y | | | | | | | | | | | |
| | trihydroxy-6- | | | | | | | | | | | | | |
| | methylhexahydroindolizin- | | | | | | | | | | | | | |
| | 5(1H)-one | | | | | | | | | | | | | |
| 491 | 5-[(2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl]pentanoic acid | | | | | | | | | | | | | |
| 492 | (1R,2S,6R,8R,8aR)-6- | indolizidine | y | | | | | | | | | | | |
| | (2- | | | | | | | | | | | | | |
| | hydroxyethyl)octahydroindolizine- | | | | | | | | | | | | | |
| | 1,2,8-triol | | | | | | | | | | | | | |
| 493 | (1R,2S,6S,8R,8aR)-6- | indolizidine | y | | | | | | | | | | | |
| | (2- | | | | | | | | | | | | | |
| | hydroxyethyl)octahydroindolizine- | | | | | | | | | | | | | |
| | 1,2,8-triol | | | | | | | | | | | | | |
| 494 | 3-[(2R,3R,4R,5R)-3,4- | pyrrolidine | | | | | | | | | y | | | |
| | dihydroxy-2,5- | | | | | | | | | | | | | |
| | bis(hydroxymethyl)pyrrolidin- | | | | | | | | | | | | | |
| | 1-yl]propanoic acid | | | | | | | | | | | | | |
| 495 | (2S,3S,3aS,6S,7S,7aS)- | pyrrolidine | | | | y | y | y | | | y | | | |
| | 2-(hydroxymethyl)-1- | | | | | | | | | | | | | |
| | (methylsulfonyl)octahydropyrano[3, | | | | | | | | | | | | | |
| | 2-b]pyrrole- | | | | | | | | | | | | | |
| | 3,6,7-triol | | | | | | | | | | | | | |
| 496 | (3S,3aS,5S,6R,7R,7aS)- | pyrrolidine | y | y | | | | | | | | | y | |
| | 5-(hydroxymethyl)-1- | | | | | | | | | | | | | |
| | (methylsulfonyl)octahydropyrano[3, | | | | | | | | | | | | | |
| | 2-b]pyrrole- | | | | | | | | | | | | | |
| | 3,6,7-triol | | | | | | | | | | | | | |
| 497 | 3-[(2S,4R)-4-hydroxy-2- | pyrrolidine | | | | | | | | | | y | | y |
| | (hydroxymethyl)pyrrolidin- | | | | | | | | | | | | | |
| | 1-yl]propanoic acid | | | | | | | | | | | | | |
| 498 | [(2S,4R)-4-hydroxy-2- | pyrrolidine | | | | | | | | | | y | | y |
| | (hydroxymethyl)pyrrolidin- | | | | | | | | | | | | | |
| | 1-yl]acetic acid | | | | | | | | | | | | | |
| 499 | 4-[(2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl]butanoic acid | | | | | | | | | | | | | |
| 500 | (2S,3S,4S)-1-benzyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 501 | (2S,3S,4S)-2- | pyrrolidine | | | y | | | | | y | | | | |
| | (hydroxymethyl)-2- | | | | | | | | | | | | | |
| | methylpyrrolidine-3,4- | | | | | | | | | | | | | |
| | diol | | | | | | | | | | | | | |
| 502 | (2R,3S,4S)-N-benzyl- | pyrrolidine | | | y | | | | | y | | | | |
| | 3,4-dihydroxy-2- | | | | | | | | | | | | | |
| | methylpyrrolidine-2- | | | | | | | | | | | | | |
| | carboxamide | | | | | | | | | | | | | |
| 503 | N-{[(3S,4S,5R)-1- | piperidine | | | | | | | | | | y | | |
| | benzyl-4,5- | | | | | | | | | | | | | |
| | dihydroxypiperidin-3- | | | | | | | | | | | | | |
| | yl]methyl}acetamide | | | | | | | | | | | | | |
| 504 | (2R,3S,4S)-3,4- | pyrrolidine | | | y | | | | | y | | | | |
| | dihydroxy-2- | | | | | | | | | | | | | |
| | methylpyrrolidine-2- | | | | | | | | | | | | | |
| | carboxylic acid | | | | | | | | | | | | | |
| 505 | (3aR,4S,6aS)-4- | pyrrolidine | | | y | | | | | y | | | | |
| | (azidomethyl)-5-benzyl- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrole | | | | | | | | | | | | | |
| 506 | (2S,3R,4S)-2- | pyrrolidine | | | y | | | | | y | | | | |
| | (azidomethyl)-1- | | | | | | | | | | | | | |
| | benzylpyrrolidine-3,4- | | | | | | | | | | | | | |
| | diol | | | | | | | | | | | | | |
| 507 | ((3aR,4S,6aS)-5-benzyl- | pyrrolidine | | | y | | | | | y | | | | |
| | 2,2-dimethyltetrahydro- | | | | | | | | | | | | | |
| | 3aH-[1,3]dioxolo[4,5- | | | | | | | | | | | | | |
| | c]pyrrol-4- | | | | | | | | | | | | | |
| | yl)methanamine | | | | | | | | | | | | | |
| 508 | N-(((3aR,4S,6aS)-5- | pyrrolidine | | | y | | | | | y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 509 | (2R,3R,4R,5S)-2- | piperidine | | | | | y | y | | | | | | |
| | (hydroxymethyl)-1-(2- | | | | | | | | | | | | | |
| | morpholinoethyl)piperidine- | | | | | | | | | | | | | |
| | 3,4,5-triol | | | | | | | | | | | | | |
| 510 | (2R,3R,4R,5S)-1- | piperidine | | | | | y | y | | | | | | |
| | benzyl-2- | | | | | | | | | | | | | |
| | (hydroxymethyl)piperidine- | | | | | | | | | | | | | |
| | 3,4,5-triol | | | | | | | | | | | | | |
| 511 | N-(((2R,3R,4S)-3,4- | pyrrolidine | | | | | | | | | | y | | |
| | dihydroxy-1-(9- | | | | | | | | | | | | | |
| | hydroxynonyl)pyrrolidin- | | | | | | | | | | | | | |
| | 2-yl)methyl)acetamide | | | | | | | | | | | | | |
| 512 | N-(((2R,3R,4S)-3,4- | pyrrolidine | | | | | | | | | | y | | |
| | dihydroxy-1- | | | | | | | | | | | | | |
| | nonylpyrrolidin-2- | | | | | | | | | | | | | |
| | yl)methyl)acetamide | | | | | | | | | | | | | |
| 513 | (2R,3R,4S)-2- | pyrrolidine | | | | | | | | | | y | | |
| | (aminomethyl)pyrrolidine- | | | | | | | | | | | | | |
| | 3,4-diol | | | | | | | | | | | | | |
| 514 | (2R,3R,4R,5R)-1- | pyrrolidine | | | | | | | | | y | | | |
| | benzyl-2,5- | | | | | | | | | | | | | |
| | bis(hydroxymethyl)pyrrolidine- | | | | | | | | | | | | | |
| | 3,4-diol | | | | | | | | | | | | | |
| 515 | (2R,3R,4R,5R)-2,5- | pyrrolidine | | | | | | | | | y | | | |
| | bis(hydroxymethyl)-1- | | | | | | | | | | | | | |
| | methylpyrrolidine-3,4- | | | | | | | | | | | | | |
| | diol | | | | | | | | | | | | | |
| 516 | N-(((2R,3R,4S)-3,4- | pyrrolidine | | | | | | | | | | y | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)acetamide | | | | | | | | | | | | | |
| 517 | N-(((2R,3R,4S)-3,4- | pyrrolidine | | | | | | | | | | y | | |
| | dihydroxy-1-(2- | | | | | | | | | | | | | |
| | hydroxyethyl)pyrrolidin- | | | | | | | | | | | | | |
| | 2-yl)methyl)acetamide | | | | | | | | | | | | | |
| 518 | N-(((2R,3R,4S)-1- | pyrrolidine | | | | | | | | | | y | | |
| | (biphenyl-4-ylmethyl)- | | | | | | | | | | | | | |
| | 3,4-dihydroxypyrrolidin- | | | | | | | | | | | | | |
| | 2-yl)methyl)acetamide | | | | | | | | | | | | | |
| 519 | N-(((2R,3R,4S)-1-butyl- | pyrrolidine | | | | | | | | | | y | | |
| | 3,4-dihydroxypyrrolidin- | | | | | | | | | | | | | |
| | 2-yl)methyl)acetamide | | | | | | | | | | | | | |
| 520 | N-(((2R,3R,4S)-3,4- | pyrrolidine | | | | | | | | | | y | | |
| | dihydroxy-1-(2- | | | | | | | | | | | | | |
| | morpholinoethyl)pyrrolidin- | | | | | | | | | | | | | |
| | 2- | | | | | | | | | | | | | |
| | yl)methyl)acetamide | | | | | | | | | | | | | |
| 521 | 3-((2R,3R,4S)-2- | pyrrolidine | | | | | | | | | | y | | |
| | (acetamidomethyl)-3,4- | | | | | | | | | | | | | |
| | dihydroxypyrrolidin-1- | | | | | | | | | | | | | |
| | yl)propanamide | | | | | | | | | | | | | |
| 522 | (1R,2S,3S)-1- | pyrrolidine | | y | | | y | y | | | | | y | |
| | [(2R,3S,4S)-3,4- | | | | | | | | | | | | | |
| | dihydroxypyrrolidin-2- | | | | | | | | | | | | | |
| | yl]butane-1,2,3,4-tetrol | | | | | | | | | | | | | |
| 523 | (2R,3R,4R,5S)-2- | piperidine | | | | | y | y | | | | | | |
| | (hydroxymethyl)-1-(2- | | | | | | | | | | | | | |
| | (piperidin-1- | | | | | | | | | | | | | |
| | yl)ethyl)piperidine-3,4,5- | | | | | | | | | | | | | |
| | triol | | | | | | | | | | | | | |
| 524 | (2R,3R,4R,5S)-1- | piperidine | | | | | y | y | | | | | | |
| | (biphenyl-4-ylmethyl)-2- | | | | | | | | | | | | | |
| | (hydroxymethyl)piperidine- | | | | | | | | | | | | | |
| | 3,4,5-triol | | | | | | | | | | | | | |
| 525 | (1R,2S,5R,8S,8aS)-5- | indolizidine | | | | | | | | | y | | | |
| | methyloctahydroindolizine- | | | | | | | | | | | | | |
| | 1,2,8-triyl triacetate | | | | | | | | | | | | | |
| 526 | (1R,2S,3R)-1- | pyrrolidine | | | | y | y | y | | | y | | | |
| | ((2R,3R,4R)-1-benzyl- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 527 | (1R,2S,3R)-1- | pyrrolidine | | | | y | | | | | y | | | |
| | ((2R,3R,4S)-3,4- | | | | | | | | | | | | | |
| | dihydroxy-1- | | | | | | | | | | | | | |
| | nonylpyrrolidin-2- | | | | | | | | | | | | | |
| | yl)butane-1,2,3,4-tetraol | | | | | | | | | | | | | |
| 528 | (1R,2S,3R)-1- | pyrrolidine | | | | y | | | | | y | | | |
| | ((2R,3R,4S)-1- | | | | | | | | | | | | | |
| | (biphenyl-4-ylmethyl)- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 529 | (1R,2S,3R)-1- | pyrrolidine | | | | y | | | | | y | | | |
| | ((2R,3R,4S)-3,4- | | | | | | | | | | | | | |
| | dihydroxy-1-(9- | | | | | | | | | | | | | |
| | hydroxynonyl)pyrrolidin- | | | | | | | | | | | | | |
| | 2-yl)butane-1,2,3,4- | | | | | | | | | | | | | |
| | tetraol | | | | | | | | | | | | | |
| 530 | 2-((2R,3R,4S)-3,4- | pyrrolidine | | | | y | | | | | y | | | |
| | dihydroxy-2- | | | | | | | | | | | | | |
| | ((1R,2S,3R)-1,2,3,4- | | | | | | | | | | | | | |
| | tetrahydroxybutyl)pyrrolidin- | | | | | | | | | | | | | |
| | 1-yl)acetic acid | | | | | | | | | | | | | |
| 531 | (1R,2S,3R)-1- | pyrrolidine | | | | y | | | | | y | | | |
| | ((2R,3R,4S)-1butyl-3,4- | | | | | | | | | | | | | |
| | dihydroxypyrrolidin-2- | | | | | | | | | | | | | |
| | yl)butane-1,2,3,4-tetraol | | | | | | | | | | | | | |
| 532 | (1R,2S,3R)-1- | pyrrolidine | | | | y | | | | | y | | | |
| | ((2R,3R,4S)-1-benzyl- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 533 | (1R,2S,3R)-1- | pyrrolidine | | | | y | | | | | y | | | |
| | ((2R,3R,4S)-3,4- | | | | | | | | | | | | | |
| | dihydroxy-1-(2- | | | | | | | | | | | | | |
| | hydroxyethyl)pyrrolidin- | | | | | | | | | | | | | |
| | 2-yl)butane-1,2,3,4- | | | | | | | | | | | | | |
| | tetraol | | | | | | | | | | | | | |
| 534 | (1R,2S,5R,6R,7S,8R,8aR)- | indolizidine | | y | | | | | | | y | | y | |
| | 5- | | | | | | | | | | | | | |
| | methyloctahydroindolizine- | | | | | | | | | | | | | |
| | 1,2,6,7,8-pentaol | | | | | | | | | | | | | |
| 535 | (1R,2S,3R)-1- | pyrrolidine | | | | y | | | | | y | | | |
| | ((2R,3R,4S)-3,4- | | | | | | | | | | | | | |
| | dihydroxypyrrolidin-2- | | | | | | | | | | | | | |
| | yl)butane-1,2,3,4-tetraol | | | | | | | | | | | | | |
| 536 | (1R,2S,3R)-1- | pyrrolidine | | | | y | | | | | y | | | |
| | ((2R,3R,4S)-3,4- | | | | | | | | | | | | | |
| | dihydroxy-1-(2-(2- | | | | | | | | | | | | | |
| | methoxyethoxy)ethyl)pyrrolidin- | | | | | | | | | | | | | |
| | 2-yl)butane- | | | | | | | | | | | | | |
| | 1,2,3,4-tetraol | | | | | | | | | | | | | |
| 537 | N-(((2R,3R,4S)-3,4- | pyrrolidine | | | | | | | | | | y | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)acetamide | | | | | | | | | | | | | |
| 538 | N-butyl-2- | pyrrolidine | | y | | | | | | | | | y | |
| | ((2R,3S,4R,5R)-3,4- | | | | | | | | | | | | | |
| | dihydroxy-5- | | | | | | | | | | | | | |
| | (hydroxymethyl)-1- | | | | | | | | | | | | | |
| | nonylpyrrolidin-2- | | | | | | | | | | | | | |
| | yl)acetamide | | | | | | | | | | | | | |
| 539 | 2-((2R,3S,4R,5R)-1- | pyrrolidine | | y | | | | | | | | | y | |
| | benzyl-3,4-dihydroxy-5- | | | | | | | | | | | | | |
| | (hydroxymethyl)pyrrolidin- | | | | | | | | | | | | | |
| | 2-yl)-N- | | | | | | | | | | | | | |
| | butylacetamide | | | | | | | | | | | | | |
| 540 | N-butyl-2- | pyrrolidine | | y | | | | | | | | | y | |
| | ((2R,3S,4R,5R)-3,4- | | | | | | | | | | | | | |
| | dihydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 2-yl)acetamide | | | | | | | | | | | | | |
| 541 | N-butyl-2- | pyrrolidine | | y | | | | | | | | | y | |
| | ((2R,3S,4R,5R)-1-butyl- | | | | | | | | | | | | | |
| | 3,4-dihydroxy-5- | | | | | | | | | | | | | |
| | (hydroxymethyl)pyrrolidin- | | | | | | | | | | | | | |
| | 2-yl)acetamide | | | | | | | | | | | | | |
| 542 | N-(((2R,3R,4S)-1-(2- | pyrrolidine | | | | | | | | | | y | | |
| | (dimethylamino)ethyl)- | | | | | | | | | | | | | |
| | 3,4-dihydroxypyrrolidin- | | | | | | | | | | | | | |
| | 2-yl)methyl)acetamide | | | | | | | | | | | | | |
| 543 | N-butyl-2- | pyrrolidine | | | | | | | | | y | | | |
| | ((2R,3S,4R,5R)-3,4- | | | | | | | | | | | | | |
| | dihydroxy-5- | | | | | | | | | | | | | |
| | (hydroxymethyl)pyrrolidin- | | | | | | | | | | | | | |
| | 2-yl)acetamide | | | | | | | | | | | | | |
| 544 | 2-((2R,3R,4S)-2- | pyrrolidine | | | | | | | | | | y | | |
| | (acetamidomethyl)-3,4- | | | | | | | | | | | | | |
| | dihydroxypyrrolidin-1- | | | | | | | | | | | | | |
| | yl)acetic acid | | | | | | | | | | | | | |
| 545 | (3R,5S)-5- | pyrrolidine | | | | | | | | | | y | | y |
| | (acetamidomethyl)-1-(2- | | | | | | | | | | | | | |
| | acetoxyethyl)pyrrolidin- | | | | | | | | | | | | | |
| | 3-yl acetate | | | | | | | | | | | | | |
| 546 | (3R,5S)-5- | pyrrolidine | | | | | | | | | | y | | y |
| | (acetamidomethyl)-1- | | | | | | | | | | | | | |
| | butylpyrrolidin-3-yl | | | | | | | | | | | | | |
| | acetate | | | | | | | | | | | | | |
| 547 | (3R,5S)-5- | pyrrolidine | | | | | | | | | | y | | y |
| | (acetamidomethyl)-1- | | | | | | | | | | | | | |
| | nonylpyrrolidin-3-yl | | | | | | | | | | | | | |
| | acetate | | | | | | | | | | | | | |
| 548 | N-(((2S,4R)-4-hydroxy- | pyrrolidine | | | | | | | | | | | | |
| | 1-(2- | | | | | | | | | | | | | |
| | hydroxyethyl)pyrrolidin- | | | | | | | | | | | | | |
| | 2-yl)methyl)acetamide | | | | | | | | | | | | | |
| 549 | N-(((2S,4R)-1-butyl-4- | pyrrolidine | | | | | | | | | | y | | y |
| | hydroxypyrrolidin-2- | | | | | | | | | | | | | |
| | yl)methyl)acetamide | | | | | | | | | | | | | |
| 550 | (2R,3R,4R,5S)-2- | piperidine | | | | | y | y | | | | | | |
| | (hydroxymethyl)-1- | | | | | | | | | | | | | |
| | nonylpiperidine-3,4,5- | | | | | | | | | | | | | |
| | triol | | | | | | | | | | | | | |
| 551 | azetidin-3-ol | other | | | | | | | | | | | | |
| 552 | (3S,4S)-tert-butyl 4- | piperidine | | | | | | | | | | | | |
| | bromo-3- | | | | | | | | | | | | | |
| | hydroxypiperidine-1- | | | | | | | | | | | | | |
| | carboxylate | | | | | | | | | | | | | |
| 553 | (R)-tert-butyl 3- | piperidine | | | | | | | | | | | | |
| | (hydroxymethyl)piperidine- | | | | | | | | | | | | | |
| | 1-carboxylate | | | | | | | | | | | | | |
| 554 | (S)-tert-butyl 3- | piperidine | | | | | | | | | | | | |
| | (hydroxymethyl)piperidine- | | | | | | | | | | | | | |
| | 1-carboxylate | | | | | | | | | | | | | |
| 555 | (2R,3R,4R,5R)-2,5- | pyrrolidine | | | | | | | | | | | y | |
| | bis(hydroxymethyl)-1- | | | | | | | | | | | | | |
| | nonylpyrrolidine-3,4-diol | | | | | | | | | | | | | |
| 556 | (2R,3R,4R,5R)-1-(2- | pyrrolidine | | | | | | | | | | | y | |
| | (benzyloxy)ethyl)-2,5- | | | | | | | | | | | | | |
| | bis(hydroxymethyl)pyrrolidine- | | | | | | | | | | | | | |
| | 3,4-diol | | | | | | | | | | | | | |
| 557 | (2R,3R,4R,5R)-2,5- | pyrrolidine | | | | | | | | | | | y | |
| | bis(hydroxymethyl)-1-(9- | | | | | | | | | | | | | |
| | hydroxynonyl)pyrrolidine- | | | | | | | | | | | | | |
| | 3,4-diol | | | | | | | | | | | | | |
| 558 | (2R,3R,4R,5R)-1- | pyrrolidine | | | | | | | | | | | y | |
| | (biphenyl-4-ylmethyl)- | | | | | | | | | | | | | |
| | 2,5- | | | | | | | | | | | | | |
| | bis(hydroxymethyl)pyrrolidine- | | | | | | | | | | | | | |
| | 3,4-diol | | | | | | | | | | | | | |
| 559 | (2R,3R,4R,5R)-2,5- | pyrrolidine | | | | | | | | | | | y | |
| | bis(hydroxymethyl)-1-(2- | | | | | | | | | | | | | |
| | morpholinoethyl)pyrrolidine- | | | | | | | | | | | | | |
| | 3,4-diol | | | | | | | | | | | | | |
| 560 | (2R,3R,4R,5R)-2,5- | pyrrolidine | | | | | | | | | | | y | |
| | bis(hydroxymethyl)-1-(2- | | | | | | | | | | | | | |
| | (piperidin-1- | | | | | | | | | | | | | |
| | yl)ethyl)pyrrolidine-3,4- | | | | | | | | | | | | | |
| | diol | | | | | | | | | | | | | |
| 561 | (2S,3S,4S,5S)-2-((R)-4- | pyrrolidine | | | | | | | | | | | y | |
| | aminopentyl)-5- | | | | | | | | | | | | | |
| | (hydroxymethyl)pyrrolidine- | | | | | | | | | | | | | |
| | 3,4-diol | | | | | | | | | | | | | |
| 562 | (2S,3S,4S,5S)-2-((S)-4- | pyrrolidine | | | | | | | | | | | y | |
| | aminopentyl)-5- | | | | | | | | | | | | | |
| | (hydroxymethyl)pyrrolidine- | | | | | | | | | | | | | |
| | 3,4-diol | | | | | | | | | | | | | |
| 563 | N-((3R,4S,5R)-4,5- | piperidine | | | y | | | | | | | y | | |
| | dihydroxypiperidin-3- | | | | | | | | | | | | | |
| | yl)acetamide | | | | | | | | | | | | | |
| 564 | (2R,3R,4R)-1-(biphenyl- | pyrrolidine | | | y | | | | | y | | | | |
| | 4-ylmethyl)-2- | | | | | | | | | | | | | |
| | (hydroxymethyl)pyrrolidine- | | | | | | | | | | | | | |
| | 3,4-diol | | | | | | | | | | | | | |
| 565 | (R)-piperidin-3- | piperidine | | | | | | | | | | | | |
| | ylmethanol | | | | | | | | | | | | | |
| 566 | (2R,3R,4R)-1-benzyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 567 | (2R,3R,4R,5R)-2,5- | pyrrolidine | | | | | | | | | y | | | |
| | bis(hydroxymethyl)-1-(2- | | | | | | | | | | | | | |
| | (2- | | | | | | | | | | | | | |
| | methoxyethoxy)ethyl)pyrrolidine- | | | | | | | | | | | | | |
| | 3,4-diol | | | | | | | | | | | | | |
| 568 | (2R,3R,4R)-2- | pyrrolidine | | | y | | | | | y | | | | |
| | (hydroxymethyl)-1- | | | | | | | | | | | | | |
| | nonylpyrrolidine-3,4-diol | | | | | | | | | | | | | |
| 569 | (2R,3R,4R)-2- | pyrrolidine | | | y | | | | | y | | | | |
| | (hydroxymethyl)-1-(9- | | | | | | | | | | | | | |
| | hydroxynonyl)pyrrolidine- | | | | | | | | | | | | | |
| | 3,4-diol | | | | | | | | | | | | | |
| 570 | ((3aS,4S,6aR)-5-benzyl- | pyrrolidine | | | | | | | | | | | y | |
| | 2,2-dimethyltetrahydro- | | | | | | | | | | | | | |
| | 3aH-[1,3]dioxolo[4,5- | | | | | | | | | | | | | |
| | c]pyrrol-4-yl)methanol | | | | | | | | | | | | | |
| 571 | (2R,3R,4R)-2- | pyrrolidine | | | y | | | | | y | | | | |
| | (hydroxymethyl)-1-(2-(2- | | | | | | | | | | | | | |
| | methoxyethoxy)ethyl)pyrrolidine- | | | | | | | | | | | | | |
| | 3,4-diol | | | | | | | | | | | | | |
| 572 | (2R,3R,4R)-2- | pyrrolidine | | | y | | | | | y | | | | |
| | (hydroxymethyl)-1-(2- | | | | | | | | | | | | | |
| | morpholinoethyl)pyrrolidine- | | | | | | | | | | | | | |
| | 3,4-diol | | | | | | | | | | | | | |
| 573 | (2R,3R,4R)-2- | pyrrolidine | | | y | | | | | y | | | | |
| | (hydroxymethyl)-1-(2- | | | | | | | | | | | | | |
| | (piperidin-1- | | | | | | | | | | | | | |
| | yl)ethyl)pyrrolidine-3,4- | | | | | | | | | | | | | |
| | diol | | | | | | | | | | | | | |
| 574 | 3-((2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl)propanamide | | | | | | | | | | | | | |
| 575 | (3aR,7R,7aR)-7- | piperidine | | | | | | | | | | | y | |
| | hydroxy-3a- | | | | | | | | | | | | | |
| | (hydroxymethyl)-2,2- | | | | | | | | | | | | | |
| | dimethyltetrahydro- | | | | | | | | | | | | | |
| | [1,3]dioxolo[4,5- | | | | | | | | | | | | | |
| | c]pyridin-4(3aH)-one | | | | | | | | | | | | | |
| 576 | (3aS,4R,7R,7aR)-4- | piperidine | | y | | | | | | | | | y | |
| | (hydroxymethyl)-2,2- | | | | | | | | | | | | | |
| | dimethylhexahydro- | | | | | | | | | | | | | |
| | [1,3]dioxolo[4,5- | | | | | | | | | | | | | |
| | c]pyridin-7-ol | | | | | | | | | | | | | |
| 577 | (3aR,4S,6aS)—N-benzyl- | pyrrolidine | | | | | | | | | | y | | |
| | 2,2,4,6a- | | | | | | | | | | | | | |
| | tetramethyltetrahydro- | | | | | | | | | | | | | |
| | 3aH-[1,3]dioxolo[4,5- | | | | | | | | | | | | | |
| | c]pyrrole-4-carboxamide | | | | | | | | | | | | | |
| 578 | (3aS,7S,7aR)-7- | piperidine | | | | | | | | | | y | | |
| | (azidomethyl)-5-benzyl- | | | | | | | | | | | | | |
| | 2,2,3a- | | | | | | | | | | | | | |
| | trimethylhexahydro- | | | | | | | | | | | | | |
| | [1,3]dioxolo[4,5- | | | | | | | | | | | | | |
| | c]pyridine | | | | | | | | | | | | | |
| 579 | (3aS,4R,7R,7aR)-tert- | piperidine | | y | | | | | | | | | y | |
| | butyl 7-hydroxy-2,2,4- | | | | | | | | | | | | | |
| | trimethyltetrahydro- | | | | | | | | | | | | | |
| | [1,3]dioxolo[4,5- | | | | | | | | | | | | | |
| | c]pyridine-5(6H)- | | | | | | | | | | | | | |
| | carboxylate | | | | | | | | | | | | | |
| 580 | tert-butyl 5-hydroxy-5,6- | piperidine | | | | | | | | | | | | |
| | dihydropyridine-1(2H)- | | | | | | | | | | | | | |
| | carboxylate | | | | | | | | | | | | | |
| 581 | N-butyl-2- | pyrrolidine | | y | | | | | | | | | y | |
| | ((2R,3S,4R,5R)-3,4- | | | | | | | | | | | | | |
| | dihydroxy-5- | | | | | | | | | | | | | |
| | (hydroxymethyl)-1-(2-(2- | | | | | | | | | | | | | |
| | methoxyethoxy)ethyl)pyrrolidin- | | | | | | | | | | | | | |
| | 2-yl)acetamide | | | | | | | | | | | | | |
| 582 | N-butyl-2- | pyrrolidine | | y | | | | | | | | | y | |
| | ((2R,3S,4R,5R)-3,4- | | | | | | | | | | | | | |
| | dihydroxy-5- | | | | | | | | | | | | | |
| | (hydroxymethyl)-1-(9- | | | | | | | | | | | | | |
| | hydroxynonyl)pyrrolidin- | | | | | | | | | | | | | |
| | 2-yl)acetamide | | | | | | | | | | | | | |
| 583 | N-(((2S,3R,4S)-3,4- | pyrrolidine | | | y | | | | | y | | | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)acetamide | | | | | | | | | | | | | |
| 584 | N-(((2S,3R,4S)-1- | pyrrolidine | | | y | | | | | y | | | | |
| | (biphenyl-4-ylmethyl)- | | | | | | | | | | | | | |
| | 3,4-dihydroxypyrrolidin- | | | | | | | | | | | | | |
| | 2-yl)methyl)acetamide | | | | | | | | | | | | | |
| 585 | 2-((2R,3S,4R,5R)-1- | pyrrolidine | | y | | | | | | | | | y | |
| | (biphenyl-4-ylmethyl)- | | | | | | | | | | | | | |
| | 3,4-dihydroxy-5- | | | | | | | | | | | | | |
| | (hydroxymethyl)pyrrolidin- | | | | | | | | | | | | | |
| | 2-yl)-N- | | | | | | | | | | | | | |
| | butylacetamide | | | | | | | | | | | | | |
| 586 | N-(((2R,3R,4S)-1- | pyrrolidine | | | | | | | | | | y | | |
| | benzyl-3,4- | | | | | | | | | | | | | |
| | dihydroxypyrrolidin-2- | | | | | | | | | | | | | |
| | yl)methyl)benzamide | | | | | | | | | | | | | |
| 587 | N-(((2S,3R,4S)-3,4- | pyrrolidine | | | y | | | | | y | | | | |
| | dihydroxy-1- | | | | | | | | | | | | | |
| | nonylpyrrolidin-2- | | | | | | | | | | | | | |
| | yl)methyl)acetamide | | | | | | | | | | | | | |
| 588 | ((3aS,4S,6aR)-2,2- | pyrrolidine | | | | | | | | | | y | | |
| | dimethyltetrahydro-3aH- | | | | | | | | | | | | | |
| | [1,3]dioxolo[4,5-c]pyrrol- | | | | | | | | | | | | | |
| | 4-yl)methanol | | | | | | | | | | | | | |
| 589 | (1R,2S,3R)-1-((3R,4R)- | pyrrolidine | | | | y | y | y | | | y | | | |
| | 3,4-dihydroxy-1-(2- | | | | | | | | | | | | | |
| | hydroxyethyl)pyrrolidin- | | | | | | | | | | | | | |
| | 2-yl)butane-1,2,3,4- | | | | | | | | | | | | | |
| | tetraol | | | | | | | | | | | | | |
| 590 | (1R,2S,3R)-1-((3R,4R)- | pyrrolidine | | | | y | y | y | | | y | | | |
| | 1-(biphenyl-4-ylmethyl)- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 591 | (1R,2S,3R)-1-((3R,4R)- | pyrrolidine | | | | y | y | y | | | y | | | |
| | 3,4-dihydroxy-1-(9- | | | | | | | | | | | | | |
| | hydroxynonyl)pyrrolidin- | | | | | | | | | | | | | |
| | 2-yl)butane-1,2,3,4- | | | | | | | | | | | | | |
| | tetraol | | | | | | | | | | | | | |
| 592 | (1R,2S,3R)-1-((3R,4R)- | pyrrolidine | | | | y | y | y | | | y | | | |
| | 3,4-dihydroxy-1-(2-(2- | | | | | | | | | | | | | |
| | methoxyethoxy)ethyl)pyrrolidin- | | | | | | | | | | | | | |
| | 2-yl)butane- | | | | | | | | | | | | | |
| | 1,2,3,4-tetraol | | | | | | | | | | | | | |
| 593 | (1R,2S,3R)-1-((3R,4R)- | pyrrolidine | | | | y | y | y | | | y | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 594 | (1R,2S,3R)-1-((3R,4R)- | pyrrolidine | | | | y | y | y | | | y | | | |
| | 1-butyl-3,4- | | | | | | | | | | | | | |
| | dihydroxypyrrolidin-2- | | | | | | | | | | | | | |
| | yl)butane-1,2,3,4-tetraol | | | | | | | | | | | | | |
| 595 | (1R,2S,3R)-1-((3R,4R)- | pyrrolidine | | | | y | y | y | | | y | | | |
| | 3,4-dihydroxy-1- | | | | | | | | | | | | | |
| | nonylpyrrolidin-2- | | | | | | | | | | | | | |
| | yl)butane-1,2,3,4-tetraol | | | | | | | | | | | | | |
| 596 | (5R,6R,7S,8R)-5- | piperidine | | | | | | | | | y | | | |
| | methyl-5,6,7,8- | | | | | | | | | | | | | |
| | tetrahydrotetrazolo[1,5- | | | | | | | | | | | | | |
| | a]pyridine-6,7,8-triol | | | | | | | | | | | | | |
| 597 | N-(((2S,3R,4S)-1- | pyrrolidine | | | y | | | | | y | | | | |
| | benzyl-3,4- | | | | | | | | | | | | | |
| | dihydroxypyrrolidin-2- | | | | | | | | | | | | | |
| | yl)methyl)benzamide | | | | | | | | | | | | | |
| 598 | N-(((2S,3R,4S)-1- | pyrrolidine | | | y | | | | | y | | | | |
| | benzyl-3,4- | | | | | | | | | | | | | |
| | dihydroxypyrrolidin-2- | | | | | | | | | | | | | |
| | yl)methyl)acetamide | | | | | | | | | | | | | |
| 599 | N-(((3aR,4S,6aS)-5- | pyrrolidine | | | y | | | | | y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)benzamide | | | | | | | | | | | | | |
| 600 | N-(((2S,3R,4S)-3,4- | pyrrolidine | | | y | | | | | y | | | | |
| | dihydroxy-1-(2- | | | | | | | | | | | | | |
| | hydroxyethyl)pyrrolidin- | | | | | | | | | | | | | |
| | 2-yl)methyl)acetamide | | | | | | | | | | | | | |
| 601 | N-(((2S,3R,4S)-3,4- | pyrrolidine | | | y | | | | | y | | | | |
| | dihydroxypyrrolidin-2- | | | | | | | | | | | | | |
| | yl)methyl)acetamide | | | | | | | | | | | | | |
| 602 | N-(((2S,3R,4S)-3,4- | pyrrolidine | | | y | | | | | y | | | | |
| | dihydroxypyrrolidin-2- | | | | | | | | | | | | | |
| | yl)methyl)benzamide | | | | | | | | | | | | | |
| 603 | N-(((2S,3R,4S)-1-butyl- | pyrrolidine | | | y | | | | | y | | | | |
| | 3,4-dihydroxypyrrolidin- | | | | | | | | | | | | | |
| | 2-yl)methyl)acetamide | | | | | | | | | | | | | |
| 604 | (2S,3R,4S)-2- | pyrrolidine | | | y | | | | | y | | | | |
| | (aminomethyl)-1- | | | | | | | | | | | | | |
| | benzylpyrrolidine-3,4- | | | | | | | | | | | | | |
| | diol | | | | | | | | | | | | | |
| 605 | ((3aS,4S,6aR)-5- | pyrrolidine | | | | | | | | | | y | | |
| | (biphenyl-4-ylmethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4-yl)methanol | | | | | | | | | | | | | |
| 606 | (2S,3S,4R)-1-(biphenyl- | pyrrolidine | | y | | | | | | | | | y | |
| | 4-ylmethyl)-2-((S)-1,2- | | | | | | | | | | | | | |
| | dihydroxyethyl)pyrrolidine- | | | | | | | | | | | | | |
| | 3,4-diol | | | | | | | | | | | | | |
| 607 | N-(((2S,3R,4S)-1-butyl- | pyrrolidine | | | y | | | | | y | | | | |
| | 3,4-dihydroxypyrrolidin- | | | | | | | | | | | | | |
| | 2-yl)methyl)benzamide | | | | | | | | | | | | | |
| 608 | N-(((3aR,4S,6aS)-5- | pyrrolidine | | | y | | | | | y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamide | | | | | | | | | | | | | |
| 609 | N-(((3aR,4S,6aS)-2,2- | pyrrolidine | | | y | | | | | y | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 610 | N-(((3aR,4S,6aS)-5- | pyrrolidine | | | y | | | | | y | | | | |
| | (biphenyl-4-ylmethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4- | | | | | | | | | | | | | |
| | yl)methyl)acetamide | | | | | | | | | | | | | |
| 611 | N-(((3aR,4S,6aS)-2,2- | pyrrolidine | | | y | | | | | y | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamide | | | | | | | | | | | | | |
| 612 | N-(((2S,3R,4S)-3,4- | pyrrolidine | | | y | | | | | y | | | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)benzamide | | | | | | | | | | | | | |
| 613 | N-(((2S,3R,4S)-3,4- | pyrrolidine | | | y | | | | | y | | | | |
| | dihydroxy-1-(9- | | | | | | | | | | | | | |
| | hydroxynonyl)pyrrolidin- | | | | | | | | | | | | | |
| | 2-yl)methyl)benzamide | | | | | | | | | | | | | |
| 614 | N-(((2R,3R,4S)-3,4- | pyrrolidine | | | | | | | | | | y | | |
| | dihydroxypyrrolidin-2- | | | | | | | | | | | | | |
| | yl)methyl)benzamide | | | | | | | | | | | | | |
| 615 | N-(((2S,3R,4S)-1-(2- | pyrrolidine | | | y | | | | | y | | | | |
| | (dimethylamino)ethyl)- | | | | | | | | | | | | | |
| | 3,4-dihydroxypyrrolidin- | | | | | | | | | | | | | |
| | 2-yl)methyl)acetamide | | | | | | | | | | | | | |
| 616 | N-(((2S,3R,4S)-3,4- | pyrrolidine | | | y | | | | | y | | | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)acetamide | | | | | | | | | | | | | |
| 617 | N-(((2R,3R,4S)-1-butyl- | pyrrolidine | | | | | | | | | | y | | |
| | 3,4-dihydroxypyrrolidin- | | | | | | | | | | | | | |
| | 2-yl)methyl)benzamide | | | | | | | | | | | | | |
| 618 | N-(((2S,3R,4S)-3,4- | pyrrolidine | | | y | | | | | y | | | | |
| | dihydroxy-1-(2- | | | | | | | | | | | | | |
| | morpholinoethyl)pyrrolidin- | | | | | | | | | | | | | |
| | 2- | | | | | | | | | | | | | |
| | yl)methyl)acetamide | | | | | | | | | | | | | |
| 619 | N-(((2R,3R,4S)-1- | pyrrolidine | | | | | | | | | | y | | |
| | benzyl-3,4- | | | | | | | | | | | | | |
| | dihydroxypyrrolidin-2- | | | | | | | | | | | | | |
| | yl)methyl)-2,2,2- | | | | | | | | | | | | | |
| | trifluoroacetamide | | | | | | | | | | | | | |
| 620 | N-butyl-2- | pyrrolidine | | y | | | | | | | | | y | |
| | ((2R,3S,4R,5R)-3,4- | | | | | | | | | | | | | |
| | dihydroxy-5- | | | | | | | | | | | | | |
| | (hydroxymethyl)-1-(2- | | | | | | | | | | | | | |
| | morpholinoethyl)pyrrolidin- | | | | | | | | | | | | | |
| | 2-yl)acetamide | | | | | | | | | | | | | |
| 621 | N-(((2S,3R,4S)-3,4- | pyrrolidine | | | y | | | | | y | | | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)benzamide | | | | | | | | | | | | | |
| 622 | N-(((2S,3R,4S)-3,4- | pyrrolidine | | | y | | | | | y | | | | |
| | dihydroxy-1- | | | | | | | | | | | | | |
| | nonylpyrrolidin-2- | | | | | | | | | | | | | |
| | yl)methyl)benzamide | | | | | | | | | | | | | |
| 623 | (2S,3S,4R)-1-(biphenyl- | pyrrolidine | | | | | | | | | | y | | |
| | 4-ylmethyl)-2- | | | | | | | | | | | | | |
| | (hydroxymethyl)pyrrolidine- | | | | | | | | | | | | | |
| | 3,4-diol | | | | | | | | | | | | | |
| 624 | (1R,2S,3R)-1-((3R,4S)- | pyrrolidine | | | | y | | | | | y | | | |
| | 3,4-dihydroxy-1- | | | | | | | | | | | | | |
| | methylpyrrolidin-2- | | | | | | | | | | | | | |
| | yl)butane-1,2,3,4-tetraol | | | | | | | | | | | | | |
| 625 | (1R,2S,3R)-1-((3R,4R)- | pyrrolidine | | | | y | y | y | | | y | | | |
| | 3,4-dihydroxy-1- | | | | | | | | | | | | | |
| | methylpyrrolidin-2- | | | | | | | | | | | | | |
| | yl)butane-1,2,3,4-tetraol | | | | | | | | | | | | | |
| 626 | (1R,2S,3R)-1-((3R,4R)- | pyrrolidine | | | | y | y | y | | | y | | | |
| | 3,4-dihydroxy-1-(2- | | | | | | | | | | | | | |
| | morpholinoethyl)pyrrolidin- | | | | | | | | | | | | | |
| | 2-yl)butane-1,2,3,4- | | | | | | | | | | | | | |
| | tetraol | | | | | | | | | | | | | |
| 627 | (1R,2S,3R)-1-((3R,4R)- | pyrrolidine | | | | y | y | y | | | y | | | |
| | 3,4-dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)butane-1,2,3,4-tetraol | | | | | | | | | | | | | |
| 628 | N-(((2R,3R,4S)-3,4- | pyrrolidine | | | | | | | | | | y | | |
| | dihydroxy-1- | | | | | | | | | | | | | |
| | methylpyrrolidin-2- | | | | | | | | | | | | | |
| | yl)methyl)benzamide | | | | | | | | | | | | | |
| 629 | N-(((3aR,4S,6aS)-2,2- | pyrrolidine | | | y | | | | | y | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)benzamide | | | | | | | | | | | | | |
| 630 | N-(((3aR,4S,6aS)-2,2- | pyrrolidine | | | y | | | | | y | | | | |
| | dimethyl-5- | | | | | | | | | | | | | |
| | nonyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 631 | 2,2,2-trifluoro-N- | pyrrolidine | | | y | | | | | y | | | | |
| | (((3aR,4S,6aS)-5-(2-(2- | | | | | | | | | | | | | |
| | methoxyethoxy)ethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4- | | | | | | | | | | | | | |
| | yl)methyl)acetamide | | | | | | | | | | | | | |
| 632 | N-(((3aR,4S,6aS)-5- | pyrrolidine | | | y | | | | | y | | | | |
| | (biphenyl-4-ylmethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4-yl)methyl)- | | | | | | | | | | | | | |
| | 2,2,2-trifluoroacetamide | | | | | | | | | | | | | |
| 633 | N-(((2S,3R,4S)-3,4- | pyrrolidine | | | y | | | | | y | | | | |
| | dihydroxy-1-(2- | | | | | | | | | | | | | |
| | morpholinoethyl)pyrrolidin- | | | | | | | | | | | | | |
| | 2- | | | | | | | | | | | | | |
| | yl)methyl)benzamide | | | | | | | | | | | | | |
| 634 | N-(((2S,3R,4S)-3,4- | pyrrolidine | | | y | | | | | y | | | | |
| | dihydroxy-1-(9- | | | | | | | | | | | | | |
| | hydroxynonyl)pyrrolidin- | | | | | | | | | | | | | |
| | 2-yl)methyl)acetamide | | | | | | | | | | | | | |
| 635 | (1S,2S,3S,6R,7R,7aR)- | pyrrolizidine | | | | | y | y | y | | | | | |
| | 1,6,7-trihydroxy-3- | | | | | | | | | | | | | |
| | (hydroxymethyl)hexahydro- | | | | | | | | | | | | | |
| | 1H-pyrrolizin-2-yl | | | | | | | | | | | | | |
| | methanesulfonate | | | | | | | | | | | | | |
| 636 | (3R,4S,5S)-5- | piperidine | | | | | | | | | | y | | |
| | (aminomethyl)piperidine- | | | | | | | | | | | | | |
| | 3,4-diol | | | | | | | | | | | | | |
| 637 | N-{[(3R,4S,5R)-4,5- | piperidine | | | | | | | | | | y | | |
| | dihydroxypiperidin-3- | | | | | | | | | | | | | |
| | yl]methyl}acetamide | | | | | | | | | | | | | |
| 638 | (2S,4R)-4-hydroxy-1,1- | pyrrolidine | | | | | | | | | | | | |
| | dimethylpyrrolidinium-2- | | | | | | | | | | | | | |
| | carboxylate | | | | | | | | | | | | | |
| 639 | N-((3R,4S,5R)-5- | piperidine | | | | | | | | | | | | |
| | (benzyloxy)-4- | | | | | | | | | | | | | |
| | hydroxypiperidin-3- | | | | | | | | | | | | | |
| | yl)acetamide | | | | | | | | | | | | | |
| 640 | (3S,4S,5S)-1-(2- | piperidine | | | | | | | | | | | | |
| | hydroxyethyl)-5- | | | | | | | | | | | | | |
| | (hydroxymethyl)piperidine- | | | | | | | | | | | | | |
| | 3,4-diol | | | | | | | | | | | | | |
| 641 | (3S,4S,5S)-5- | piperidine | | | | | | | | | | | | |
| | (hydroxymethyl)piperidine- | | | | | | | | | | | | | |
| | 3,4-diol | | | | | | | | | | | | | |
| 642 | (1S,2R,3S,4R,5R)- | other | | | | | Y | Y | Y | | | | | |
| | 2,3,4-trihydroxy-N-(N′- | | | | | | | | | | | | | |
| | octylthiocarbamoyl)-6- | | | | | | | | | | | | | |
| | oxa-nor-tropane | | | | | | | | | | | | | |
| 643 | (5R,6R,7S,8R,8aR)- | other | | | | | Y | Y | Y | | | | | |
| | 5,6,7,8-Tetrahydroxy-3- | | | | | | | | | | | | | |
| | octylimino-2- | | | | | | | | | | | | | |
| | oxaindolizidine | | | | | | | | | | | | | |
| 644 | (1S,2R,3S,4R,5R)—N- | other | | | | | Y | Y | Y | | | | | |
| | (N′-Butylthiocarbamoyl)- | | | | | | | | | | | | | |
| | 2,3,4-trihydroxy-6-oxa- | | | | | | | | | | | | | |
| | nor-tropane | | | | | | | | | | | | | |
| 645 | (3Z,5R,6R,7S,8R,8aR)- | other | | | | | Y | Y | Y | | | | | |
| | 3- | | | | | | | | | | | | | |
| | (octylimino)hexahydro[1, | | | | | | | | | | | | | |
| | 3]thiazolo[3,4- | | | | | | | | | | | | | |
| | a]pyridine-5,6,7,8-tetrol | | | | | | | | | | | | | |
| 646 | N-(((2R,3R,4S)-3,4- | pyrrolidine | | | | | | | | | | Y | | |
| | dihydroxy-1- | | | | | | | | | | | | | |
| | nonylpyrrolidin-2- | | | | | | | | | | | | | |
| | yl)methyl)benzamide | | | | | | | | | | | | | |
| 647 | N-(((2R,3R,4S)-3,4- | pyrrolidine | | | | | | | | | | Y | | |
| | dihydroxy-1-(2- | | | | | | | | | | | | | |
| | hydroxyethyl)pyrrolidin- | | | | | | | | | | | | | |
| | 2-yl)methyl)benzamide | | | | | | | | | | | | | |
| 648 | N-(((2R,3R,4S)-3,4- | pyrrolidine | | | | | | | | | | Y | | |
| | dihydroxy-1-(9- | | | | | | | | | | | | | |
| | hydroxynonyl)pyrrolidin- | | | | | | | | | | | | | |
| | 2-yl)methyl)benzamide | | | | | | | | | | | | | |
| 649 | N-(((2R,3R,4S)-1- | pyrrolidine | | | | | | | | | | Y | | |
| | (biphenyl-4-ylmethyl)- | | | | | | | | | | | | | |
| | 3,4-dihydroxypyrrolidin- | | | | | | | | | | | | | |
| | 2-yl)methyl)benzamide | | | | | | | | | | | | | |
| 650 | N-(((2R,3R,4S)-3,4- | pyrrolidine | | | | | | | | | | Y | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)benzamide | | | | | | | | | | | | | |
| 651 | N-(((2R,3R,4S)-3,4- | pyrrolidine | | | | | | | | | | Y | | |
| | dihydroxy-1-(2- | | | | | | | | | | | | | |
| | morpholinoethyl)pyrrolidin- | | | | | | | | | | | | | |
| | 2- | | | | | | | | | | | | | |
| | yl)methyl)benzamide | | | | | | | | | | | | | |
| 652 | N-(((3aR,4S,6aS)-5- | pyrrolidine | | | Y | | | | | Y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)biphenyl-4- | | | | | | | | | | | | | |
| | carboxamide | | | | | | | | | | | | | |
| 653 | N-(((2R,3R,4S)-3,4- | pyrrolidine | | | | | | | | | | Y | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)benzamide | | | | | | | | | | | | | |
| 654 | N-(((2R,3R,4S)-1-(2- | pyrrolidine | | | | | | | | | | Y | | |
| | (dimethylamino)ethyl)- | | | | | | | | | | | | | |
| | 3,4-dihydroxypyrrolidin- | | | | | | | | | | | | | |
| | 2-yl)methyl)benzamide | | | | | | | | | | | | | |
| 655 | 2-((2R,3R,4S)-2- | pyrrolidine | | | | | | | | | | Y | | |
| | (benzamidomethyl)-3,4- | | | | | | | | | | | | | |
| | dihydroxypyrrolidin-1- | | | | | | | | | | | | | |
| | yl)acetic acid | | | | | | | | | | | | | |
| 656 | N-(((2R,3R,4S)-3,4- | pyrrolidine | | | | | | | | | | Y | | |
| | dihydroxy-1- | | | | | | | | | | | | | |
| | methylpyrrolidin-2- | | | | | | | | | | | | | |
| | yl)methyl)acetamide | | | | | | | | | | | | | |
| 657 | N-(((2S,3R,4S)-1- | pyrrolidine | | | Y | | | | | Y | | | | |
| | benzyl-3,4- | | | | | | | | | | | | | |
| | dihydroxypyrrolidin-2- | | | | | | | | | | | | | |
| | yl)methyl)biphenyl-4- | | | | | | | | | | | | | |
| | carboxamide | | | | | | | | | | | | | |
| 658 | N-(((2S,3R,4S)-1-butyl- | pyrrolidine | | | Y | | | | | Y | | | | |
| | 3,4-dihydroxypyrrolidin- | | | | | | | | | | | | | |
| | 2-yl)methyl)biphenyl-4- | | | | | | | | | | | | | |
| | carboxamide | | | | | | | | | | | | | |
| 659 | N-(((2S,3R,4S)-3,4- | pyrrolidine | | | Y | | | | | Y | | | | |
| | dihydroxy-1- | | | | | | | | | | | | | |
| | nonylpyrrolidin-2- | | | | | | | | | | | | | |
| | yl)methyl)biphenyl-4- | | | | | | | | | | | | | |
| | carboxamide | | | | | | | | | | | | | |
| 660 | N-(((2S,3R,4S)-3,4- | pyrrolidine | | | Y | | | | | Y | | | | |
| | dihydroxy-1-(9- | | | | | | | | | | | | | |
| | hydroxynonyl)pyrrolidin- | | | | | | | | | | | | | |
| | 2-yl)methyl)biphenyl-4- | | | | | | | | | | | | | |
| | carboxamide | | | | | | | | | | | | | |
| 661 | N-(((3aR,4S,6aS)-5-(9- | pyrrolidine | | | Y | | | | | Y | | | | |
| | hydroxynonyl)-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)benzamide | | | | | | | | | | | | | |
| 662 | N-(((3aR,4S,6aS)-5- | pyrrolidine | | | Y | | | | | Y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamide | | | | | | | | | | | | | |
| 663 | 2,2,2-trifluoro-N- | pyrrolidine | | | Y | | | | | Y | | | | |
| | (((3aR,4S,6aS)-5-(9- | | | | | | | | | | | | | |
| | hydroxynonyl)-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 664 | N-(((2S,3R,4S)-1- | pyrrolidine | | | Y | | | | | Y | | | | |
| | (biphenyl-4-ylmethyl)- | | | | | | | | | | | | | |
| | 3,4-dihydroxypyrrolidin- | | | | | | | | | | | | | |
| | 2-yl)methyl)benzamide | | | | | | | | | | | | | |
| 665 | 3-((2S,3R,4S)-2- | pyrrolidine | | | Y | | | | | Y | | | | |
| | (acetamidomethyl)-3,4- | | | | | | | | | | | | | |
| | dihydroxypyrrolidin-1- | | | | | | | | | | | | | |
| | yl)propanamide | | | | | | | | | | | | | |
| 666 | N-(((2S,3R,4S)-1-(3- | pyrrolidine | | | Y | | | | | Y | | | | |
| | amino-3-oxopropyl)-3,4- | | | | | | | | | | | | | |
| | dihydroxypyrrolidin-2- | | | | | | | | | | | | | |
| | yl)methyl)benzamide | | | | | | | | | | | | | |
| 667 | N-(((2S,3R,4S)-1-(2- | pyrrolidine | | | Y | | | | | Y | | | | |
| | (dimethylamino)ethyl)- | | | | | | | | | | | | | |
| | 3,4-dihydroxypyrrolidin- | | | | | | | | | | | | | |
| | 2-yl)methyl)benzamide | | | | | | | | | | | | | |
| 668 | N-(((3aR,4R,6aS)-5- | pyrrolidine | | | | | | | | | | Y | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)butyramide | | | | | | | | | | | | | |
| 669 | N-(((2R,3R,4S)-1- | pyrrolidine | | | | | | | | | | Y | | |
| | benzyl-3,4- | | | | | | | | | | | | | |
| | dihydroxypyrrolidin-2- | | | | | | | | | | | | | |
| | yl)methyl)butyramide | | | | | | | | | | | | | |
| 670 | N-(((2S,3R,4S)-3,4- | pyrrolidine | | | Y | | | | | Y | | | | |
| | dihydroxypyrrolidin-2- | | | | | | | | | | | | | |
| | yl)methyl)biphenyl-4- | | | | | | | | | | | | | |
| | carboxamide | | | | | | | | | | | | | |
| 671 | (3aS,4R,6aR)-4- | pyrrolidine | | | Y | | | | | Y | | | | |
| | (azidomethyl)-2,2- | | | | | | | | | | | | | |
| | dimethyltetrahydro-3aH- | | | | | | | | | | | | | |
| | [1,3]dioxolo[4,5- | | | | | | | | | | | | | |
| | c]pyrrole | | | | | | | | | | | | | |
| 672 | N-(((3aR,4R,6aS)-5- | pyrrolidine | | | | | | | | | | Y | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)biphenyl-4- | | | | | | | | | | | | | |
| | carboxamide | | | | | | | | | | | | | |
| 673 | N-(((2R,3R,4S)-1- | pyrrolidine | | | | | | | | | | Y | | |
| | benzyl-3,4- | | | | | | | | | | | | | |
| | dihydroxypyrrolidin-2- | | | | | | | | | | | | | |
| | yl)methyl)biphenyl-4- | | | | | | | | | | | | | |
| | carboxamide | | | | | | | | | | | | | |
| 674 | N-(((2S,3R,4S)-3,4- | pyrrolidine | | | Y | | | | | Y | | | | |
| | dihydroxypyrrolidin-2- | | | | | | | | | | | | | |
| | yl)methyl)-2,2,2- | | | | | | | | | | | | | |
| | trifluoroacetamide | | | | | | | | | | | | | |
| 675 | N-(((3aR,4S,6aS)-2,2- | pyrrolidine | | | Y | | | | | Y | | | | |
| | dimethyl-5-(2- | | | | | | | | | | | | | |
| | morpholinoethyl)tetrahydro- | | | | | | | | | | | | | |
| | 3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamid | | | | | | | | | | | | | |
| 676 | N-(((3aR,4S,6aS)-5- | pyrrolidine | | | Y | | | | | Y | | | | |
| | butyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamide | | | | | | | | | | | | | |
| 677 | N-(((3aR,4S,6aS)-2,2- | pyrrolidine | | | Y | | | | | Y | | | | |
| | dimethyl-5-(2-(piperidin- | | | | | | | | | | | | | |
| | 1-yl)ethyl)tetrahydro- | | | | | | | | | | | | | |
| | 3aH-[1,3]dioxolo[4,5- | | | | | | | | | | | | | |
| | c]pyrrol-4-yl)methyl)- | | | | | | | | | | | | | |
| | 2,2,2-trifluoroacetamide | | | | | | | | | | | | | |
| 678 | N-(((3aR,4S,6aS)-5-(2- | pyrrolidine | | | Y | | | | | Y | | | | |
| | (dimethylamino)ethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4-yl)methyl)- | | | | | | | | | | | | | |
| | 2,2,2-trifluoroacetamide | | | | | | | | | | | | | |
| 679 | N-(((2S,3R,4S)-3,4- | pyrrolidine | | | Y | | | | | Y | | | | |
| | dihydroxy-1- | | | | | | | | | | | | | |
| | methylpyrrolidin-2- | | | | | | | | | | | | | |
| | yl)methyl)acetamide | | | | | | | | | | | | | |
| 680 | (2S,3R,4R)-2- | pyrrolidine | | | | Y | | | | | Y | | | |
| | [(2R,3S,4R)-3,4- | | | | | | | | | | | | | |
| | dihydroxytetrahydrofuran- | | | | | | | | | | | | | |
| | 2-yl]pyrrolidine-3,4- | | | | | | | | | | | | | |
| | diol | | | | | | | | | | | | | |
| 681 | (2R,3R,4R)-1-butyl-2- | piperidine | | | Y | | | | | | | | | Y |
| | (hydroxymethyl)piperidine- | | | | | | | | | | | | | |
| | 3,4-diol | | | | | | | | | | | | | |
| 682 | N-(((2S,3R,4S)-1-butyl- | pyrrolidine | | | Y | | | | | Y | | | | |
| | 3,4-dihydroxypyrrolidin- | | | | | | | | | | | | | |
| | 2-yl)methyl)-2,2,2- | | | | | | | | | | | | | |
| | trifluoroacetamide | | | | | | | | | | | | | |
| 683 | N-(((2S,3R,4S)-3,4- | pyrrolidine | | | Y | | | | | Y | | | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)-2,2,2- | | | | | | | | | | | | | |
| | trifluoroacetamide | | | | | | | | | | | | | |
| 684 | tert-butyl | pyrrolidine | | | Y | | | | | Y | | | | |
| | ((3aR,4S,6aS)-5-(2- | | | | | | | | | | | | | |
| | hydroxyethyl)-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methylcarbamate | | | | | | | | | | | | | |
| 685 | dimethyl 1-(((2S,3R,4S)- | pyrrolidine | | | Y | | | | | Y | | | | |
| | 1-benzyl-3,4- | | | | | | | | | | | | | |
| | dihydroxypyrrolidin-2- | | | | | | | | | | | | | |
| | yl)methyl)-1H-1,2,3- | | | | | | | | | | | | | |
| | triazole-4,5- | | | | | | | | | | | | | |
| | dicarboxylate | | | | | | | | | | | | | |
| 686 | (2S,3R,4S)-2- | pyrrolidine | | | Y | | | | | Y | | | | |
| | (aminomethyl)-1- | | | | | | | | | | | | | |
| | (biphenyl-4- | | | | | | | | | | | | | |
| | ylmethyl)pyrrolidine-3,4- | | | | | | | | | | | | | |
| | diol | | | | | | | | | | | | | |
| 687 | (2R,3S,4R)-2- | pyrrolidine | | | | | | | | Y | | | | |
| | (aminomethyl)-1- | | | | | | | | | | | | | |
| | benzylpyrrolidine-3,4- | | | | | | | | | | | | | |
| | diol | | | | | | | | | | | | | |
| 688 | N-(((2S,3R,4S)-3,4- | pyrrolidine | | | | | | | | Y | | | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)biphenyl-4- | | | | | | | | | | | | | |
| | carboxamide | | | | | | | | | | | | | |
| 689 | N-(((2R,3R,4S)-1-butyl- | pyrrolidine | | | | | | | | | | Y | | |
| | 3,4-dihydroxypyrrolidin- | | | | | | | | | | | | | |
| | 2-yl)methyl)butyramide | | | | | | | | | | | | | |
| 690 | (2R,3S,4R)-2- | pyrrolidine | | | Y | | | | | Y | | | | |
| | (aminomethyl)pyrrolidine- | | | | | | | | | | | | | |
| | 3,4-diol | | | | | | | | | | | | | |
| 691 | N-((2R,3R)-3- | pyrrolidine | | | | | | | | | | | | Y |
| | ((2R,3R,4R)-3,4- | | | | | | | | | | | | | |
| | dihydroxypyrrolidin-2- | | | | | | | | | | | | | |
| | yl)-2,3- | | | | | | | | | | | | | |
| | dihydroxypropyl)acetamide | | | | | | | | | | | | | |
| 692 | (1R,2R)-1-((2R,3R,4R)- | pyrrolidine | | | | Y | Y | Y | | | | | | |
| | 3,4-dihydroxy-1- | | | | | | | | | | | | | |
| | nonylpyrrolidin-2- | | | | | | | | | | | | | |
| | yl)propane-1,2,3-triol | | | | | | | | | | | | | |
| 693 | (1R,2R)-1-((2R,3R,4R)- | pyrrolidine | | | | Y | Y | Y | | | | | | |
| | 3,4-dihydroxy-1-(2-(2- | | | | | | | | | | | | | |
| | methoxyethoxy)ethyl)pyrrolidin- | | | | | | | | | | | | | |
| | 2-yl)propane- | | | | | | | | | | | | | |
| | 1,2,3-triol | | | | | | | | | | | | | |
| 694 | tert-butyl 4- | pyrrolidine | | | | Y | | | | | Y | | | |
| | (((2R,3R,4S)-3,4- | | | | | | | | | | | | | |
| | dihydroxy-2- | | | | | | | | | | | | | |
| | ((1R,2S,3R)-1,2,3,4- | | | | | | | | | | | | | |
| | tetrahydroxybutyl)pyrrolidin- | | | | | | | | | | | | | |
| | 1- | | | | | | | | | | | | | |
| | yl)methyl)piperidine-1- | | | | | | | | | | | | | |
| | carboxylate | | | | | | | | | | | | | |
| 695 | tert-butyl 4-(((3R,4R)- | pyrrolidine | | | | Y | | Y | | | Y | | Y | |
| | 3,4-dihydroxy-2- | | | | | | | | | | | | | |
| | ((1R,2S,3R)-1,2,3,4- | | | | | | | | | | | | | |
| | tetrahydroxybutyl)pyrrolidin- | | | | | | | | | | | | | |
| | 1- | | | | | | | | | | | | | |
| | yl)methyl)piperidine-1- | | | | | | | | | | | | | |
| | carboxylate | | | | | | | | | | | | | |
| 696 | (1R,2S,3R)-1-((3R,4R)- | pyrrolidine | | | | Y | | Y | | | Y | | Y | |
| | 1-(2- | | | | | | | | | | | | | |
| | (dimethylamino)ethyl)- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 697 | N-((2R,3R)-3- | pyrrolidine | | | | Y | Y | Y | | | | | | |
| | ((2S,3R,4R)-1-benzyl- | | | | | | | | | | | | | |
| | 3,4-dihydroxypyrrolidin- | | | | | | | | | | | | | |
| | 2-yl)-2,3- | | | | | | | | | | | | | |
| | dihydroxypropyl)acetamide | | | | | | | | | | | | | |
| 698 | (2S,3R,4R)-1-benzyl-2- | pyrrolidine | | | | Y | Y | Y | | | | | | |
| | ((1R,2R)-3- | | | | | | | | | | | | | |
| | (benzylamino)-1,2- | | | | | | | | | | | | | |
| | dihydroxypropyl)pyrrolidine- | | | | | | | | | | | | | |
| | 3,4-diol | | | | | | | | | | | | | |
| 699 | (1R,2R)-1-((2R,3R,4R)- | pyrrolidine | | | | Y | Y | Y | | | | | | |
| | 3,4-dihydroxypyrrolidin- | | | | | | | | | | | | | |
| | 2-yl)propane-1,2,3-triol | | | | | | | | | | | | | |
| 700 | (2S,3R,4S)-1-benzyl-2- | pyrrolidine | | | | | | | | | Y | | | |
| | ((S)-2-(benzylamino)-1- | | | | | | | | | | | | | |
| | hydroxyethyl)pyrrolidine- | | | | | | | | | | | | | |
| | 3,4-diol | | | | | | | | | | | | | |
| 701 | N-(((2S,3R,4S)-1- | pyrrolidine | | | Y | | | | | Y | | | | |
| | (biphenyl-4-ylmethyl)- | | | | | | | | | | | | | |
| | 3,4-dihydroxypyrrolidin- | | | | | | | | | | | | | |
| | 2-yl)methyl)biphenyl-4- | | | | | | | | | | | | | |
| | carboxamide | | | | | | | | | | | | | |
| 702 | N-(((2S,3R,4S)-3,4- | pyrrolidine | | | Y | | | | | Y | | | | |
| | dihydroxy-1-(2- | | | | | | | | | | | | | |
| | morpholinoethyl)pyrrolidin- | | | | | | | | | | | | | |
| | 2-yl)methyl)biphenyl- | | | | | | | | | | | | | |
| | 4-carboxamide | | | | | | | | | | | | | |
| 703 | (1R,2R)-1-((2R,3R,4R)- | pyrrolidine | | | | Y | Y | Y | | | | | | |
| | 1-benzyl-3,4- | | | | | | | | | | | | | |
| | dihydroxypyrrolidin-2- | | | | | | | | | | | | | |
| | yl)propane-1,2,3-triol | | | | | | | | | | | | | |
| 704 | (2R,3R,4R)-1-benzyl-2- | pyrrolidine | | | | Y | Y | Y | | | | | | |
| | ((4R,5R)-5- | | | | | | | | | | | | | |
| | ((benzylamino)methyl)- | | | | | | | | | | | | | |
| | 2,2-dimethyl-1,3- | | | | | | | | | | | | | |
| | dioxolan-4- | | | | | | | | | | | | | |
| | yl)pyrrolidine-3,4-diol | | | | | | | | | | | | | |
| 705 | (1R,2R)-1-((2R,3R,4R)- | pyrrolidine | | | | Y | Y | Y | | | | | | |
| | 1-(2- | | | | | | | | | | | | | |
| | (dimethylamino)ethyl)- | | | | | | | | | | | | | |
| | 3,4-dihydroxypyrrolidin- | | | | | | | | | | | | | |
| | 2-yl)propane-1,2,3-triol | | | | | | | | | | | | | |
| 706 | (1R,2R)-1-((2R,3R,4R)- | pyrrolidine | | | | Y | Y | Y | | | | | | |
| | 3,4-dihydroxy-1-(2- | | | | | | | | | | | | | |
| | hydroxyethyl)pyrrolidin- | | | | | | | | | | | | | |
| | 2-yl)propane-1,2,3-triol | | | | | | | | | | | | | |
| 707 | (2S,2′S,3R,3′R,4S,4′S)- | pyrrolidine | | | | Y | | | | | Y | | | |
| | 2,2′-(1R,1′R,2R,2′R)- | | | | | | | | | | | | | |
| | 3,3′-azanediylbis(1,2- | | | | | | | | | | | | | |
| | dihydroxypropane-3,1- | | | | | | | | | | | | | |
| | diyl))bis(1- | | | | | | | | | | | | | |
| | benzylpyrrolidine-3,4- | | | | | | | | | | | | | |
| | diol) | | | | | | | | | | | | | |
| 708 | (2S,3R,4S)-2-((S)-2- | pyrrolidine | | | | | | | | | Y | | | |
| | amino-1- | | | | | | | | | | | | | |
| | hydroxyethyl)pyrrolidine- | | | | | | | | | | | | | |
| | 3,4-diol | | | | | | | | | | | | | |
| 709 | N-((S)-2-((2S,3R,4S)- | pyrrolidine | | | | | | | | | Y | | | |
| | 3,4-dihydroxypyrrolidin- | | | | | | | | | | | | | |
| | 2-yl)-2- | | | | | | | | | | | | | |
| | hydroxyethyl)acetamide | | | | | | | | | | | | | |
| 710 | (2R,4S)-methyl 4- | pyrrolidine | | | | | | | | | | Y | | Y |
| | hydroxypyrrolidine-2- | | | | | | | | | | | | | |
| | carboxylate | | | | | | | | | | | | | |
| 711 | (3R,4R,5R,6R)- | azepane | | | | | | | | | Y | | | |
| | azepane-3,4,5,6-tetraol | | | | | | | | | | | | | |
| 712 | N-butyl-2- | pyrrolidine | | Y | | | | | | | | | Y | |
| | ((2R,3S,4R,5R)-3,4- | | | | | | | | | | | | | |
| | dihydroxy-5- | | | | | | | | | | | | | |
| | (hydroxymethyl)-1-(2- | | | | | | | | | | | | | |
| | (piperidin-1- | | | | | | | | | | | | | |
| | yl)ethyl)pyrrolidin-2- | | | | | | | | | | | | | |
| | yl)acetamide | | | | | | | | | | | | | |
| 713 | (2R,4S)-1-tert-butyl 2- | pyrrolidine | | | | | | | | | | Y | | Y |
| | methyl 4- | | | | | | | | | | | | | |
| | hydroxypyrrolidine-1,2- | | | | | | | | | | | | | |
| | dicarboxylate | | | | | | | | | | | | | |
| 714 | 2-{[(2R,3R,6R)-6-ethyl- | piperidine | | | | | | | | | | | | |
| | 3-hydroxypiperidin-2- | | | | | | | | | | | | | |
| | yl]methoxy}-6- | | | | | | | | | | | | | |
| | (hydroxymethyl)tetrahydro- | | | | | | | | | | | | | |
| | 2H-pyran-3,4,5-triol | | | | | | | | | | | | | |
| 715 | (1S,6R,7R,8S,8aR)- | indolizidine | | | | | | | | | | | | |
| | octahydroindolizine- | | | | | | | | | | | | | |
| | 1,6,7,8-tetraol | | | | | | | | | | | | | |
| 716 | 3-((2S,4S)-4-azido-2- | pyrrolidine | | | Y | | | | | Y | | | | |
| | (hydroxymethyl)pyrrolidin- | | | | | | | | | | | | | |
| | 1-yl)propan-1-ol | | | | | | | | | | | | | |
| 717 | 3-((2R,4R)-4-azido-2- | pyrrolidine | | | Y | | | | | Y | | | | |
| | (hydroxymethyl)pyrrolidin- | | | | | | | | | | | | | |
| | 1-yl)propan-1-ol | | | | | | | | | | | | | |
| 718 | N-(((3aR,4R,6aS)-2,2- | pyrrolidine | | | | | | | | | | Y | | |
| | dimethyltetrahydro-3aH- | | | | | | | | | | | | | |
| | [1,3]dioxolo[4,5-c]pyrrol- | | | | | | | | | | | | | |
| | 4-yl)methyl)butyramide | | | | | | | | | | | | | |
| 719 | (7S,8R,8aS)-methyl 7,8- | other | | | Y | | | | | Y | | | | |
| | dihydroxy-4-oxo- | | | | | | | | | | | | | |
| | 4,6,7,8,8a,9- | | | | | | | | | | | | | |
| | hexahydropyrrolo[1,2- | | | | | | | | | | | | | |
| | d][1,2,3]triazolo[1,5- | | | | | | | | | | | | | |
| | a]pyrazine-3- | | | | | | | | | | | | | |
| | carboxylate | | | | | | | | | | | | | |
| 720 | (2S,3R,4S)-2- | pyrrolidine | | | Y | | | | | Y | | | | |
| | (aminomethyl)-1-(2- | | | | | | | | | | | | | |
| | hydroxyethyl)pyrrolidine- | | | | | | | | | | | | | |
| | 3,4-diol | | | | | | | | | | | | | |
| 721 | (2R,3R,4R)-4-azido-1- | pyrrolidine | | | Y | | | | | Y | | | | |
| | (2-hydroxyethyl)-2- | | | | | | | | | | | | | |
| | (hydroxymethyl)pyrrolidin- | | | | | | | | | | | | | |
| | 3-ol | | | | | | | | | | | | | |
| 722 | (2S,3S,4S)-4-azido-1- | pyrrolidine | | | Y | | | | | Y | | | | |
| | (2-hydroxyethyl)-2- | | | | | | | | | | | | | |
| | (hydroxymethyl)pyrrolidin- | | | | | | | | | | | | | |
| | 3-ol | | | | | | | | | | | | | |
| 723 | 2-((2R,4S)-4-azido-2- | pyrrolidine | | | | | | | | | | Y | | Y |
| | (hydroxymethyl)pyrrolidin- | | | | | | | | | | | | | |
| | 1-yl)ethanol | | | | | | | | | | | | | |
| 724 | (2R,3R,4R,5S)-2- | piperidine | | | | | Y | Y | | | | | | |
| | (hydroxymethyl)-1-(9- | | | | | | | | | | | | | |
| | hydroxynonyl)piperidine- | | | | | | | | | | | | | |
| | 3,4,5-triol | | | | | | | | | | | | | |
| 725 | (2R,3R,4R,5S)-2- | piperidine | | | | | Y | Y | | | | | | |
| | (hydroxymethyl)-1-(2-(2- | | | | | | | | | | | | | |
| | methoxyethoxy)ethyl)piperidine- | | | | | | | | | | | | | |
| | 3,4,5-triol | | | | | | | | | | | | | |
| 726 | (2R,3R,4R,5S)-1-(2- | piperidine | | | | | Y | Y | | | | | | |
| | (dimethylamino)ethyl)-2- | | | | | | | | | | | | | |
| | (hydroxymethyl)piperidine- | | | | | | | | | | | | | |
| | 3,4,5-triol | | | | | | | | | | | | | |
| 727 | N-((3S,5S)-3,5- | piperidine | | | Y | | | | | Y | | | | |
| | dihydroxypiperidin-4- | | | | | | | | | | | | | |
| | yl)acetamide | | | | | | | | | | | | | |
| 728 | (2S,3S,4S,5S)-2-butyl- | pyrrolidine | | | | | Y | Y | | | | | | |
| | 5- | | | | | | | | | | | | | |
| | (hydroxymethyl)pyrrolidine- | | | | | | | | | | | | | |
| | 3,4-diol | | | | | | | | | | | | | |
| 729 | (2S,3S,4S,5S)-2,5- | pyrrolidine | | | | | Y | Y | | | | | | |
| | bis(hydroxymethyl)pyrrolidine- | | | | | | | | | | | | | |
| | 3,4-diol | | | | | | | | | | | | | |
| 730 | N-((3R,4S,5R)-4,5- | piperidine | | | Y | | | | | Y | | | | |
| | dihydroxy-1- | | | | | | | | | | | | | |
| | methylpiperidin-3- | | | | | | | | | | | | | |
| | yl)acetamide | | | | | | | | | | | | | |
| 731 | N-((3R,4S,5R)-4,5- | piperidine | | | Y | | | | | Y | | | | |
| | dihydroxy-1- | | | | | | | | | | | | | |
| | nonylpiperidin-3- | | | | | | | | | | | | | |
| | yl)acetamide | | | | | | | | | | | | | |
| 732 | (2S,3S,4S,5S)-2-ethyl- | pyrrolidine | | | | | | | | | Y | | | |
| | 5- | | | | | | | | | | | | | |
| | (hydroxymethyl)pyrrolidine- | | | | | | | | | | | | | |
| | 3,4-diol | | | | | | | | | | | | | |
| 733 | N-((3S,4R,5R)-4,5- | piperidine | | | | | | | | | | | | Y |
| | dihydroxypiperidin-3- | | | | | | | | | | | | | |
| | yl)acetamide | | | | | | | | | | | | | |
| 734 | (3R,4S,5R,6S)-1-(2- | azepane | | | | Y | | | | | | | | |
| | hydroxyethyl)azepane- | | | | | | | | | | | | | |
| | 3,4,5,6-tetraol | | | | | | | | | | | | | |
| 735 | (3R,4S,5R,6S)-1- | azepane | | | | Y | | | | | | | | |
| | butylazepane-3,4,5,6- | | | | | | | | | | | | | |
| | tetraol | | | | | | | | | | | | | |
| 736 | (3R,4S,5R,6S)-1- | azepane | | | | Y | | | | | | | | |
| | nonylazepane-3,4,5,6- | | | | | | | | | | | | | |
| | tetraol | | | | | | | | | | | | | |
| 737 | (3R,4S,5R,6S)-1-(9- | azepane | | | | Y | | | | | | | | |
| | hydroxynonyl)azepane- | | | | | | | | | | | | | |
| | 3,4,5,6-tetraol | | | | | | | | | | | | | |
| 738 | (3R,4S,5R,6S)-1- | azepane | | | | Y | | | | | | | | |
| | (biphenyl-4- | | | | | | | | | | | | | |
| | ylmethyl)azepane- | | | | | | | | | | | | | |
| | 3,4,5,6-tetraol | | | | | | | | | | | | | |
| 739 | (3R,4S,5R,6S)-1-(2- | azepane | | | | Y | | | | | | | | |
| | (dimethylamino)ethyl)azepane- | | | | | | | | | | | | | |
| | 3,4,5,6-tetraol | | | | | | | | | | | | | |
| 740 | (3R,4S,5R,6S)-1- | azepane | | | | Y | | | | | | | | |
| | benzylazepane-3,4,5,6- | | | | | | | | | | | | | |
| | tetraol | | | | | | | | | | | | | |
| 741 | (R)-(1-butylpiperidin-3- | azepane | | | | | | | | | | | | |
| | yl)methanol | | | | | | | | | | | | | |
| 742 | (3R,4S,5R,6S)- | azepane | | | | Y | | | | | | | | |
| | azepane-3,4,5,6-tetraol | | | | | | | | | | | | | |
| 743 | (2R,3R,4R,5R)-1-(2- | pyrrolidine | | | | | | | | | Y | | | |
| | (dimethylamino)ethyl)- | | | | | | | | | | | | | |
| | 2,5- | | | | | | | | | | | | | |
| | bis(hydroxymethyl)pyrrolidine- | | | | | | | | | | | | | |
| | 3,4-diol | | | | | | | | | | | | | |
| 744 | (R)-2-(3- | piperidine | | | | | | | | | | | | |
| | (hydroxymethyl)piperidin- | | | | | | | | | | | | | |
| | 1-yl)ethanol | | | | | | | | | | | | | |
| 745 | (2R,3R,4R,5R)-1-butyl- | pyrrolidine | | | | | | | | | Y | | | |
| | 2,5- | | | | | | | | | | | | | |
| | bis(hydroxymethyl)pyrrolidine- | | | | | | | | | | | | | |
| | 3,4-diol | | | | | | | | | | | | | |
| 746 | (R)-(1-nonylpiperidin-3- | piperidine | | | | | | | | | | | | |
| | yl)methanol | | | | | | | | | | | | | |
| 747 | (R)-(1-(2-(2- | piperidine | | | | | | | | | | | | |
| | methoxyethoxy)ethyl)piperidin- | | | | | | | | | | | | | |
| | 3-yl)methanol | | | | | | | | | | | | | |
| 748 | 1-(biphenyl-4- | other | | | | | | | | | | | | |
| | ylmethyl)azetidin-3-ol | | | | | | | | | | | | | |
| 749 | 1-(9- | other | | | | | | | | | | | | |
| | hydroxynonyl)azetidin- | | | | | | | | | | | | | |
| | 3-ol | | | | | | | | | | | | | |
| 750 | (1R,4S,7R)-2-oxa-5- | pyrrolidine | | | Y | | | | | Y | | | | |
| | azabicyclo[2.2.1]heptan- | | | | | | | | | | | | | |
| | 7-ol | | | | | | | | | | | | | |
| 751 | (7S,8R,8aR)- | pyrrolidine | | | | | | | | | | | Y | |
| | octahydropyrrolo[1,2- | | | | | | | | | | | | | |
| | a]pyrazine-7,8-diol | | | | | | | | | | | | | |
| 752 | (2S,3R,4S,5R)-1,2- | piperidine | | | | Y | | | | | | | | |
| | dimethylpiperidine- | | | | | | | | | | | | | |
| | 3,4,5-triol | | | | | | | | | | | | | |
| 753 | (6S,7R,8R,8aR)-6,7,8- | piperidine | | | | | Y | Y | | | | | | |
| | trihydroxytetrahydro-1H- | | | | | | | | | | | | | |
| | oxazolo[3,4-a]pyridin- | | | | | | | | | | | | | |
| | 3(5H)-one | | | | | | | | | | | | | |
| 754 | (2R,3R,4R)-1-(2- | piperidine | | | Y | | | | | Y | | | | |
| | hydroxyethyl)-2- | | | | | | | | | | | | | |
| | (hydroxymethyl)piperidine- | | | | | | | | | | | | | |
| | 3,4-diol | | | | | | | | | | | | | |
| 755 | (2R,3R,4R)-2- | piperidine | | | Y | | | | | | | | | |
| | (hydroxymethyl)-1-(2- | | | | | | | | | | | | | |
| | methoxyethyl)piperidine- | | | | | | | | | | | | | |
| | 3,4-diol | | | | | | | | | | | | | |
| 756 | (2R,3R,4R,5S)-1-ethyl- | piperidine | | | | | Y | Y | | | | | | |
| | 2- | | | | | | | | | | | | | |
| | (hydroxymethyl)piperidine- | | | | | | | | | | | | | |
| | 3,4,5-triol | | | | | | | | | | | | | |
| 757 | (1R,2R,3R,4R)-1-butyl- | piperidine | | | Y | | | | | Y | | | | |
| | 3,4-dihydroxy-2- | | | | | | | | | | | | | |
| | (hydroxymethyl)piperidine | | | | | | | | | | | | | |
| | 1-oxide | | | | | | | | | | | | | |
| 758 | (2S,4S,5S)-4,5- | piperidine | | | | Y | Y | Y | | | | | | |
| | dihydroxy-1- | | | | | | | | | | | | | |
| | methylpiperidine-2- | | | | | | | | | | | | | |
| | carboxylic acid | | | | | | | | | | | | | |
| 759 | (4aR,7S,8R,8aR)-5- | piperidine | | | | | Y | Y | | | | | | |
| | benzyl-2,2- | | | | | | | | | | | | | |
| | dimethylhexahydro-4H- | | | | | | | | | | | | | |
| | [1,3]dioxino[5,4- | | | | | | | | | | | | | |
| | b]pyridine-7,8-diol | | | | | | | | | | | | | |
| 760 | (2R,4aR,7S,8R,8aR)- | piperidine | | | | | Y | Y | | | | | | |
| | benzyl 7,8-dihydroxy-2- | | | | | | | | | | | | | |
| | phenyltetrahydro-4H- | | | | | | | | | | | | | |
| | [1,3]dioxino[5,4- | | | | | | | | | | | | | |
| | b]pyridine-5(4aH)- | | | | | | | | | | | | | |
| | carboxylate | | | | | | | | | | | | | |
| 761 | (3R,4R,5R,6R)-1-(2-(2- | azepane | | | | | | | | | Y | | | |
| | methoxyethoxy)ethyl)azepane- | | | | | | | | | | | | | |
| | 3,4,5,6-tetraol | | | | | | | | | | | | | |
| 762 | (3R,4R,5R,6R)-1- | azepane | | | | | | | | | Y | | | |
| | (biphenyl-4- | | | | | | | | | | | | | |
| | ylmethyl)azepane- | | | | | | | | | | | | | |
| | 3,4,5,6-tetraol | | | | | | | | | | | | | |
| 763 | (3R,4R,5R,6R)-1-(9- | azepane | | | | | | | | | Y | | | |
| | hydroxynonyl)azepane- | | | | | | | | | | | | | |
| | 3,4,5,6-tetraol | | | | | | | | | | | | | |
| 764 | (3R,4R,5R,6R)-1- | azepane | | | | | | | | | Y | | | |
| | butylazepane-3,4,5,6- | | | | | | | | | | | | | |
| | tetraol | | | | | | | | | | | | | |
| 765 | 2-((3R,4R,5R,6R)- | azepane | | | | | | | | | Y | | | |
| | 3,4,5,6- | | | | | | | | | | | | | |
| | tetrahydroxyazepan-1- | | | | | | | | | | | | | |
| | yl)acetic acid | | | | | | | | | | | | | |
| 766 | (3R,4R,5R,6R)-1-(5- | azepane | | | | | | | | | Y | | | |
| | (adamantan-1-yl- | | | | | | | | | | | | | |
| | methoxy)- | | | | | | | | | | | | | |
| | pentyl)azepane-3,4,5,6- | | | | | | | | | | | | | |
| | tetraol | | | | | | | | | | | | | |
| 767 | ((2R,4S)-4- | pyrrolidine | | | | | | | | | | Y | | Y |
| | azidopyrrolidin-2- | | | | | | | | | | | | | |
| | yl)methanol | | | | | | | | | | | | | |
| 768 | (2R,4S)-tert-butyl 4- | pyrrolidine | | | | | | | | | | Y | | Y |
| | azido-2- | | | | | | | | | | | | | |
| | (hydroxymethyl)pyrrolidine- | | | | | | | | | | | | | |
| | 1-carboxylate | | | | | | | | | | | | | |
| 769 | (3R,4R,5S,6R)-3,4,5,6- | azepane | | | | | Y | Y | | | | | | |
| | tetrahydroxyazepan-2- | | | | | | | | | | | | | |
| | one | | | | | | | | | | | | | |
| 770 | (3R,4S,5S,6S)- | azepane | | | | | Y | Y | | | | | | |
| | azepane-3,4,5,6-tetraol | | | | | | | | | | | | | |
| 771 | N-((3R,5R)-3,5- | piperidine | | | Y | | | | | Y | | | | |
| | dihydroxypiperidin-4- | | | | | | | | | | | | | |
| | yl)acetamide | | | | | | | | | | | | | |
| 772 | N-((3R,4S,5S)-4,5- | piperidine | | | | | | | | | | | | Y |
| | dihydroxy-1- | | | | | | | | | | | | | |
| | methylpiperidin-3- | | | | | | | | | | | | | |
| | yl)acetamide | | | | | | | | | | | | | |
| 773 | N-((3R,4S,5S)-1-butyl- | piperidine | | | | | | | | | | | | Y |
| | 4,5-dihydroxypiperidin- | | | | | | | | | | | | | |
| | 3-yl)acetamide | | | | | | | | | | | | | |
| 774 | N-((3R,4S,5S)-4,5- | piperidine | | | | | | | | | | | | Y |
| | dihydroxy-1- | | | | | | | | | | | | | |
| | nonylpiperidin-3- | | | | | | | | | | | | | |
| | yl)acetamide | | | | | | | | | | | | | |
| 775 | N-((3S,5S)-3,5- | piperidine | | | Y | | | | | Y | | | | |
| | dihydroxy-1- | | | | | | | | | | | | | |
| | methylpiperidin-4- | | | | | | | | | | | | | |
| | yl)acetamide | | | | | | | | | | | | | |
| 776 | N-((3S,5S)-1-butyl-3,5- | piperidine | | | Y | | | | | Y | | | | |
| | dihydroxypiperidin-4- | | | | | | | | | | | | | |
| | yl)acetamide | | | | | | | | | | | | | |
| 777 | N-((3S,5S)-3,5- | piperidine | | | Y | | | | | Y | | | | |
| | dihydroxy-1- | | | | | | | | | | | | | |
| | nonylpiperidin-4- | | | | | | | | | | | | | |
| | yl)acetamide | | | | | | | | | | | | | |
| 778 | N-((3S,4R,5R)-4,5- | piperidine | | | | | | | | | | | | Y |
| | dihydroxy-1- | | | | | | | | | | | | | |
| | methylpiperidin-3- | | | | | | | | | | | | | |
| | yl)acetamide | | | | | | | | | | | | | |
| 779 | N-((3S,4R,5R)-1-butyl- | piperidine | | | | | | | | | | | | Y |
| | 4,5-dihydroxypiperidin- | | | | | | | | | | | | | |
| | 3-yl)acetamide | | | | | | | | | | | | | |
| 780 | N-((3S,4R,5R)-4,5- | piperidine | | | | | | | | | | | | Y |
| | dihydroxy-1- | | | | | | | | | | | | | |
| | nonylpiperidin-3- | | | | | | | | | | | | | |
| | yl)acetamide | | | | | | | | | | | | | |
| 781 | N-((3R,5R)-3,5- | piperidine | | | Y | | | | | Y | | | | |
| | dihydroxy-1- | | | | | | | | | | | | | |
| | methylpiperidin-4- | | | | | | | | | | | | | |
| | yl)acetamide | | | | | | | | | | | | | |
| 782 | N-((3R,5R)-1-butyl-3,5- | piperidine | | | Y | | | | | Y | | | | |
| | dihydroxypiperidin-4- | | | | | | | | | | | | | |
| | yl)acetamide | | | | | | | | | | | | | |
| 783 | N-((3R,5R)-3,5- | piperidine | | | Y | | | | | Y | | | | |
| | dihydroxy-1- | | | | | | | | | | | | | |
| | nonylpiperidin-4- | | | | | | | | | | | | | |
| | yl)acetamide | | | | | | | | | | | | | |
| 784 | N-((3R,4S,5R)-1-butyl- | piperidine | | | Y | | | | | Y | | | | |
| | 4,5-dihydroxypiperidin- | | | | | | | | | | | | | |
| | 3-yl)acetamide | | | | | | | | | | | | | |
| 785 | N-((3S,4r,5R)-3,5- | piperidine | | | | | | | | | | | | Y |
| | dihydroxypiperidin-4- | | | | | | | | | | | | | |
| | yl)acetamide | | | | | | | | | | | | | |
| 786 | N-((3S,4r,5R)-3,5- | piperidine | | | | | | | | | | | | Y |
| | dihydroxy-1- | | | | | | | | | | | | | |
| | methylpiperidin-4- | | | | | | | | | | | | | |
| | yl)acetamide | | | | | | | | | | | | | |
| 787 | N-((3S,4r,5R)-1-butyl- | piperidine | | | | | | | | | | | | Y |
| | 3,5-dihydroxypiperidin- | | | | | | | | | | | | | |
| | 4-yl)acetamide | | | | | | | | | | | | | |
| 788 | (2R,3S,4R,5R)-2- | pyrrolidine | | | | Y | | | | | | | | |
| | (hydroxymethyl)-5- | | | | | | | | | | | | | |
| | methylpyrrolidine-3,4- | | | | | | | | | | | | | |
| | diol | | | | | | | | | | | | | |
| 789 | N-((3S,4r,5R)-3,5- | piperidine | | | | | | | | | | | | Y |
| | dihydroxy-1- | | | | | | | | | | | | | |
| | nonylpiperidin-4- | | | | | | | | | | | | | |
| | yl)acetamide | | | | | | | | | | | | | |
| 790 | N-((3R,4R,5S)-3- | piperidine | | | | | | | | | | | | Y |
| | (benzyloxy)-1-butyl-5- | | | | | | | | | | | | | |
| | hydroxypiperidin-4- | | | | | | | | | | | | | |
| | yl)acetamide | | | | | | | | | | | | | |
| 791 | (2S,3R,4S,5S)-2- | piperidine | | Y | | | | | | | | | Y | |
| | (hydroxymethyl)piperidine- | | | | | | | | | | | | | |
| | 3,4,5-triol | | | | | | | | | | | | | |
| 792 | (2R,3R,4R,5S)-1-(5- | piperidine | | | | | Y | Y | | | | | | |
| | (adamantan-1-yl- | | | | | | | | | | | | | |
| | methoxy)-pentyl)2- | | | | | | | | | | | | | |
| | (hydroxymethyl)- | | | | | | | | | | | | | |
| | piperidine-3,4,5-triol | | | | | | | | | | | | | |
| 793 | (3R,4R,5R,6R)-3,4,5,6- | azepane | Y | | | | | | | | | | | |
| | tetrahydroxyazepan-2- | | | | | | | | | | | | | |
| | one | | | | | | | | | | | | | |
| 794 | (3R,4R,5R,6R)-1- | azepane | | | | | | | | | Y | | | |
| | nonylazepane-3,4,5,6- | | | | | | | | | | | | | |
| | tetraol | | | | | | | | | | | | | |
| 795 | (2R,3R,4S,5R)-2- | pyrrolidine | | Y | | | | | | | | | Y | |
| | benzyl-5- | | | | | | | | | | | | | |
| | (hydroxymethyl)pyrrolidine- | | | | | | | | | | | | | |
| | 3,4-diol | | | | | | | | | | | | | |
| 796 | (2S,3S,4R)-2-((R)-1,2- | pyrrolidine | Y | | | | | | | | | | | |
| | dihydroxyethyl)-1- | | | | | | | | | | | | | |
| | methylpyrrolidine-3,4- | | | | | | | | | | | | | |
| | diol | | | | | | | | | | | | | |
| 797 | (2S,3R,4S,5R,6R)- | piperidine | | Y | | | Y | Y | | | | | Y | |
| | 3,4,5-trihydroxy-2,6- | | | | | | | | | | | | | |
| | bis(hydroxymethyl)piperidinium | | | | | | | | | | | | | |
| | chloride | | | | | | | | | | | | | |
| 798 | (2S,3R,4R,5R,6R)-2- | piperidine | | | | Y | | | | | Y | | | |
| | ethyl-6- | | | | | | | | | | | | | |
| | (hydroxymethyl)piperidine- | | | | | | | | | | | | | |
| | 3,4,5-triol | | | | | | | | | | | | | |
| 799 | (2R,3S,4R)-1-benzyl-2- | pyrrolidine | | | | | | | | | Y | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 800 | (1S,2R,7R,7aR)- | pyrrolizidine | | | | | | | | | Y | | | |
| | hexahydro-1H- | | | | | | | | | | | | | |
| | pyrrolizine-1,2,7-triol | | | | | | | | | | | | | |
| 801 | (2R,3S,4R)-2-((R)-1- | pyrrolidine | | | | | | | | | Y | | | |
| | hydroxyethyl)pyrrolidine- | | | | | | | | | | | | | |
| | 3,4-diol | | | | | | | | | | | | | |
| 802 | N-((3S,4R,5R,6R)-4,5- | piperidine | | | | | Y | Y | | | | | | |
| | dihydroxy-6- | | | | | | | | | | | | | |
| | (hydroxymethyl)piperidin- | | | | | | | | | | | | | |
| | 3-yl)acetamide | | | | | | | | | | | | | |
| 803 | (2S,3S,4R)-2-((S)-2- | pyrrolidine | | | | | | | | | Y | | | |
| | fluoro-1- | | | | | | | | | | | | | |
| | hydroxyethyl)pyrrolidine- | | | | | | | | | | | | | |
| | 3,4-diol | | | | | | | | | | | | | |
| 804 | (2S,3R,4R,5S)-1- | pyrrolidine | | | | | | | Y | | | | | |
| | benzyl-2,5- | | | | | | | | | | | | | |
| | bis(hydroxymethyl)pyrrolidine- | | | | | | | | | | | | | |
| | 3,4-diol | | | | | | | | | | | | | |
| 805 | (1S,2S,3S,5R,8aS)-3- | indolizidine | | | | | | | | | Y | | | |
| | (hydroxymethyl)-5- | | | | | | | | | | | | | |
| | methyloctahydroindolizine- | | | | | | | | | | | | | |
| | 1,2-diol | | | | | | | | | | | | | |
| 806 | (2S,3R,4R,5R)-2- | piperidine | | | | | Y | | Y | | | | | |
| | (hydroxymethyl)piperidine- | | | | | | | | | | | | | |
| | 3,4,5-triol | | | | | | | | | | | | | |
| 807 | (2R,3R,4R)-1-(4- | pyrrolidine | | | Y | | | | | Y | | | | |
| | chlorobenzyl)-2- | | | | | | | | | | | | | |
| | (hydroxymethyl)pyrrolidine- | | | | | | | | | | | | | |
| | 3,4-diol | | | | | | | | | | | | | |
| 808 | (2R,3R,4R)-2- | pyrrolidine | | | Y | | | | | Y | | | | |
| | (hydroxymethyl)-1-(3- | | | | | | | | | | | | | |
| | phenylpropyl)pyrrolidine- | | | | | | | | | | | | | |
| | 3,4-diol | | | | | | | | | | | | | |
| 809 | (2R,3R,4S)-2-((R)-1,2- | pyrrolidine | | Y | | | | | | | | | Y | |
| | dihydroxyethyl)pyrrolidine- | | | | | | | | | | | | | |
| | 3,4-diol | | | | | | | | | | | | | |
| 810 | (2R,3R,4S)-1-benzyl-2- | pyrrolidine | | Y | | | | | | | | | Y | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 811 | (2R,3R,4R,5S)-2- | piperidine | Y | | | | | | | | | | | |
| | (hydroxymethyl)-5- | | | | | | | | | | | | | |
| | methylpiperidine-3,4,5- | | | | | | | | | | | | | |
| | triol | | | | | | | | | | | | | |
| 812 | (2S,3R,4R)-1-benzyl-2- | pyrrolidine | | | | | | | Y | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 813 | (2S,3R,4R)-2-((S)-1,2- | pyrrolidine | | | | | | | Y | | | | | |
| | dihydroxyethyl)pyrrolidine- | | | | | | | | | | | | | |
| | 3,4-diol | | | | | | | | | | | | | |
| 814 | (1R,4S,7R)-2-benzyl- | pyrrolidine | | | Y | | | | | Y | | | | |
| | 2,5- | | | | | | | | | | | | | |
| | diazabicyclo[2.2.1]heptan- | | | | | | | | | | | | | |
| | 7-ol | | | | | | | | | | | | | |
| 815 | (3S,4S,5S,6S)- | azepane | | | | | | | | | Y | | | |
| | azepane-3,4,5,6-tetraol | | | | | | | | | | | | | |
| 816 | (2R,3S,4S)-1-butyl-2- | piperidine | | | | | | | | | | | | Y |
| | (hydroxymethyl)piperidine- | | | | | | | | | | | | | |
| | 3,4-diol | | | | | | | | | | | | | |
| 817 | (2R,3S,4R)-2-((R)-1,2- | pyrrolidine | | | | | Y | Y | | | | | | |
| | dihydroxyethyl)pyrrolidine- | | | | | | | | | | | | | |
| | 3,4-diol | | | | | | | | | | | | | |
| 818 | (3R,4S,5R,6R)- | azepane | | Y | | | | | | | | | Y | |
| | azepane-3,4,5,6-tetraol | | | | | | | | | | | | | |
| 819 | (2S,3S,4S)-2- | pyrrolidine | | | Y | | | | | Y | | | | |
| | (hydroxymethyl)-1-(3- | | | | | | | | | | | | | |
| | phenylpropyl)pyrrolidine- | | | | | | | | | | | | | |
| | 3,4-diol | | | | | | | | | | | | | |
| 820 | (2S,3R,4R)-1-butyl-2- | pyrrolidine | | | | | | | Y | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 821 | (2S,3R,4R)-2-((S)-1,2- | pyrrolidine | | | | | | | Y | | | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 822 | (2R,3R,4R,5S)-1-hexyl- | piperidine | | | | | Y | Y | | | | | | |
| | 2- | | | | | | | | | | | | | |
| | (hydroxymethyl)piperidine- | | | | | | | | | | | | | |
| | 3,4,5-triol | | | | | | | | | | | | | |
| 823 | (2S,3R,4S)-2- | pyrrolidine | | | Y | | | | | Y | | | | |
| | (hydroxymethyl)-1-(4- | | | | | | | | | | | | | |
| | methoxybenzyl)pyrrolidine- | | | | | | | | | | | | | |
| | 3,4-diol | | | | | | | | | | | | | |
| 824 | (3R,4S,5S,6R)- | azepane | | | | | | | Y | | | | | |
| | azepane-3,4,5,6-tetraol | | | | | | | | | | | | | |
| 825 | (2R,3S,4S)-1-benzyl-2- | pyrrolidine | | | | | | | Y | | | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 826 | (2R,3S,4S,5R)-2,5- | pyrrolidine | | | | | | | Y | | | | | |
| | bis(hydroxymethyl)pyrrolidine- | | | | | | | | | | | | | |
| | 3,4-diol | | | | | | | | | | | | | |
| 827 | N-((3S,4S,5R)-4,5- | piperidine | | | | | | | | | | Y | | |
| | dihydroxypiperidin-3- | | | | | | | | | | | | | |
| | yl)acetamide | | | | | | | | | | | | | |
| 828 | N-((3R,4R,5S)-4,5- | piperidine | | | | | | | | | | Y | | |
| | dihydroxypiperidin-3- | | | | | | | | | | | | | |
| | yl)acetamide | | | | | | | | | | | | | |
| 829 | (1S,2S,3R,6S,9aS)-6- | quinolizidine | | | | | Y | Y | | | | | | |
| | methyloctahydro-1H- | | | | | | | | | | | | | |
| | quinolizine-1,2,3-triol | | | | | | | | | | | | | |
| 830 | N-((3S,4S,5R)-4,5- | piperidine | | | | | | | | | | Y | | |
| | dihydroxy-1- | | | | | | | | | | | | | |
| | methylpiperidin-3- | | | | | | | | | | | | | |
| | yl)acetamide | | | | | | | | | | | | | |
| 831 | N-((3S,4S,5R)-4,5- | piperidine | | | | | | | | | | Y | | |
| | dihydroxy-1- | | | | | | | | | | | | | |
| | nonylpiperidin-3- | | | | | | | | | | | | | |
| | yl)acetamide | | | | | | | | | | | | | |
| 832 | N-((3R,4R,5S)-4,5- | piperidine | | | | | | | | | | Y | | |
| | dihydroxy-1- | | | | | | | | | | | | | |
| | methylpiperidin-3- | | | | | | | | | | | | | |
| | yl)acetamide | | | | | | | | | | | | | |
| 833 | N-((3R,4R,5S)-1-butyl- | piperidine | | | | | | | | | | Y | | |
| | 4,5-dihydroxypiperidin- | | | | | | | | | | | | | |
| | 3-yl)acetamide | | | | | | | | | | | | | |
| 834 | N-((3S,4S,5R)-1-butyl- | piperidine | | | | | | | | | | Y | | |
| | 4,5-dihydroxypiperidin- | | | | | | | | | | | | | |
| | 3-yl)acetamide | | | | | | | | | | | | | |
| 835 | (3R,4S,5S)-5- | piperidine | | | | | | | | | | Y | | |
| | aminopiperidine-3,4-diol | | | | | | | | | | | | | |
| 836 | 2-((3R,4r,5S)-3,4,5- | piperidine | | | | | | | | | | | | Y |
| | trihydroxypiperidin-1- | | | | | | | | | | | | | |
| | yl)acetonitrile | | | | | | | | | | | | | |
| 837 | (3R,4r,5S)-1-(2- | piperidine | | | | | | | | | | | | Y |
| | hydroxyethyl)piperidine- | | | | | | | | | | | | | |
| | 3,4,5-triol | | | | | | | | | | | | | |
| 838 | (3R,4r,5S)-1-(2-(2- | piperidine | | | | | | | | | | | | Y |
| | methoxyethoxy)ethyl)piperidine- | | | | | | | | | | | | | |
| | 3,4,5-triol | | | | | | | | | | | | | |
| 839 | (2R,3R,4R,5R)-2-((R)- | piperidine | | | | Y | | | | | Y | | | |
| | 1,2- | | | | | | | | | | | | | |
| | dihydroxyethyl)piperidine- | | | | | | | | | | | | | |
| | 3,4,5-triol | | | | | | | | | | | | | |
| 840 | (2R,3R,4S,5R)-2-((R)- | piperidine | | Y | | | | | | | | | Y | |
| | 1,2- | | | | | | | | | | | | | |
| | dihydroxyethyl)piperidine- | | | | | | | | | | | | | |
| | 3,4,5-triol | | | | | | | | | | | | | |
| 841 | (2R,3R,4R,5S)-2- | pyrrolidine | | | | | Y | Y | | | | | | |
| | (hydroxymethyl)-5- | | | | | | | | | | | | | |
| | methylpyrrolidine-3,4- | | | | | | | | | | | | | |
| | diol | | | | | | | | | | | | | |
| 842 | (2R,3S,4R)-2-((S)-1,2- | pyrrolidine | | | | | | | | | Y | | | |
| | dihydroxyethyl)-1- | | | | | | | | | | | | | |
| | methylpyrrolidine-3,4- | | | | | | | | | | | | | |
| | diol | | | | | | | | | | | | | |
| 843 | (3R,4R,5R)-3- | piperidine | | | Y | | | | | Y | | | | |
| | (hydroxymethyl)piperazine- | | | | | | | | | | | | | |
| | 4,5-diol | | | | | | | | | | | | | |
| 844 | (4R,5R,6R)-6- | piperidine | | | Y | | | | | Y | | | | |
| | (hydroxymethyl)-1- | | | | | | | | | | | | | |
| | methylpiperazine-4,5- | | | | | | | | | | | | | |
| | diol | | | | | | | | | | | | | |
| 845 | retronecine N-oxide | pyrrolizidine | | | | | | | | | | | | |
| 846 | 1-((3R,4R,5R)-4,5- | piperidine | | | Y | | | | | Y | | | | |
| | dihydroxy-3- | | | | | | | | | | | | | |
| | (hydroxymethyl)piperazin- | | | | | | | | | | | | | |
| | 1-yl)ethanone | | | | | | | | | | | | | |
| 847 | (2S,3R,4R,5R)-2-((R)- | piperidine | | | | | Y | Y | | | | | | |
| | 1,2- | | | | | | | | | | | | | |
| | dihydroxyethyl)piperidine- | | | | | | | | | | | | | |
| | 3,4,5-triol | | | | | | | | | | | | | |
| 848 | (2R,3S,4S)-2-((R)-1,2- | pyrrolidine | | | | | | | Y | | | | | |
| | dihydroxyethyl)pyrrolidine- | | | | | | | | | | | | | |
| | 3,4-diol | | | | | | | | | | | | | |
| 849 | (1S,2S,8R,8aS)- | indolizidine | | | | Y | | | | | | | | |
| | octahydroindolizine- | | | | | | | | | | | | | |
| | 1,2,8-triol | | | | | | | | | | | | | |
| 850 | N-((3R,4R,5R,6R)-4,5- | piperidine | | | | | Y | Y | | | | | | |
| | dihydroxy-6- | | | | | | | | | | | | | |
| | (hydroxymethyl)-2- | | | | | | | | | | | | | |
| | oxopiperidin-3- | | | | | | | | | | | | | |
| | yl)acetamide | | | | | | | | | | | | | |
| 851 | (2R,3S,4R,5R)-2-((S)- | pyrrolidine | | Y | | | | | | | Y | | Y | |
| | 1,2-dihydroxyethyl)-5- | | | | | | | | | | | | | |
| | (hydroxymethyl)pyrrolidine- | | | | | | | | | | | | | |
| | 3,4-diol | | | | | | | | | | | | | |
| 852 | (3R,5R)-1- | piperidine | | | Y | | | | | Y | | | | |
| | hexylpiperidine-3,4,5- | | | | | | | | | | | | | |
| | triol | | | | | | | | | | | | | |
| 853 | (3R,4r,5S)-1- | piperidine | | | | | | | | | | | | Y |
| | hexylpiperidine-3,4,5- | | | | | | | | | | | | | |
| | triol | | | | | | | | | | | | | |
| 854 | (1R,2R,3R,7S,7aR)-3- | pyrrolizidine | | Y | Y | | | | | Y | Y | Y | Y | |
| | ((allylamino)methyl)hexahydro- | | | | | | | | | | | | | |
| | 1H-pyrrolizine- | | | | | | | | | | | | | |
| | 1,2,7-triol | | | | | | | | | | | | | |
| 855 | 2-((1R,2R,3R,7S,7aR)- | pyrrolizidine | | Y | Y | | | | | Y | Y | Y | Y | |
| | 1,2,7- | | | | | | | | | | | | | |
| | trihydroxyhexahydro- | | | | | | | | | | | | | |
| | 1H-pyrrolizin-3- | | | | | | | | | | | | | |
| | yl)acetonitrile | | | | | | | | | | | | | |
| 856 | (3S,5S)-1- | piperidine | | | Y | | | | | Y | | | | |
| | hexylpiperidine-3,4,5- | | | | | | | | | | | | | |
| | triol | | | | | | | | | | | | | |
| 857 | (1R,2R,3R,7S,7aR)-3- | pyrrolizidine | | Y | Y | | | | | Y | Y | Y | Y | |
| | ((benzylamino)methyl)hexahydro- | | | | | | | | | | | | | |
| | 1H- | | | | | | | | | | | | | |
| | pyrrolizine-1,2,7-triol | | | | | | | | | | | | | |
| 858 | (2R,3S,4R,5S)-1-(2- | piperidine | | | | Y | | | | | | | | |
| | hydroxyethyl)-2- | | | | | | | | | | | | | |
| | methylpiperidine-3,4,5- | | | | | | | | | | | | | |
| | triol | | | | | | | | | | | | | |
| 859 | (2R,3S,4R,5S)-1-butyl- | piperidine | | | | Y | | | | | | | | |
| | 2-methylpiperidine- | | | | | | | | | | | | | |
| | 3,4,5-triol | | | | | | | | | | | | | |
| 860 | (2R,3S,4R,5S)-1-(2-(2- | piperidine | | | | Y | | | | | | | | |
| | methoxyethoxy)ethyl)-2- | | | | | | | | | | | | | |
| | methylpiperidine-3,4,5- | | | | | | | | | | | | | |
| | triol | | | | | | | | | | | | | |
| 861 | 2-((2R,3S,4R,5S)-3,4,5- | piperidine | | | | Y | | | | | | | | |
| | trihydroxy-2- | | | | | | | | | | | | | |
| | methylpiperidin-1- | | | | | | | | | | | | | |
| | yl)acetic acid | | | | | | | | | | | | | |
| 862 | (2R,3S,4R,5S)-1-(6- | piperidine | | | | Y | | | | | | | | |
| | hydroxyhexyl)-2- | | | | | | | | | | | | | |
| | methylpiperidine-3,4,5- | | | | | | | | | | | | | |
| | triol | | | | | | | | | | | | | |
| 863 | (2R,3S,4R,5S)-2- | piperidine | | | | Y | | | | | | | | |
| | methyl-1-(2- | | | | | | | | | | | | | |
| | morpholinoethyl)piperidine- | | | | | | | | | | | | | |
| | 3,4,5-triol | | | | | | | | | | | | | |
| 864 | (2R,3S,4R,5S)-2- | piperidine | | | | Y | | | | | | | | |
| | methyl-1-(2-(piperidin-1- | | | | | | | | | | | | | |
| | yl)ethyl)piperidine-3,4,5- | | | | | | | | | | | | | |
| | triol | | | | | | | | | | | | | |
| 865 | (2R,3S,4R,5S)-1-(2- | piperidine | | | | Y | | | | | | | | |
| | (dimethylamino)ethyl)-2- | | | | | | | | | | | | | |
| | methylpiperidine-3,4,5- | | | | | | | | | | | | | |
| | triol | | | | | | | | | | | | | |
| 866 | (2R,3S,4R,5S)-1-(6- | piperidine | | | | Y | | | | | | | | |
| | (2,5- | | | | | | | | | | | | | |
| | dimethylphenoxy)hexyl)- | | | | | | | | | | | | | |
| | 2-methylpiperidine- | | | | | | | | | | | | | |
| | 3,4,5-triol | | | | | | | | | | | | | |
| 867 | (2R,3S,4R,5S)-2- | piperidine | | | | Y | | | | | | | | |
| | methyl-1-(6-((1r,4R)-4- | | | | | | | | | | | | | |
| | methylcyclohexyloxy)hexyl)piperidine- | | | | | | | | | | | | | |
| | 3,4,5-triol | | | | | | | | | | | | | |
| 868 | 2-((3R,4r,5S)-3,4,5- | piperidine | | | | | | | | | | | | Y |
| | trihydroxypiperidin-1- | | | | | | | | | | | | | |
| | yl)acetic acid | | | | | | | | | | | | | |
| 869 | N-((3R,4S,5S)-4,5- | piperidine | | | | | | | | | | | | Y |
| | dihydroxypiperidin-3- | | | | | | | | | | | | | |
| | yl)acetamide | | | | | | | | | | | | | |
| 870 | N-((3S,4R,5S)-4,5- | piperidine | | | Y | | | | | Y | | | | |
| | dihydroxy-1- | | | | | | | | | | | | | |
| | methylpiperidin-3- | | | | | | | | | | | | | |
| | yl)acetamide | | | | | | | | | | | | | |
| 871 | N-((3S,4R,5S)-1-butyl- | piperidine | | | Y | | | | | Y | | | | |
| | 4,5-dihydroxypiperidin- | | | | | | | | | | | | | |
| | 3-yl)acetamide | | | | | | | | | | | | | |
| 872 | (2S,3S,4S,5S)-2-(4- | piperidine | | | | | | | | | y | | | |
| | methoxyphenyl)piperidine- | | | | | | | | | | | | | |
| | 3,4,5-triol | | | | | | | | | | | | | |
| 873 | (2S,3S,4S,5S)-2-(4- | piperidine | | | | | | | | | y | | | |
| | hydroxyphenyl)piperidine- | | | | | | | | | | | | | |
| | 3,4,5-triol | | | | | | | | | | | | | |
| 874 | (2S,3S,4S,5S)-2- | piperidine | | | | | | | | | y | | | |
| | phenylpiperidine-3,4,5- | | | | | | | | | | | | | |
| | triol | | | | | | | | | | | | | |
| 875 | (2S,4S,5S)-1-butyl-4,5- | piperidine | | | | y | y | y | | | | | | |
| | dihydroxypiperidine-2- | | | | | | | | | | | | | |
| | carboxylic acid | | | | | | | | | | | | | |
| 876 | (2S,4S,5S)-4,5- | piperidine | | | | y | y | y | | | | | | |
| | dihydroxy-1- | | | | | | | | | | | | | |
| | nonylpiperidine-2- | | | | | | | | | | | | | |
| | carboxylic acid | | | | | | | | | | | | | |
| 877 | (2R,3S,4S,5S)-3,4- | pyrrolidine | | | | | | | | | y | | | |
| | dihydroxy-5- | | | | | | | | | | | | | |
| | (hydroxymethyl)-1- | | | | | | | | | | | | | |
| | methylpyrrolidine-2- | | | | | | | | | | | | | |
| | carboxylic acid | | | | | | | | | | | | | |
| 878 | (2R,3S,4S,5S)-1-butyl- | pyrrolidine | | | | | | | | | y | | | |
| | 3,4-dihydroxy-5- | | | | | | | | | | | | | |
| | (hydroxymethyl)pyrrolidine- | | | | | | | | | | | | | |
| | 2-carboxylic acid | | | | | | | | | | | | | |
| 879 | (2R,3S,4S,5S)-3,4- | pyrrolidine | | | | | | | | | y | | | |
| | dihydroxy-5- | | | | | | | | | | | | | |
| | (hydroxymethyl)-1- | | | | | | | | | | | | | |
| | nonylpyrrolidine-2- | | | | | | | | | | | | | |
| | carboxylic acid | | | | | | | | | | | | | |
| 880 | (2S,3R,4R,5S)-3,4,5- | piperidine | | | | | y | y | | | | | | |
| | trihydroxy-1- | | | | | | | | | | | | | |
| | nonylpiperidine-2- | | | | | | | | | | | | | |
| | carboxylic acid | | | | | | | | | | | | | |
| 881 | (2S,3S,4S,5S)-2,5- | pyrrolidine | | | | | | | | | y | | | |
| | bis(hydroxymethyl)-1- | | | | | | | | | | | | | |
| | methylpyrrolidine-3,4- | | | | | | | | | | | | | |
| | diol | | | | | | | | | | | | | |
| 882 | (2S,3S,4S,5S)-1-butyl- | pyrrolidine | | | | | | | | | y | | | |
| | 2,5- | | | | | | | | | | | | | |
| | bis(hydroxymethyl)pyrrolidine- | | | | | | | | | | | | | |
| | 3,4-diol | | | | | | | | | | | | | |
| 883 | (2S,3S,4S,5S)-2,5- | pyrrolidine | | | | | | | | | y | | | |
| | bis(hydroxymethyl)-1- | | | | | | | | | | | | | |
| | nonylpyrrolidine-3,4-diol | | | | | | | | | | | | | |
| 884 | (2S,3R,4R,5S)-1-ethyl- | piperidine | | | | | y | y | | | | | | |
| | 3,4,5- | | | | | | | | | | | | | |
| | trihydroxypiperidine-2- | | | | | | | | | | | | | |
| | carboxylic acid | | | | | | | | | | | | | |
| 885 | (2S,3R,4R,5S)-3,4,5- | piperidine | | | | | y | y | | | | | | |
| | trihydroxy-1- | | | | | | | | | | | | | |
| | propylpiperidine-2- | | | | | | | | | | | | | |
| | carboxylic acid | | | | | | | | | | | | | |
| 886 | (2S,3R,4R,5S)-3,4,5- | piperidine | | | | | y | y | | | | | | |
| | trihydroxy-1- | | | | | | | | | | | | | |
| | pentylpiperidine-2- | | | | | | | | | | | | | |
| | carboxylic acid | | | | | | | | | | | | | |
| 887 | (3R,4R,5S)-1-(6- | piperidine | | | | | | | | | | | | y |
| | ((1r,4R)-4- | | | | | | | | | | | | | |
| | methylcyclohexyloxy)hexyl)piperidine- | | | | | | | | | | | | | |
| | 3,4,5-triol | | | | | | | | | | | | | |
| 888 | (2S,3R,4R,5S)-3,4,5- | piperidine | | | | | y | y | | | | | | |
| | trihydroxy-1- | | | | | | | | | | | | | |
| | methylpiperidine-2- | | | | | | | | | | | | | |
| | carboxylic acid | | | | | | | | | | | | | |
| | hydrochloride | | | | | | | | | | | | | |
| 889 | (2S,3R,4R,5S)-1-butyl- | piperidine | | | | | y | y | | | | | | |
| | 3,4,5- | | | | | | | | | | | | | |
| | trihydroxypiperidine-2- | | | | | | | | | | | | | |
| | carboxylic acid | | | | | | | | | | | | | |
| | hydrochloride | | | | | | | | | | | | | |
| 890 | (2S,3R,4R,5S)-3,4,5- | piperidine | | | | | y | y | | | | | | |
| | trihydroxypiperidine-2- | | | | | | | | | | | | | |
| | carboxamide | | | | | | | | | | | | | |
| 891 | (2S,3R,4R,5S)-3,4,5- | piperidine | | | | | y | y | | | | | | |
| | trihydroxy-N- | | | | | | | | | | | | | |
| | methylpiperidine-2- | | | | | | | | | | | | | |
| | carboxamide | | | | | | | | | | | | | |
| 892 | (1R,2S,3R,5R,8aR)-3- | indolizidine | | y | | | | | | | y | | | y |
| | (hydroxymethyl)-5- | | | | | | | | | | | | | |
| | methyloctahydroindolizine- | | | | | | | | | | | | | |
| | 1,2-diol | | | | | | | | | | | | | |

### E. Chemical Synthesis

I. General considerations

Generally applicable strategies for the synthesis of iminosugars and iminosugar libraries are described by La Feria et al. (2007) In “Iminosugars: From synthesis to therapeutic applications”, Wiley ISBN 978-0-470-03391-3; Compain and Martin (Eds.) pp 25-61. These general techniques find application in the synthesis of a wide range of compounds for use according to the invention, including monocyclics, 1-N-iminosugars, bicyclic compounds and iminosugar conjugates. This disclosure is hereby incorporated herein by reference.

### II. Synthesis of Iminosugar C-Glycosides

Generally applicable strategies for the synthesis of iminosugar C-glycosides are described by Compain (2007) In “Iminosugars: From synthesis to therapeutic applications”, Wiley ISBN 978-0-470-03391-3; Compain and Martin (Eds.) pp 63-86. These general techniques find application in the synthesis of a wide range of iminosugar C-glycosides for use according to the invention and the disclosure is hereby incorporated herein by reference.

### III. Synthesis of Imino-C-Disaccharides and Analogues

Generally applicable strategies for the synthesis of imino-C-disaccharides and various analogues are described by Vogel et al. (2007) In “Iminosugars: From synthesis to therapeutic applications”, Wiley ISBN 978-0-470-03391-3; Compain and Martin (Eds.) pp 87-130 the disclosure of which is hereby incorporated herein by reference.

### IV. Synthesis of Polyhydroxylated Iminosugars

The synthesis of polyhydroxylated iminosugars can be carried out by protecting or differentiating the reactivity of the oxygen functions. Bell et al. (1997) Tetrahedron Letters 38(33): 5869-72 describe the synthesis of four diastereoisomers of casuarine from eight carbon sugar lactones by reduction of open chain azidodimesylates by Suzuki-Takaoka reduction to allow the formation of the pyrrolizidine nucleus by bicyclisation.

Another approach is based on tandem [4+2]/[3+2] nitroalkene cycloadditions. It has been used for the synthesis of several pyrrolizidine and indolizidines iminosugars with up to four contiguous stereogenic centres (see Denmark and Hurd (1999) Organic Letters 1(8): 1311-14). The method was later extended by the same workers to the synthesis of (+)-casuarine by the intermolecular [3+2] cycloaddition of a suitable substituted dipolarophile and a flexible, heavily substituted nitronate.

WO2006/008493 (the content of which relating to synthetic schemes for producing iminosugars is hereby incorporated by reference) describes the synthesis of polyhydroxylated pyrrolizidine and indolizidine compounds without protecting all of the free hydroxyl groups, so achieving considerably shortened synthetic schemes. Moreover, the use of intermediates having free hydroxyl groups provides a mechanism for controlling the product distribution, stereospecificity and yield via complex formation at the free hydroxyl groups. According to WO2006/008493, polyhydroxylated bicyclic (for example pyrrolizidine, indolizidine or quinolizidine) iminosugars can be produced by cyclisation of a pyrrolidine or piperidine intermediate having three or more free hydroxyl groups. The application of a cyclisation step to an intermediate having three or more free hydroxyl groups eliminates the need for selective protection, deprotection and/or activation at these sites.

### V. Synthesis of Iminosugar Acids

The ISAs described herein may be made by conventional methods. Methods of making heteroaromatic ring systems are well known in the art. In particular, methods of synthesis are discussed in Taylor et al. (2005) Tetrahedron: 61(40) 9611-9617 and in Comprehensive Heterocyclic Chemistry, Vol. 1 (Eds.: A R Katritzky, C W Rees), Pergamon Press, Oxford, 1984 and Comprehensive Heterocyclic Chemistry II: A Review of the Literature 1982-1995 The Structure, Reactions, Synthesis, and Uses of Heterocyclic Compounds, Alan R. Katritzky (Editor), Charles W. Rees (Editor), E. F. V. Scriven (Editor), Pergamon Pr, June 1996. Other general resources which would aid synthesis of the compounds of interest include March's Advanced Organic Chemistry: Reactions, Mechanisms, and Structure, Wiley-Interscience; 5th edition (Jan. 15, 2001). Some exemplary synthetic schemes for producing ISAs for use according to the invention are shown below:

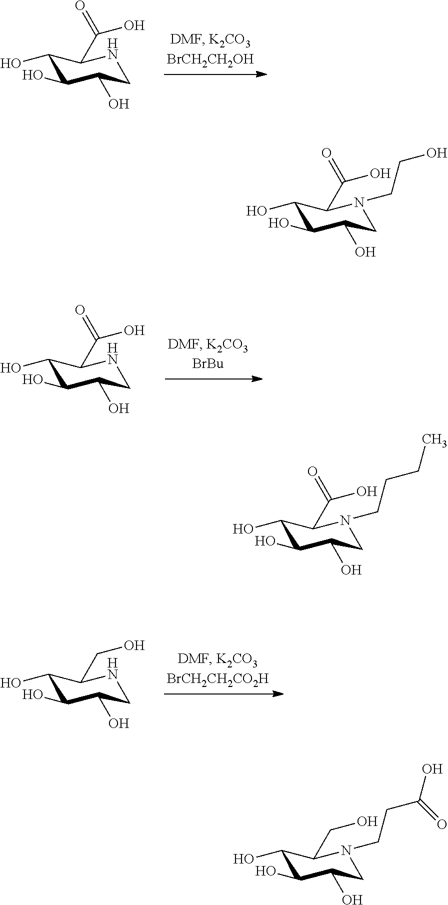

### VI. Synthesis of Nortropanes

Generally applicable strategies for the synthesis of nortropanes are described by Skaanderup and Madsen (2003) Journal of Organic Chemistry 68(6): 2115-2122 the disclosure of which is hereby incorporated herein by reference.

### VII. Synthesis of Azepanes

Generally applicable strategies for the synthesis of azepanes are described by Li et al. (2007) Chemical Communications (Cambridge, United Kingdom) (2): 183-185 the disclosure of which is hereby incorporated herein by reference.

### VIII. Synthesis of Pyrrolidines

Generally applicable strategies for the synthesis of pyrrolidines are described by Rountree et al. (2007) Tetrahedron Letters 48: 4287-4291 and Behr and Guillerm (2007) Tetrahedron Letters 48(13), 2369-2372 the disclosure of which is hereby incorporated herein by reference.

### IX. Synthesis of Piperidines

Generally applicable strategies for the synthesis of piperidines are described by Mane et al. (2008) Journal of Organic Chemistry 73 (8): 3284-3287 and Rengasamy et al. (2008) Journal of Organic Chemistry 73(7): 2898-2901 the disclosure of which is hereby incorporated herein by reference.

### X. Synthesis of Pyrrolizidines

Generally applicable strategies for the synthesis of pyrrolizidines are described in Pyrrolizidine Alkaloids, pp 617-653, in The Way of Synthesis, Tomas Hudlicky and Josephine W. Reed, 2007, Wiley, ISBN: 978-3-527-31444-7 and by Van Ameijde et al. (2006) Tetrahedron: Asymmetry 17: 2702-2713, the disclosure of which is hereby incorporated herein by reference.

### XI. Synthesis of Indolizidines

Generally applicable strategies for the synthesis of indolizidines are described in Abrams et al. (2008) Journal of Organic Chemistry 73 (5): 1935-1940 and Kumar et al. (2008) Organic & Biomolecular Chemistry 6(4): 703-711, the disclosure of which is hereby incorporated herein by reference.

### XII. Synthesis of Quinolizidines

Generally applicable strategies for the synthesis of quinolizidines are described in Pasniczek et al. (2007) Journal of Carbohydrate Chemistry 26(3): 195-211 and Kumar et al. (2008) Organic & Biomolecular Chemistry 6(4): 703-711, the disclosure of which is hereby incorporated herein by reference.

### XIII. Synthesis of 4-Membered Monocycles

Generally applicable strategies for the synthesis of 4-membered monocycles are described in Evans et al. (2008) Journal of Medicinal Chemistry 51(4): 948-956, the disclosure of which is hereby incorporated herein by reference.

### XIV. Synthesis of 9-Membered Monocycles

Generally applicable strategies for the synthesis of 9-membered monocycles are described in Leonard and Swann (1952) Journal of the American Chemical Society 74: 4620-4, the disclosure of which is hereby incorporated herein by reference.

### XV. Synthesis of 10-Membered Monocycles

Generally applicable strategies for the synthesis of 10-membered monocycles are described by Arata and Kobayashi (1972) Chemical & Pharmaceutical Bulletin 20(2): 325-9, the disclosure of which is hereby incorporated herein by reference.

### XVI. Synthesis of 4,6 Fused Bicyclics

Generally applicable strategies for the synthesis of 4,6 fused bicyclics are described in Pandey et al. (2006) Tetrahedron Letters 47(45): 7923-7926, the disclosure of which is hereby incorporated herein by reference.

### XVII. Synthesis of 4,7 Fused Bicyclics

Generally applicable strategies for the synthesis of 4,7 fused bicyclics are described in Alcaide and Saez (2005) European Journal of Organic Chemistry (8): 1680-1693, the disclosure of which is hereby incorporated herein by reference.

### XVIII. Synthesis of 5,7 Fused Bicyclics

Generally applicable strategies for the synthesis of 5,7 fused bicyclics are described in Bande et al. (2007) Tetrahedron: Asymmetry 18(10): 1176-1182, the disclosure of which is hereby incorporated herein by reference.

### XIX. Synthesis of 1,2 Piperazines

Generally applicable strategies for the synthesis of 1,2-piperazines are described in Ernholt et al. (1999) Synlett. 701-704, Liang et al (1999) J. Org. Chem., 64 (23), 8485-8488, Ernholt et al. (2000) Chem. Eur. J., 6(2) 278-287, Jensen et al. (2001) J. Chem. Soc., Perkin Trans. 1, 905-909 and Jensen et al. (2002) J. Chem. Soc., Perkin Trans. 1, 1190-1198 the disclosure of which is hereby incorporated herein by reference.

### F. Purification from Botanic Sources

### I. General

Botanic and microbial sources for a wide range of different iminosugars are described in Watson et al., (2001) Phytochemistry 56: 265-295. Iminosugar acids also have a wide distribution in plants such as in Stevia, Gymnema, Citrus, Lycium species, leguminous spp. e.g. Aspalanthus linearis (Rooibos), Lotus species and Castanospermum australe (Fabaceae), Cucurbitaceae species and Andrographis paniculata (Acanthaceae). The distribution of iminosugar acids in microorganisms is not known but they are likely to be present.

II. Purification of Iminosugars and Iminosugar Acids from Botanic Sources

The compounds described herein for use according to the invention may be isolated from natural sources. For example, plant material from botanic sources such as Stevia species can be used as starting material for the isolation and purification of both iminosugars and iminosugar acids for use according to the invention. Microorganisms such as Bacillus, Streptomyces and Metarrhizium species can be used for isolation of iminosugars. The natural iminosugars and iminosugar acids of the invention are water-soluble and can be concentrated by using strongly acidic cation exchange resins to which they bind with the iminosugar acids then concentrated subsequently by binding them to strongly basic anion exchange resins. The iminosugars are not strongly retained on the anion exchange resins whereas the iminosugar acids are. Purification of the iminosugars and iminosugar acids can then be achieved by using a series of cation and anion exchange resins selected by those experienced in the art. Size exclusion methods can also be used to concentrate them. Thus, it will be appreciated that those skilled in the art can readily purify and isolate the iminosugar and iminosugar acids of the invention using standard techniques.

### Posology

The compounds of the present invention can be administered by oral or parenteral routes, including intravenous, intramuscular, intraperitoneal, subcutaneous, transdermal, airway (aerosol), rectal, vaginal and topical (including buccal and sublingual) administration.

The amount administered can vary widely according to the particular dosage unit employed, the period of treatment, the age and sex of the patient treated, the nature and extent of the disorder treated, and the particular compound selected.

Moreover, the compounds of the invention can be used in conjunction with other agents known to be useful in the treatment of diseases or disorders arising from protein folding abnormalities (as described infra) and in such embodiments the dose may be adjusted accordingly.

In general, the effective amount of the compound administered will generally range from about 0.01 mg/kg to 500 mg/kg daily. A unit dosage may contain from 0.05 to 500 mg of the compound, and can be taken one or more times per day. The compound can be administered with a pharmaceutical carrier using conventional dosage unit forms either orally, parenterally, or topically, as described below.

The preferred route of administration is oral administration. In general a suitable dose will be in the range of 0.01 to 500 mg per kilogram body weight of the recipient per day, preferably in the range of 0.1 to 50 mg per kilogram body weight per day and most preferably in the range 1 to 5 mg per kilogram body weight per day.

The desired dose is preferably presented as a single dose for daily administration. However, two, three, four, five or six or more sub-doses administered at appropriate intervals throughout the day may also be employed. These sub-doses may be administered in unit dosage forms, for example, containing 0.001 to 100 mg, preferably 0.01 to 10 mg, and most preferably 0.5 to 1.0 mg of active ingredient per unit dosage form.

### Formulation

The compound for use according to the invention may take any form. It may be synthetic, purified or isolated from natural sources.

When isolated from a natural source, the compound for use according to the invention may be purified. In embodiments where the compound is formulated together with a pharmaceutically acceptable excipient, any suitable excipient may be used, including for example inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc.

The pharmaceutical compositions may take any suitable form, and include for example tablets, elixirs, capsules, solutions, suspensions, powders, granules and aerosols.

The pharmaceutical composition may take the form of a kit of parts, which kit may comprise the composition of the invention together with instructions for use and/or a plurality of different components in unit dosage form.

Tablets for oral use may include the compound for use according to the invention, mixed with pharmaceutically acceptable excipients, such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatin capsules in which the compound for use according to the invention is mixed with a solid diluent, and soft gelatin capsules wherein the active ingredient is mixed with water or an oil such as peanut oil, liquid paraffin or olive oil.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate.

The compounds of the invention may also be presented as liposome formulations.

For oral administration the compound can be formulated into solid or liquid preparations such as capsules, pills, tablets, troches, lozenges, melts, powders, granules, solutions, suspensions, dispersions or emulsions (which solutions, suspensions dispersions or emulsions may be aqueous or non-aqueous). The solid unit dosage forms can be a capsule which can be of the ordinary hard- or soft-shelled gelatin type containing, for example, surfactants, lubricants, and inert fillers such as lactose, sucrose, calcium phosphate, and cornstarch.

In another embodiment, the compounds of the invention are tableted with conventional tablet bases such as lactose, sucrose, and cornstarch in combination with binders such as acacia, cornstarch, or gelatin, disintegrating agents intended to assist the break-up and dissolution of the tablet following administration such as potato starch, alginic acid, corn starch, and guar gum, lubricants intended to improve the flow of tablet granulations and to prevent the adhesion of tablet material to the surfaces of the tablet dies and punches, for example, talc, stearic acid, or magnesium, calcium, or zinc stearate, dyes, coloring agents, and flavoring agents intended to enhance the aesthetic qualities of the tablets and make them more acceptable to the patient.

Suitable excipients for use in oral liquid dosage forms include diluents such as water and alcohols, for example, ethanol, benzyl alcohol, and the polyethylene alcohols, either with or without the addition of a pharmaceutically acceptably surfactant, suspending agent or emulsifying agent.

The compounds of the invention may also be administered parenterally, that is, subcutaneously, intravenously, intramuscularly, or interperitoneally.

In such embodiments, the compound is provided as injectable doses in a physiologically acceptable diluent together with a pharmaceutical carrier (which can be a sterile liquid or mixture of liquids). Suitable liquids include water, saline, aqueous dextrose and related sugar solutions, an alcohol (such as ethanol, isopropanol, or hexadecyl alcohol), glycols (such as propylene glycol or polyethylene glycol), glycerol ketals (such as 2,2-dimethyl-1,3-dioxolane-4-methanol), ethers (such as poly(ethylene-glycol) 400), an oil, a fatty acid, a fatty acid ester or glyceride, or an acetylated fatty acid glyceride with or without the addition of a pharmaceutically acceptable surfactant (such as a soap or a detergent), suspending agent (such as pectin, carhomers, methylcellulose, hydroxypropylmethylcellulose, or carboxymethylcellulose), or emulsifying agent and other pharmaceutically adjuvants. Suitable oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate.

Suitable soaps include fatty alkali metal, ammonium, and triethanolamine salts and suitable detergents include cationic detergents, for example, dimethyl dialkyl ammonium halides, alkyl pyridinium halides, and alkylamines acetates; anionic detergents, for example, alkyl, aryl, and olefin sulphonates, alkyl, olefin, ether, and monoglyceride sulphates, and sulphosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention will typically contain from about 0.5 to about 25% by weight of the compound for use according to the invention in solution. Preservatives and buffers may also be used. In order to minimize or eliminate irritation at the site of injection, such compositions may contain a non-ionic surfactant having a hydrophile-lipophile balance (HLB) of from about 12 to about 17. The quantity of surfactant in such formulations ranges from about 5 to about 15% by weight. The surfactant can be a single component having the above HLB or can be a mixture of two or more components having the desired HLB. Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The compound for use according to the invention may also be administered topically, and when done so the carrier may suitably comprise a solution, ointment or gel base. The base, for example, may comprise one or more of the following: petrolatum, lanolin, polyethylene glycols, bee wax, mineral oil, diluents such as water and alcohol, and emulsifiers and stabilizers. Topical formulations may contain a concentration of the compound from about 0.1 to about 10% w/v (weight per unit volume).

When used adjunctively, the compound for use according to the invention may be formulated for use with one or more other drug(s). Thus, adjunctive use may be reflected in a specific unit dosage designed to be compatible (or to synergize) with the other drug(s), or in formulations in which the compound is admixed with one or more enzymes. Adjunctive uses may also be reflected in the composition of the pharmaceutical kits of the invention, in which the compounds of the invention is co-packaged (e.g. as part of an array of unit doses) with the enzymes. Adjunctive use may also be reflected in information and/or instructions relating to the co-administration of the compound and/or enzyme.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

BVDV Plaque Assay: In the absence of a suitable cell culture system able to support replication of human HCV, bovine diarrhoea virus (BVDV) is an accepted cell culture model. HCV and BVDV share a significant degree of local protein homology, a common replication strategy and probably the same subcellular location for viral envelopment. The ability of a compound of the invention to exert a direct anti-BVDV effect in vitro can therefore be tested and activity demonstrated in a BVDV plaque inhibition assay (as detailed below).

The materials and procedures were as described in Whitby et al. (2004) Antiviral Chemistry and Chemotherapy 15: 141-151. In brief, MDBK cells were seeded in 96 well plates and allowed to reach confluency. Monolayers were exposed to between 14 and 45 plaque forming units of BVDV and adsorption allowed to take place. Infected monolayers were then exposed to the test compound, medium added containing low gelling-point agarose and allowed to set. The plates were then incubated for 4 days post infection, fixed in 5% formalin and stained with 0.5% neutral red after removal of the agarose layer. Anti-viral activity was measured by determination of plaque inhibition and expressed as IC₅₀ values. Castanospermine, a known viral inhibitor, was used as a positive control.

### Examples 1-3

### Anti-BVDV Activity

The hepatitis C virus (HCV) was first identified in 1989 and it has since become clear that this virus is responsible for most cases of post-transfusion non-A, non-B hepatitis. Indeed, HCV is now recognised as one of the commonest infections causing chronic liver disease and The World Health Organisation estimates that 170 million people are chronically infected. HCV infection results in a chronic infection in 85% of infected patients and approximately 20-30% of these will progress to cirrhosis and end stage liver disease, frequently complicated by hepatocellular carcinoma.

The study of HCV has been hampered by the inability to propagate the virus efficiently in cell culture. However, in the absence of a suitable cell culture system able to support replication of human HCV, bovine diarrhoea virus (BVDV) is an accepted cell culture model. HCV and BVDV share a significant degree of local protein homology, a common replication strategy and probably the same subcellular location for viral envelopment.

The ability of various compounds of the invention to exert a direct anti-BVDV effect in vitro was therefore tested and activity demonstrated in a BVDV plaque inhibition assay (as detailed below).

For these assays a confluent monolayer of MDBK cells is produced in a flat bottomed well of a tissue culture plate. The monolayer is infected with BVDV. Sufficient virus is added to eventually form approximately 20-30 plaques. After allowing approximately 1 hr for the virus to infect, the cells are washed and liquid agar is added and allowed to set as a thin layer over the cell surface (the ‘overlay’). The infected cells are then left for a period of days to allow the virus to replicate and cells to shed virus, detach or lyse. Cells in the immediate vicinity of the initial virus infection are therefore infected—localized by the agar layer. Hence a clear plaque devoid of cells is eventually formed which after staining uninfected cells around it with neutral red is visible and can be scored.

The test compound is added at appropriate dilutions with the virus. An antiviral effect of the compound is scored by the reduction of plaque number or size. The concentration of compound required to produce a 50% (IC₅₀) reduction of plaque number or size is noted. Controls of no compound added are included. A control of a known antiviral compound (castanospermine) is carried out to calibrate the antiviral activity.

Castanospermine (Compound 104), a known viral inhibitor (see infra), was used as a positive control. The following data was obtained:

| Compound | IC₅₀ (μg/ml) |
|---|---|
| 62 | 16.7 |
| 63 | 87.2 |
| 167 | 57.6 |
| 104 (prior art | 15.6 |
| positive control) | |

### Example 4

### Toxicity Assay

The compounds tested above were assayed for toxicity using a standard ‘XTT’ colorimetric assay. In this assay the test compound, in the absence of virus was added to the cell monolayer. The cells and compound (and controls of cells without compound) were incubated for a period equivalent to the time required for viral plaques to be formed in the standard antiviral assay. XTT reagents are then added. XTT is metabolized by the mitochondria of viable cells producing an increase in absorbance at 450 nm. The effect of toxic compounds is to reduce this metabolism and generate less absorbance at 450 nm.

All compounds assayed at 200 μg/ml showed approximately 15% reduction in absorbance with respect to no compound controls. This is in the range of a designation of ‘not toxic’ in this assay.

### Example 5

### Anti-BVDV Activity

The hepatitis C virus (HCV) was first identified in 1989 and it has since become clear that this virus is responsible for most cases of post-transfusion non-A, non-B hepatitis. Indeed, HCV is now recognised as one of the commonest infections causing chronic liver disease and The World Health Organisation estimates that 170 million people are chronically infected. HCV infection results in a chronic infection in 85% of infected patients and approximately 20-30% of these will progress to cirrhosis and end stage liver disease, frequently complicated by hepatocellular carcinoma.

The study of HCV has been hampered by the inability to propagate the virus efficiently in cell culture. However, in the absence of a suitable cell culture system able to support replication of human HCV, bovine diarrhoea virus (BVDV) is an accepted cell culture model. HCV and BVDV share a significant degree of local protein homology, a common replication strategy and probably the same subcellular location for viral envelopment.

The ability of compound 23 of the invention to exert a direct anti-BVDV effect in vitro was therefore tested and activity demonstrated in a BVDV plaque inhibition assay (as detailed below).

Plaque Assay: The materials and procedures were as described in Whitby et al. (2004) Antiviral Chemistry and Chemotherapy 15: 141-151.

In brief, MDBK cells were seeded in 96 well plates and allowed to reach confluency. Monolayers were exposed to between 14 and 45 plaque forming units of BVDV and adsorption allowed to take place. Infected monolayers were then exposed to the test compound, medium added containing low gelling-point agarose and allowed to set. The plates were then incubated for 4 days post infection, fixed in 5% formalin and stained with 0.5% neutral red after removal of the agarose layer. Anti-viral activity was measured by determination of plaque inhibition and expressed as IC₅₀ values. Castanospermine (Compound 104), a known viral inhibitor, was used as a positive control.

### Results:

| | Dose | Dose | % untreated |
|---|---|---|---|
| Test compound | (μg/ml) | (mM) | control |
| Compound 23 | 500 | 2.60 | 22 |
| | 250 | 1.30 | 35 |
| | 125 | 0.65 | 47 |
| | 63 | 0.33 | 56 |
| | 32 | 0.17 | 83 |
| Castanospermine | 100.0 | 0.53 | 1.1 |
| (compound 104) | | | |
| | 50.0 | 0.26 | 12 |
| | 25.0 | 0.13 | 46 |
| | 12.5 | 0.07 | 67 |
| | 6.0 | 0.03 | 98 |

| | IC₅₀ | IC₅₀ |
|---|---|---|
| Test compound | (μg/ml) | (μM) |
| Compound 23 | 80 | 420 |
| Castanospermine | 22.5 | 120 |
| (compound 104) | | |

The results show that the test compound of the invention exhibits good antiviral activity against BVDV. No cytotoxicity was noted.

### Example 6

### Inhibition of Glycosidase Activity

Inhibition of the N-linked glycan trimming enzymes alpha-Glucosidases I and II or alpha-Mannosidases I and II are thought to be related to the anti-viral activity of castanospermine (Compound 104) and 1-deoxynojirimycin (Compound 193). Glycosidase assays were conducted on iminosugars exhibiting anti-viral activity. The data suggest that anti-viral activity can also be independent of inhibition of the above trimming glycosidases. All enzymes were purchased from Sigma, as were the appropriate p-nitrophenyl substrates. Assays were carried out in microtitre plates. Enzymes were assayed in 0.1M citric acid/0.2M di-sodium hydrogen phosphate (McIlvaine) buffers at the optimum pH for the enzyme. All assays were carried out at 20° C. For screening assays the incubation assay consisted of 10 μl of enzyme solution, 10 μl of inhibitor solution (made up in water at 5 mM) and 50 μl of the appropriate 5 mM p-nitrophenyl substrate (3.57 mM final conc.) made up in McIlvaine buffer at the optimum pH for the enzyme.

The reactions were stopped with 0.4M glycine (pH 10.4) during the exponential phase of the reaction, which was determined at the beginning of the assay using blanks with water, which were incubated for a range of time periods to measure the reaction rate using 5 mM substrate solution. Endpoint absorbances were read at 405 nm with a Bio-rad microtitre plate reader (Benchmark). Water was substituted for the inhibitors in the blanks.

The enzymes tested are shown in the table below.

| Enzyme | Source | pH | Conc. | Substrate |
|---|---|---|---|---|
| α-D-glucosidase | Saccharomyces cerevisiae | 6.0 | 0.1 unit/ml | PNP-α-D-glucopyranoside |
| | (Baker's yeast), rice (Oryza | | | |
| | sativa), Bacillus | | | |
| | stearothermophilus | | | |
| β-D-glucosidase | Almonds (Prunus sp.) | 5.0 | 0.2 unit/ml | PNP-β-D-glucopyranoside |
| α-D- | Green coffee beans (Coffea | 6.5 | 1 unit/ml | PNP-α-D-galactopyranoside |
| galactosidase | sp.) | | | |
| β-D- | Bovine liver | 7.3 | 0.1 unit/ml | PNP-β-D-galactopyranoside |
| galactosidase | | | | |
| α-D- | Jack beans (Canavalia | 4.5 | 0.1 unit/ml | PNP-α-D-mannopyranoside |
| mannosidase | ensiformis) | | | |
| α-L-fucosidase | Bovine kidney | 5.5 | 0.4 units/ml | PNP-α-L-fucopyranoside |
| N-acetyl-β-D- | Bovine kidney | 4.25 | 0.1 unit/ml | PNP-N-acetyl-β-D- |
| glucosaminidase | | | | glucosminide |
| Naringinase | Penecillium decumbens | 4.0 | 1 unit/ml | PNP-α-L-rhamnopyranoside |

The results (% inhibition) for these anti-BVDV compounds (all at 1 mg/ml) are shown in the table below:

Cpd #

| Assay | 23 | 62 | 63 | 104 | 167 | 193 |
|---|---|---|---|---|---|---|
| α-gluc (yeast) | 0 | 0 | 0 | 0 | 0 | 35 |
| α-gluc (rice) | 66 | 32 | 41 | 90 | 58 | 100 |
| α-gluc | 0 | 65 | 36 | 0 | 35 | 100 |
| (Bacillus) | | | | | | |
| β-glucosidase | 28 | 100 | 93 | 88 | 22 | 64 |
| α- | 0 | 0 | 0 | 0 | 32 | 0 |
| galactosidase | | | | | | |
| β- | 53 | 100 | 93 | 16 | 0 | 0 |
| galactosidase | | | | | | |
| α- | 0 | 0 | 0 | 0 | 0 | 0 |
| mannosidase | | | | | | |
| α-fucosidase | 0 | 0 | 0 | 0 | 28 | 0 |
| Naringinase | 0 | 37 | 0 | 39 | 50 | 0 |
| N-acetyl-β- | 0 | 20 | 0 | 0 | 19 | 0 |
| gluc | | | | | | |

### Example 7

### Anti-HCV Activity

Anti-HCV activity was assessed using the internally quenched 5-FAM/QXLTM520 Fluorescence Resonance Energy Transfer (FRET) assay described in Yu et al. (2009) Development of a Cell-Based Hepatitis C Virus (HCV) Infection FRET Assay for High Throughput Antiviral Compound Screening Antimicrob Agents Chemother. doi:10.1128/AAC.00495-09 (and see also Zhong et al., (2005) Robust hepatitis C virus infection in vitro Proc Natl Acad Sci USA.: 102(26):9294-9).

The peptide substrate for the NS3 protease FRET assay is an internally quenched peptide with a fluorescent donor (FAM) and acceptor (QXL) on opposing sides of the NS3 protease cleavage site. The donor absorbs energy at 480 nm and emits energy (i.e. fluorescence) at 520 nm. However, when in close contact on an intact peptide, the acceptor absorbs the 520 nM energy emitted by the donor preventing fluorescence. Cleavage of the peptide increases the distance between the fluorophores resulting in proportional FAM fluorescence.

Synchronized, non-dividing human hepatoma-derived DMSO-Huh7 cells were infected with HCV at 0.05 ffu/cell. Compounds were added co-infection and were replenished every 2 days over the 6 day assay. Day 6 p.i., cultures assayed for HCV NS3 protein levels by FRET. Cells infected with increasing doses of HCV at day 3 p.i. exhibited FRET signals proportional to multiplicity of infection (MOI).

The following compounds exhibited anti-HCV activity in the screen described above:

TABLE 2
Anti-HCV compounds

| Cmpd | | Ave % |
|---|---|---|
| # | Chemical name | inhibition |
| 5 | (2R,3S,4S)-4-hydroxy-2-(4-methoxybenzyl)pyrrolidin-3-yl acetate | +++++ |
| 332 | ((2S,4S)-4-acetamido-1-nonylpyrrolidin-2-yl)methyl acetate | +++++ |
| 622 | N-(((2S,3R,4S)-3,4-dihydroxy-1-nonylpyrrolidin-2- | +++++ |
| | yl)methyl)benzamide | |
| 652 | N-(((3aR,4S,6aS)-5-benzyl-2,2-dimethyltetrahydro-3aH- | +++++ |
| | [1,3]dioxolo[4,5-c]pyrrol-4-yl)methyl)biphenyl-4-carboxamide | |
| 670 | N-(((2S,3R,4S)-3,4-dihydroxypyrrolidin-2-yl)methyl)biphenyl-4- | +++++ |
| | carboxamide | |
| 717 | ′3-((2R,4R)-4-azido-2-(hydroxymethyl)pyrrolidin-1-yl)propan-1-ol | +++++ |
| 348 | N-((3S,5S)-5-(hydroxymethyl)-1-nonylpyrrolidin-3-yl)acetamide | ++++ |
| 547 | (3R,5S)-5-(acetamidomethyl)-1-nonylpyrrolidin-3-yl acetate | ++++ |
| 559 | (2R,3R,4R,5R)-1-(biphenyl-4-ylmethyl)-2,5- | ++++ |
| | bis(hydroxymethyl)pyrrolidine-3,4-diol | |
| 561 | (2S,3S,4S,5S)-2-((R)-4-aminopentyl)-5-(hydroxymethyl)pyrrolidine- | ++++ |
| | 3,4-diol | |
| 562 | (2S,3S,4S,5S)-2-((S)-4-aminopentyl)-5-(hydroxymethyl)pyrrolidine- | ++++ |
| | 3,4-diol | |
| 563 | N-((3R,4S,5R)-4,5-dihydroxypiperidin-3-yl)acetamide | ++++ |
| 702 | N-(((2S,3R,4S)-3,4-dihydroxy-1-(2-morpholinoethyl)pyrrolidin-2- | ++++ |
| | yl)methyl)biphenyl-4-carboxamide | |
| 704 | (2R,3R,4R)-1-benzyl-2-((4R,5R)-5-((benzylamino)methyl)-2,2- | ++++ |
| | dimethyl-1,3-dioxolan-4-yl)pyrrolidine-3,4-diol | |
| 748 | 1-(biphenyl-4-ylmethyl)azetidin-3-ol | ++++ |
| 767 | ((2R,4S)-4-azidopyrrolidin-2-yl)methanol | ++++ |
| 794 | (3R,4R,5R,6R)-1-nonylazepane-3,4,5,6-tetraol | ++++ |
| 87 | (3R,4R,5R)-3,4,5-trihydroxypiperidine-3-carboxylic acid | +++ |
| 145 | (2R,3S,4R)-2-((S)-1,2-dihydroxyethyl)-1-(2-hydroxyethyl)pyrrolidine- | +++ |
| | 3,4-diol | |
| 316 | (2S,3S,4R)-2-((R)-1,2-dihydroxyethyl)-1-nonylpyrrolidine-3,4-diol | +++ |
| 404 | (1R,2R)-1-((2R,3R,4S)-3,4-dihydroxy-1-nonylpyrrolidin-2-yl)propane- | +++ |
| | 1,2,3-triol | |
| 427 | (2S,3S,4R)-2-((S)-1,2-dihydroxyethyl)-1-nonylpyrrolidine-3,4-diol | +++ |
| 506 | (2S,3R,4S)-2-(azidomethyl)-1-benzylpyrrolidine-3,4-diol | +++ |
| 512 | N-(((2R,3R,4S)-3,4-dihydroxy-1-nonylpyrrolidin-2- | +++ |
| | yl)methyl)acetamide | |
| 558 | (2R,3R,4R,5R)-1-(biphenyl-4-ylmethyl)-2,5- | +++ |
| | bis(hydroxymethyl)pyrrolidine-3,4-diol | |
| 587 | N-(((2S,3R,4S)-3,4-dihydroxy-1-nonylpyrrolidin-2- | +++ |
| | yl)methyl)acetamide | |
| 633 | N-(((2S,3R,4S)-3,4-dihydroxy-1-(2-morpholinoethyl)pyrrolidin-2- | +++ |
| | yl)methyl)benzamide | |
| 649 | N-(((2R,3R,4S)-1-(biphenyl-4-ylmethyl)-3,4-dihydroxypyrrolidin-2- | +++ |
| | yl)methyl)benzamide | |
| 660 | N-(((2S,3R,4S)-3,4-dihydroxy-1-(9-hydroxynonyl)pyrrolidin-2- | +++ |
| | yl)methyl)biphenyl-4-carboxamide | |
| 686 | (2S,3R,4S)-2-(aminomethyl)-1-(biphenyl-4-ylmethyl)pyrrolidine-3,4- | +++ |
| | diol | |
| 688 | N-(((2S,3R,4S)-3,4-dihydroxy-1-(2-(2- | +++ |
| | methoxyethoxy)ethyl)pyrrolidin-2-yl)methyl)biphenyl-4-carboxamide | |
| 736 | (3R,4S,5R,6S)-1-nonylazepane-3,4,5,6-tetraol | +++ |
| 762 | (3R,4R,5R,6R)-1-(biphenyl-4-ylmethyl)azepane-3,4,5,6-tetraol | +++ |
| 777 | N-((3S,5S)-3,5-dihydroxy-1-nonylpiperidin-4-yl)acetamide | +++ |
| 783 | N-((3R,5R)-3,5-dihydroxy-1-nonylpiperidin-4-yl)acetamide | +++ |
| 789 | N-((3S,4r,5R)-3,5-dihydroxy-1-nonylpiperidin-4-yl)acetamide | +++ |
| 15 | (2R,3S,4R,5R,6R)-2,6-bis(hydroxymethyl)piperidine-3,4,5-triol | ++ |
| 46 | (1S,6S,7S,8R)-1,7,8-trihydroxyoctahydroindolizin-6-yl butyrate | ++ |
| 72 | (2S,3S,4S,5R)-2-ethylpiperidine-3,4,5-triol | ++ |
| 81 | (1R,2R,3S,4R,6S,7R,7aR)-1,2,6,7-tetrahydroxy-3- | ++ |
| | (hydroxymethyl)octahydropyrrolizine 4-oxide | |
| 118 | (2R,3R,4R,5S)-1-butyl-2-(hydroxymethyl)piperidine-3,4,5-triol | ++ |
| 119 | (2S,3S,4S,5R)-2-(hydroxymethyl)-5-methylpyrrolidine-3,4-diol | ++ |
| 132 | (1R,2R,3R,6S,7S,7aR)-3-(butyryloxymethyl)hexahydro-1H- | ++ |
| | pyrrolizine-1,2,6,7-tetrayl tetrabutyrate | |
| 136 | (1R,2R,3S,6S,7S,7aR)-3-(acetoxymethyl)hexahydro-1H-pyrrolizine- | ++ |
| | 1,2,6,7-tetrayl tetraacetate | |
| 144 | (1R,2R,3S,6S,7R,7aR)-3-(acetoxymethyl)hexahydro-1H-pyrrolizine- | ++ |
| | 1,2,6,7-tetrayl tetraacetate | |
| 149 | (1S,2R,3R,5S,7R,7aR)-3-(hydroxymethyl)-5-methylhexahydro-1H- | ++ |
| | pyrrolizine-1,2,7-triol | |
| 153 | (1R,2S,8S,8aS)-octahydroindolizine-1,2,8-triol | ++ |
| 155 | (2S,3R,4S)-2-((S)-1,2-dihydroxyethyl)pyrrolidine-3,4-diol | ++ |
| 162 | (2S,3R,4S)-1-butyl-2-(hydroxymethyl)pyrrolidine-3,4-diol | ++ |
| 275 | ((2S,4S)-4-azido-1-butylpyrrolidin-2-yl)methanol | ++ |
| 338 | N-((3S,4R,5S)-4-hydroxy-5-(hydroxymethyl)-1-nonylpyrrolidin-3- | ++ |
| | yl)acetamide | |
| 483 | ((3aR,4R,6aS)-5-benzyl-2,2-dimethyltetrahydro-3aH-[1,3]dioxolo[4,5- | ++ |
| | c]pyrrol-4-yl)methanamine | |
| 499 | 4-[(2R,3R,4R)-3,4-dihydroxy-2-(hydroxymethyl)pyrrolidin-1- | ++ |
| | yl]butanoic acid | |
| 500 | (2S,3S,4S)-1-benzyl-2-(hydroxymethyl)pyrrolidine-3,4-diol | ++ |
| 504 | (2R,3S,4S)-3,4-dihydroxy-2-methylpyrrolidine-2-carboxylic acid | ++ |
| 511 | N-(((2R,3R,4S)-3,4-dihydroxy-1-(9-hydroxynonyl)pyrrolidin-2- | ++ |
| | yl)methyl)acetamide | |
| 530 | 2-((2R,3R,4S)-3,4-dihydroxy-2-((1R,2S,3R)-1,2,3,4- | ++ |
| | tetrahydroxybutyl)pyrrolidin-1-yl)acetic acid | |
| 546 | (3R,5S)-5-(acetamidomethyl)-1-butylpyrrolidin-3-yl acetate | ++ |
| 550 | (2R,3R,4R,5S)-2-(hydroxymethyl)-1-nonylpiperidine-3,4,5-triol | ++ |
| 557 | (2R,3R,4R,5R)-2,5-bis(hydroxymethyl)-1-(9- | ++ |
| | hydroxynonyl)pyrrolidine-3,4-diol | |
| 616 | N-(((2S,3R,4S)-3,4-dihydroxy-1-(2-(piperidin-1-yl)ethyl)pyrrolidin-2- | ++ |
| | yl)methyl)acetamide | |
| 623 | (2S,3S,4R)-1-(biphenyl-4-ylmethyl)-2-(hydroxymethyl)pyrrolidine-3,4- | ++ |
| | diol | |
| 630 | N-(((3aR,4S,6aS)-2,2-dimethyl-5-nonyltetrahydro-3aH- | ++ |
| | [1,3]dioxolo[4,5-c]pyrrol-4-yl)methyl)acetamide | |
| 632 | N-(((3aR,4S,6aS)-5-(biphenyl-4-ylmethyl)-2,2-dimethyltetrahydro- | ++ |
| | 3aH-[1,3]dioxolo[4,5-c]pyrrol-4-yl)methyl)-2,2,2-trifluoroacetamide | |
| 635 | (1S,2S,3S,6R,7R,7aR)-1,6,7-trihydroxy-3- | ++ |
| | (hydroxymethyl)hexahydro-1H-pyrrolizin-2-yl methanesulfonate | |
| 667 | N-(((2S,3R,4S)-1-(2-(dimethylamino)ethyl)-3,4-dihydroxypyrrolidin-2- | ++ |
| | yl)methyl)benzamide | |
| 669 | N-(((2R,3R,4S)-1-benzyl-3,4-dihydroxypyrrolidin-2- | ++ |
| | yl)methyl)butyramide | |
| 671 | (3aS,4R,6aR)-4-(azidomethyl)-2,2-dimethyltetrahydro-3aH- | ++ |
| | [1,3]dioxolo[4,5-c]pyrrole | |
| 673 | N-(((2R,3R,4S)-1-benzyl-3,4-dihydroxypyrrolidin-2- | ++ |
| | yl)methyl)biphenyl-4-carboxamide | |
| 674 | N-(((2S,3R,4S)-3,4-dihydroxypyrrolidin-2-yl)methyl)-2,2,2- | ++ |
| | trifluoroacetamide | |
| 675 | N-(((3aR,4S,6aS)-2,2-dimethyl-5-(2-morpholinoethyl)tetrahydro-3aH- | ++ |
| | [1,3]dioxolo[4,5-c]pyrrol-4-yl)methyl)-2,2,2-trifluoroacetamid | |
| 676 | N-(((3aR,4S,6aS)-5-butyl-2,2-dimethyltetrahydro-3aH- | ++ |
| | [1,3]dioxolo[4,5-c]pyrrol-4-yl)methyl)-2,2,2-trifluoroacetamide | |
| 677 | N-(((3aR,4S,6aS)-2,2-dimethyl-5-(2-(piperidin-1-yl)ethyl)tetrahydro- | ++ |
| | 3aH-[1,3]dioxolo[4,5-c]pyrrol-4-yl)methyl)-2,2,2-trifluoroacetamide | |
| 678 | N-(((3aR,4S,6aS)-5-(2-(dimethylamino)ethyl)-2,2-dimethyltetrahydro- | ++ |
| | 3aH-[1,3]dioxolo[4,5-c]pyrrol-4-yl)methyl)-2,2,2-trifluoroacetamide | |
| 698 | (2S,3R,4R)-1-benzyl-2-((1R,2R)-3-(benzylamino)-1,2- | ++ |
| | dihydroxypropyl)pyrrolidine-3,4-diol | |
| 700 | (2S,3R,4S)-1-benzyl-2-((S)-2-(benzylamino)-1- | ++ |
| | hydroxyethyl)pyrrolidine-3,4-diol | |
| 703 | (1R,2R)-1-((2R,3R,4R)-1-benzyl-3,4-dihydroxypyrrolidin-2- | ++ |
| | yl)propane-1,2,3-triol | |
| 713 | (2R,4S)-1-tert-butyl 2-methyl 4-hydroxypyrrolidine-1,2-dicarboxylate | ++ |
| 721 | (2R,3R,4R)-4-azido-1-(2-hydroxyethyl)-2-(hydroxymethyl)pyrrolidin- | ++ |
| | 3-ol | |
| 731 | N-((3R,4S,5R)-4,5-dihydroxy-1-nonylpiperidin-3-yl)acetamide | ++ |
| 780 | N-((3S,4R,5R)-4,5-dihydroxy-1-nonylpiperidin-3-yl)acetamide | ++ |
| 14 | (1R,2R,3R,5R,7aR)-3-(hydroxymethyl)-5-methylhexahydro-1H- | + |
| | pyrrolizine-1,2-diol | |
| 23 | (2S,3R,4S,5S,6S)-2-ethyl-6-(hydroxymethyl)piperidine-3,4,5-triol | + |
| 62 | (2R,3R,4R,5R)-2-(hydroxymethyl)-5-((R)-1- | + |
| | hydroxypropyl)pyrrolidine-3,4-diol | |
| 63 | (2R,3R,4R,5R)-2-((1R)-1,2-dihydroxypropyl)-5- | + |
| | (hydroxymethyl)pyrrolidine-3,4-diol | |
| 477 | trans-4-Hydroxy-D-proline | + |
| 689 | N-(((2R,3R,4S)-1-butyl-3,4-dihydroxypyrrolidin-2- | + |
| | yl)methyl)butyramide | |
| 756 | (2R,3R,4R,5S)-1-ethyl-2-(hydroxymethyl)piperidine-3,4,5-triol | + |
| 774 | N-((3R,4S,5S)-4,5-dihydroxy-1-nonylpiperidin-3-yl)acetamide | + |
| 799 | (2R,3S,4R)-1-benzyl-2-((S)-1,2-dihydroxyethyl)pyrrolidine-3,4-diol | + |

Key: +++++: ≧80%
++++: 60-79%
+++: 40-59%
++: 20-39%
+: ≦19%

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

We claim:

1-44. (canceled)

45. A compound of Formula (1) 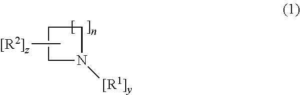
in which
n represents an integer from 1 to 7, provided that where n>1 the ring may also contain at least one unsaturated C—C bond;
z represents an integer from 1 to (n+2);
y represents 1 or 2;
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl;
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal);
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
or a pharmaceutically acceptable salt or derivative thereof, for the treatment of an infection with, or disease caused by, a flavivirus.

46. The compound of claim 45 wherein n=1 to 5.

47. The compound of claim 46 wherein n is 2 or 3.

48. The compound of claim 45 having three, four or more rings.

49. The compound of claim 45 wherein z=2 to (n+2).

50. The compound of claim 49 wherein z is (n+2).

51. A pyrrolidine compound of Formula (1) 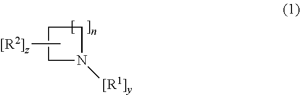
in arabinose and/or lyxose stereochemical configuration, in which
n is 2
z represents an integer from 1 to (n+2)
y represents 1 or 2
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
or a pharmaceutically acceptable salt or derivative thereof, for the treatment of an infection with, or disease caused by, HCV.

52. The compound of claim 45 having at least two R² substituents, one being OH and the other being hydroxymethyl.

53. The compound of claim 51 having at least two R² substituents, one being OH and the other being hydroxymethyl.

54. The compound of claim 45 which is: (a) selected from compounds 1 to 892 of Table 1, or a pharmaceutically acceptable salt or derivative thereof; or (b) the anti-HCV compounds listed in Table 2, or a pharmaceutically acceptable salt or derivative thereof.

55. The compound of claim 45 wherein the flavivirus is a member of the genus Pestivirus or Flavivirus.

56. The compound of claim 45 wherein the flavivirus is a member of the genus Hepacivirus.

57. The compound of claim 56 wherein the virus is HCV.

58. The compound of claim 57 wherein the virus is selected from HCV genotypes 1, 2, 3, 4, 5 and 6.

59. A method for the treatment of an infection with, or disease caused by, a flavivirus in a subject, comprising administering an effective amount of a compound as defined in claim 45 to said subject.

60. A method for the treatment of an infection with, or disease caused by, a flavivirus in a subject, comprising administering an effective amount of a compound as defined in claim 53 to said subject.
